(19) United States
(12) Patent Application Publication
(10) Pub. No.: US 2021/0371576 A1
(43) Pub. Date: Dec. 2, 2021

(54) INDOLE-BASED COMPOUNDS

(71) Applicants: RECTICEL NV, Brussel (BE); UNIVERSITEIT GENT, Gent (BE)

(72) Inventors: Lucie IMBERNON, Wetteren (BE); Joke DE GEETER, Wetteren (BE); Filip DU PREZ, Gent (BE); Nancy HAUCOURT, Wetteren (BE); Duchan LAPLACE, Scheldewindeke (BE); Geert SNELLINGS, Wetteren (BE); Pieter VAN TITTELBOOM, Wetteren (BE); Johan WINNE, Melle (BE)

(21) Appl. No.: 16/980,308

(22) PCT Filed: Mar. 14, 2019
(86) PCT No.: PCT/EP2019/056481

§ 371 Date: Sep. 11, 2020

(30) Foreign Application Priority Data
Mar. 14, 2018 (EP) 18161838.0

(51) Int. Cl. C08G 18/38; C07D 209/12; C07D 249/12; C08G 18/48; C08G 18/66; C08G 18/24; C08G 18/76; C08J 9/12

(52) U.S. Cl. CPC C08G 18/3851; C07D 209/12; C07D 249/12; C08G 18/4825; C08G 18/4833; C08G 18/4808; C08J 2203/10; C08G 18/244; C08G 18/7671; C08J 9/125; C08G 2110/0083; C08J 2375/12; C08G 18/6666

(57) ABSTRACT

The present invention relates to a compound of formula (I) or a stereoisomer, enantiomer, racemic, or tautomer thereof, formula (I) wherein r, R¹, R², R³, R⁴, R⁵, R⁶ and L¹ have the meaning defined in the claims and the description. The present invention also relates to a process for the preparation of the compound of formula (I). The present invention also relates to a composition comprising the compound of formula (I). The present invention also relates to a polyurethane or polyurethane prepolymer obtainable by contacting at least one isocyanate with at least one compound of formula (I), or with a composition comprising a compound of formula (I). The present invention relates to the use of such polyurethane or polyurethane prepolymer, or a compound of formula (I), or a composition comprising a compound of formula (I), for the preparation of foams, adhesives, coatings, or elastomers. The present invention also relates to an article comprising a compound of formula (I), or a composition a compound of formula (I).

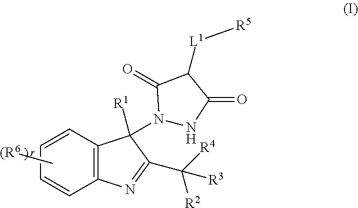

### FIELD OF THE INVENTION

The invention pertains to indole-based compounds and polyurethane functionalized with indole-based compounds, a process for their preparation and applications thereof.

### BACKGROUND OF THE INVENTION

Polyurethane (PU) is the generic name for polymers possessing urethane bonds. A urethane bond is formed via an addition reaction between an isocyanate group and a compound possessing active hydrogen, such as a hydroxyl group.

Polyurethanes (PU) are widely used in flexible, semi-rigid and rigid foams, durable elastomers and high performance adhesives and sealants, fibers, seals, gaskets, condoms, carpet underlayment, and hard plastic parts.

Over three quarters of the consumption of polyurethane products is in the form of foams, with flexible and rigid types being roughly equal in market size. Polyurethane materials are also used in coatings and varnishes used in furniture manufacture, carpentry or woodworking. Polyurethane is also used as an adhesive, especially as a woodworking glue. Its main advantage over more traditional wood glues is its water resistance. It is also used in making solid tires. Modern roller blading and skateboarding became economical only with the introduction of tough, abrasion-resistant polyurethane parts.

Polyurethane products can be thermoset or thermoplastic materials. As known by the skilled in the art, thermoset materials are different from thermoplastic materials because the former do not have a melting temperature Tₘ above which the material will start to flow and can therefore not be processed on equipment specific for thermoplastics such as injection moulding equipment.

The chemical difference between thermoset and thermoplastic materials is that the former are formed of a permanent covalently crosslinked structure, i.e. they have covalent bonds between the different polymer strands called crosslinks. Although these permanent crosslinks result in an excellent durability, abrasion resistance, heat resistance and load bearing capacity of the thermoset PU materials, they do not allow the thermoset material to be recycled, reshaped or reprocessed by melting, in contrast to thermoplastic materials. This represents a major problem, considering the current trends to go for more sustainable materials.

Therefore, there remains a need for polyurethane thermosets that overcome the aforementioned issues. It is an object of the present invention to provide polyurethane with improved reprocessing properties. It is an object of the present invention to provide a new compound that can improve the reprocessing properties of polyurethane (both thermoplastic and thermoset polyurethane).

### SUMMARY OF THE INVENTION

The present inventors have now surprisingly found that by incorporating a 1,2,4-triazoline-3,5-dione-indole adduct (TAD-indole adduct) into a polyurethane network, the resulting thermoset polyurethane material can be reprocessed by heating above a specific temperature. The resulting polyurethane thermoset has the advantages of conventional polyurethane thermosets over polyurethane thermoplasts, as set out above, with the added advantage that it also shows reprocessable properties, which makes the material suitable for reprocessing, for healable and self-healable applications, for thermoforming, as well as for recycling purposes. Furthermore, the incorporation of a 1,2,4-triazoline-3,5-dione-indole adduct (TAD-indole adduct) into a thermoplastic polyurethane can aid in modifying the melting behavior of the polymer.

According to a first aspect of the present invention, a compound of formula (I) is provided or a stereoisomer, enantiomer, racemic, or tautomer thereof,

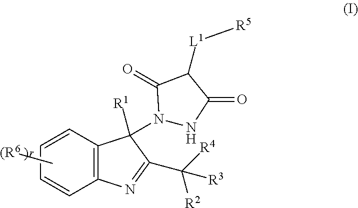

wherein,
r is an integer selected from 0, 1, 2, 3 or 4;
R¹ is selected from the group consisting of C₁₋₁₀alkyl; C₂₋₁₀alkenyl; C₂₋₁₀alkynyl; C₆₋₁₂aryl; C₃₋₈cycloalkyl; C₆₋₁₂arylC₁₋₆alkyl; heterocyclyl; heteroaryl; heterocyclylC₁₋₆alkyl; heteroarylC₁₋₈alkyl; —OR¹⁹; —SR¹⁹; NR²⁰R²¹; —C(O)NR²⁰R²¹; OH, SH, COOH, NH₂; NHR²⁰; and a polymeric group;
   and wherein said C₁₋₁₀alkyl; C₂₋₁₀alkenyl; C₂₋₁₀alkynyl, C₆₋₁₂aryl, C₃₋₈cycloalkyl; C₆₋₁₂arylC₁₋₆alkyl, heterocyclyl; heteroaryl; heterocyclylC₁₋₆alkyl; or heteroarylC₁₋₆alkyl; can be unsubstituted or substituted with one or more Z¹;
   and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
   or R¹ is a group of formula (i);

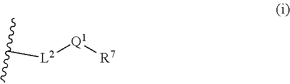

wherein the wavy line ( [symbol] ) indicates the point of attachment to the indole ring of formula (I);
L² is selected from the group consisting of C₁₋₁₀alkylene; C₃₋₆cycloalkylene; C₂₋₁₀alkenylene; C₂₋₁₀alkynylene; C₆₋₁₂arylene; and C₆₋₁₂arylC₁₋₆alkylene;
   and wherein said C₁₋₁₀alkylene; C₃₋₆cycloalkylene; C₂₋₁₀alkenylene; C₂₋₁₀alkynylene; C₆₋₁₂arylene or C₆₋₁₂arylC₁₋₆alkylene, can be unsubstituted or substituted with one or more Z²;
Q¹ is a single bond or selected from the group consisting of C₁₋₁₀alkylene; C₃₋₆cycloalkylene; —O—; —S—; —O—C₁₋₁₀alkylene; and —S—C₁₋₁₀alkylene; wherein each left side of said groups is attached to L² and the right side thereof is attached to R⁷;
   and wherein said C₁₋₁₀alkylene and said C₃₋₆cycloalkylene; can be unsubstituted or substituted with one or more Z³;
R⁷ is selected from the group consisting of hydrogen; OH, SH, COOH, NH₂; NHR²⁰, halo; nitro; cyano; —C(O)NR²⁰R²¹; C₁₋₂₀alkyl, C₂₋₂₀alkenyl; C₂₋₂₀alkynyl; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
   wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl; can be unsubstituted or substituted with one or more Z⁴;
   and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
or R⁷ is a group of formula (ii);

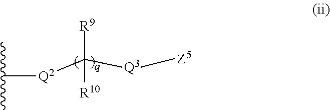

wherein the wavy line ( [symbol] ) indicates the point of attachment to Q¹;
q is an integer selected from 0; 1, 2, 3 or 4;
Q² is a single bond or selected from the group consisting of CR¹¹R¹²; O, S, and NR¹³;
Q³ is a single bond or selected from the group consisting of hydroxyC₁₋₁₀alkylene; aminoC₁₋₁₀alkylene; mercaptoC₁₋₁₀alkylene; C₁₋₂₀alkylene; C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₆alkylene; and heteroarylC₁₋₆alkylene;
wherein said C₁₋₂₀alkylene, C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₈alkylene; or heteroarylC₁₋₆alkylene, can be unsubstituted or substituted with one or more Z^{5a};
wherein if q is 0, then one of Q² and Q³ is not a single bond;
each R⁹ and R¹⁰ is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
each R¹¹ and R¹² is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
R¹³ is selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; and heteroarylC₁₋₆alkyl;
R² is selected from the group consisting of halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; heteroarylC₁₋₆alkyl; and a polymeric group;
R³ is selected from the group consisting of halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; heteroarylC₁₋₆alkyl; and a polymeric group;
R⁴ is selected from the group consisting of halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; heteroarylC₁₋₆alkyl; and a polymeric group; wherein for R², R³ and R⁴, each independently; said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl, heteroarylC₁₋₆alkyl, can be unsubstituted or substituted with one or more Z⁶;
   and wherein said polymeric is selected from the group comprising polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
or R² and R³ and R⁴ together with the carbon atom to which they are attached can form a saturated or unsaturated 4-, 5-, 6-, 7- or 8-membered ring;
   wherein said saturated or unsaturated 4-, 5-, 6-, 7- or 8-membered ring optionally comprises one or more heteroatoms, said heteroatoms being each independently selected from O, S and N;
   wherein said saturated or unsaturated 4-, 5-, 6-, 7- or 8-membered ring can be unsubstituted or substituted with one or more R⁸;
each R⁸ is independently selected from the group consisting of hydroxyC₁₋₁₀alkoxy; aminoC₁₋₁₀alkoxy; mercaptoC₁₋₁₀alkoxy; C₁₋₁₀alkyl; C₂₋₁₀alkenyl; C₂₋₁₀alkynyl; C₆₋₁₂arylC₁₋₆alkyl; C₃₋₈cycloalkyl; C₆₋₁₂arylC₁₋₆alkyl; heterocyclyl; heteroaryl; heterocyclylC₁₋₆alkyl; heteroarylC₁₋₈alkyl; OH; SH; COOH; NH₂; NHR²⁰; halogen; haloC₁₋₆alkyl; haloC₁₋₆alkyloxy; —OR¹⁹; —SR¹⁹; nitro; NR²⁰R²¹; —C(O)NR²⁰R²¹H cyano; and a polymeric group;
   wherein said hydroxyC₁₋₁₀alkoxy; aminoC₁₋₁₀alkoxy; mercaptoC₁₋₁₀alkoxy; C₁₋₁₀alkyl; C₂₋₁₀alkenyl; C₂₋₁₀alkynyl; C₆₋₁₂arylC₁₋₆alkyl; C₃₋₈cycloalkyl; C₆₋₁₂arylC₁₋₆alkyl; heterocyclyl; heteroaryl; heterocyclylC₁₋₆alkyl; or heteroarylC₁₋₆alkyl, can be unsubstituted or substituted with one or more Z⁷;
   and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
   or R⁸ is a group of formula (iii);

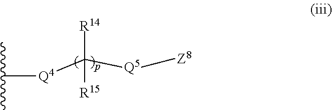

wherein the wavy line ( [symbol] ) indicates the point of attachment to the ring formed by R² and R³ and R⁴ together;
p is an integer selected from 0; 1, 2, 3 or 4;
Q⁴ is a single bond or selected from the group consisting of CR¹⁶R¹⁷; O, S, and NR¹⁸;
Q⁵ is a single bond or selected from the group consisting of hydroxyC₁₋₁₀alkylene; aminoC₁₋₁₀alkylene; mercaptoC₁₋₁₀alkylene; C₁₋₂₀alkylene, C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₆alkylene; and heteroarylC₁₋₆alkylene;
wherein said C₁₋₂₀alkylene, C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₆alkylene; or heteroarylC₁₋₆alkylene, can be unsubstituted or substituted with one or more Z^{8a};
wherein if p is 0, then one of Q⁴ and Q⁵ is not a single bond;
each R¹⁴ and R¹⁵ is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
each R¹⁶ and R¹⁷ is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
R¹⁸ is selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; and heteroarylC₁₋₆alkyl;
each R⁶ is independently selected from the group consisting of hydrogen, halogen; C₁₋₁₀alkyl; C₂₋₁₀alkenyl; C₂₋₁₀alkynyl; C₆₋₁₂arylC₁₋₆alkyl; haloC₁₋₆alkyl; haloC₁₋₆alkyloxy; —OR¹⁹; —SR¹⁹; nitro; NR²⁰R²¹; —C(O)NR²⁰R²¹; cyano; and a polymeric group;
   wherein said C₁₋₁₀alkyl; C₂₋₁₀alkenyl; C₂₋₁₀alkynyl; or C₆₋₁₂arylC₁₋₆alkyl, can be unsubstituted or substituted with one or more Z⁹;
   and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
L¹ is selected from the group consisting of C₁₋₁₀alkyleneC₃₋₈cycloalkylene; C₃₋₈cycloalkyleneC₁₋₁₀alkylene; C₁₋₁₀alkylene; C₃₋₈cycloalkylene; C₃₋₈cycloalkyleneC₁₋₁₀alkylC₃₋₈cycloalkylene; C₂₋₁₀alkenylene; C₂₋₁₀alkynylene; C₆₋₁₂arylene; C₆₋₁₂aryleneC₁₋₆alkylene; C₆₋₁₂aryleneC₁₋₆alkylC₆₋₁₂arylene; and C₁₋₆alkyleneC₆₋₁₂arylC₁₋₆alkylene;
   wherein said C₁₋₁₀alkyleneC₃₋₈cycloalkylene; C₃₋₈cycloalkyleneC₁₋₁₀alkylene; C₁₋₁₀alkylene; C₃₋₈cycloalkylene; C₃₋₈cycloalkyleneC₁₋₁₀alkylC₃₋₈cycloalkylene; C₂₋₁₀alkenylene; C₂₋₁₀alkynylene; C₆₋₁₂arylene; C₆₋₁₂aryleneC₁₋₆alkylene; C₆₋₁₂aryleneC₁₋₆alkylC₆₋₁₂arylene; or C₁₋₆alkyleneC₆₋₁₂arylC₁₋₆alkylene, can be unsubstituted or substituted with one or more Z^{1a};
or L¹ is selected from a group of formula (iv);

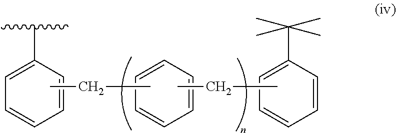

wherein the wavy line ( [symbol] ) indicates the point of attachment to the nitrogen of the formula (I) and the crossed line ( [symbol] ) indicates the point of attachment to R⁵;
wherein n is an integer selected from 0, 1, 2, 3, 4, 5, 6, 7, 8, 9, or 10;
R⁵ is selected from the group consisting of OH, SH, COOH, NH₂, and NHR²⁰;
or R⁵ is a group of formula (v);

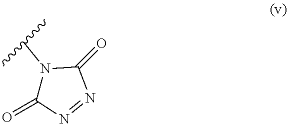 [SMILES: *N1C(=O)N=NC1=O]

wherein the wavy line ( [symbol] ) indicates the point of attachment to L¹;
or R⁵ is a group of formula (vi);

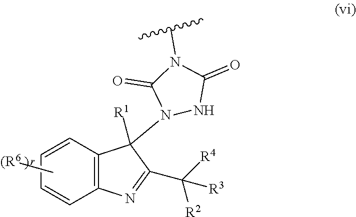

wherein the wavy line ( [symbol] ) indicates the point of attachment to L¹, and r, R¹, R², R³, R⁴ and R⁶ are as defined for structure (I);
each R¹⁹ is independently selected from the group consisting of C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl;
   wherein said C₁₋₁₀alkyl, C₂₋₂₀alkenyl, C₂₋₂₀alkynyl, C₆₋₁₂arylC₁₋₆alkyl, heterocyclylC₁₋₆alkyl; heteroarylC₁₋₆alkyl, optionally comprise one or more heteroatoms in the alkyl, alkenyl or alkynyl moiety, said heteroatoms being each independently selected from O, S and N;
   wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl, can be unsubstituted or substituted with one or more OH, SH, COOH, or NH₂;
each R²⁰ is independently selected from the group consisting of C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl;
   wherein said C₁₋₁₀alkyl, C₂₋₂₀alkenyl, C₂₋₂₀alkynyl, C₆₋₁₂arylC₁₋₆alkyl, heterocyclylC₁₋₆alkyl; heteroarylC₁₋₆alkyl, optionally comprise one or more heteroatoms in the alkyl, alkenyl or alkynyl moiety, said heteroatoms being each independently selected from O, S and N;
   wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl, can be unsubstituted or substituted with one or more OH, SH, COOH, or NH₂;
each R²¹ is independently selected from the group consisting of hydrogen, C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl;
   wherein said C₁₋₁₀alkyl, C₂₋₂₀alkenyl, C₂₋₂₀alkynyl, C₆₋₁₂arylC₁₋₆alkyl, heterocyclylC₁₋₆alkyl; heteroarylC₁₋₆alkyl, optionally comprise one or more heteroatoms in the alkyl, alkenyl or alkynyl moiety, said heteroatoms being each independently selected from O, S and N;
   wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl, can be unsubstituted or substituted with one or more OH, SH, COOH, or NH₂;
or wherein R²⁰ and R²¹ together with the atom to which they are attached form a 5-, 6-, or 7-membered heterocycle.
each Z^{1a} is selected from the group consisting of C₁₋₆alkyl; C₂₋₆alkenyl; C₂₋₆alkynyl; haloC₁₋₆alkyl; haloC₁₋₆alkyloxy; halogen; isocyanate;
or Z^{1a} is a group of formula (vii);

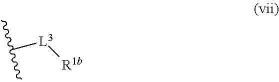

wherein the wavy line ( [symbol] ) indicates the point of attachment to L¹;
   L³ is a single bond or is selected from the group consisting of C₁₋₁₀alkylene; C₂₋₁₀alkenylene; C₂₋₁₀alkynylene; and C₆₋₁₂aryl;
   R^{1b} is a group of formula (viii);

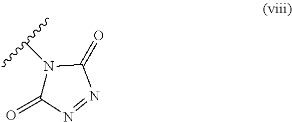 [SMILES: *N1C(=O)N=NC1=O]

wherein the wavy line ( [symbol] ) indicates the point of attachment to L³;
or R^{1b} is a group of formula (ix);

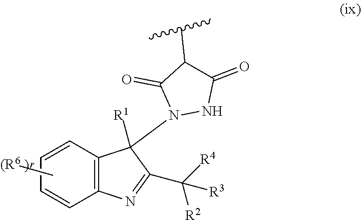

wherein the wavy line ( [symbol] ) indicates the point of attachment to L³ and r, R¹, R², R³, R⁴ and R⁶ are as defined for structure (I);
Z¹, Z², Z³, Z⁴, Z⁶ are each independently selected from the group consisting of OH; SH; COOH; NH₂; NHR²⁰; halogen; C₁₋₆alkyl; haloC₁₋₆alkyl; haloC₁₋₆alkyloxy; —OR¹⁹; —SR¹⁹; nitro; NR²⁰R²¹; —C(O)NR²⁰R²¹; cyano;
Z⁵, Z^{5a}, Z⁷, Z⁸, Z^{8a}, Z⁹ are each independently selected from the group consisting of OH; hydrogen; SH; COOH; NH₂; NHR²⁰; halogen; C₁₋₆alkyl; haloC₁₋₆alkyl; haloC₁₋₆alkyloxy; —OR¹⁹; —SR¹⁹; nitro; NR²⁰R²¹; —C(O)NR²⁰R²¹; cyano; and a polymeric group;
   and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
and when none of R¹, R², R³, R⁴, R⁶, R⁷, R⁸, R⁹, R¹⁹, R¹¹, R¹², R¹⁴, R¹⁵, R¹⁶, R¹⁷, Z⁵, Z^{5a}, Z⁷, Z⁸, Z^{8a} or Z⁹ is a polymeric group, then at least one of R⁷, R⁸, Z¹, Z², Z³, Z⁴, Z⁵, Z^{5a}, Z⁶, Z⁷, Z⁸, Z^{8a} or Z⁹ is selected from the group consisting of OH, SH, COOH, NH₂, and NHR²⁰.

According to a second aspect of the present invention, a process for preparing a compound according to the first aspect of the invention is provided. The process comprises the step of contacting at least one compound of formula (II) with at least one compound of formula (III), thereby obtaining a compound of formula (I), wherein r, R¹, R², R³, R⁴, R⁵, R⁶, L¹, have the same meaning as defined herein,

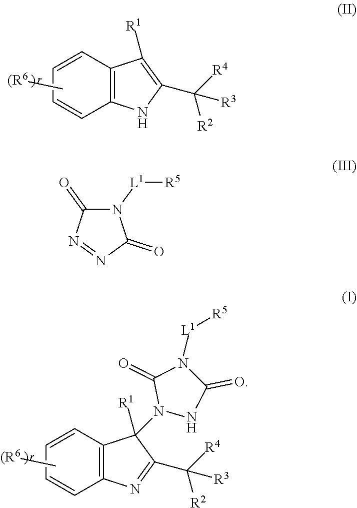

The present invention also encompasses a compound of formula (I) according to the first aspect of the invention obtainable by contacting a compound of formula (II) with a compound of formula (III).

According to a third aspect, the invention encompasses a compound of formula (A) or a stereoisomer, enantiomer, racemic, or tautomer thereof,

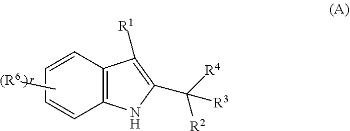

wherein,
r is an integer selected from 0, 1, 2, 3 or 4;
R¹ is selected from the group consisting of C₁₋₁₀alkyl; C₂₋₁₀alkenyl; C₂₋₁₀alkynyl; C₆₋₁₂aryl; C₃₋₈cycloalkyl; C₆₋₁₂arylC₁₋₆alkyl; heterocyclyl; heteroaryl; heterocyclylC₁₋₆alkyl; heteroarylC₁₋₈alkyl; —OR¹⁹; —SR¹⁹; NR²⁰R²¹; —C(O)NR²⁰R²¹; OH, SH, COOH, NH₂; NHR²⁰; and a polymeric group;
and wherein said C₁₋₁₀alkyl; C₂₋₁₀alkenyl; C₂₋₁₀alkynyl, C₆₋₁₂aryl, C₃₋₈cycloalkyl; C₆₋₁₂arylC₁₋₆alkyl, heterocyclyl; heteroaryl; heterocyclylC₁₋₆alkyl; or heteroarylC₁₋₆alkyl; can be unsubstituted or substituted with one or more Z¹;
   and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
   or R¹ is a group of formula (i);

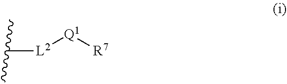

wherein the wavy line ( [symbol] ) indicates the point of attachment to the indole ring of formula (I);
L² is selected from the group consisting of C₁₋₁₀alkylene; C₃₋₆cycloalkylene; C₂₋₁₀alkenylene; C₂₋₁₀alkynylene; C₆₋₁₂arylene; and C₆₋₁₂arylC₁₋₆alkylene;
   and wherein said C₁₋₁₀alkylene; C₃₋₆cycloalkylene; C₂₋₁₀alkenylene; C₂₋₁₀alkynylene; C₆₋₁₂arylene or C₆₋₁₂arylC₁₋₆alkylene, can be unsubstituted or substituted with one or more Z²;
Q¹ is a single bond or selected from the group consisting of C₁₋₁₀alkylene; C₃₋₆cycloalkylene; —O—; —S—; —O—C₁₋₁₀alkylene; and —S—C₁₋₁₀alkylene; wherein each left side of said groups is attached to L² and the right side thereof is attached to R⁷;
   and wherein said C₁₋₁₀alkylene and said C₃₋₆cycloalkylene; can be unsubstituted or substituted with one or more Z³;
R⁷ is selected from the group consisting of hydrogen; OH, SH, COOH, NH₂; NHR²⁰, halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
   wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₈alkyl, heterocyclyl; heteroaryl; heterocyclylC₁₋₆alkyl; and heteroarylC₁₋₆alkyl; can be unsubstituted or substituted with one or more Z⁴;
   and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
or R⁷ is a group of formula (ii);

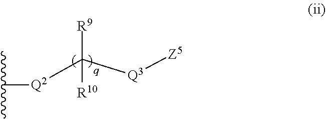

wherein the wavy line ( [symbol] ) indicates the point of attachment to Q¹;
q is an integer selected from 0; 1, 2, 3 or 4;
Q² is a single bond or selected from the group consisting of CR¹¹R¹²; O, S, and NR¹³;
Q³ is a single bond or selected from the group consisting of hydroxyC₁₋₁₀alkylene; aminoC₁₋₁₀alkylene; mercaptoC₁₋₁₀alkylene; C₁₋₂₀alkylene; C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₆alkylene; and heteroarylC₁₋₆alkylene;
wherein said C₁₋₂₀alkylene, C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₈alkylene; or heteroarylC₁₋₆alkylene, can be unsubstituted or substituted with one or more Z^{5a};
wherein if q is 0, then one of Q² and Q³ is not a single bond;
each R⁹ and R¹⁰ is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
each R¹¹ and R¹² is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
R¹³ is selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; and heteroarylC₁₋₆alkyl;
R² is selected from the group consisting of halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; heteroarylC₁₋₆alkyl; and a polymeric group;
R³ is selected from the group consisting of halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; heteroarylC₁₋₆alkyl; and a polymeric group;

R⁴ is selected from the group consisting of halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; heteroarylC₁₋₆alkyl; and a polymeric group; wherein for R², R³ and R⁴, each independently; said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl, heteroarylC₁₋₆alkyl, can be unsubstituted or substituted with one or more Z⁶;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
or R² and R³ and R⁴ together with the carbon atom to which they are attached can form a saturated or unsaturated 4-, 5-, 6-, 7- or 8-membered ring;
   wherein said saturated or unsaturated 4-, 5-, 6-, 7- or 8-membered ring optionally comprises one or more heteroatoms, said heteroatoms being each independently selected from O, S and N;
   wherein said saturated or unsaturated 4-, 5-, 6-, 7- or 8-membered ring can be unsubstituted or substituted with one or more R⁸;
each R⁸ is independently selected from the group consisting of hydroxyC₁₋₁₀alkoxy; aminoC₁₋₁₀alkoxy; mercaptoC₁₋₁₀alkoxy; C₁₋₁₀alkyl; C₂₋₁₀alkenyl; C₂₋₁₀alkynyl; C₆₋₁₂arylC₁₋₆alkyl; C₃₋₈cycloalkyl; C₆₋₁₂arylC₁₋₆alkyl; heterocyclyl; heteroaryl; heterocyclylC₁₋₆alkyl; heteroarylC₁₋₈alkyl; OH; SH; COOH; NH₂; NHR²⁰; halogen; haloC₁₋₆alkyl; haloC₁₋₆alkyloxy; —OR¹⁹; —SR¹⁹; nitro; NR²⁰R²¹; —C(O)NR²⁰R²¹; cyano; and a polymeric group;
   wherein said C₁₋₁₀alkyl; C₂₋₁₀alkenyl; C₂₋₁₀alkynyl; C₆₋₁₂arylC₁₋₆alkyl; C₃₋₈cycloalkyl; C₆₋₁₂arylC₁₋₆alkyl; heterocyclyl; heteroaryl; heterocyclylC₁₋₆alkyl; or heteroarylC₁₋₆alkyl, can be unsubstituted or substituted with one or more Z⁷;
   and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
   or R⁸ is a group of formula (iii);

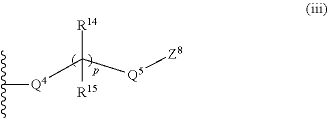

wherein the wavy line ( [symbol] ) indicates the point of attachment to the ring formed by R² and R³ and R⁴ together;
p is an integer selected from 0; 1, 2, 3 or 4;
Q⁴ is a single bond or selected from the group consisting of CR¹⁶R¹⁷; O, S, and NR¹⁸;
Q⁵ is a single bond or selected from the group consisting of hydroxyC₁₋₁₀alkylene; aminoC₁₋₁₀alkylene; mercaptoC₁₋₁₀alkylene; C₁₋₂₀alkylene, C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₆alkylene; and heteroarylC₁₋₆alkylene;
wherein said C₁₋₂₀alkylene, C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₆alkylene; or heteroarylC₁₋₆alkylene, can be unsubstituted or substituted with one or more Z^{8a};
wherein if p is 0, then one of Q⁴ and Q⁵ is not a single bond;
each R¹⁴ and R¹⁵ is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
each R¹⁶ and R¹⁷ is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
R¹⁸ is selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; and heteroarylC₁₋₆alkyl;
each R⁶ is independently selected from the group consisting of hydrogen, halogen; C₁₋₁₀alkyl; C₂₋₁₀alkenyl; C₂₋₁₀alkynyl; C₆₋₁₂arylC₁₋₆alkyl; haloC₁₋₆alkyl; haloC₁₋₆alkyloxy; —OR¹⁹; —SR¹⁹; nitro; NR²⁰R²¹; —C(O)NR²⁰R²¹; cyano; and a polymeric group;
   wherein said C₁₋₁₀alkyl; C₂₋₁₀alkenyl; C₂₋₁₀alkynyl; or C₆₋₁₂arylC₁₋₆alkyl, can be unsubstituted or substituted with one or more Z⁹;
   and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
each R¹⁹ is independently selected from the group consisting of C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl;
   wherein said C₁₋₁₀alkyl, C₂₋₂₀alkenyl, C₂₋₂₀alkynyl, C₆₋₁₂arylC₁₋₆alkyl, heterocyclylC₁₋₆alkyl; heteroarylC₁₋₆alkyl, optionally comprise one or more heteroatoms in the alkyl, alkenyl or alkynyl moiety, said heteroatoms being each independently selected from O, S and N; wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl, can be unsubstituted or substituted with one or more OH, SH, COOH, or NH₂;
each R²⁰ is independently selected from the group consisting of C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl;
   wherein said C₁₋₁₀alkyl, C₂₋₂₀alkenyl, C₂₋₂₀alkynyl, C₆₋₁₂arylC₁₋₆alkyl, heterocyclylC₁₋₆alkyl; heteroarylC₁₋₆alkyl, optionally comprise one or more heteroatoms in the alkyl, alkenyl or alkynyl moiety, said heteroatoms being each independently selected from O, S and N;
   wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl, can be unsubstituted or substituted with one or more OH, SH, COOH, or NH₂;
each R²¹ is independently selected from the group consisting of hydrogen; C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl;
   wherein said C₁₋₁₀alkyl, C₂₋₂₀alkenyl, C₂₋₂₀alkynyl, C₆₋₁₂arylC₁₋₆alkyl, heterocyclylC₁₋₆alkyl; heteroarylC₁₋₆alkyl, optionally comprise one or more heteroatoms in the alkyl, alkenyl or alkynyl moiety, said heteroatoms being each independently selected from O, S and Nor wherein R²⁰ and R²¹ together with the atom to which they are attached form a 5-, 6-, or 7-membered heterocycle;
   wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl, can be unsubstituted or substituted with one or more OH, SH, COOH, or NH₂;
Z¹, Z², Z³, Z⁴, Z⁶ are each independently selected from the group consisting of OH; SH; COOH; NH₂; NHR²⁰; halogen; C₁₋₆alkyl; haloC₁₋₆alkyl; haloC₁₋₆alkyloxy; —OR¹⁹; —SR¹⁹; nitro; NR²⁰R²¹; —C(O)NR²⁰R²¹; cyano;
Z⁵, Z^{5a}, Z⁷, Z⁸, Z^{5a}, Z⁹ are independently selected from the group consisting of OH; hydrogen; SH; COOH; NH₂; NHR²⁰; halogen; C₁₋₆alkyl; haloC₁₋₆alkyl; haloC₁₋₆alkyloxy; —OR¹⁹; —SR¹⁹; nitro; NR²⁰R²¹; —C(O)NR²⁰R²¹; cyano; and a polymeric group;
   and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
wherein none of R¹, R², R³, R⁴, R⁵, R⁷, R⁸, R⁹, R¹⁰, R¹¹, R¹², R¹⁴, R¹⁵, R¹⁶, R¹⁷, Z⁵, Z^{5a}, Z⁷, Z⁸, Z^{8a} or Z⁹ is a polymeric group, then at least one of R⁷, R⁸, Z¹, Z², Z³, Z⁴, Z⁵, Z^{5a}, Z⁶, Z⁷, Z⁸ Z⁸a or Z⁹ is selected from the group consisting of OH, SH, COOH, NH₂, and NHR²⁰.

According to a fourth aspect, the invention encompasses a composition comprising a compound according to the first aspect of the invention.

According to a fifth aspect, the invention also encompasses a polyurethane or polyurethane prepolymer obtainable by contacting at least one isocyanate with at least one compound according to the first aspect of the invention, or with at least one compound according to the third aspect of the invention, or with a composition as defined in the fourth aspect of the invention.

In a sixth aspect, the invention encompasses the use of a compound according to the first aspect of the invention, or of a compound according to the third aspect of the invention, or of a composition according to the fourth aspect of the invention, or of a polyurethane or polyurethane prepolymer according to the fifth aspect of the invention, for the preparation of polyurethane foams, including but not limited to flexible foams, semi-rigid foams or rigid foams, adhesives, coatings, sealants, aerogels or elastomers (including non-cellular and microcellular elastomers).

The invention also encompasses an article comprising a compound according to the first aspect of the invention, or a compound according to the third aspect of the invention, or a composition as defined in the fourth aspect of the invention, or a polyurethane or polyurethane prepolymer as defined in the fifth aspect of the invention.

### BRIEF DESCRIPTION OF THE FIGURES

FIG. 1 represents a graph plotting the density of the polyurethane foams against the reprocessing temperature, wherein the reprocessing was performed at a pressure of 6 bar, for 10 minutes.

FIG. 2 represents a scheme of the oxidation set up employed in the synthesis of compounds according to the invention.

### DETAILED DESCRIPTION OF THE INVENTION

Before the present invention is described, it is to be understood that this invention is not limited to particular processes, methods, and compounds described, as such processes, methods, and compounds may, of course, vary. It is also to be understood that the terminology used herein is not intended to be limiting, since the scope of the present invention will be limited only by the appended claims.

When describing the compounds and processes of the invention, the terms used are to be construed in accordance with the following definitions, unless a context dictates otherwise.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The term “about” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

As used herein, the term “and/or,” when used in a list of two or more items, means that any one of the listed items can be employed by itself or any combination of two or more of the listed items can be employed. For example, if a list is described as comprising group A, B, and/or C, the list can comprise A alone; B alone; C alone; A and B in combination; A and C in combination, B and C in combination; or A, B, and C in combination.

The recitation of numerical ranges by endpoints includes all numbers and fractions subsumed within the respective ranges, as well as the recited endpoints.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention.

When describing the present invention, the terms used are to be construed in accordance with the following definitions, unless a context dictates otherwise.

Whenever the term “substituted” is used herein, it is meant to indicate that one or more hydrogens on the atom indicated in the expression using “substituted” is replaced with a selection from the indicated group, provided that the indicated atom's normal valency is not exceeded, and that the substitution results in a chemically stable compound, i.e. a compound that is sufficiently robust to survive isolation from a reaction mixture.

Where groups can be substituted, such groups may be substituted with one or more, and preferably one, two or three substituents. Substituents may be selected from but not limited to, for example, the group comprising OH; SH; COOH; NH₂; NHR²⁰; halogen; C₁₋₆alkyl; haloC₁₋₆alkyl; haloC₁₋₆alkyloxy; —OR¹⁹; —SR¹⁹; nitro; NR²⁰R²¹; cyano.

The terminology “optionally comprises one or more heteroatoms, said heteroatoms being selected from the atoms O, S, or N” as used herein, refers to a group where one or more carbon atoms are replaced by at least one oxygen, nitrogen or sulfur atom and thus includes, depending on the group to which is referred, heteroalkyl, heteroalkenyl, heteroalkynyl, heteroalkylene, heteroalkenylene, heteroalkynylene, cycloheteroalkyl, cycloheteroalkenyl, cycloheteroalkynyl, heteroaryl, arylheteroalkyl(ene), heteroarylalkyl(ene), heteroarylheteroalkyl(ene), arylheteroalkenyl(ene), heteroarylalkenyl(ene), heteroarylheteroalkenyl(ene), heteroarylheteroalkenyl(ene), arylheteroalkynyl(ene), heteroarylalkynyl(ene), heteroarylheteroalkynyl(ene), and compounds wherein the replaced carbon atom is a terminal carbon atom, among others. This term therefore comprises, depending on the group to which is referred, as an example “heteroalkyl” such as alkoxy, alkoxyalkyl, mono- or di-alkylamino, mono- or di-alkylaminoalkyl, alkylthio, alkylthioalkyl;

“heteroalkenyl” such as alkenyloxy, alkenyloxyalkenyl, mono- or di-alkenylamino, mono- or di-alkenylaminoalkenyl, alkenylthio, alkenylthioalkenyl, and “heteroalkynyl” such as alkynyloxy, alkynyloxyalkynyl, mono- or di-alkynylamino, mono- or di-alkynylaminoalkynyl, alkynylthio, alkynylthioalkynyl. This term also comprises alcohols, amines and thiols. As an example, the terminology “alkyl optionally comprises one or more heteroatoms, said heteroatoms being selected from the atoms of O, S, or N” therefore refers to heteroalkyl, meaning an alkyl which comprises one or more heteroatoms in the hydrocarbon chain, whereas the heteroatoms may be positioned at the beginning of the hydrocarbon chain, in the hydrocarbon chain or at the end of the hydrocarbon chain. Examples of heteroalkyl include alkoxy, alkoxyalkyl, alkylamino, alkylaminoalkyl, alkylthio, alkylthioalkyl, such as methoxy, methylthio, ethoxy, propoxy, CH₃—O—CH₂—, CH₃—S—CH₂—, CH₃—CH₂—O—CH₂—, CH₃NH—, (CH₃)₂N—, (CH₃)₂—CH₂—NHCH₂—CH₂—, among many other examples. As an example, the terminology “arylalkyl optionally comprises one or more heteroatoms in the alkyl chain, said heteroatoms being selected from the atoms O, S, or N” therefore refers to aryl-heteroalkyl, meaning an arylalkyl which comprises one or more heteroatoms in the hydrocarbon chain, whereas the heteroatoms may be positioned at the beginning of the hydrocarbon chain, in the hydrocarbon chain or at the end of the hydrocarbon chain. “Aryl-heteroalkyl” thus includes aryloxy, arylalkoxy, aryl-alkyl-NH— and the like and examples are phenyloxy, benzyloxy, aryl-CH₂—S—CH₂—, aryl-CH₂—O—CH₂—, aryl-NH—CH₂— among many other examples.

The term “halo” or “halogen” as a group or part of a group is generic for fluoro, chloro, bromo, iodo.

The term “amino” refers to the group NH₂.

The term “hydroxyl” or “hydroxy” as used herein refers to the group —OH.

The term “mercapto” refers to the group SH.

The term “nitro” as used herein refers to the group —NO₂.

The term “cyano” as used herein refers to the group —CN.

The term “isocyanate” as used herein refers to the group —NCO.

The term “carboxy” or “carboxyl” or “hydroxycarbonyl” as used herein refers to the group —CO₂H.

The term “alkyl”, as a group or part of a group, refers to a hydrocarbyl group of Formula CₙH₂ₙ₊₁ wherein n is a number of at least 1. Alkyl groups may be linear, or branched and may be substituted as indicated herein. Generally, the alkyl groups comprise from 1 to 20 carbon atoms, preferably from 1 to 12 carbon atoms, preferably from 1 to 10 carbon atoms, preferably from 1 to 6 carbon atoms, more preferably 1, 2, 3, 4, 5, 6 carbon atoms. When a subscript is used herein following a carbon atom, the subscript refers to the number of carbon atoms that the named group may contain. For example, the term “C₁₋₂₀alkyl”, as a group or part of a group, refers to a hydrocarbyl group of Formula CₙH₂ₙ₊₁ wherein n is a number ranging from 1 to 20.

Thus, for example, C₁₋₂₀alkyl groups include all linear, or branched alkyl groups having 1 to 20 carbon atoms, and thus includes for example methyl, ethyl, n-propyl, i-propyl, 2-methyl-ethyl, butyl and its isomers (e.g. n-butyl, i-butyl and t-butyl); pentyl and its isomers, hexyl and its isomers, heptyl and its isomers, octyl and its isomers, nonyl and its isomers, decyl and its isomers, undecyl and its isomers, dodecyl and its isomers, tridecyl and its isomers, tetradecyl and its isomers, pentadecyl and its isomers, hexadecyl and its isomers, heptadecyl and its isomers, octadecyl and its isomers, nonadecyl and its isomers, icosyl and its isomers, and the like. For example, C₁₋₁₀alkyl includes all linear, or branched alkyl groups having 1 to 10 carbon atoms, and thus includes for example methyl, ethyl, n-propyl, i-propyl, 2-methyl-ethyl, butyl and its isomers (e.g. n-butyl, i-butyl and t-butyl); pentyl and its isomers, hexyl and its isomers, heptyl and its isomers, octyl and its isomers, nonyl and its isomers, decyl and its isomers and the like.

For example, C₁₋₆alkyl includes all linear, or branched alkyl groups having 1 to 6 carbon atoms, and thus includes for example methyl, ethyl, n-propyl, i-propyl, 2-methyl-ethyl, butyl and its isomers (e.g. n-butyl, i-butyl and t-butyl); pentyl and its isomers, hexyl and its isomers. When the suffix “ene” is used in conjunction with an alkyl group, i.e. “alkylene”, this is intended to mean the alkyl group as defined herein having two single bonds as points of attachment to other groups. Non-limiting examples of alkylene groups includes methylene, ethylene, methylmethylene, propylene, ethylethylene, and 1,2-dimethylethylene. Similarly, where alkenyl groups as defined herein and alkynyl groups as defined herein, respectively, are divalent groups having single bonds for attachment to two other groups, they are termed “alkenylene” and “alkynylene” respectively.

The term “C₁₋₁₀alkoxy” or “C₁₋₁₀alkyloxy”, as a group or part of a group, refers to a group having the formula OR^{b} wherein R^{b} is C₁₋₁₀alkyl as defined herein above. Non-limiting examples of suitable C₁₋₁₀alkoxy include methoxy, ethoxy, propoxy, isopropoxy, butoxy, isobutoxy, sec-butoxy, tert-butoxy, pentyloxy and hexyloxy.

The term “hydroxyC₁₋₁₀alkoxy”, as a group or part of a group, means a C₁₋₁₀alkoxy, wherein at least one hydrogen atom is replaced by at least one hydroxyl group as defined herein.

The term “aminoC₁₋₁₀alkoxy”, as a group or part of a group, means a C₁₋₁₀alkoxy, wherein at least one hydrogen atom is replaced by at least one amino group as defined herein.

The term “mercaptoC₁₋₁₀alkoxy”, as a group or part of a group, means a C₁₋₁₀alkoxy, wherein at least one hydrogen atom is replaced by at least one thiol group as defined herein.

The term “C₂₋₂₀alkenyl” as a group or part of a group, refers to an unsaturated hydrocarbyl group, which may be linear, branched or cyclic, comprising one or more carbon-carbon double bonds. Alkenyl groups thus preferably comprise between 2 and 20 carbon atoms, preferably between 2 and 10 carbon atoms, still more preferably between 2 and 6 carbon atoms. Examples of alkenyl groups are ethenyl, 2-propenyl, 2-butenyl, 3-butenyl, 2-pentenyl and its isomers, 2-hexenyl and its isomers, 2,4-pentadienyl and the like.

The term “C₂₋₂₀alkynyl” as a group or part of a group, refers to a class of monovalent unsaturated hydrocarbyl groups, wherein the unsaturation arises from the presence of one or more carbon-carbon triple bonds. Alkynyl groups thus preferably comprise between 2 and 20 carbon atoms, preferably between 2 and 10 carbon atoms, still more preferably between 2 and 6 carbon atoms. Non limiting examples of alkynyl groups are ethynyl, 2-propynyl, 2-butynyl, 3-butynyl, 2-pentynyl and its isomers, 2-hexynyl and its isomers-and the like.

The term “cycloalkyl”, as a group or part of a group, refers to a cyclic alkyl group, that is a monovalent, saturated, hydrocarbyl group having 1 or more cyclic structure, and comprising from 3 to 12 carbon atoms, more preferably from 3 to 9 carbon atoms, more preferably from 3 to 6 carbon atoms, still more preferably from 5 to 6 carbon atoms. Cycloalkyl includes all saturated hydrocarbon groups containing 1 or more rings, including monocyclic or bicyclic groups. The further rings of multi-ring cycloalkyls may be either fused, bridged and/or joined through one or more spiro atoms. The term “C₃₋₆cycloalkyl”, refers to a cyclic alkyl group comprising from 3 to 6 carbon atoms, more preferably from 5 to 6 carbon atoms. Examples of C₃₋₆cycloalkyl groups include but are not limited to cyclopropyl, cyclobutyl, cyclopentyl, cyclohexyl. Cycloalkyl groups may also be considered to be a subset of homocyclic rings discussed hereinafter. When the suffix “ene” is used in conjunction with a cycloalkyl group, i.e. cycloalkylene, this is intended to mean the cycloalkyl group as defined herein having two single bonds as points of attachment to other groups.

The term “homocyclic ring” as a group or part of a group, refers to a ring wherein the ring atoms comprise only carbon atoms. Non limiting examples of homocyclic rings include cycloalkyl, cycloalkenyl, with cycloalkyl being preferred. Where a ring carbon atom is replaced with a heteroatom, preferably nitrogen, oxygen of sulfur, the heteroatom-containing ring resultant from such a replacement is referred to herein as a heterocyclic ring. More than one carbon atom in a ring may be replaced so forming heterocyclic ring having a plurality of heteroatoms.

The term “haloC₁₋₆alkyl” as a group or part of a group, refers to a C₁₋₆alkyl group having the meaning as defined above wherein one or more hydrogens are replaced with one or more halogen as defined above. Non-limiting examples of such haloalkyl groups include chloromethyl, 1-bromoethyl, fluoromethyl, difluoromethyl, trifluoromethyl, 1,1,1-trifluoroethyl and the like.

The term “hydroxyC₁₋₁₀alkylene”, as a group or part of a group, means a C₁₋₁₀alkylene, wherein at least one hydrogen atom is replaced by at least one hydroxyl group as defined herein.

The term “aminoC₁₋₁₀alkylene”, as a group or part of a group, means a C₁₋₁₀alkylene, wherein at least one hydrogen atom is replaced by at least one amino group as defined herein.

The term “mercaptoC₁₋₁₀alkylene”, as a group or part of a group, means a C₁₋₁₀alkylene, wherein at least one hydrogen atom is replaced by at least one thiol group as defined herein.

The term “C₃₋₈cycloalkyleneC₁₋₁₀alkylene”, as a group or part of a group, means a C₁₋₁₀alkylene, wherein at least one hydrogen atom is replaced by at least one C₃₋₈cycloalkylene as defined herein.

The term, “C₁₋₁₀alkyleneC₃₋₈cycloalkylene” as a group or part of a group, means a C₃₋₈cycloalkylene, wherein at least one hydrogen atom is replaced by at least one C₁₋₁₀alkylene as defined herein.

The term, “C₃₋₈cycloalkyleneC₁₋₁₀alkylC₃₋₈cycloalkylene” as a group or part of a group, means a C₃₋₈cycloalkylene, wherein at least one hydrogen atom is replaced by at least one C₃₋₈cycloalkyleneC₁₋₁₀alkyl as defined herein.

The term “C₆₋₁₂aryl”, as a group or part of a group, refers to a polyunsaturated, aromatic hydrocarbyl group having a single ring (i.e. phenyl) or multiple aromatic rings fused together (e.g. naphthalene), or linked covalently, typically containing 6 to 12 atoms; wherein at least one ring is aromatic. The aromatic ring may optionally include one to two additional rings (either cycloalkyl, heterocyclyl or heteroaryl) fused thereto. Examples of suitable aryl include C₈₋₁₀aryl, more preferably C₆₋₈aryl. Non-limiting examples of C₆₋₁₂aryl comprise phenyl, biphenylyl, biphenylenyl, or 1- or 2-naphthanelyl; 5- or 6-tetralinyl, 1-, 2-, 3-, 4-, 5-, 6-, 7- or 8-azulenyl, 4-, 5-, 6 or 7-indenyl, 4- or 5-indanyl, 5-, 6-, 7- or 8-tetrahydronaphthyl, 1,2,3,4-tetrahydronaphthyl, and 1,4-dihydronaphthyl. When the suffix “ene” is used in conjunction with an aryl group, this is intended to mean the aryl group as defined herein having two single bonds as points of attachment to other groups, such as phenylene, biphenylylene, naphthylene, indenylene, and the like. Where a carbon atom in an aryl group is replaced with a heteroatom, the resultant ring is referred to herein as a heteroaryl ring.

The term “C₆₋₁₂arylC₁₋₁₂alkyl”, as a group or part of a group, means a C₁₋₁₂alkyl as defined herein, wherein at least one hydrogen atom is replaced by at least one C₆₋₁₂aryl as defined herein. Non-limiting examples of C₆₋₁₂arylC₁₋₆alkyl group include benzyl, phenethyl, dibenzylmethyl, methylphenylmethyl, 3-(2-naphthyl)-butyl, and the like.

The term “C₆₋₁₂aryleneC₁₋₆alkylene”, as a group or part of a group, means a C₁₋₆alkylene, wherein at least one hydrogen atom is replaced by at least one C₆₋₁₂arylene as defined herein.

The term “C₁₋₆alkylC₆₋₁₂arylC₁₋₆alkyl”, as a group or part of a group, means a C₁₋₆alkyl, wherein at least one hydrogen atom is replaced by at least one C₁₋₆alkylC₆₋₁₂aryl as defined herein.

The term “C₁₋₆alkyleneC₆₋₁₂arylC₁₋₆alkylene”, as a group or part of a group, means a C₁₋₈alkylene, wherein at least one hydrogen atom is replaced by at least one C₁₋₆alkyleneC₆₋₁₂aryl as defined herein.

The term “C₆₋₁₂arylC₁₋₁₂alkylC₆₋₁₂aryl”, as a group or part of a group, means a C₆₋₁₂aryl, wherein at least one hydrogen atom is replaced by at least one C₆₋₁₂arylC₁₋₁₂alkyl as defined herein.

The term “C₆₋₁₂aryleneC₁₋₁₂alkylC₆₋₁₂arylene”, as a group or part of a group, means a C₆₋₁₂arylene, wherein at least one hydrogen atom is replaced by at least one C₆₋₁₂aryleneC₁₋₁₂alkyl as defined herein.

The terms “heterocyclyl” or “heterocyclo”, as a group or part of a group, refer to non-aromatic, fully saturated or partially unsaturated cyclic groups (for example, 3 to 7 member monocyclic, 7 to 11 member bicyclic, or containing a total of 3 to 10 ring atoms) which have at least one heteroatom in at least one carbon atom-containing ring. Each ring of the heterocyclic group containing a heteroatom may have 1, 2, 3 or 4 heteroatoms selected from N, O and/or S, where the N and S heteroatoms may optionally be oxidized and the N heteroatoms may optionally be quaternized. The heterocyclic group may be attached at any heteroatom or carbon atom of the ring or ring system, where valence allows. The rings of multi-ring heterocycles may be fused, bridged and/or joined through one or more spiro atoms.

Non limiting exemplary heterocyclic groups include aziridinyl, oxiranyl, thiiranyl, piperidinyl, azetidinyl, 2-imidazolinyl, pyrazolidinyl imidazolidinyl, isoxazolinyl, oxazolidinyl, isoxazolidinyl, thiazolidinyl, isothiazolidinyl, piperidinyl, succinimidyl, 3H-indolyl, indolinyl, isoindolinyl, 2H-pyrrolyl, 1-pyrrolinyl, 2-pyrrolinyl, 3-pyrrolinyl, pyrrolidinyl, 4H-quinolizinyl, 2-oxopiperazinyl, piperazinyl, homopiperazinyl, 2-pyrazolinyl, 3-pyrazolinyl, tetrahydro-2H-pyranyl, 2H-pyranyl, 4H-pyranyl, 3,4-dihydro-2H-pyranyl, oxetanyl, thietanyl, 3-dioxolanyl, 1,4-dioxanyl, 2,5-dioximidazolidinyl, 2-oxopiperidinyl, 2-oxopyrrolodinyl, indolinyl, tetrahydropyranyl, tetrahydrofuranyl, tetrahydrothiophenyl, tetrahydroquinolinyl, tetrahydroisoquinolin-1-yl, tetrahydroisoquinolin-2-yl, tetrahydroisoquinolin-3-yl, tetrahydroisoquinolin-4-yl, thiomorpholin-4-yl, thiomorpholin-4-ylsulfoxide, thiomorpholin-4-ylsulfone, 1,3-dioxolanyl, 1,4-oxathianyl, 1,4-dithianyl, 1,3,5-trioxanyl, 1H-pyrrolizinyl, tetrahydro-1,1-dioxothiophenyl, N-formylpiperazinyl, and morpholin-4-yl.

The term “heteroaryl” as a group or part of a group, refers but is not limited to 5 to 12 atom aromatic rings or ring systems containing 1 to 2 rings which are fused together or linked covalently, typically containing 5 to 12 atoms; at least one of which is aromatic in which one or more carbon atoms in one or more of these rings can be replaced by N, O and/or S atoms where the N and S heteroatoms may optionally be oxidized and the N heteroatoms may optionally be quaternized. Such rings may be fused to an aryl, cycloalkyl, heteroaryl or heterocyclyl ring. Non-limiting examples of such heteroaryl, include: pyrrolyl, furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, oxazinyl, dioxinyl, thiazinyl, triazinyl, imidazo[2,1-b][1,3]thiazolyl, thieno[3,2-b]furanyl, thieno[3,2-b]thiophenyl, thieno[2,3-d][1,3]thiazolyl, thieno[2,3-d]imidazolyl, tetrazolo[1,5-a]pyridinyl, indolyl, indolizinyl, isoindolyl, benzofuranyl, isobenzofuranyl, benzothiophenyl, isobenzothiophenyl, indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1,2,3-benzothiadiazolyl, 2,1,3-benzothiadiazolyl, thienopyridinyl, purinyl, imidazo[1,2-a]pyridinyl, 6-oxo-pyridazin-1(6H)-yl, 2-oxopyridin-1(2H)-yl, 6-oxo-pyridazin-1(6H)-yl, 2-oxopyridin-1(2H)-yl, 1,3-benzodioxolyl, quinolinyl, isoquinolinyl, cinnolinyl, quinazolinyl, quinoxalinyl,

The term “pyrrolyl” (also called azolyl) as used herein includes pyrrol-1-yl, pyrrol-2-yl and pyrrol-3-yl. The term “furanyl” (also called “fury)”) as used herein includes furan-2-yl and furan-3-yl (also called furan-2-yl and furan-3-yl). The term “thiophenyl” (also called “thienyl”) as used herein includes thiophen-2-yl and thiophen-3-yl (also called thien-2-yl and thien-3-yl). The term “pyrazolyl” (also called 1H-pyrazolyl and 1,2-diazolyl) as used herein includes pyrazol-1-yl, pyrazol-3-yl, pyrazol-4-yl and pyrazol-5-yl. The term “imidazolyl” as used herein includes imidazol-1-yl, imidazol-2-yl, imidazol-4-yl and imidazol-5-yl. The term “oxazolyl” (also called 1,3-oxazolyl) as used herein includes oxazol-2-yl; oxazol-4-yl and oxazol-5-yl. The term “isoxazolyl” (also called 1,2-oxazolyl), as used herein includes isoxazol-3-yl, isoxazol-4-yl, and isoxazol-5-yl. The term “thiazolyl” (also called 1,3-thiazolyl), as used herein includes thiazol-2-yl, thiazol-4-yl and thiazol-5-yl (also called 2-thiazolyl, 4-thiazolyl and 5-thiazolyl). The term “isothiazolyl” (also called 1,2-thiazolyl) as used herein includes isothiazol-3-yl, isothiazol-4-yl, and isothiazol-5-yl. The term “triazolyl” as used herein includes 1H-triazolyl and 4H-1,2,4-triazolyl, “1H-triazolyl” includes 1H-1,2,3-triazol-1-yl, 1H-1,2,3-triazol-4-yl, 1H-1,2,3-triazol-5-yl, 1H-1,2,4-triazol-1-yl, 1H-1,2,4-triazol-3-yl and 1H-1,2,4-triazol-5-yl. “4H-1,2,4-triazolyl” includes 4H-1,2,4-triazol-4-yl, and 4H-1,2,4-triazol-3-yl. The term “oxadiazolyl” as used herein includes 1,2,3-oxadiazol-4-yl, 1,2,3-oxadiazol-5-yl, 1,2,4-oxadiazol-3-yl, 1,2,4-oxadiazol-5-yl, 1,2,5-oxadiazol-3-yl and 1,3,4-oxadiazol-2-yl. The term “thiadiazolyl” as used herein includes 1,2,3-thiadiazol-4-yl, 1,2,3-thiadiazol-5-yl, 1,2,4-thiadiazol-3-yl, 1,2,4-thiadiazol-5-yl, 1,2,5-thiadiazol-3-yl (also called furazan-3-yl) and 1,3,4-thiadiazol-2-yl. The term “tetrazolyl” as used herein includes 1H-tetrazol-1-yl, 1H-tetrazol-5-yl, 2H-tetrazol-2-yl, and 2H-tetrazol-5-yl. The term “oxatriazolyl” as used herein includes 1,2,3,4-oxatriazol-5-yl and 1,2,3,5-oxatriazol-4-yl. The term “thiatriazolyl” as used herein includes 1,2,3,4-thiatriazol-5-yl and 1,2,3,5-thiatriazol-4-yl. The term “pyridinyl” (also called “pyridyl”) as used herein includes pyridin-2-yl, pyridin-3-yl and pyridin-4-yl (also called 2-pyridyl, 3-pyridyl and 4-pyridyl). The term “pyrimidyl” as used herein includes pyrimid-2-yl, pyrimid-4-yl, pyrimid-5-yl and pyrimid-6-yl. The term “pyrazinyl” as used herein includes pyrazin-2-yl and pyrazin-3-yl. The term “pyridazinyl as used herein includes pyridazin-3-yl and pyridazin-4-yl. The term “oxazinyl” (also called “1,4-oxazinyl”) as used herein includes 1,4-oxazin-4-yl and 1,4-oxazin-5-yl. The term “dioxinyl” (also called “1,4-dioxinyl”) as used herein includes 1,4-dioxin-2-yl and 1,4-dioxin-3-yl. The term “thiazinyl” (also called “1,4-thiazinyl”) as used herein includes 1,4-thiazin-2-yl, 1,4-thiazin-3-yl, 1,4-thiazin-4-yl, 1,4-thiazin-5-yl and 1,4-thiazin-6-yl. The term “triazinyl” as used herein includes 1,3,5-triazin-2-yl, 1,2,4-triazin-3-yl, 1,2,4-triazin-5-yl, 1,2,4-triazin-6-yl, 1,2,3-triazin-4-yl and 1,2,3-triazin-5-yl. The term “imidazo[2,1-b][1,3]thiazolyl” as used herein includes imidazo[2,1-b][1,3]thiazol-2-yl, imidazo[2,1-b][1,3]thiazol-3-yl, imidazo[2,1-b][1,3]thiazol-5-yl and imidazo[2,1-b][1,3]thiazol-6-yl. The term “thieno[3,2-b]furanyl” as used herein includes thieno[3,2-b]furan-2-yl, thieno[3,2-b]furan-3-yl, thieno[3,2-b]furan-4-yl, and thieno[3,2-b]furan-5-yl. The term “thieno[3,2-b]thiophenyl” as used herein includes thieno[3,2-b]thien-2-yl, thieno[3,2-b]thien-3-yl, thieno[3,2-b]thien-5-yl and thieno[3,2-b]thien-6-yl. The term “thieno[2,3-d][1,3]thiazolyl” as used herein includes thieno[2,3-d][1,3]thiazol-2-yl, thieno[2,3-d][1,3]thiazol-5-yl and thieno[2,3-d][1,3]thiazol-6-yl. The term “thieno[2,3-d]imidazolyl” as used herein includes thieno[2,3-d]imidazol-2-yl, thieno[2,3-d]imidazol-4-yl and thieno[2,3-d]imidazol-5-yl. The term “tetrazolo[1,5-a]pyridinyl” as used herein includes tetrazolo[1,5-a]pyridine-5-yl, tetrazolo[1,5-a]pyridine-6-yl, tetrazolo[1,5-a]pyridine-7-yl, and tetrazolo[1,5-a]pyridine-8-yl. The term “indolyl” as used herein includes indol-1-yl, indol-2-yl, indol-3-yl,-indol-4-yl, indol-5-yl, indol-6-yl and indol-7-yl. The term “indolizinyl” as used herein includes indolizin-1-yl, indolizin-2-yl, indolizin-3-yl, indolizin-5-yl, indolizin-6-yl, indolizin-7-yl, and indolizin-8-yl. The term “isoindolyl” as used herein includes isoindol-1-yl, isoindol-2-yl, isoindol-3-yl, isoindol-4-yl, isoindol-5-yl, isoindol-6-yl and isoindol-7-yl. The term “benzofuranyl” (also called benzo[b]furanyl) as used herein includes benzofuran-2-yl, benzofuran-3-yl, benzofuran-4-yl, benzofuran-5-yl, benzofuran-6-yl and benzofuran-7-yl. The term “isobenzofuranyl” (also called benzo[c]furanyl) as used herein includes isobenzofuran-1-yl, isobenzofuran-3-yl, isobenzofuran-4-yl, isobenzofuran-5-yl, isobenzofuran-6-yl and isobenzofuran-7-yl. The term “benzothiophenyl” (also called benzo[b]thienyl) as used herein includes 2-benzo[b]thiophenyl, 3-benzo[b]thiophenyl, 4-benzo[b]thiophenyl, 5-benzo[b]thiophenyl, 6-benzo[b]thiophenyl and -7-benzo[b]thiophenyl (also called benzothien-2-yl, benzothien-3-yl, benzothien-4-yl, benzothien-5-yl, benzothien-6-yl and benzothien-7-yl). The term “isobenzothiophenyl” (also called benzo[c]thienyl) as used herein includes isobenzothien-1-yl, isobenzothien-3-yl, isobenzothien-4-yl, isobenzothien-5-yl, isobenzothien-6-yl and isobenzothien-7-yl. The term “indazolyl” (also called 1H-indazolyl or 2-azaindolyl) as used herein includes 1H-indazol-1-yl, 1H-indazol-3-yl, 1H-indazol-4-yl, 1H-indazol-5-yl, 1H-indazol-6-yl, 1H-indazol-7-yl, 2H-indazol-2-yl, 2H-indazol-3-yl, 2H-indazol-4-yl, 2H-indazol-5-yl, 2H-indazol-6-yl, and 2H-indazol-7-yl. The term “benzimidazolyl” as used herein includes benzimidazol-1-yl, benzimidazol-2-yl, benzimidazol-4-yl, benzimidazol-5-yl, benzimidazol-6-yl and benzimidazol-7-yl. The term “1,3-benzoxazolyl” as used herein includes 1,3-benzoxazol-2-yl, 1,3-benzoxazol-4-yl, 1,3-benzoxazol-5-yl, 1,3-benzoxazol-6-yl and 1,3-benzoxazol-7-yl. The term “1,2-benzisoxazolyl” as used herein includes 1,2-benzisoxazol-3-yl, 1,2-benzisoxazol-4-yl, 1,2-benzisoxazol-5-yl, 1,2-benzisoxazol-6-yl and 1,2-benzisoxazol-7-yl. The term “2,1-benzisoxazolyl” as used herein includes 2,1-benzisoxazol-3-yl, 2,1-benzisoxazol-4-yl, 2,1-benzisoxazol-5-yl, 2,1-benzisoxazol-6-yl and 2,1-benzisoxazol-7-yl. The term “1,3-benzothiazolyl” as used herein includes 1,3-benzothiazol-2-yl, 1,3-benzothiazol-4-yl, 1,3-benzothiazol-5-yl, 1,3-benzothiazol-6-yl and 1,3-benzothiazol-7-yl. The term “1,2-benzoisothiazolyl” as used herein includes 1,2-benzisothiazol-3-yl, 1,2-benzisothiazol-4-yl, 1,2-benzisothiazol-5-yl, 1,2-benzisothiazol-6-yl and 1,2-benzisothiazol-7-yl. The term “2,1-benzoisothiazolyl” as used herein includes 2,1-benzisothiazol-3-yl, 2,1-benzisothiazol-4-yl, 2,1-benzisothiazol-5-yl, 2,1-benzisothiazol-6-yl and 2,1-benzisothiazol-7-yl. The term “benzotriazolyl” as used herein includes benzotriazol-1-yl, benzotriazol4-yl, benzotriazol-5-yl, benzotriazol-6-yl and benzotriazol-7-yl. The term “1,2,3-benzoxadiazolyl” as used herein includes 1,2,3-benzoxadiazol-4-yl, 1,2,3-benzoxadiazol-5-yl, 1,2,3-benzoxadiazol-6-yl and 1,2,3-benzoxadiazol-7-yl. The term “2,1,3-benzoxadiazolyl” as used herein includes 2,1,3-benzoxadiazol-4-yl, 2,1,3-benzoxadiazol-5-yl, 2,1,3-benzoxadiazol-6-yl and 2,1,3-benzoxadiazol-7-yl. The term “1,2,3-benzothiadiazolyl” as used herein includes 1,2,3-benzothiadiazol-4-yl, 1,2,3-benzothiadiazol-5-yl, 1,2,3-benzothiadiazol-6-yl and 1,2,3-benzothiadiazol-7-yl. The term “2,1,3-benzothiadiazolyl” as used herein includes 2,1,3-benzothiadiazol-4-yl, 2,1,3-benzothiadiazol-5-yl, 2,1,3-benzothiadiazol-6-yl and 2,1,3-benzothiadiazol-7-yl. The term “thienopyridinyl” as used herein includes thieno[2,3-b]pyridinyl, thieno[2,3-c]pyridinyl, thieno[3,2-c]pyridinyl and thieno[3,2-b]pyridinyl. The term “purinyl” as used herein includes purin-2-yl, purin-6-yl, purin-7-yl and purin-8-yl. The term “imidazo[1,2-a]pyridinyl”, as used herein includes imidazo[1,2-a]pyridin-2-yl, imidazo[1,2-a]pyridin-3-yl, imidazo[1,2-a]pyridin-4-yl, imidazo[1,2-a]pyridin-5-yl, imidazo[1,2-a]pyridin-6-yl and imidazo[1,2-a]pyridin-7-yl. The term “1,3-benzodioxolyl”, as used herein includes 1,3-benzodioxol-4-yl, 1,3-benzodioxol-5-yl, 1,3-benzodioxol-6-yl, and 1,3-benzodioxol-7-yl. The term “quinolinyl” as used herein includes quinolin-2-yl, quinolin-3-yl, quinolin-4-yl, quinolin-5-yl, quinolin-6-yl, quinolin-7-yl and quinolin-8-yl. The term “isoquinolinyl” as used herein includes isoquinolin-1-yl, isoquinolin-3-yl, isoquinolin-4-yl, isoquinolin-5-yl, isoquinolin-6-yl, isoquinolin-7-yl and isoquinolin-8-yl. The term “cinnolinyl” as used herein includes cinnolin-3-yl, cinnolin-4-yl, cinnolin-5-yl, cinnolin-6-yl, cinnolin-7-yl and cinnolin-8-yl. The term “quinazolinyl” as used herein includes quinazolin-2-yl, quiriazolin-4-yl, quinazolin-5-yl, quinazolin-6-yl, quinazolin-7-yl and quinazolin-8-yl. The term “quinoxalinyl”. as used herein includes quinoxalin-2-yl, quinoxalin-5-yl, and quinoxalin-6-yl.

The term “heteroalkyl”, as a group or part of a group, also encompasses groups of Formula —X—R^{f} or —R^{e}—X—R^{f}, and alkyl substituted with one or more groups of formula —X—R^{f} or —R^{e}—X—R^{f}, wherein R^{e} is as defined above for C₁₋₆alkylene and X is NR^{c}, S or O, and R^{c} is selected from hydrogen, or C₁₋₆alkyl, and R^{f} is hydrogen, C₁₋₆acyl, C₁₋₆alkyl, C₃₋₆cycloalkyl; Representative examples include, but are not limited to, 2-hydroxyethyl, 3-hydroxypropyl, 2-hydroxy-1-hydroxymethylethyl, 2,3-dihydroxypropyl, 1-hydroxymethylethyl, 3-hydroxybutyl, 2,3-dihydroxybutyl, 2-hydroxy-1-methylpropyl, 2-aminoethyl, 3-aminopropyl, and the like.

The term “heterocyclylC₁₋₆alkyl”, as a group or part of a group, refers to a group or formula —R^{e}—R^{g} wherein R^{e} is C₁₋₆alkylene and R^{g} is a heterocyclyl group as defined herein.

The term “polymeric group” as used herein also encompasses oligomeric groups, and refers to a compound containing more than 8 repeat units, preferably more than 10, preferably more than 20, preferably more than 50, preferably more than 100 repeat units. The polymeric group may be any polymer in which the monomer reactive groups include epoxies, amines, vinyl groups, methacrylates, isobornyl acrylates; alcohols, carboxyl groups, aldehydes, or isocyanates, and the like. In some embodiments, the polymer group may include a polyurethane; or an isocyanate-containing prepolymer, an hydroxyl containing prepolymer, a polyacrylate such as polymethylacrylate, or poly(ethyl acrylate); a poly(alkylacrylate) such as poly(methyl methacrylate); a polyurethane; a poly(isobornyl acrylate); a polyamide such as nylon; a polyester such as poly(ethylene terephthalate) and polycaprolactone; a polycarbonate; a polyether; an epoxy polymer; a vinyl ester polymer; a polyimide such as polypyromellitimide (for example KAPTAN); a phenol-formaldehyde polymer such as BAKELITE; an amine-formaldehyde polymer such as a melamine polymer; a polysulfone; a poly(acrylonitrile-butadiene-styrene) (ABS); a polyolefin such as polyethylene, polystyrene, polyacrylonitrile, a polyvinyl, polyvinyl chloride and poly(DCPD); a polysilane such as poly(carborane-siloxane); a polyphosphazene and/or hydrogenated derivatives thereof.

The term “polyurethane” as used herein encompasses polymers composed of organic units joined by carbamate (urethane) bonds (—NH—CO—O—) in the framework. Generally, the framework of the polymer may optionally contain, in addition to the urethane bonds, ester bonds, ether bonds, urea bonds, urea-based urethane bonds, isocyanurate bonds, and the like. When one of R¹, R², R³, R⁴, R⁶, R⁷, R⁸, R⁹, R¹⁰, R¹¹, R¹², R¹⁴, R¹⁵, R¹⁶, R¹⁷, Z⁵, Z^{5a}, Z⁷, Z⁸, Z^{8a} or Z⁹ as defined herein is selected as a polyurethane polymeric group, it is understood that a carbamate (urethane) group is directly attached to the rest of the compound of Formulae (I), (IB), (A) or (A2) and any subgroup thereof.

The term “leaving group” as used herein means a chemical group which is susceptible to be displaced by a nucleophile or cleaved off or hydrolyzed in basic or acidic conditions. In a particular embodiment, a leaving group is selected from a halogen atom (e.g., Cl, Br, I) or a sulfonate (e.g., mesylate, tosylate, triflate).

Whenever used in the present invention the term “compounds of the invention” or a similar term is meant to include the compounds of general Formulae (I), (IB), (A) or (A2) and any subgroup thereof. This term also encompasses N-oxides form thereof, as well as quaternized nitrogen analogues. The N-oxide forms of said compounds are meant to comprise compounds wherein one or several nitrogen atoms are oxidized to the so-called N-oxide.

The terms described above and others used in the specification are well understood to those in the art.

Preferred statements (features), and embodiments of the compounds and processes of this invention are now set forth, each statements and embodiments of the invention so defined may be combined with any other statement and/or embodiments unless clearly indicated to the contrary. In particular, any feature indicated as being preferred or advantageous may be combined with any other feature or features indicated as being preferred or advantageous.

Numbered statements of this invention are:

1. A compound of formula (I) or a stereoisomer, enantiomer, racemic, or tautomer thereof,

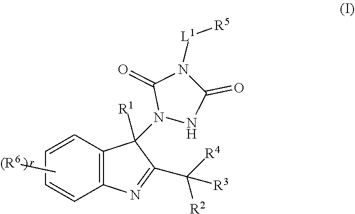

wherein,
r is an integer selected from 0, 1, 2, 3 or 4;
R¹ is selected from the group consisting of C₁₋₁₀alkyl; C₂₋₁₀alkenyl; C₂₋₁₀alkynyl; C₆₋₁₂aryl; C₃₋₈cycloalkyl; C₆₋₁₂arylC₁₋₆alkyl; heterocyclyl; heteroaryl; heterocyclylC₁₋₆alkyl; heteroarylC₁₋₆alkyl; —OR¹⁹; —SR¹⁹; NR²⁰R²¹; —C(O)NR²⁰R²¹; OH, SH, COOH, NH₂; NHR²⁰; and a polymeric group;
   and wherein said C₁₋₁₀alkyl; C₂₋₁₀alkenyl; C₂₋₁₀alkynyl, C₆₋₁₂aryl, C₃₋₈cycloalkyl; C₆₋₁₂arylC₁₋₆alkyl, heterocyclyl; heteroaryl; heterocyclylC₁₋₆alkyl; or heteroarylC₁₋₆alkyl; can be unsubstituted or substituted with one or more Z¹;
   and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
   or R¹ is a group of formula (i);

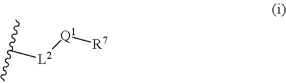

wherein the wavy line ( [symbol] ) indicates the point of attachment to the indole ring of formula (I);
L² is selected from the group consisting of C₁₋₁₀alkylene; C₃₋₆cycloalkylene; C₂₋₁₀alkenylene; C₂₋₁₀alkynylene; C₆₋₁₂arylene; and C₆₋₁₂arylC₁₋₆alkylene;
   and wherein said C₁₋₁₀alkylene; C₃₋₆cycloalkylene; C₂₋₁₀alkenylene; C₂₋₁₀alkynylene; C₆₋₁₂arylene or C₆₋₁₂arylC₁₋₆alkylene, can be unsubstituted or substituted with one or more Z²;
Q¹ is a single bond or selected from the group consisting of C₁₋₁₀alkylene; C₃₋₆cycloalkylene; —O—; —S—; —O—C₁₋₁₀alkylene; and —S—C₁₋₁₀alkylene; wherein each left side of said groups is attached to L² and the right side thereof is attached to R⁷;
   and wherein said C₁₋₁₀alkylene and said C₃₋₆cycloalkylene; can be unsubstituted or substituted with one or more Z³;
R⁷ is selected from the group consisting of hydrogen; OH, SH, COOH, NH₂; NHR²⁰, halo; nitro; cyano; —C(O)NR²⁰R²¹; C₁₋₂₀alkyl, C₂₋₂₀alkenyl; C₂₋₂₀alkynyl; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
   wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl; can be unsubstituted or substituted with one or more Z⁴;
   and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
or R⁷ is a group of formula (ii);

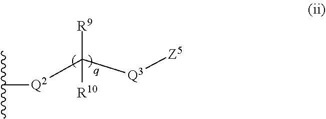

wherein the wavy line ( [symbol] ) indicates the point of attachment to Q¹;
q is an integer selected from 0; 1, 2, 3 or 4;
Q² is a single bond or selected from the group consisting of CR¹¹R¹²; O, S, and NR¹³;
Q³ is a single bond or selected from the group consisting of hydroxyC₁₋₁₀alkylene; aminoC₁₋₁₀alkylene; mercaptoC₁₋₁₀alkylene; C₁₋₂₀alkylene; C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₆alkylene; and heteroarylC₁₋₆alkylene;
wherein said C₁₋₂₀alkylene, C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₈alkylene; or heteroarylC₁₋₆alkylene, can be unsubstituted or substituted with one or more Z^{5a};
wherein if q is 0, then one of Q² and Q³ is not a single bond;
each R⁹ and R¹⁰ is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
each R¹¹ and R¹² is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
R¹³ is selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; and heteroarylC₁₋₆alkyl;
R² is selected from the group consisting of halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; heteroarylC₁₋₆alkyl; and a polymeric group;
R³ is selected from the group consisting of halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; heteroarylC₁₋₆alkyl; and a polymeric group;
R⁴ is selected from the group consisting of halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; heteroarylC₁₋₆alkyl; and a polymeric group;
   wherein for R², R³ and R⁴, each independently; said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl, heteroarylC₁₋₆alkyl, can be unsubstituted or substituted with one or more Z⁶;
   and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
or R² and R³ and R⁴ together with the carbon atom to which they are attached can form a saturated or unsaturated 4-, 5-, 6-, 7- or 8-membered ring;
   wherein said saturated or unsaturated 4-, 5-, 6-, 7- or 8-membered ring optionally comprises one or more heteroatoms, said heteroatoms being each independently selected from O, S and N;
   wherein said saturated or unsaturated 4-, 5-, 6-, 7- or 8-membered ring can be unsubstituted or substituted with one or more R⁸;
each R⁸ is independently selected from the group consisting of hydroxyC₁₋₁₀alkoxy; aminoC₁₋₁₀alkoxy; mercaptoC₁₋₁₀alkoxy; C₁₋₁₀alkyl; C₂₋₁₀alkenyl; C₂₋₁₀alkynyl; C₆₋₁₂arylC₁₋₆alkyl; C₃₋₈cycloalkyl; C₆₋₁₂arylC₁₋₆alkyl; heterocyclyl; heteroaryl; heterocyclylC₁₋₆alkyl; heteroarylC₁₋₆alkyl; OH; SH; COOH; NH₂; NHR²⁰; halogen; haloC₁₋₆alkyl; haloC₁₋₆alkyloxy; —OR¹⁹; —SR¹⁹; nitro; NR²⁰R²¹; —C(O)NR²⁰R²¹; cyano; and a polymeric group;
   wherein said hydroxyC₁₋₁₀alkoxy; aminoC₁₋₁₀alkoxy; mercaptoC₁₋₁₀alkoxy; C₁₋₁₀alkyl; C₂₋₁₀alkenyl; C₂₋₁₀alkynyl; C₆₋₁₂arylC₁₋₆alkyl; C₃₋₈cycloalkyl; C₆₋₁₂arylC₁₋₆alkyl; heterocyclyl; heteroaryl; heterocyclylC₁₋₆alkyl; or heteroarylC₁₋₆alkyl, can be unsubstituted or substituted with one or more Z⁷;
   and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
   or R⁸ is a group of formula (iii);

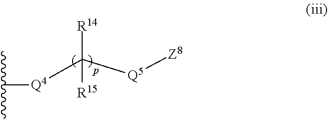

wherein the wavy line ( [symbol] ) indicates the point of attachment to the ring formed by R² and R³ and R⁴ together;
p is an integer selected from 0; 1, 2, 3 or 4;
Q⁴ is a single bond or selected from the group consisting of CR¹⁶R¹⁷; O, S, and NR¹⁸;
Q⁵ is a single bond or selected from the group consisting of hydroxyC₁₋₁₀alkylene; aminoC₁₋₁₀alkylene; mercaptoC₁₋₁₀alkylene; C₁₋₂₀alkylene, C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₆alkylene; and heteroarylC₁₋₆alkylene;
wherein said C₁₋₂₀alkylene, C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₈alkylene; or heteroarylC₁₋₆alkylene, can be unsubstituted or substituted with one or more Z^{8a};
wherein if p is 0, then one of Q⁴ and Q⁵ is not a single bond;
each R¹⁴ and R¹⁵ is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
each R¹⁶ and R¹⁷ is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
R¹⁸ is selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; and heteroarylC₁₋₆alkyl;
each R⁶ is independently selected from the group consisting of hydrogen, halogen; C₁₋₁₀alkyl; C₂₋₁₀alkenyl; C₂₋₁₀alkynyl; C₆₋₁₂arylC₁₋₆alkyl; haloC₁₋₆alkyl; haloC₁₋₆alkyloxy; —OR¹⁹; —SR¹⁹; nitro; NR²⁰R²¹; —C(O)NR²⁰R²¹; cyano; and a polymeric group;
   wherein said C₁₋₁₀alkyl; C₂₋₁₀alkenyl; C₂₋₁₀alkynyl; or C₆₋₁₂arylC₁₋₆alkyl, can be unsubstituted or substituted with one or more Z⁹;
   and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
L¹ is selected from the group consisting of C₁₋₁₀alkyleneC₃₋₈cycloalkylene; C₃₋₈cycloalkyleneC₁₋₁₀alkylene; C₁₋₁₀alkylene; C₃₋₈cycloalkylene; C₃₋₈cycloalkyleneC₁₋₁₀alkylC₃₋₈cycloalkylene; C₂₋₁₀alkenylene; C₂₋₁₀alkynylene; C₆₋₁₂arylene; C₆₋₁₂aryleneC₁₋₆alkylene; C₆₋₁₂aryleneC₁₋₆alkylC₆₋₁₂arylene; and C₁₋₆alkyleneC₆₋₁₂arylC₁₋₆alkylene;
   wherein said C₁₋₁₀alkyleneC₃₋₈cycloalkylene; C₃₋₈cycloalkyleneC₁₋₁₀alkylene; C₁₋₁₀alkylene; C₃₋₈cycloalkylene; C₃₋₈cycloalkyleneC₁₋₁₀alkylC₃₋₈cycloalkylene; C₂₋₁₀alkenylene; C₂₋₁₀alkynylene; C₆₋₁₂arylene; C₆₋₁₂aryleneC₁₋₆alkylene; C₆₋₁₂aryleneC₁₋₆alkylC₆₋₁₂arylene; or C₁₋₆alkyleneC₆₋₁₂arylC₁₋₆alkylene, can be unsubstituted or substituted with one or more Z^{1a};
or L¹ is selected from a group of formula (iv);

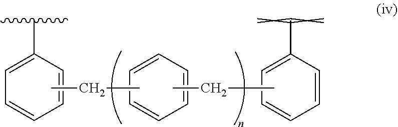

wherein the wavy line ( [symbol] ) indicates the point of attachment to the nitrogen of the formula (I) and the crossed line ( [symbol] ) indicates the point of attachment to R⁵;
wherein n is an integer selected from 0, 1, 2, 3, 4, 5, 6, 7, 8, 9, or 10;
R⁵ is selected from the group consisting of OH, SH, COOH, NH₂, and NHR²⁰;
or R⁵ is a group of formula (v);

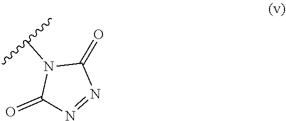 [SMILES: *N1C(=O)N=NC1=O]

wherein the wavy line ( [symbol] ) indicates the point of attachment to L¹;
or R⁵ is a group of formula (vi);

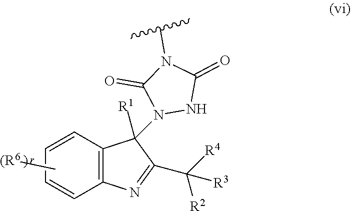

wherein the wavy line ( [symbol] ) indicates the point of attachment to L¹, and r, R¹, R², R³, R⁴ and R⁶ are as defined for structure (I);
each R¹⁹ is independently selected from the group consisting of C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl;
   wherein said C₁₋₁₀alkyl, C₂₋₂₀alkenyl, C₂₋₂₀alkynyl, C₆₋₁₂arylC₁₋₆alkyl, heterocyclylC₁₋₆alkyl; heteroarylC₁₋₆alkyl, optionally comprise one or more heteroatoms in the alkyl, alkenyl or alkynyl moiety, said heteroatoms being each independently selected from O, S and N;
   wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl, can be unsubstituted or substituted with one or more OH, SH, COOH, or NH₂;
each R²⁰ is independently selected from the group consisting of C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl;
   wherein said C₁₋₁₀alkyl, C₂₋₂₀alkenyl, C₂₋₂₀alkynyl, C₆₋₁₂arylC₁₋₆alkyl, heterocyclylC₁₋₆alkyl; heteroarylC₁₋₆alkyl, optionally comprise one or more heteroatoms in the alkyl, alkenyl or alkynyl moiety, said heteroatoms being each independently selected from O, S and N;
   wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl, can be unsubstituted or substituted with one or more OH, SH, COOH, or NH₂;
each R²¹ is independently selected from the group consisting of hydrogen, C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl;
   wherein said C₁₋₁₀alkyl, C₂₋₂₀alkenyl, C₂₋₂₀alkynyl, C₆₋₁₂arylC₁₋₆alkyl, heterocyclylC₁₋₆alkyl; heteroarylC₁₋₆alkyl, optionally comprise one or more heteroatoms in the alkyl, alkenyl or alkynyl moiety, said heteroatoms being each independently selected from O, S and N;
   wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl, can be unsubstituted or substituted with one or more OH, SH, COOH, or NH₂;
or wherein R²⁰ and R²¹ together with the atom to which they are attached form a 5-, 6-, or 7-membered heterocycle.
each Z^{1a} is selected from the group consisting of C₁₋₆alkyl; C₂₋₆alkenyl; C₂₋₆alkynyl; haloC₁₋₆alkyl; haloC₁₋₆alkyloxy; halogen; isocyanate;
or Z^{1a} is a group of formula (vii);

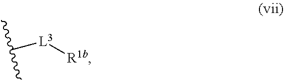

wherein the wavy line ( [symbol] ) indicates the point of attachment to L¹;
   L³ is a single bond or is selected from the group consisting of C₁₋₁₀alkylene; C₂₋₁₃alkenylene; C₂₋₁₃alkynylene; and C₆₋₁₂aryl;

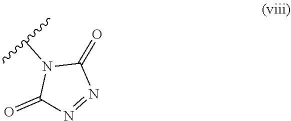 [SMILES: *N1C(=O)N=NC1=O]

wherein the wavy line ( [symbol] ) indicates the point of attachment to L³;
or R^{1b} is a group of formula (ix);

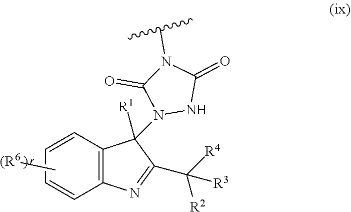

wherein the wavy line ( [symbol] ) indicates the point of attachment to L³ and r, R¹, R², R³, R⁴ and R⁶ are as defined for structure (I);
Z¹, Z², Z³, Z⁴, Z⁶ are each independently selected from the group consisting of OH; SH; COOH; NH₂; NHR²⁰; halogen; C₁₋₆alkyl; haloC₁₋₆alkyl; haloC₁₋₆alkyloxy; —OR¹⁹; —SR¹⁹; nitro; NR²⁰R²¹; —C(O)NR²⁰R²¹; cyano;
Z⁵, Z^{5a}, Z⁷, Z⁸, Z^{8a}, Z⁹ are each independently selected from the group consisting of OH; hydrogen; SH; COOH; NH₂; NHR²⁰; halogen; C₁₋₆alkyl; haloC₁₋₆alkyl; haloC₁₋₆alkyloxy; —OR¹⁹; —SR¹⁹; nitro; NR²⁰R²¹; —C(O)NR²⁰R²¹; cyano; and a polymeric group;
   and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
and when none of R¹, R², R³, R⁴, R⁶, R⁷, R⁸, R⁹, R¹⁹, R¹¹, R¹², R¹⁴, R¹⁵, R¹⁶, R¹⁷, Z⁵, Z^{5a}, Z⁷, Z⁸, Z^{8a} or Z⁹ is a polymeric group, then at least one of R⁷, R⁸, Z¹, Z², Z³, Z⁴, Z⁵, Z^{5a}, Z⁶, Z⁷, Z⁸, Z^{8a} or Z⁹ is selected from the group consisting of OH, SH, COOH, NH₂, and NHR²⁰.

2. The compound according to statement 1, wherein,
R¹ is selected from the group consisting of C₁₋₁₀alkyl; C₆₋₁₂aryl; C₃₋₈cycloalkyl; C₆₋₁₂arylC₁₋₆alkyl; —OR¹⁹; —SR¹⁹; NR²⁰R²¹; —C(O)NR²⁰R²¹; OH, SH, COOH, NH₂; NHR²⁰; and a polymeric group, wherein said C₁₋₁₀alkyl; C₆₋₁₂aryl; C₃₋₈cycloalkyl; or C₆₋₁₂arylC₁₋₆alkyl, can be unsubstituted or substituted with one or more Z¹;
R² is selected from the group consisting of halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; C₆₋₁₂arylC₁₋₆alkyl; and a polymeric group,
R³ is selected from the group consisting of halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; C₆₋₁₂arylC₁₋₆alkyl; and a polymeric group,
R⁴ is selected from the group consisting of halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; C₆₋₁₂arylC₁₋₆alkyl; and a polymeric group, wherein for R², R³ and R⁴, each independently; said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, can be unsubstituted or substituted with one or more Z⁶;
or R² and R³ and R⁴ together with the carbon atom to which they are attached form a saturated or unsaturated 4-, 5-, or 6-membered ring;
wherein said saturated or unsaturated 4-, 5-, or 6-membered ring can be unsubstituted or substituted with one or more R⁸;
each R⁸ is independently selected from the group consisting of hydroxyC₁₋₁₀alkoxy; aminoC₁₋₁₀alkoxy; mercaptoC₁₋₁₀alkoxy; C₁₋₁₀alkyl; C₂₋₁₀alkenyl; C₂₋₁₀alkynyl; C₆₋₁₂arylC₁₋₆alkyl; C₃₋₈cycloalkyl; C₆₋₁₂arylC₁₋₆alkyl; heterocyclyl; heteroaryl; heterocyclylC₁₋₆alkyl; heteroarylC₁₋₆alkyl; —OR¹⁹; —SR¹⁹; and a polymeric group;
   wherein said hydroxyC₁₋₁₀alkoxy; aminoC₁₋₁₀alkoxy; mercaptoC₁₋₁₀alkoxy; C₁₋₁₀alkyl; C₂₋₁₀alkenyl; C₂₋₁₀alkynyl; C₆₋₁₂arylC₁₋₆alkyl; C₃₋₈cycloalkyl; C₆₋₁₂arylC₁₋₆alkyl; heterocyclyl; heteroaryl; heterocyclylC₁₋₆alkyl; or heteroarylC₁₋₆alkyl, can be unsubstituted or substituted with one or more Z⁷;
      or R⁸ is a group of formula (iii);

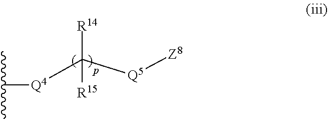

wherein the wavy line ( [symbol] ) indicates the point of attachment to the ring formed by R² and R³ and R⁴ together;
p is an integer selected from 0; 1, 2, or 3; Q⁴ is a single bond or selected from the group consisting of CR¹⁶R¹⁷; O, S, and NR¹⁸;
Q⁵ is a single bond or selected from the group consisting of hydroxyC₁₋₁₀alkylene; aminoC₁₋₁₀alkylene; mercaptoC₁₋₁₀alkylene; C₁₋₂₀alkylene, C₆₋₁₂arylene, C₃₋₈cycloalkylene; C₆₋₁₂arylC₁₋₆alkylene; heterocyclylene; heteroarylene; heterocyclylC₁₋₆alkylene; and heteroarylC₁₋₆alkylene;
wherein said C₁₋₂₀alkylene, C₆₋₁₂arylene, C₃₋₈cycloalkylene; C₆₋₁₂arylC₁₋₆alkylene; heterocyclylene; heteroarylene; heterocyclylC₁₋₆alkylene; or heteroarylC₁₋₆alkylene; can be unsubstituted or substituted with one or more Z^{8a};
wherein if p is 0, then one of Q⁴ and Q⁵ is not a single bond;
each R¹⁴ and R¹⁵ is independently selected from the group consisting of hydrogen; C₁₋₂₀alkyl, hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
   each R¹⁶ and R¹⁷ is independently selected from the group consisting of hydrogen; C₁₋₂₀alkyl, hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
   R¹⁸ is selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; and heteroarylC₁₋₆alkyl;
each R⁶ is independently selected from the group consisting of hydrogen, halogen; C₁₋₁₀alkyl; C₆₋₁₂arylC₁₋₆alkyl; haloC₁₋₆alkyl; haloC₁₋₆alkyloxy; —OR¹⁹; —SR¹⁹; nitro; NR²⁰R²¹; —C(O)NR²⁰R²¹; cyano; and a polymeric group;
   wherein said C₁₋₁₀alkyl; or C₆₋₁₂arylC₁₋₆alkyl, can be unsubstituted or substituted with one or more Z⁹;
L¹ is selected from the group consisting of C₁₋₁₀alkyleneC₃₋₈cycloalkylene; C₃₋₈cycloalkyleneC₁₋₁₀alkylene; C₁₋₁₀alkylene; C₃₋₈cycloalkylene; C₃₋₈cycloalkyleneC₁₋₁₀alkylC₃₋₈cycloalkylene; C₆₋₁₂arylene; C₆₋₁₂aryleneC₁₋₆alkylene; C₆₋₁₂aryleneC₁₋₆alkyleneC₆₋₁₂arylene; and C₁₋₆alkyleneC₆₋₁₂arylC₁₋₆alkylene;
   wherein said C₁₋₁₀alkyleneC₃₋₈cycloalkylene; C₃₋₈cycloalkyleneC₁₋₁₀alkylene; C₁₋₁₀alkylene; C₃₋₈cycloalkylene; C₃₋₈cycloalkyleneC₁₋₁₀alkylC₃₋₈cycloalkylene; C₆₋₁₂arylene; C₆₋₁₂aryleneC₁₋₆alkylene; C₆₋₁₂aryleneC₁₋₆alkyleneC₆₋₁₂arylene; or C₁₋₆alkyleneC₆₋₁₂arylC₁₋₆alkylene, can be unsubstituted or substituted with one or more Z^{1a};
R⁵ is selected from the group consisting of OH, SH, COOH, NH₂, and NHR²⁰;
or R⁵ is a group of formula (v);

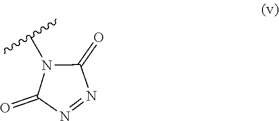 [SMILES: *N1C(=O)N=NC1=O]

wherein the wavy line ( [symbol] ) indicates the point of attachment to L¹;
or R⁵ is a group of formula (vi);

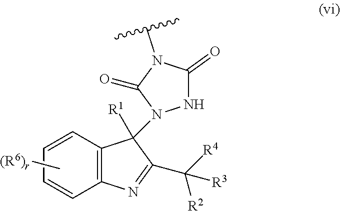

wherein the wavy line ( [symbol] ) indicates the point of attachment to L¹, and r, R¹, R², R³, R⁴ and R⁶ are as defined for structure (I);
each R¹⁹ is independently selected from the group consisting of C₁₋₂₀alkyl, C₆₋₁₂aryl, C₃₋₈cycloalkyl, and C₆₋₁₂arylC₁₋₆alkyl;
   wherein said C₁₋₂₀alkyl, C₆₋₁₂aryl, C₃₋₈cycloalkyl, C₆₋₁₂arylC₁₋₆alkyl, can be unsubstituted or substituted with one or more OH, SH, COOH, or NH₂;
each R²⁰ is independently selected from the group consisting of C₁₋₂₀alkyl, C₆₋₁₂aryl, C₃₋₈cycloalkyl, and C₆₋₁₂arylC₁₋₆alkyl;
   wherein said C₁₋₂₀alkyl, C₆₋₁₂aryl, C₃₋₈cycloalkyl, C₆₋₁₂arylC₁₋₆alkyl, can be unsubstituted or substituted with one or more OH, SH, COOH, or NH₂;
each R²¹ is independently selected from the group consisting of hydrogen; C₁₋₂₀alkyl, C₆₋₁₂aryl, C₃₋₈cycloalkyl, and C₆₋₁₂arylC₁₋₆alkyl;
   wherein said C₁₋₂₀alkyl, C₆₋₁₂aryl, C₃₋₈cycloalkyl, C₆₋₁₂arylC₁₋₆alkyl, can be unsubstituted or substituted with one or more OH, SH, COOH, or NH₂;
each Z^{1a} is selected from the group consisting of C₁₋₆alkyl; haloC₁₋₆alkyl; haloC₁₋₆alkyloxy; halogen; isocyanate;
or Z^{1a} is a group of formula (vii);

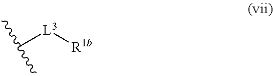

wherein the wavy line ( [symbol] ) indicates the point of attachment to L¹;
   L³ is a single bond or is selected from the group consisting of C₁₋₁₀alkylene; C₂₋₁₀alkenylene; C₂₋₁₀alkynylene; and C₆₋₁₂aryl;
   R^{1b} is a group of formula (viii);

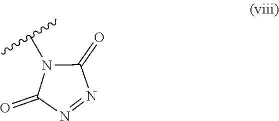 [SMILES: *N1C(=O)N=NC1=O]

wherein the wavy line ( [symbol] ) indicates the point of attachment to L³;
or R^{1b} is a group of formula (ix);

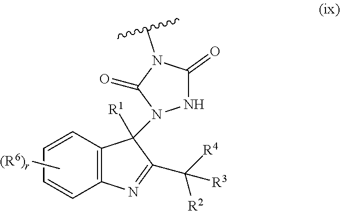

wherein the wavy line ( [symbol] ) indicates the point of attachment to L³ and r, R¹, R², R³, R⁴ and R⁶ are as defined for structure (I);
Z¹, Z⁶ are each independently selected from the group consisting of OH; SH; COOH; NH₂; NHR²⁰; halogen; C₁₋₆alkyl; haloC₁₋₆alkyl; haloC₁₋₆alkyloxy; —OR¹⁹; —SR¹⁹; nitro; NR²⁰R²¹; —C(O)NR²⁰R²¹; cyano;
Z⁷, Z⁸, Z^{8a}, Z⁹ are each selected from the group consisting of OH; hydrogen; SH; COOH; NH₂; NHR²⁰; halogen; C₁₋₆alkyl; haloC₁₋₆alkyl; haloC₁₋₆alkyloxy; —OR¹⁹; —SR¹⁹; nitro; NR²⁰R²¹; —C(O)NR²⁰R²¹; cyano; and a polymeric group.

3. The compound according to any one of statements 1 or 2, wherein,
R¹ is selected from the group consisting of C₁₋₁₀alkyl; C₆₋₁₂aryl; C₃₋₈cycloalkyl; C₆₋₁₂arylC₁₋₆alkyl; and a polymeric group, wherein said C₁₋₁₀alkyl; C₆₋₁₂aryl; C₃₋₈cycloalkyl; or C₆₋₁₂arylC₁₋₆alkyl, can be unsubstituted or substituted with one or more Z¹;
R² is selected from the group consisting of halo; nitro; cyano; —C(O)NR²⁰R²¹; C₁₋₂₀alkyl; C₆₋₁₂aryl; C₃₋₈cycloalkyl; C₆₋₁₂arylC₁₋₆alkyl; and a polymeric group,
R³ is selected from the group consisting of halo; nitro; cyano; —C(O)NR²⁰R²¹; C₁₋₂₀alkyl; C₆₋₁₂aryl; C₃₋₈cycloalkyl; C₆₋₁₂arylC₁₋₆alkyl; and a polymeric group,
R⁴ is selected from the group consisting of halo; nitro; cyano; —C(O)NR²⁰R²¹; C₁₋₂₀alkyl; C₆₋₁₂aryl; C₃₋₈cycloalkyl; C₆₋₁₂arylC₁₋₆alkyl; and a polymeric group,
   wherein for R², R³ and R⁴, each independently; said C₁₋₂₀alkyl, C₆₋₁₂aryl, C₃₋₈cycloalkyl, C₆₋₁₂arylC₁₋₆alkyl, can be unsubstituted or substituted with one or more Z⁶;
or R² and R³ and R⁴ together with the carbon atom to which they are attached form a saturated or unsaturated 5-, or 6-membered ring;
wherein said saturated or unsaturated 5-, or 6-membered ring can be unsubstituted or substituted with one or more R⁸;
each R⁸ is independently selected from the group consisting of hydroxyC₁₋₁₀alkoxy; aminoC₁₋₁₀alkoxy; mercaptoC₁₋₁₀alkoxy; C₁₋₁₀alkyl; C₆₋₁₂arylC₁₋₆alkyl; C₃₋₈cycloalkyl; C₆₋₁₂arylC₁₋₆alkyl; heterocyclyl; heteroaryl; heterocyclylC₁₋₆alkyl; heteroarylC₁₋₆alkyl; —OR¹⁹; —SR¹⁹; and a polymeric group;
   wherein said hydroxyC₁₋₁₀alkoxy; aminoC₁₋₁₀alkoxy; mercaptoC₁₋₁₀alkoxy; C₁₋₁₀alkyl; C₆₋₁₂arylC₁₋₆alkyl; C₃₋₈cycloalkyl; C₆₋₁₂arylC₁₋₆alkyl; heterocyclyl; heteroaryl; heterocyclylC₁₋₆alkyl; or heteroarylC₁₋₆alkyl, can be unsubstituted or substituted with one or more Z⁷;
   or R⁸ is a group of formula (iii);

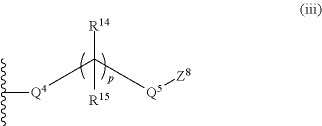

wherein the wavy line ( [symbol] ) indicates the point of attachment to the ring formed by R² and R³ and R⁴ together;
p is an integer selected from 1, 2, or 3;
Q⁴ is a single bond or selected from the group consisting of O, S, and NR¹⁸;
Q⁵ is a single bond or selected from the group consisting of hydroxyC₁₋₁₀alkylene; aminoC₁₋₁₀alkylene; mercaptoC₁₋₁₀alkylene; C₁₋₂₀alkylene, C₆₋₁₂arylene, C₃₋₈cycloalkylene;
wherein said C₁₋₂₀alkylene, C₆₋₁₂arylene, or C₃₋₈cycloalkylene; can be unsubstituted or substituted with one or more Z^{8a};
each R¹⁴ and R¹⁵ is independently selected from the group consisting of hydrogen; C₁₋₂₀alkyl, hydroxyC₁₋₁₀alkoxy; aminoC₁₋₁₀alkoxy; mercaptoC₁₋₁₀alkoxy; and a polymeric group;
R¹⁸ is selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; and a polymeric group;
each R⁶ is independently selected from the group consisting of hydrogen, halogen; C₁₋₁₀alkyl; C₆₋₁₂arylC₁₋₆alkyl; haloC₁₋₆alkyl; haloC₁₋₆alkyloxy; —OR¹⁹; —SR¹⁹; nitro; NR²⁰R²¹; —C(O)NR²⁰R²¹; cyano; and a polymeric group;
   wherein said C₁₋₁₀alkyl; or C₆₋₁₂arylC₁₋₆alkyl, can be unsubstituted or substituted with one or more Z⁹;
   L¹ is selected from the group consisting of C₁₋₁₀alkyleneC₃₋₈cycloalkylene; C₃₋₈cycloalkyleneC₁₋₁₀alkylene; C₁₋₁₀alkylene; C₃₋₈cycloalkylene; C₃₋₈cycloalkyleneC₁₋₁₀alkylC₃₋₈cycloalkylene; C₆₋₁₂arylene; C₆₋₁₂aryleneC₁₋₆alkylene; and C₆₋₁₂aryleneC₁₋₆alkylC₆₋₁₂arylene;
   wherein said C₁₋₁₀alkyleneC₃₋₈cycloalkylene; C₃₋₈cycloalkyleneC₁₋₁₀alkylene; C₁₋₁₀alkylene; C₃₋₈cycloalkylene; C₃₋₈cycloalkyleneC₁₋₁₀alkylC₃₋₈cycloalkylene; C₆₋₁₂arylene; C₆₋₁₂aryleneC₁₋₆alkylene; or C₆₋₁₂aryleneC₁₋₆alkylC₆₋₁₂arylene, can be unsubstituted or substituted with one or more Z^{1a};
R⁵ is selected from the group consisting of OH, SH, COOH, NH₂, and NHR²⁰;
or R⁵ is a group of formula (v);

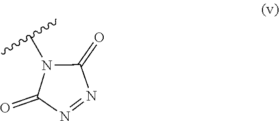 [SMILES: *N1C(=O)N=NC1=O]

wherein the wavy line ( [symbol] ) indicates the point of attachment to L¹;
or R⁵ is a group of formula (vi);

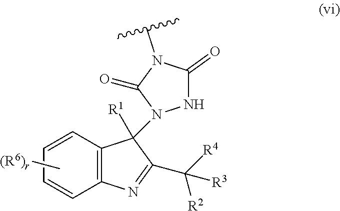

wherein the wavy line ( [symbol] ) indicates the point of attachment to L¹ and r, R¹, R², R³, R⁴ and R⁶ are as defined for structure (I);
each R¹⁹ is independently selected from the group consisting of C₁₋₂₀alkyl, C₆₋₁₂aryl, C₃₋₈cycloalkyl, and C₆₋₁₂arylC₁₋₆alkyl;
   wherein said C₁₋₂₀alkyl, C₆₋₁₂aryl, C₃₋₈cycloalkyl, C₆₋₁₂arylC₁₋₆alkyl, can be unsubstituted or substituted with one or more OH, SH, COOH, or NH₂;
each R²⁰ is independently selected from the group consisting of C₁₋₂₀alkyl, C₆₋₁₂aryl, C₃₋₈cycloalkyl, and C₆₋₁₂arylC₁₋₆alkyl;
   wherein said C₁₋₂₀alkyl, C₆₋₁₂aryl, C₃₋₈cycloalkyl, C₆₋₁₂arylC₁₋₆alkyl, can be unsubstituted or substituted with one or more OH, SH, COOH, or NH₂;
each R²¹ is independently selected from the group consisting of hydrogen; C₁₋₂₀alkyl, C₆₋₁₂aryl, C₃₋₈cycloalkyl, and C₆₋₁₂arylC₁₋₆alkyl;
   wherein said C₁₋₂₀alkyl, C₆₋₁₂aryl, C₃₋₈cycloalkyl, C₆₋₁₂arylC₁₋₆alkyl, can be unsubstituted or substituted with one or more OH, SH, COOH, or NH₂;
each Z^{1a} is selected from the group consisting of C₁₋₆alkyl; haloC₁₋₆alkyl; haloC₁₋₆alkyloxy; halogen;

or Z^{1a} is a group of formula (vii);

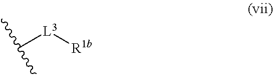

wherein the wavy line ( [symbol] ) indicates the point of attachment to L¹;
   L³ is a single bond or is selected from the group consisting of C₁₋₁₀alkylene; C₂₋₁₀alkenylene; C₂₋₁₀alkynylene; and C₆₋₁₂aryl;
   R^{1b} is a group of formula (viii);

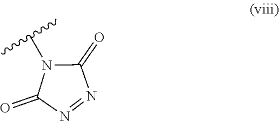 [SMILES: *N1C(=O)N=NC1=O]

wherein the wavy line ( [symbol] ) indicates the point of attachment to L³;
or R^{1b} is a group of formula (ix);

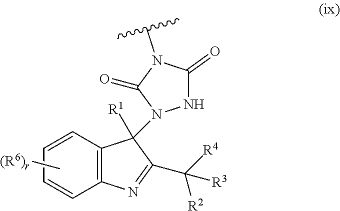

wherein the wavy line ( [symbol] ) indicates the point of attachment to L³ and r, R¹, R², R³, R⁴ and R⁶ are as defined for structure (I);
Z¹ are each independently selected from the group consisting of OH; SH; COOH; NH₂; NHR²⁰; halogen; C₁₋₆alkyl; —OR¹⁹; —SR¹⁹; nitro; NR²⁰R²¹; —C(O)NR²⁰R²¹; cyano;
Z⁷, Z⁸, Z^{8a}, Z⁹ are each selected from the group consisting of OH; hydrogen; SH; COOH; NH₂; NHR²⁰; halogen; C₁₋₆alkyl; —OR¹⁹; —SR¹⁹; nitro; NR²⁰R²¹; —C(O)NR²⁰R²¹; cyano; and a polymeric group.

4. The compound according to any one of statements 1 to 3, wherein
R¹ is selected from the group consisting of C₁₋₁₀alkyl; C₃₋₈cycloalkyl; and a polymeric group, wherein said C₁₋₁₀alkyl; or C₃₋₈cycloalkyl; can be unsubstituted or substituted with one or more Z¹;
R² is selected from the group consisting of halo; nitro; cyano; —C(O)NR²⁰R²¹; C₁₋₂₀alkyl; C₆₋₁₂aryl; C₃₋₈cycloalkyl; and a polymeric group,
R³ is selected from the group consisting of halo; nitro; cyano; —C(O)NR²⁰R²¹; C₁₋₂₀alkyl; C₆₋₁₂aryl; C₃₋₈cycloalkyl; and a polymeric group,
R⁴ is selected from the group consisting of halo; nitro; cyano; —C(O)NR²⁰R²¹; C₁₋₂₀alkyl; C₆₋₁₂aryl; C₃₋₈cycloalkyl; and a polymeric group,
   wherein for R², R³ and R⁴, each independently; said C₁₋₂₀alkyl, C₆₋₁₂aryl, or C₃₋₈cycloalkyl, can be unsubstituted or substituted with one or more Z⁶;
or R² and R³ and R⁴ together with the carbon atom to which they are attached form a saturated or unsaturated 5-, or 6-membered ring;
wherein said saturated or unsaturated 5-, or 6-membered ring can be unsubstituted or substituted with one or more R⁸;
each R⁸ is independently selected from the group consisting of hydroxyC₁₋₁₀alkoxy; aminoC₁₋₁₀alkoxy; mercaptoC₁₋₁₀alkoxy; C₁₋₁₀alkyl; C₆₋₁₂arylC₁₋₆alkyl; C₃₋₈cycloalkyl; C₆₋₁₂arylC₁₋₆alkyl; heterocyclylC₁₋₆alkyl; heteroarylC₁₋₆alkyl; —OR¹⁹; —SR¹⁹; and a polymeric group;
   wherein said hydroxyC₁₋₁₀alkoxy; aminoC₁₋₁₀alkoxy; mercaptoC₁₋₁₀alkoxy; C₁₋₁₀alkyl; C₆₋₁₂arylC₁₋₆alkyl; C₃₋₈cycloalkyl; C₆₋₁₂arylC₁₋₆alkyl; heterocyclylC₁₋₆alkyl; or heteroarylC₁₋₆alkyl, can be unsubstituted or substituted with one or more Z⁷;
   or R⁸ is a group of formula (iii);

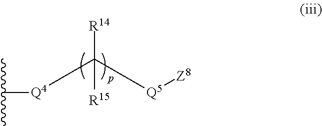

wherein the wavy line ( [symbol] ) indicates the point of attachment to the ring formed by R² and R³ and R⁴ together;
p is an integer selected from 1, 2, or 3;
Q⁴ is a single bond or selected from the group consisting of O, S, and NR¹⁸;
Q⁵ is a single bond or selected from the group consisting of hydroxyC₁₋₁₀alkylene; aminoC₁₋₁₀alkylene; mercaptoC₁₋₁₀alkylene; C₁₋₂₀alkylene, C₃₋₈cycloalkylene;
wherein said C₁₋₂₀alkylene, or C₃₋₈cycloalkylene; can be unsubstituted or substituted with one or more Z^{8a};
each R¹⁴ and R¹⁵ is independently selected from the group consisting of hydrogen; C₁₋₂₀alkyl, hydroxyC₁₋₁₀alkoxy; and a polymeric group;
R¹⁸ is selected from the group consisting of hydrogen; C₁₋₂₀alkyl, C₂₋₂₀alkenyl; C₂₋₂₀alkynyl; hydroxyC₁₋₁₀alkoxy; and a polymeric group;
each R⁶ is independently selected from the group consisting of hydrogen, halogen; C₁₋₁₀alkyl; C₆₋₁₂arylC₁₋₆alkyl; haloC₁₋₆alkyl; haloC₁₋₆alkyloxy; —C(O)NR²⁰R²¹; and a polymeric group; wherein said C₁₋₁₀alkyl; or C₆₋₁₂arylC₁₋₆alkyl, can be unsubstituted or substituted with one or more Z⁹;
L¹ is selected from the group consisting of C₁₋₁₀alkyleneC₃₋₈cycloalkylene; C₃₋₈cycloalkyleneC₁₋₁₀alkylene; C₁₋₁₀alkylene; C₃₋₈cycloalkylene; C₃₋₈cycloalkyleneC₁₋₁₀alkylC₃₋₈cycloalkylene; and C₆₋₁₂arylene;
   wherein said C₁₋₁₀alkyleneC₃₋₈cycloalkylene; C₃₋₈cycloalkyleneC₁₋₁₀alkylene; C₁₋₁₀alkylene; C₃₋₈cycloalkylene; C₃₋₈cycloalkyleneC₁₋₁₀alkylC₃₋₈cycloalkylene; or C₆₋₁₂arylene, can be unsubstituted or substituted with one or more Z^{1a};
R⁵ is selected from the group consisting of OH, SH, COOH, NH₂, and NHR²⁰;
or R⁵ is a group of formula (v);

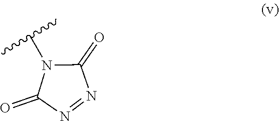 [SMILES: *N1C(=O)N=NC1=O]

wherein the wavy line ( [symbol] ) indicates the point of attachment to L¹;
or R⁵ is a group of formula (vi);

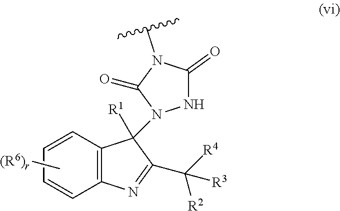

wherein the wavy line ( [symbol] ) indicates the point of attachment to L¹, and r, R¹, R², R³, R⁴ and R⁶ are as defined for structure (I);
each R¹⁹ is independently selected from the group consisting of C₁₋₂₀alkyl, C₃₋₈cycloalkyl, and C₆₋₁₂arylC₁₋₆alkyl;
   wherein said C₁₋₂₀alkyl, C₃₋₈cycloalkyl, C₆₋₁₂arylC₁₋₆alkyl, can be unsubstituted or substituted with one or more OH, SH, COOH, or NH₂;
each R²⁰ is independently selected from the group consisting of C₁₋₂₀alkyl, C₃₋₈cycloalkyl, and C₆₋₁₂arylC₁₋₆alkyl;
   wherein said C₁₋₂₀alkyl, C₃₋₈cycloalkyl, C₆₋₁₂arylC₁₋₆alkyl, can be unsubstituted or substituted with one or more OH, SH, COOH, or NH₂;
each R²¹ is independently selected from the group consisting of hydrogen; C₁₋₂₀alkyl, C₃₋₈cycloalkyl, and C₆₋₁₂arylC₁₋₆alkyl;
   wherein said C₁₋₂₀alkyl, C₃₋₈cycloalkyl, C₆₋₁₂arylC₁₋₆alkyl, can be unsubstituted or substituted with one or more OH, SH, COOH, or NH₂;
each Z^{1a} is selected from the group consisting of C₁₋₆alkyl; haloC₁₋₆alkyl; haloC₁₋₆alkyloxy; halogen;
or Z^{1a} is a group of formula (vii);

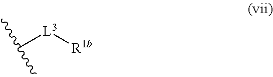

wherein the wavy line ( [symbol] ) indicates the point of attachment to L¹;
   L³ is a single bond or is selected from the group consisting of C₁₋₁₀alkylene; C₂₋₁₀alkenylene; C₂₋₁₀alkynylene; and C₆₋₁₂aryl;
   R^{1b} is a group of formula (viii);

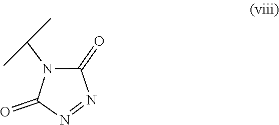

wherein the wavy line ( [symbol] ) indicates the point of attachment to L³;
or R^{1b} is a group of formula (ix);

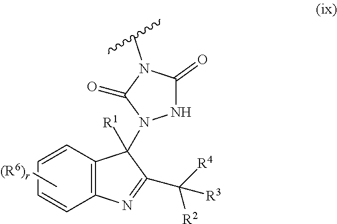

wherein the wavy line ( [symbol] ) indicates the point of attachment to L³ and r, R¹, R², R³, R⁴ and R⁶ are as defined for structure (I);
Z¹ are each independently selected from the group consisting of OH; SH; COOH; NH₂;

NHR²⁰; halogen; C₁₋₆alkyl; —OR¹⁹; —SR¹⁹; nitro; NR²⁰R²¹; —C(O)NR²⁰R²¹; cyano;
Z⁷, Z⁸, Z^{8a}, Z⁹ are each selected from the group consisting of OH; hydrogen; SH; COOH; NH₂; NHR²⁰; halogen; C₁₋₆alkyl; —OR¹⁹; —SR¹⁹; nitro; NR²⁰R²¹; —C(O)NR²⁰R²¹; cyano; and a polymeric group.

5. The compound according to any one of statements 1 to 4, wherein
R¹ is selected from the group consisting of C₁₋₁₀alkyl; and a polymeric group, wherein said C₁₋₁₀alkyl can be unsubstituted or substituted with one or more Z¹;
R² is selected from the group consisting of halo; nitro; cyano; —C(O)NR²⁰R²¹; C₁₋₂₀alkyl; C₆₋₁₂aryl; C₃₋₈cycloalkyl; and a polymeric group,
R³ is selected from the group consisting of halo; nitro; cyano; —C(O)NR²⁰R²¹; C₁₋₂₀alkyl; C₆₋₁₂aryl; C₃₋₈cycloalkyl; and a polymeric group,
R⁴ is selected from the group consisting of halo; nitro; cyano; —C(O)NR²⁰R²¹; C₁₋₂₀alkyl; C₆₋₁₂aryl; C₃₋₈cycloalkyl; and a polymeric group,
   wherein for R², R³ and R⁴, each independently; said C₁₋₂₀alkyl, C₆₋₁₂aryl, or C₃₋₈cycloalkyl, can be unsubstituted or substituted with one or more Z⁶;
or R² and R³ and R⁴ together with the carbon atom to which they are attached form a saturated or unsaturated 5-, or 6-membered ring;
wherein said saturated or unsaturated 5-, or 6-membered ring can be unsubstituted or substituted with one or more R⁸;
each R⁸ is independently selected from the group consisting of hydroxyC₁₋₁₀alkoxy; aminoC₁₋₁₀alkoxy; mercaptoC₁₋₁₀alkoxy; C₁₋₁₀alkyl; C₃₋₈cycloalkyl; C₆₋₁₂arylC₁₋₆alkyl; heterocyclylC₁₋₆alkyl; heteroarylC₁₋₆alkyl; —OR¹⁹; —SR¹⁹; and a polymeric group;
   wherein said hydroxyC₁₋₁₀alkoxy; aminoC₁₋₁₀alkoxy; mercaptoC₁₋₁₀alkoxy; C₁₋₁₀alkyl; C₃₋₈cycloalkyl; C₆₋₁₂arylC₁₋₆alkyl; heterocyclylC₁₋₆alkyl; or heteroarylC₁₋₆alkyl, can be unsubstituted or substituted with one or more Z⁷;
   or R⁸ is a group of formula (iii);

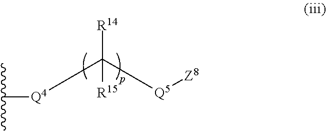

wherein the wavy line ( [symbol] ) indicates the point of attachment to the ring formed by R² and R³ and R⁴ together;
   p is an integer selected from 1, 2, or 3;
   Q⁴ is a single bond or selected from the group consisting of O, S, and NR¹⁸;
   Q⁵ is a single bond or selected from the group consisting of hydroxyC₁₋₁₀alkylene; aminoC₁₋₁₀alkylene; mercaptoC₁₋₁₀alkylene; C₁₋₂₀alkylene, C₃₋₈cycloalkylene;
   wherein said C₁₋₂₀alkylene, or C₃₋₈cycloalkylene; can be unsubstituted or substituted with one or more Z^{8a};
   each R¹⁴ and R¹⁵ is independently selected from the group consisting of hydrogen; C₁₋₂₀alkyl, hydroxyC₁₋₁₀alkoxy; and a polymeric group;
   R¹⁸ is selected from the group consisting of hydrogen; C₁₋₂₀alkyl, C₂₋₂₀alkenyl; C₂₋₂₀alkynyl; hydroxyC₁₋₁₀alkoxy; and a polymeric group;
each R⁶ is independently selected from the group consisting of hydrogen, halogen; C₁₋₁₀alkyl; C₆₋₁₂arylC₁₋₆alkyl; haloC₁₋₆alkyl; haloC₁₋₆alkyloxy; —C(O)NR²⁰R²¹; and a polymeric group; wherein said C₁₋₁₀alkyl; or C₆₋₁₂arylC₁₋₆alkyl, can be unsubstituted or substituted with one or more Z⁹;
   L¹ is selected from the group consisting of C₁₋₁₀alkyleneC₃₋₈cycloalkylene; C₃₋₈cycloalkyleneC₁₋₁₀alkylene; C₁₋₁₀alkylene; C₃₋₈cycloalkylene; C₃₋₈cycloalkyleneC₁₋₁₀alkylC₃₋₈cycloalkylene; and C₆₋₁₂arylene;
   wherein said C₁₋₁₀alkyleneC₃₋₈cycloalkylene; C₃₋₈cycloalkyleneC₁₋₁₀alkylene; C₁₋₁₀alkylene; C₃₋₈cycloalkylene; C₃₋₈cycloalkyleneC₁₋₁₀alkylC₃₋₈cycloalkylene; or C₆₋₁₂arylene, can be unsubstituted or substituted with one or more Z^{1a};
R⁵ is selected from the group consisting of OH, SH, COOH, NH₂, and NHR²⁰;
or R⁵ is a group of formula (vi);

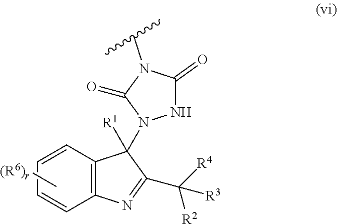

wherein the wavy line ( [symbol] ) indicates the point of attachment to L¹, and r, R¹, R², R³, R⁴ and R⁶ are as defined for structure (I);
each R¹⁹ is independently selected from the group consisting of C₁₋₂₀alkyl, C₃₋₈cycloalkyl, and C₆₋₁₂arylC₁₋₆alkyl;
   wherein said C₁₋₂₀alkyl, C₃₋₈cycloalkyl, C₆₋₁₂arylC₁₋₆alkyl, can be unsubstituted or substituted with one or more OH, SH, COOH, or NH₂;
each R²⁰ is independently selected from the group consisting of C₁₋₂₀alkyl, C₃₋₈cycloalkyl, and C₆₋₁₂arylC₁₋₆alkyl;
   wherein said C₁₋₂₀alkyl, C₃₋₈cycloalkyl, C₆₋₁₂arylC₁₋₆alkyl, can be unsubstituted or substituted with one or more OH, SH, COOH, or NH₂;
each R²¹ is independently selected from the group consisting of hydrogen; C₁₋₂₀alkyl, C₃₋₈cycloalkyl, and C₆₋₁₂arylC₁₋₆alkyl;
   wherein said C₁₋₂₀alkyl, C₃₋₈cycloalkyl, C₆₋₁₂arylC₁₋₆alkyl, can be unsubstituted or substituted with one or more OH, SH, COOH, or NH₂;
each Z^{1a} is selected from the group consisting of C₁₋₆alkyl; haloC₁₋₆alkyl; haloC₁₋₆alkyloxy; halogen;
Z¹ are each independently selected from the group consisting of OH; SH; COOH; NH₂; NHR²⁰; halogen; C₁₋₆alkyl; —OR¹⁹; —SR¹⁹; nitro; NR²⁰R²¹; —C(O)NR²⁰R²¹; cyano;
Z⁷, Z⁸, Z^{8a}, Z⁹ are each selected from the group consisting of OH; hydrogen; SH; COOH; NH₂; NHR²⁰; halogen; C₁₋₆alkyl; —OR¹⁹; —SR¹⁹; nitro; NR²⁰R²¹; —C(O)NR²⁰R²¹; cyano; and a polymeric group.

6. The compound according to any one of statements 1 to 5, having structural formula (IB)

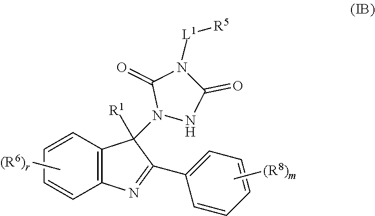

wherein m is an integer selected from 0, 1, 2, 3 or 4; R¹, R⁵, R⁶, R⁸, L¹, and r have the same meaning as defined in statements 1-5.

7. The compound according to statement 6, wherein m is an integer selected from 0, 1, 2 or 3; preferably m is selected from 0, 1 or 2; preferably m is selected from 1 or 2; preferably m is 1.

8. The compound according to any one of statements 1 to 7, wherein R⁸ is substituted with two Z⁷.

9. The compound according to any one of statements 1 to 8, wherein each Z⁷ is independently a polymeric group; wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group.

10. The compound according to any one of statements 1 to 9, wherein Z⁸ and Z^{8a} are independently selected from a polymeric group; wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group.

11. The compound according to any one of statements 1 to 10, wherein when none R¹, R², R³, R⁴, R⁵, R⁷, R⁸, R⁹, R¹⁰, R¹¹, R¹², R¹⁴, R¹⁵, R¹⁶, R¹⁷, Z⁵, Z^{5a}, Z⁷, Z⁸, Z^{8a} or Z⁹ is a polymeric group, then at least one of R⁷, R⁸, Z¹, Z², Z³, Z⁴, Z⁵, Z^{5a}, Z⁶, Z⁷, Z⁸, Z⁸a or Z⁹ is selected from the group consisting of OH, SH, COOH, NH₂, and NHR²⁰; preferably two or more of R⁷, R⁸, Z¹, Z², Z³, Z⁴, Z⁵, Z^{5a}, Z⁶, Z⁷, Z⁸, Z^{8a} or Z⁹ are selected from the group consisting of OH, SH, COOH, NH₂, and NHR²⁰; preferably three R⁷, R⁸, Z¹, Z², Z³, Z⁴, Z⁵, Z^{5a}, Z⁶, Z⁷, Z⁸, Z^{8a} or Z⁹ are selected from the group consisting of OH, SH, COOH, NH₂, and NHR²⁰; preferably four of R⁷, R⁸, Z¹, Z², Z³, Z⁴, Z⁵, Z^{5a}, Z⁶, Z⁷, Z⁸, Z^{8a} or Z⁹ are selected from the group consisting of OH, SH, COOH, NH₂, and NHR²⁰.

12. A process for the preparation of a compound according to any one of statements 1 to 11, comprising the step of contacting at least one compound of formula (II) with at least one compound of formula (III), thereby obtaining a compound of formula (I), wherein r, R¹, R², R³, R⁴, R⁵, R⁶, L¹, have the same meaning as defined in any one of statements 1 to 11;

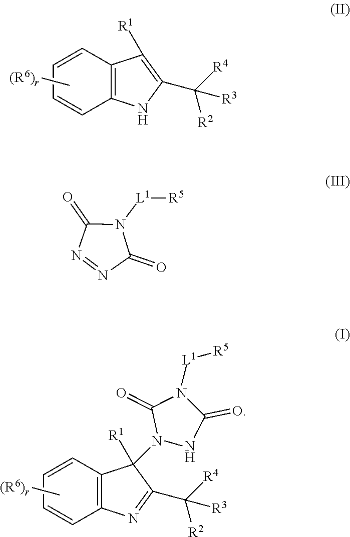

13. A process according to statement 12, wherein the compound of formula (III) has structural formula (IIIA)

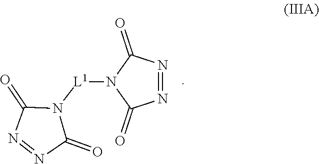

14. Process according to any one of statements 12-13, wherein the molar ratio of compound (II) to compound (III) is at least 2.0; preferably at least 2.4; preferably at least 2.6; preferably at least 2.8; preferably at least 3.0; preferably at least 3.4; preferably at least 3.6; preferably at least 3.8; preferably at least 4.0.

15. A compound of formula (A) or a stereoisomer, enantiomer, racemic, or tautomer thereof,

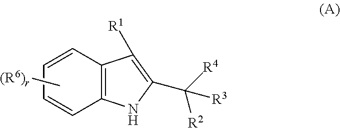

wherein,
r is an integer selected from 0, 1, 2, 3 or 4;
R¹ is selected from the group consisting of C₁₋₁₀alkyl; C₂₋₁₀alkenyl; C₂₋₁₀alkynyl; C₆₋₁₂aryl; C₃₋₈cycloalkyl; C₆₋₁₂arylC₁₋₆alkyl; heterocyclyl; heteroaryl; heterocyclylC₁₋₆alkyl; heteroarylC₁₋₈alkyl; —OR¹⁹; —SR¹⁹; NR²⁰R²¹; —C(O)NR²⁰R²¹; OH, SH, COOH, NH₂; NHR²⁰; and a polymeric group;
and wherein said C₁₋₁₀alkyl; C₂₋₁₀alkenyl; C₂₋₁₀alkynyl, C₆₋₁₂aryl, C₃₋₈cycloalkyl; C₆₋₁₂arylC₁₋₆alkyl, heterocyclyl; heteroaryl; heterocyclylC₁₋₆alkyl; or heteroarylC₁₋₆alkyl; can be unsubstituted or substituted with one or more Z¹;
   and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
   or R¹ is a group of formula (i);

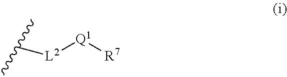

wherein the wavy line ( [symbol] ) indicates the point of attachment to the indole ring of formula (I);
L² is selected from the group consisting of C₁₋₁₀alkylene; C₃₋₆cycloalkylene; C₂₋₁₀alkenylene; C₂₋₁₀alkynylene; C₆₋₁₂arylene; and C₆₋₁₂arylC₁₋₆alkylene;
   and wherein said C₁₋₁₀alkylene; C₃₋₆cycloalkylene; C₂₋₁₀alkenylene; C₂₋₁₀alkynylene; C₆₋₁₂arylene or C₆₋₁₂arylC₁₋₆alkylene, can be unsubstituted or substituted with one or more Z²;
Q¹ is a single bond or selected from the group consisting of C₁₋₁₀alkylene; C₃₋₆cycloalkylene; —O—; —S—; —O—C₁₋₁₀alkylene; and —S—C₁₋₁₀alkylene; wherein each left side of said groups is attached to L² and the right side thereof is attached to R⁷;
   and wherein said C₁₋₁₀alkylene and said C₃₋₆cycloalkylene; can be unsubstituted or substituted with one or more Z³;
R⁷ is selected from the group consisting of hydrogen; OH, SH, COOH, NH₂; NHR²⁰, halo; nitro; cyano; —C(O)NR²⁰R²¹; C₁₋₂₀alkyl, C₂₋₂₀alkenyl; C₂₋₂₀alkynyl; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
   wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₈alkyl, heterocyclyl; heteroaryl; heterocyclylC₁₋₆alkyl; and heteroarylC₁₋₆alkyl; can be unsubstituted or substituted with one or more Z⁴;
   and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
or R⁷ is a group of formula (ii);

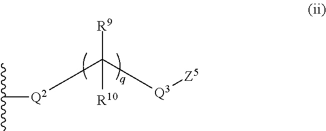

wherein the wavy line ( [symbol] ) indicates the point of attachment to Q¹;
q is an integer selected from 0; 1, 2, 3 or 4;
Q² is a single bond or selected from the group consisting of CR¹¹R¹²; O, S, and NR¹³; Q³ is a single bond or selected from the group consisting of hydroxyC₁₋₁₀alkylene; aminoC₁₋₁₀alkylene; mercaptoC₁₋₁₀alkylene; C₁₋₂₀alkylene; C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₆alkylene; and heteroarylC₁₋₆alkylene;
wherein said C₁₋₂₀alkylene, C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₈alkylene; or heteroarylC₁₋₆alkylene, can be unsubstituted or substituted with one or more Z^{5a};
wherein if q is 0, then one of Q² and Q³ is not a single bond;
each R⁹ and R¹⁰ is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
each R¹¹ and R¹² is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
R¹³ is selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; and heteroarylC₁₋₆alkyl;
R² is selected from the group consisting of halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; heteroarylC₁₋₆alkyl; and a polymeric group;
R³ is selected from the group consisting of halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; heteroarylC₁₋₆alkyl; and a polymeric group;
R⁴ is selected from the group consisting of halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; heteroarylC₁₋₆alkyl; and a polymeric group; wherein for R², R³ and R⁴, each independently; said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl, heteroarylC₁₋₆alkyl, can be unsubstituted or substituted with one or more Z⁶;
   and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
or R² and R³ and R⁴ together with the carbon atom to which they are attached can form a saturated or unsaturated 4-, 5-, 6-, 7- or 8-membered ring;
   wherein said saturated or unsaturated 4-, 5-, 6-, 7- or 8-membered ring optionally comprises one or more heteroatoms, said heteroatoms being each independently selected from O, S and N;
   wherein said saturated or unsaturated 4-, 5-, 6-, 7- or 8-membered ring can be unsubstituted or substituted with one or more R⁸;
each R⁸ is independently selected from the group consisting of hydroxyC₁₋₁₀alkoxy; aminoC₁₋₁₀alkoxy; mercaptoC₁₋₁₀alkoxy; C₁₋₁₀alkyl; C₂₋₁₀alkenyl; C₂₋₁₀alkynyl; C₆₋₁₂arylC₁₋₆alkyl; C₃₋₈cycloalkyl; C₆₋₁₂arylC₁₋₆alkyl; heterocyclyl; heteroaryl; heterocyclylC₁₋₆alkyl; heteroarylC₁₋₈alkyl; OH; SH; COOH; NH₂; NHR²⁰; halogen; haloC₁₋₆alkyl; haloC₁₋₆alkyloxy; —OR¹⁹; —SR¹⁹; nitro; NR²⁰R²¹; —C(O)NR²⁰R²¹; cyano; and a polymeric group;
   wherein said C₁₋₁₀alkyl; C₂₋₁₀alkenyl; C₂₋₁₀alkynyl; C₆₋₁₂arylC₁₋₆alkyl; C₃₋₈cycloalkyl; C₆₋₁₂arylC₁₋₆alkyl; heterocyclyl; heteroaryl; heterocyclylC₁₋₆alkyl; or heteroarylC₁₋₆alkyl, can be unsubstituted or substituted with one or more Z⁷;
   and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
   or R⁸ is a group of formula (iii);

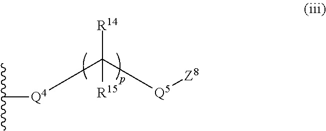

wherein the wavy line ( [symbol] ) indicates the point of attachment to the ring formed by R² and R³ and R⁴ together;
p is an integer selected from 0; 1, 2, 3 or 4;
Q⁴ is a single bond or selected from the group consisting of CR¹⁶R¹⁷; O, S, and NR¹⁸;
Q⁵ is a single bond or selected from the group consisting of hydroxyC₁₋₁₀alkylene; aminoC₁₋₁₀alkylene; mercaptoC₁₋₁₀alkylene; C₁₋₂₀alkylene, C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₆alkylene; and heteroarylC₁₋₆alkylene;
wherein said C₁₋₂₀alkylene, C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₆alkylene; or heteroarylC₁₋₆alkylene, can be unsubstituted or substituted with one or more Z^{8a};
wherein if p is 0, then one of Q⁴ and Q⁵ is not a single bond;
each R¹⁴ and R¹⁵ is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
each R¹⁶ and R¹⁷ is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
R¹⁸ is selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; and heteroarylC₁₋₆alkyl;
each R⁶ is independently selected from the group consisting of hydrogen, halogen; C₁₋₁₀alkyl; C₂₋₁₀alkenyl; C₂₋₁₀alkynyl; C₆₋₁₂arylC₁₋₆alkyl; haloC₁₋₆alkyl; haloC₁₋₆alkyloxy; —OR¹⁹; —SR¹⁹; nitro; NR²⁰R²¹; —C(O)NR²⁰R²¹; cyano; and a polymeric group;
   wherein said C₁₋₁₀alkyl; C₂₋₁₀alkenyl; C₂₋₁₀alkynyl; or C₆₋₁₂arylC₁₋₆alkyl, can be unsubstituted or substituted with one or more Z⁹;
   and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
each R¹⁹ is independently selected from the group consisting of C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl;
   wherein said C₁₋₁₀alkyl, C₂₋₂₀alkenyl, C₂₋₂₀alkynyl, C₆₋₁₂arylC₁₋₆alkyl, heterocyclylC₁₋₆alkyl; heteroarylC₁₋₆alkyl, optionally comprise one or more heteroatoms in the alkyl, alkenyl or alkynyl moiety, said heteroatoms being each independently selected from O, S and N;
   wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl, can be unsubstituted or substituted with one or more OH, SH, COOH, or NH₂;
each R²⁰ is independently selected from the group consisting of C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl;
   wherein said C₁₋₁₀alkyl, C₂₋₂₀alkenyl, C₂₋₂₀alkynyl, C₆₋₁₂arylC₁₋₆alkyl, heterocyclylC₁₋₆alkyl; heteroarylC₁₋₆alkyl, optionally comprise one or more heteroatoms in the alkyl, alkenyl or alkynyl moiety, said heteroatoms being each independently selected from O, S and N;
   wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl, can be unsubstituted or substituted with one or more OH, SH, COOH, or NH₂;
each R²¹ is independently selected from the group consisting of hydrogen; C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl;
   wherein said C₁₋₁₀alkyl, C₂₋₂₀alkenyl, C₂₋₂₀alkynyl, C₆₋₁₂arylC₁₋₆alkyl, heterocyclylC₁₋₆alkyl; heteroarylC₁₋₆alkyl, optionally comprise one or more heteroatoms in the alkyl, alkenyl or alkynyl moiety, said heteroatoms being each independently selected from O, S and N or
   wherein R²⁰ and R²¹ together with the atom to which they are attached form a 5-, 6-, or 7-membered heterocycle;
   wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl, can be unsubstituted or substituted with one or more OH, SH, COOH, or NH₂;
Z¹, Z², Z³, Z⁴, Z⁶ are each independently selected from the group consisting of OH; SH; COOH; NH₂; NHR²⁰; halogen; C₁₋₆alkyl; haloC₁₋₆alkyl; haloC₁₋₆alkyloxy; —OR¹⁹; —SR¹⁹; nitro; NR²⁰R²¹; —C(O)NR²⁰R²¹; cyano;
Z⁵, Z^{5a}, Z⁷, Z⁸, Z^{8a}, Z⁹ are independently selected from the group consisting of OH; hydrogen; SH; COOH; NH₂; NHR²⁰; halogen; C₁₋₆alkyl; haloC₁₋₆alkyl; haloC₁₋₆alkyloxy; —OR¹⁹; —SR¹⁹; nitro; NR²⁰R²¹; —C(O)NR²⁰R²¹; cyano; and a polymeric group;
   and wherein said polymeric group selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; ethylene propylene rubber (EPM); polybutadiene; polysiloxanes; and/or hydrogenated derivatives thereof; and/or block, comb and/or star copolymers of the polymeric group;
and when none of R¹, R², R³, R⁴, R⁶, R⁷, R⁸, R⁹, R¹⁰, R¹¹, R¹², R¹⁴, R¹⁵, R¹⁶, R¹⁷, Z⁵, Z^{5a}, Z⁷, Z⁸, Z^{8a} or Z⁹ is a polymeric group, then at least one of R⁷, R⁸, Z¹, Z², Z³, Z⁴, Z⁵, Z^{5a}, Z⁶, Z⁷, Z⁸ Z^{8a} or Z⁹ is selected from the group consisting of OH, SH, COOH, NH₂, and NHR²⁰.

16. The compound according to statement 15, wherein,
R¹ is selected from the group consisting of C₁₋₁₀alkyl; C₆₋₁₂aryl; C₃₋₈cycloalkyl; C₆₋₁₂arylC₁₋₆alkyl; —OR¹⁹; —SR¹⁹; NR²⁰R²¹; —C(O)NR²⁰R²¹; OH, SH, COOH, NH₂; NHR²⁰; and a polymeric group, wherein said C₁₋₁₀alkyl; C₆₋₁₂aryl; C₃₋₈cycloalkyl; or C₆₋₁₂arylC₁₋₆alkyl, can be unsubstituted or substituted with one or more Z¹;
R² is selected from the group consisting of halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; and C₆₋₁₂arylC₁₋₆alkyl; and a polymeric group, R³ is selected from the group consisting of halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; and C₆₋₁₂arylC₁₋₆alkyl; and a polymeric group,
R⁴ is selected from the group consisting of halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; and C₆₋₁₂arylC₁₋₆alkyl; and a polymeric group,
   wherein for R², R³ and R⁴, each independently; said C₁₋₂₀alkyl, C₂₋₂₀alkenyl, C₂₋₂₀alkynyl, C₆₋₁₂aryl, C₃₋₈cycloalkyl, or C₆₋₁₂arylC₁₋₆alkyl, can be unsubstituted or substituted with one or more Z⁶;
or R² and R³ and R⁴ together with the carbon atom to which they are attached form a saturated or unsaturated 4-, 5-, or 6-membered ring;
wherein said saturated or unsaturated 4-, 5-, or 6-membered ring can be unsubstituted or substituted with one or more R⁸;
each R⁸ is independently selected from the group consisting of hydroxyC₁₋₁₀alkoxy; aminoC₁₋₁₀alkoxy; mercaptoC₁₋₁₀alkoxy; C₁₋₁₀alkyl; C₂₋₁₀alkenyl; C₂₋₁₀alkynyl; C₆₋₁₂arylC₁₋₆alkyl; C₃₋₈cycloalkyl; C₆₋₁₂arylC₁₋₆alkyl; heterocyclyl; heteroaryl; heterocyclylC₁₋₆alkyl; heteroarylC₁₋₆alkyl; —OR¹⁹; —SR¹⁹; and a polymeric group;
   wherein said hydroxyC₁₋₁₀alkoxy; aminoC₁₋₁₀alkoxy; mercaptoC₁₋₁₀alkoxy; C₁₋₁₀alkyl; C₂₋₁₀alkenyl; C₂₋₁₀alkynyl; C₆₋₁₂arylC₁₋₆alkyl; C₃₋₈cycloalkyl; C₆₋₁₂arylC₁₋₆alkyl; heterocyclyl; heteroaryl; heterocyclylC₁₋₆alkyl; or heteroarylC₁₋₆alkyl, can be unsubstituted or substituted with one or more Z⁷;
   or R⁸ is a group of formula (iii);

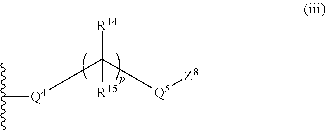

wherein the wavy line ( [symbol] ) indicates the point of attachment to the ring formed by R² and R³ and R⁴ together;
p is an integer selected from 0; 1, 2, or 3;
Q⁴ is a single bond or selected from the group consisting of CR¹⁶R¹⁷; O, S, and NR¹⁸;
Q⁵ is a single bond or selected from the group consisting of hydroxyC₁₋₁₀alkylene; aminoC₁₋₁₀alkylene; mercaptoC₁₋₁₀alkylene; C₁₋₂₀alkylene, C₆₋₁₂arylene, C₃₋₈cycloalkylene; C₆₋₁₂arylC₁₋₆alkylene; heterocyclylene; heteroarylene; heterocyclylC₁₋₈alkylene; and heteroarylC₁₋₆alkylene;
wherein said C₁₋₂₀alkylene, C₆₋₁₂arylene, C₃₋₈cycloalkylene; C₆₋₁₂arylC₁₋₆alkylene; heterocyclylene; heteroarylene; heterocyclylC₁₋₆alkylene; or heteroarylC₁₋₆alkylene; can be unsubstituted or substituted with one or more Z^{8a};
wherein if p is 0, then one of Q⁴ and Q⁵ is not a single bond;
each R¹⁴ and R¹⁵ is independently selected from the group consisting of hydrogen; C₁₋₂₀alkyl, hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; and heteroarylC₁₋₆alkyl; and a polymeric group;
each R¹⁶ and R¹⁷ is independently selected from the group consisting of hydrogen; C₁₋₂₀alkyl, hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; and heteroarylC₁₋₆alkyl; and a polymeric group;
R¹⁸ is selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; and heteroarylC₁₋₆alkyl;
each R⁶ is independently selected from the group consisting of hydrogen, halogen; C₁₋₁₀alkyl; C₆₋₁₂arylC₁₋₆alkyl; haloC₁₋₆alkyl; haloC₁₋₆alkyloxy; —OR¹⁹; —SR¹⁹; nitro; NR²⁰R²¹; —C(O)NR²⁰R²¹; cyano; and a polymeric group;
   wherein said C₁₋₁₀alkyl; or C₆₋₁₂arylC₁₋₆alkyl, can be unsubstituted or substituted with one or more Z⁹;
each R¹⁹ is independently selected from the group consisting of C₁₋₂₀alkyl, C₆₋₁₂aryl, C₃₋₈cycloalkyl, and C₆₋₁₂arylC₁₋₆alkyl;
   wherein said C₁₋₂₀alkyl, C₆₋₁₂aryl, C₃₋₈cycloalkyl, C₆₋₁₂arylC₁₋₆alkyl, can be unsubstituted or substituted with one or more OH, SH, COOH, or NH₂;
each R²⁰ is independently selected from the group consisting of C₁₋₂₀alkyl, C₆₋₁₂aryl, C₃₋₈cycloalkyl, and C₆₋₁₂arylC₁₋₆alkyl;
   wherein said C₁₋₂₀alkyl, C₆₋₁₂aryl, C₃₋₈cycloalkyl, C₆₋₁₂arylC₁₋₆alkyl, can be unsubstituted or substituted with one or more OH, SH, COOH, or NH₂;
each R²¹ is independently selected from the group consisting of hydrogen; C₁₋₂₀alkyl, C₆₋₁₂aryl, C₃₋₈cycloalkyl, and C₆₋₁₂arylC₁₋₆alkyl;
   wherein said C₁₋₂₀alkyl, C₆₋₁₂aryl, C₃₋₈cycloalkyl, C₆₋₁₂arylC₁₋₆alkyl, can be unsubstituted or substituted with one or more OH, SH, COOH, or NH₂;
Z¹, Z⁶, are each independently selected from the group consisting of OH; SH; COOH; NH₂; NHR²⁰; halogen; C₁₋₆alkyl; haloC₁₋₆alkyl; haloC₁₋₆alkyloxy; —OR¹⁹; —SR¹⁹; nitro; NR²⁰R²¹; —C(O)NR²⁰R²¹; cyano;
Z⁷, Z⁸, Z^{8a}, is each selected from the group consisting of OH; hydrogen; SH; COOH; NH₂; NHR²⁰; halogen; C₁₋₆alkyl; haloC₁₋₆alkyl; haloC₁₋₆alkyloxy; —OR¹⁹; —SR¹⁹; nitro; NR²⁰R²¹; —C(O)NR²⁰R²¹; cyano; and a polymeric group.

17. The compound according to any one of statements 15 or 16, wherein,
R¹ is selected from the group consisting of C₁₋₁₀alkyl; C₆₋₁₂aryl; C₃₋₈cycloalkyl; C₆₋₁₂arylC₁₋₆alkyl; and a polymeric group, wherein said C₁₋₁₀alkyl; C₆₋₁₂aryl; C₃₋₈cycloalkyl; or C₆₋₁₂arylC₁₋₆alkyl, can be unsubstituted or substituted with one or more Z¹;
R² is selected from the group consisting of halo; nitro; cyano; —C(O)NR²⁰R²¹; C₁₋₂₀alkyl; C₆₋₁₂aryl; C₃₋₈cycloalkyl; C₆₋₁₂arylC₁₋₆alkyl; and C₆₋₁₂arylC₁₋₆alkylC₆₋₁₂aryl; and a polymeric group,
R³ is selected from the group consisting of halo; nitro; cyano; —C(O)NR²⁰R²¹; C₁₋₂₀alkyl; C₆₋₁₂aryl; C₃₋₈cycloalkyl; C₆₋₁₂arylC₁₋₆alkyl; and C₆₋₁₂arylC₁₋₆alkylC₆₋₁₂aryl; and a polymeric group,
R⁴ is selected from the group consisting of halo; nitro; cyano; —C(O)NR²⁰R²¹; C₁₋₂₀alkyl; C₆₋₁₂aryl; C₃₋₈cycloalkyl; C₆₋₁₂arylC₁₋₆alkyl; and C₆₋₁₂arylC₁₋₆alkylC₆₋₁₂aryl; and a polymeric group,
   wherein for R², R³ and R⁴, each independently; said C₁₋₂₀alkyl, C₆₋₁₂aryl, C₃₋₈cycloalkyl, C₆₋₁₂arylC₁₋₆alkyl, can be unsubstituted or substituted with one or more Z⁶;
or R² and R³ and R⁴ together with the carbon atom to which they are attached form a saturated or unsaturated 5-, or 6-membered ring;
wherein said saturated or unsaturated 5-, or 6-membered ring can be unsubstituted or substituted with one or more R⁸;
each R⁸ is independently selected from the group consisting of C₁₋₁₀alkyl; C₂₋₁₀alkenyl; C₂₋₁₀alkynyl; C₆₋₁₂arylC₁₋₆alkyl; C₃₋₈cycloalkyl; C₆₋₁₂arylC₁₋₆alkyl; heterocyclyl; heteroaryl; heterocyclylC₁₋₆alkyl; heteroarylC₁₋₆alkyl; —OR¹⁹; —SR¹⁹; and a polymeric group;
   wherein said hydroxyC₁₋₁₀alkoxy; aminoC₁₋₁₀alkoxy; mercaptoC₁₋₁₀alkoxy; C₁₋₁₀alkyl; C₂₋₁₀alkenyl; C₂₋₁₀alkynyl; C₆₋₁₂arylC₁₋₆alkyl; C₃₋₈cycloalkyl; C₆₋₁₂arylC₁₋₆alkyl; heterocyclyl; heteroaryl; heterocyclylC₁₋₆alkyl; or heteroarylC₁₋₆alkyl, can be unsubstituted or substituted with one or more Z⁷;
   or R⁸ is a group of formula (iii);

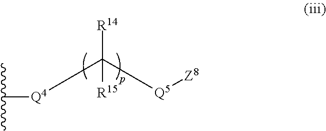

wherein the wavy line ( [symbol] ) indicates the point of attachment to the ring formed by R² and R³ and R⁴ together;
p is an integer selected from 1, 2, or 3;
Q⁴ is a single bond or selected from the group consisting of O, S, and NR¹⁸;
Q⁵ is a single bond or selected from the group consisting of hydroxyC₁₋₁₀alkylene; aminoC₁₋₁₀alkylene; mercaptoC₁₋₁₀alkylene; C₁₋₂₀alkylene, C₆₋₁₂arylene, C₃₋₈cycloalkylene;
wherein said C₁₋₂₀alkylene, C₆₋₁₂arylene, or C₃₋₈cycloalkylene; can be unsubstituted or substituted with one or more Z⁸a;
each R¹⁴ and R¹⁵ is independently selected from the group consisting of hydrogen; C₁₋₂₀alkyl, hydroxyC₁₋₁₀alkoxy; aminoC₁₋₁₀alkoxy; mercaptoC₁₋₁₀alkoxy; and a polymeric group;
R¹⁸ is selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy;
each R⁶ is independently selected from the group consisting of hydrogen, halogen; C₁₋₁₀alkyl; C₆₋₁₂arylC₁₋₆alkyl; haloC₁₋₆alkyl; haloC₁₋₆alkyloxy; —OR¹⁹; —SR¹⁹; nitro; NR²⁰R²¹; —C(O)NR²⁰R²¹; cyano; and a polymeric group;
   wherein said C₁₋₁₀alkyl; or C₆₋₁₂arylC₁₋₆alkyl, can be unsubstituted or substituted with one or more Z⁹;
each R¹⁹ is independently selected from the group consisting of C₁₋₂₀alkyl, C₆₋₁₂aryl, C₃₋₈cycloalkyl, and C₆₋₁₂arylC₁₋₆alkyl;
   wherein said C₁₋₂₀alkyl, C₆₋₁₂aryl, C₃₋₈cycloalkyl, C₆₋₁₂arylC₁₋₆alkyl, can be unsubstituted or substituted with one or more OH, SH, COOH, or NH₂;
each R²⁰ is independently selected from the group consisting of C₁₋₂₀alkyl, C₆₋₁₂aryl, C₃₋₈cycloalkyl, and C₆₋₁₂arylC₁₋₆alkyl;
   wherein said C₁₋₂₀alkyl, C₆₋₁₂aryl, C₃₋₈cycloalkyl, C₆₋₁₂arylC₁₋₆alkyl, can be unsubstituted or substituted with one or more OH, SH, COOH, or NH₂;
each R²¹ is independently selected from the group consisting of hydrogen; C₁₋₂₀alkyl, C₆₋₁₂aryl, C₃₋₈cycloalkyl, and C₆₋₁₂arylC₁₋₆alkyl;
   wherein said C₁₋₂₀alkyl, C₆₋₁₂aryl, C₃₋₈cycloalkyl, C₆₋₁₂arylC₁₋₆alkyl, can be unsubstituted or substituted with one or more OH, SH, COOH, or NH₂;
Z¹ is independently selected from the group consisting of OH; SH; COOH; NH₂; NHR²⁰; halogen; C₁₋₆alkyl; —OR¹⁹; —SR¹⁹; nitro; NR²⁰R²¹; —C(O)NR²⁰R²¹; cyano;
Z⁷, Z⁸, Z^{8a} are each selected from the group consisting of OH; hydrogen; SH; COOH; NH₂; NHR²⁰; halogen; C₁₋₆alkyl; —OR¹⁹; —SR¹⁹; nitro; NR²⁰R²¹; —C(O)NR²⁰R²¹; cyano and a polymeric group.

18. The compound according to any one of statements 15 to 17, wherein
R¹ is selected from the group consisting of C₁₋₁₀alkyl; C₃₋₈cycloalkyl; and a polymeric group, wherein said C₁₋₁₀alkyl; or C₃₋₈cycloalkyl; can be unsubstituted or substituted with one or more Z¹;
R² is selected from the group consisting of halo; nitro; cyano; —C(O)NR²⁰R²¹; C₁₋₂₀alkyl; C₆₋₁₂aryl; C₃₋₈cycloalkyl; and a polymeric group,
R³ is selected from the group consisting of halo; nitro; cyano; —C(O)NR²⁰R²¹; C₁₋₂₀alkyl; C₆₋₁₂aryl; C₃₋₈cycloalkyl; and a polymeric group,
R⁴ is selected from the group consisting of halo; nitro; cyano; —C(O)NR²⁰R²¹; C₁₋₂₀alkyl; C₆₋₁₂aryl; C₃₋₈cycloalkyl; and a polymeric group,
   wherein for R², R³ and R⁴, each independently; said C₁₋₂₀alkyl, C₆₋₁₂aryl, or C₃₋₈cycloalkyl, can be unsubstituted or substituted with one or more Z⁶;
or R² and R³ and R⁴ together with the carbon atom to which they are attached form a saturated or unsaturated 5-, or 6-membered ring;
wherein said saturated or unsaturated 5-, or 6-membered ring can be unsubstituted or substituted with one or more R⁸;
each R⁸ is independently selected from the group consisting of hydroxyC₁₋₁₀alkoxy; aminoC₁₋₁₀alkoxy; mercaptoC₁₋₁₀alkoxy; C₁₋₁₀alkyl; C₆₋₁₂arylC₁₋₆alkyl; C₃₋₈cycloalkyl; C₆₋₁₂arylC₁₋₆alkyl; heterocyclylC₁₋₆alkyl; heteroarylC₁₋₆alkyl; —OR¹⁹; —SR¹⁹; and a polymeric group; wherein said hydroxyC₁₋₁₀alkoxy; aminoC₁₋₁₀alkoxy; mercaptoC₁₋₁₀alkoxy; C₁₋₁₀alkyl; C₆₋₁₂arylC₁₋₆alkyl; C₃₋₈cycloalkyl; C₆₋₁₂arylC₁₋₆alkyl; heterocyclylC₁₋₆alkyl; or heteroarylC₁₋₆alkyl, can be unsubstituted or substituted with one or more Z⁷;
   or R⁸ is a group of formula (iii);

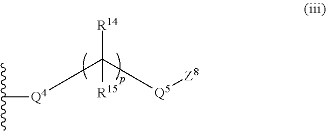

wherein the wavy line ( [symbol] ) indicates the point of attachment to the ring formed by R² and R³ and R⁴ together;
p is an integer selected from 1, 2, or 3;
Q⁴ is a single bond or selected from the group consisting of O, S, and NR¹⁸;
Q⁵ is a single bond or selected from the group consisting of hydroxyC₁₋₁₀alkylene; aminoC₁₋₁₀alkylene; mercaptoC₁₋₁₀alkylene; C₁₋₂₀alkylene, C₃₋₈cycloalkylene;
wherein said C₁₋₂₀alkylene, or C₃₋₈cycloalkylene; can be unsubstituted or substituted with one or more Z^{8a};
each R¹⁴ and R¹⁵ is independently selected from the group consisting of hydrogen; C₁₋₂₀alkyl, hydroxyC₁₋₁₀alkoxy; and a polymeric group;
R¹⁸ is selected from the group consisting of hydrogen; C₁₋₂₀alkyl, C₂₋₂₀alkenyl; C₂₋₂₀alkynyl; hydroxyC₁₋₁₀alkoxy; and a polymeric group;
each R⁶ is independently selected from the group consisting of hydrogen, halogen; C₁₋₁₀alkyl; C₆₋₁₂arylC₁₋₆alkyl; haloC₁₋₆alkyl; haloC₁₋₆alkyloxy, and C(O)NR²⁰R²¹;
   wherein said C₁₋₁₀alkyl; or C₆₋₁₂arylC₁₋₆alkyl, can be unsubstituted or substituted with one or more Z⁹;
each R¹⁹ is independently selected from the group consisting of C₁₋₂₀alkyl, C₃₋₈cycloalkyl, and C₆₋₁₂arylC₁₋₆alkyl;
   wherein said C₁₋₂₀alkyl, C₃₋₈cycloalkyl, C₆₋₁₂arylC₁₋₆alkyl, can be unsubstituted or substituted with one or more OH, SH, COOH, or NH₂;
each R²⁰ is independently selected from the group consisting of C₁₋₂₀alkyl, C₃₋₈cycloalkyl, and C₆₋₁₂arylC₁₋₆alkyl;
   wherein said C₁₋₂₀alkyl, C₆₋₁₂aryl, C₃₋₈cycloalkyl, C₆₋₁₂arylC₁₋₆alkyl, can be unsubstituted or substituted with one or more OH, SH, COOH, or NH₂;
each R²¹ is independently selected from the group consisting of hydrogen; C₁₋₂₀alkyl, O₃₋₈cycloalkyl, and C₆₋₁₂arylC₁₋₆alkyl;
   wherein said C₁₋₂₀alkyl, C₆₋₁₂aryl, C₃₋₈cycloalkyl, C₆₋₁₂arylC₁₋₆alkyl, can be unsubstituted or substituted with one or more OH, SH, COOH, or NH₂;
Z¹ is independently selected from the group consisting of OH; SH; COOH; NH₂; NHR²⁰; halogen; C₁₋₆alkyl; —OR¹⁹; —SR¹⁹; nitro; NR²⁰R²¹; —C(O)NR²⁰R²¹; cyano;
Z⁷, Z⁸, Z^{8a} are each selected from the group consisting of OH; hydrogen; SH; COOH; NH₂; NHR²⁰; halogen; C₁₋₆alkyl; —OR¹⁹; —SR¹⁹; nitro; NR²⁰R²¹; —C(O)NR²⁰R²¹; cyano; and a polymeric group.

19. The compound according to any one of statements 15 to 18, having structural formula (A2)

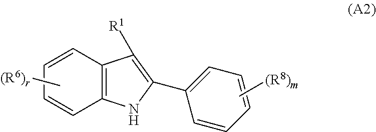

wherein m is an integer selected from 0, 1, 2, 3 or 4; R¹, R^{6′}, R⁸, and r have the same meaning as defined in statements 15-18.

20. The compound according to statement 19, wherein m is an integer selected from 0, 1, 2 or 3; preferably m is selected from 0, 1 or 2; preferably m is selected from 1 or 2; preferably m is 1.

21. The compound according to any one of statements 15 to 20, wherein when none R¹, R², R³, R⁴, R⁵, R⁷, R⁸, R⁹, R¹⁰, R¹¹, R¹², R¹⁴, R¹⁵, R¹⁶, R¹⁷, Z⁵, Z^{5a}, Z⁷, Z⁸, Z^{8a} or Z⁹ is a polymeric group, then at least one of R⁷, R⁸, Z¹, Z², Z³, Z⁴, Z⁵, Z^{5a}, Z⁶, Z⁷, Z⁸ Z⁸a or Z⁹ is selected from the group consisting of OH, SH, COOH, NH₂, and NHR²⁰; preferably two or more of R⁷, R⁸, Z¹, Z², Z³, Z⁴, Z⁵, Z^{5a}, Z⁶, Z⁷, Z⁸ or Z^{8a} are selected from the group consisting of OH, SH, COOH, NH₂, and NHR²⁰; preferably three R⁷, R⁸, Z¹, Z², Z³, Z⁴, Z⁵, Z^{5a}, Z⁶, Z⁷, Z⁸ or Z^{8a} are selected from the group consisting of OH, SH, COOH, NH₂, and NHR²⁰; preferably four of R⁷, R⁸, Z¹, Z², Z³, Z⁴, Z⁵, Z^{5a}, Z⁶, Z⁷, Z⁸ or Z^{8a} are selected from the group consisting of OH, SH, COOH, NH₂, and NHR²⁰.

22. A composition comprising a compound of formula (I) as defined in any one of statements 1 to 11.

23. A composition according to statement 22, wherein the composition further comprises at least one compound according to formula (A), as defined in any one of claims 15 to 21.

24. A composition according to statement 23, wherein molar ratio of compound (A) to compound (I) is 1.0 or more; preferably more than 1.0; preferably more than 1.2; preferably more than 1.4; preferably more than 1.6; preferably more than 2.0; preferably 2.0 or more.

25. The composition according to any one of statements 22 to 24, wherein the composition further comprises a polyol; preferably a bifunctional polyol; preferably a polyol with a number average molecular weight of less than 2000 g/mol; preferably a molecular weight of less than 1600 g/mol; preferably a molecular weight of less than 1200 g/mol; preferably a molecular weight of less than 700 g/mol.

26. The composition according to any one of statements 22 to 25, wherein the composition further comprises a dispersing agent, a surfactant; a catalyst a fire retardant, a blowing agent, an anti-oxidant, a filler, a solvent, a chain extender, or a combination thereof.

27. A polyurethane or polyurethane prepolymer obtainable by contacting at least one isocyanate with at least one compound of formula (I) as defined in any one of statements 1 to 11, or with at least one compound of formula (A) as defined in any one of statements 15 to 21, or with a composition as defined in any one of statements 22 to 26.

28. A polyurethane or polyurethane prepolymer obtainable by contacting at least one isocyanate with at least one isocyanate-reactive compound and with at least one compound of formula (I) as defined in any one of statements 1 to 11, or with at least one compound of formula (A) as defined in any one of statements 15 to 21, or with a composition as defined in any one of statements 22 to 26.

29. Use of a compound of formula (I) according to any one of statements 1 to 11, or of a compound of formula (A) according to any one of statements 15 to 21, or of a composition as defined in any one of statements 22 to 26, or of a polyurethane or polyurethane prepolymer as defined in statements 27 or 28, for the preparation of foams, adhesives, coatings, sealants, aerogels or elastomers.

30. Use according to statement 29, wherein the foam is a flexible foam, a semi-rigid foam or a rigid foam.

31. Use according to statement 29, wherein the elastomer is a non-cellular elastomer or a microcellular elastomer.

32. An article comprising a compound of formula (I) according to any one of statements 1 to 11, or a compound of formula (A) according to any one of statements 15 to 21, or a composition as defined in any one of statements 22 to 26, or a polyurethane or polyurethane prepolymer as defined in statements 27 or 28.

33. A process for reshaping and/or repairing and/or reprocessing an article comprising a compound of formula (I) according to any one of statements 1 to 11, comprising the step of thermally treating the compound of formula (I) at a temperature of at least 50° C.; preferably at a temperature of at least 60° C.; preferably at a temperature of at least at least 70° C.; preferably at a temperature of at least 80° C.; preferably at a temperature of at least 90° C.

34. A process according to statement 33, further comprising the step or applying pressure to the article.

35. A process according to statement 34, wherein a pressure of at least 4 bar is applied; preferably a pressure of at least 5 bar.

The present invention relates to a compound of formula (I) or a stereoisomer, enantiomer, racemic, or tautomer thereof, is provided herein, wherein,

r is an integer selected from 0, 1, 2, 3 or 4;

R¹ is selected from the group consisting of C₁₋₁₀alkyl; C₂₋₁₀alkenyl; C₂₋₁₀alkynyl; C₆₋₁₂aryl; C₃₋₈cycloalkyl; C₆₋₁₂arylC₁₋₆alkyl; heterocyclyl; heteroaryl; heterocyclylC₁₋₆alkyl; heteroarylC₁₋₆alkyl; —OR¹⁹; —SR¹⁹; NR²⁰R²¹; —C(O)NR²⁰R²¹; OH, SH, COOH, NH₂; NHR²⁰; and a polymeric group; for example R¹ is selected from the group consisting of C₁₋₁₀alkyl; C₂₋₁₀alkenyl; C₂₋₁₀alkynyl; C₆₋₁₂aryl; C₃₋₈cycloalkyl; C₆₋₁₂arylC₁₋₆alkyl; heterocyclyl; heteroaryl; heterocyclylC₁₋₆alkyl; heteroarylC₁₋₆alkyl; —C(O)NR²⁰R²¹; OH, SH, COOH, NH₂; NHR²⁰; and a polymeric group; and wherein said C₁₋₁₀alkyl; C₂₋₁₀alkenyl; C₂₋₁₀alkynyl, C₆₋₁₂aryl, C₃₋₈cycloalkyl; C₆₋₁₂arylC₁₋₈alkyl, heterocyclyl; heteroaryl; heterocyclylC₁₋₆alkyl; or heteroarylC₁₋₆alkyl; can be unsubstituted or substituted with one or more Z¹;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
or R¹ is a group of formula (i);

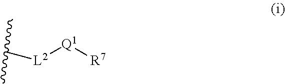

wherein the wavy line ( [symbol] ) indicates the point of attachment to the indole ring of formula (I);
L² is selected from the group consisting of C₁₋₁₀alkylene; C₃₋₆cycloalkylene; C₂₋₁₀alkenylene; C₂₋₁₀alkynylene; C₆₋₁₂arylene; and C₆₋₁₂arylC₁₋₆alkylene;
   and wherein said C₁₋₁₀alkylene; C₃₋₆cycloalkylene; C₂₋₁₀alkenylene; C₂₋₁₀alkynylene; C₆₋₁₂arylene or C₆₋₁₂arylC₁₋₆alkylene, can be unsubstituted or substituted with one or more Z²;
Q¹ is a single bond or selected from the group consisting of C₁₋₁₀alkylene; C₃₋₆cycloalkylene; —O—; —S—; —O—C₁₋₁₀alkylene; and —S—C₁₋₁₀alkylene; wherein each left side of said groups is attached to L² and the right side thereof is attached to R⁷;
   and wherein said C₁₋₁₀alkylene and said C₃₋₆cycloalkylene; can be unsubstituted or substituted with one or more Z³;
R⁷ is selected from the group consisting of hydrogen; OH, SH, COOH, NH₂; NHR²⁰, halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
   wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl; can be unsubstituted or substituted with one or more Z⁴;
   and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
or R⁷ is a group of formula (ii);

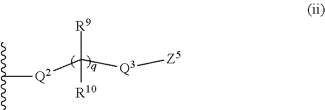

wherein the wavy line ( [symbol] ) indicates the point of attachment to Q¹;
q is an integer selected from 0; 1, 2, 3 or 4;
Q² is a single bond or selected from the group consisting of CR¹¹R¹²; O, S, and NR¹³;
Q³ is a single bond or selected from the group consisting of hydroxyC₁₋₁₀alkylene; aminoC₁₋₁₀alkylene; mercaptoC₁₋₁₀alkylene; C₁₋₂₀alkylene; C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₆alkylene; and heteroarylC₁₋₆alkylene;
   wherein said C₁₋₂₀alkylene, C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₈alkylene; or heteroarylC₁₋₆alkylene, can be unsubstituted or substituted with one or more Z^{5a};
   wherein if q is 0, then one of Q² and Q³ is not a single bond;
   each R⁹ and R¹⁰ is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
   and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;

each R¹¹ and R¹² is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;

R¹³ is selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; and heteroarylC₁₋₆alkyl;

R² is selected from the group consisting of halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; heteroarylC₁₋₆alkyl; and a polymeric group;

R³ is selected from the group consisting of halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; heteroarylC₁₋₆alkyl; and a polymeric group;

R⁴ is selected from the group consisting of halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; heteroarylC₁₋₆alkyl; and a polymeric group;
wherein for R², R³ and R⁴, each independently; said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl, heteroarylC₁₋₆alkyl, can be unsubstituted or substituted with one or more Z⁶;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;

or R² and R³ and R⁴ together with the carbon atom to which they are attached can form a saturated or unsaturated 4-, 5-, 6-, 7- or 8-membered ring;
wherein said saturated or unsaturated 4-, 5-, 6-, 7- or 8-membered ring optionally comprises one or more heteroatoms, said heteroatoms being each independently selected from O, S and N;
wherein said saturated or unsaturated 4-, 5-, 6-, 7- or 8-membered ring can be unsubstituted or substituted with one or more R⁸;

each R⁸ is independently selected from the group consisting of hydroxyC₁₋₁₀alkoxy; aminoC₁₋₁₀alkoxy; mercaptoC₁₋₁₀alkoxy; C₁₋₁₀alkyl; C₂₋₁₀alkenyl; C₂₋₁₀alkynyl; C₆₋₁₂arylC₁₋₆alkyl; C₃₋₈cycloalkyl; C₆₋₁₂arylC₁₋₆alkyl; heterocyclyl; heteroaryl; heterocyclylC₁₋₆alkyl; heteroarylC₁₋₆alkyl; OH; SH; COOH; NH₂; NHR²⁰; halogen; haloC₁₋₆alkyl; haloC₁₋₆alkyloxy; —OR¹⁹; —SR¹⁹; nitro; NR²⁰R²¹; —C(O)NR²⁰R²¹; cyano; and a polymeric group;
wherein said hydroxyC₁₋₁₀alkoxy; aminoC₁₋₁₀alkoxy; mercaptoC₁₋₁₀alkoxy; C₁₋₁₀alkyl; C₂₋₁₀alkenyl; C₂₋₁₀alkynyl; C₆₋₁₂arylC₁₋₆alkyl; C₃₋₈cycloalkyl; C₆₋₁₂arylC₁₋₆alkyl; heterocyclyl; heteroaryl; heterocyclylC₁₋₆alkyl; or heteroarylC₁₋₆alkyl, can be unsubstituted or substituted with one or more Z⁷;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;

or R⁸ is a group of formula (iii);

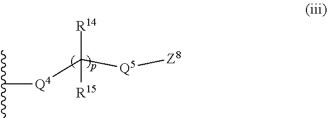

wherein the wavy line ( [symbol] ) indicates the point of attachment to the ring formed by R² and R³ and R⁴ together;
p is an integer selected from 0; 1, 2, 3 or 4;
Q⁴ is a single bond or selected from the group consisting of CR¹⁶R¹⁷; O, S, and NR¹⁸;
Q⁵ is a single bond or selected from the group consisting of hydroxyC₁₋₁₀alkylene; aminoC₁₋₁₀alkylene; mercaptoC₁₋₁₀alkylene; C₁₋₂₀alkylene, C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₆alkylene; and heteroarylC₁₋₆alkylene;
   wherein said C₁₋₂₀alkylene, C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₈alkylene; or heteroarylC₁₋₆alkylene, can be unsubstituted or substituted with one or more Z^{8a};
   wherein if p is 0, then one of Q⁴ and Q⁵ is not a single bond;
each R¹⁴ and R¹⁵ is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
   and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
each R¹⁶ and R¹⁷ is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
   and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
R¹⁸ is selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; and heteroarylC₁₋₆alkyl;
each R⁶ is independently selected from the group consisting of hydrogen, halogen; C₁₋₁₀alkyl; C₂₋₁₀alkenyl; C₂₋₁₀alkynyl; C₆₋₁₂arylC₁₋₆alkyl; haloC₁₋₆alkyl; haloC₁₋₆alkyloxy; —OR¹⁹; —SR¹⁹; nitro; NR²⁰R²¹; —C(O)NR²⁰R²¹; cyano; and a polymeric group;
   wherein said C₁₋₁₀alkyl; C₂₋₁₀alkenyl; C₂₋₁₀alkynyl; or C₆₋₁₂arylC₁₋₆alkyl, can be unsubstituted or substituted with one or more Z⁹;
   and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;

L¹ is selected from the group consisting of C₁₋₁₀alkyleneC₃₋₈cycloalkylene; C₃₋₈cycloalkyleneC₁₋₁₀alkylene; C₁₋₁₀alkylene; C₃₋₈cycloalkylene; C₃₋₈cycloalkyleneC₁₋₁₀alkylC₃₋₈cycloalkylene; C₂₋₁₀alkenylene; C₂₋₁₀alkynylene; C₆₋₁₂arylene; C₆₋₁₂aryleneC₁₋₆alkylene; C₆₋₁₂aryleneC₁₋₆alkylC₆₋₁₂arylene; and C₁₋₆alkyleneC₆₋₁₂arylC₁₋₆alkylene; wherein said C₁₋₁₀alkyleneC₃₋₈cycloalkylene; C₃₋₈cycloalkyleneC₁₋₁₀alkylene; C₁₋₁₀alkylene; C₃₋₈cycloalkylene; C₃₋₈cycloalkyleneC₁₋₁₀alkylC₃₋₈cycloalkylene; C₂₋₁₀alkenylene; C₂₋₁₀alkynylene; C₆₋₁₂arylene; C₆₋₁₂aryleneC₁₋₆alkylene; C₆₋₁₂aryleneC₁₋₆alkylC₆₋₁₂arylene; or C₁₋₆alkyleneC₆₋₁₂arylC₁₋₆alkylene, can be unsubstituted or substituted with one or more Z^{1a};
or L¹ is selected from a group of formula (iv);

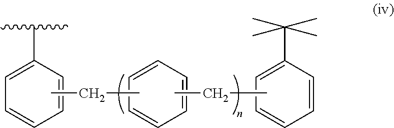

wherein the wavy line ( [symbol] ) indicates the point of attachment to the nitrogen of the formula (I) and the crossed line ( [symbol] ) indicates the point of attachment to R⁵;
wherein n is an integer selected from 0, 1, 2, 3, 4, 5, 6, 7, 8, 9, or 10;
R⁵ is selected from the group consisting of OH, SH, COOH, NH₂, and NHR²⁰;
or R⁵ is a group of formula (v);

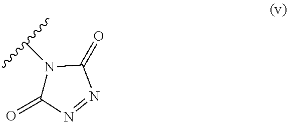 [SMILES: *N1C(=O)N=NC1=O]

wherein the wavy line ( [symbol] ) indicates the point of attachment to L¹;
or R⁵ is a group of formula (vi);

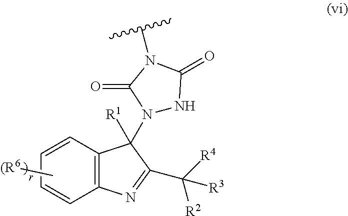

wherein the wavy line ( [symbol] ) indicates the point of attachment to L¹, and r, R¹, R², R³, R⁴ and R⁶ are as defined for structure (I);
each R¹⁹ is independently selected from the group consisting of C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl;
   wherein said C₁₋₁₀alkyl, C₂₋₂₀alkenyl, C₂₋₂₀alkynyl, C₆₋₁₂arylC₁₋₆alkyl, heterocyclylC₁₋₆alkyl; heteroarylC₁₋₆alkyl, optionally comprise one or more heteroatoms in the alkyl, alkenyl or alkynyl moiety, said heteroatoms being each independently selected from O, S and N; preferably wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl, can be unsubstituted or substituted with one or more OH, SH, COOH, or NH₂;
each R²⁰ is independently selected from the group consisting of C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl;
   wherein said C₁₋₁₀alkyl, C₂₋₂₀alkenyl, C₂₋₂₀alkynyl, C₆₋₁₂arylC₁₋₆alkyl, heterocyclylC₁₋₆alkyl; heteroarylC₁₋₆alkyl, optionally comprise one or more heteroatoms in the alkyl, alkenyl or alkynyl moiety, said heteroatoms being each independently selected from O, S and N; preferably wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl, can be unsubstituted or substituted with one or more OH, SH, COOH, or NH₂;
each R²¹ is independently selected from the group consisting of hydrogen, C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl;
   wherein said C₁₋₁₀alkyl, C₂₋₂₀alkenyl, C₂₋₂₀alkynyl, C₆₋₁₂arylC₁₋₆alkyl, heterocyclylC₁₋₆alkyl; heteroarylC₁₋₆alkyl, optionally comprise one or more heteroatoms in the alkyl, alkenyl or alkynyl moiety, said heteroatoms being each independently selected from O, S and N; preferably wherein said C₁₋₂₀alkyl, C₂₋₂₀alkenyl, C₂₋₂₀alkynyl, C₆₋₁₂aryl, C₃₋₈cycloalkyl, O₈₋₁₂arylC₁₋₆alkyl, heterocyclyl; heteroaryl; heterocyclylC₁₋₆alkyl; and heteroarylC₁₋₆alkyl, can be unsubstituted or substituted with one or more OH, SH, COOH, or NH₂;
or wherein R²⁰ and R²¹ together with the atom to which they are attached form a 5-, 6-, or 7-membered heterocycle.

each Z^{1a} is selected from the group consisting of C₁₋₆alkyl; C₂₋₆alkenyl; C₂₋₆alkynyl; haloC₁₋₆alkyl; haloC₁₋₆alkyloxy; halogen; isocyanate;

or Z^{1a} is a group of formula (vii);

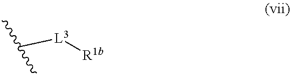

wherein the wavy line ( [symbol] ) indicates the point of attachment to L¹;
L³ is a single bond or is selected from the group consisting of C₁₋₁₀alkylene; C₂₋₁₀alkenylene; C₂₋₁₀alkynylene; and C₆₋₁₂aryl;
R^{1b} is a group of formula (viii);

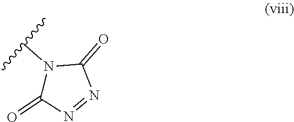 [SMILES: *N1C(=O)N=NC1=O]

wherein the wavy line ( [symbol] ) indicates the point of attachment to L³;
or R^{1b} is a group of formula (ix);

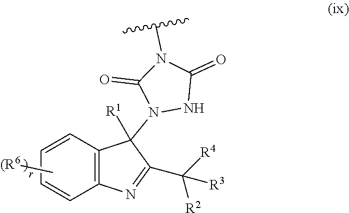

wherein the wavy line ( [symbol] ) indicates the point of attachment to L³ and r, R¹, R², R³, R⁴ and R⁶ are as defined for structure (I);

Z¹, Z², Z³, Z⁴, Z⁶, Z⁹ are each independently selected from the group consisting of OH; SH; COOH; NH₂; NHR²⁰; halogen; C₁₋₆alkyl; haloC₁₋₆alkyl; haloC₁₋₆alkyloxy; —OR¹⁹; —SR¹⁹; nitro; NR²⁰R²¹; —C(O)NR²⁰R²¹; cyano;

Z⁵, Z^{5a}, Z⁷, Z⁸, Z^{8a} are each independently selected from the group consisting of OH; hydrogen; SH; COOH; NH₂; NHR²⁰; halogen; C₁₋₆alkyl; haloC₁₋₆alkyl; haloC₁₋₆alkyloxy; —OR¹⁹; —SR¹⁹; nitro; NR²⁰R²¹; —C(O)NR²⁰R²¹; cyano; and a polymeric group;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;

and when none of R¹, R², R³, R⁴, R⁶, R⁷, R⁸, R⁹, R¹⁹, R¹¹, R¹², R¹⁴, R¹⁵, R¹⁶, R¹⁷, Z⁵, Z^{5a}, Z⁷, Z⁸, Z^{8a} or Z⁹ is a polymeric group, then at least one of R⁷, R⁸, Z¹, Z², Z³, Z⁴, Z⁵, Z^{5a}, Z⁶, Z⁷, Z⁸, Z^{8a} or Z⁹ is selected from the group consisting of OH, SH, COOH, NH₂, and NHR²⁰.

According to an embodiment, the present invention provides compounds of formula (I), (IB); and any subgroup thereof wherein,

r is an integer selected from 0, 1, 2, 3 or 4; preferably r is selected from 0, 1, 2, or 3; preferably r is selected from 0, 1 or 2; preferably r is selected from 0, or 1; more preferably r is 0;

R¹ is selected from the group consisting of C₁₋₂₀alkyl, C₂₋₂₀alkenyl; C₂₋₂₀alkynyl; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; —OR¹⁹; —SR¹⁹; NR²⁰R²¹; —C(O)NR²⁰R²¹; OH, SH, COOH, NH₂; NHR²⁰, halo; nitro; cyano; and a polymeric group; preferably R¹ is selected from the group consisting of C₁₋₂₀alkyl, C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; —OR¹⁹; —SR¹⁹; NR²⁰R²¹; —C(O)NR²⁰R²¹; OH, SH, COOH, NH₂; NHR²⁰, and a polymeric group; preferably R¹ is selected from the group consisting of C₁₋₁₀alkyl; C₂₋₁₀alkenyl; C₂₋₁₀alkynyl; C₆₋₁₂aryl; C₃₋₈cycloalkyl; C₆₋₁₂arylC₁₋₆alkyl; —OR¹⁹; —SR¹⁹; NR²⁰R²¹; —C(O)NR²⁰R²¹; OH, SH, COOH, NH₂; NHR²⁰, halo; nitro; cyano; and a polymeric group; preferably R¹ is selected from the group consisting of C₁₋₁₀alkyl; C₂₋₁₀alkenyl; C₆₋₁₂aryl; C₃₋₈cycloalkyl; C₆₋₁₂arylC₁₋₆alkyl; —OR¹⁹; —SR¹⁹; NR²⁰R²¹; and a polymeric group; preferably R¹ is selected from the group consisting of C₁₋₁₀alkyl; C₆₋₁₂aryl; C₃₋₈cycloalkyl; C₆₋₁₂arylC₁₋₆alkyl; —OR¹⁹; —SR¹⁹; NR²⁰R²¹; and a polymeric group; preferably R¹ is selected from the group consisting of C₁₋₁₀alkyl, C₃₋₈cycloalkyl; —OR¹⁹; —SR¹⁹; and NR²⁰R²¹; preferably R¹ is selected from the group consisting of C₁₋₆alkyl; C₆₋₁₂aryl; C₃₋₈cycloalkyl; and C₆₋₁₂arylC₁₋₆alkyl; preferably R¹ is selected from the group consisting of C₁₋₆alkyl and C₃₋₈cycloalkyl; preferably R¹ is C₁₋₁₀alkyl; for example R¹ is C₁₋₆alkyl; for example R¹ is C₁₋₆alkyl; and wherein said C₁₋₁₀alkyl; C₂₋₁₀alkenyl; C₂₋₁₀alkynyl, C₆₋₁₂aryl, C₃₋₈cycloalkyl; C₆₋₁₂arylC₁₋₆alkyl; heterocyclyl; heteroaryl; heterocyclylC₁₋₆alkyl; or heteroarylC₁₋₆alkyl, can be unsubstituted or substituted with one or more Z¹; preferably wherein said C₁₋₂₀alkyl, C₆₋₁₂aryl, C₃₋₈cycloalkyl; C₆₋₁₂arylC₁₋₆alkyl; heterocyclyl; heteroaryl; heterocyclylC₁₋₆alkyl; or heteroarylC₁₋₆alkyl, can be unsubstituted or substituted with one or more Z¹; preferably wherein said C₁₋₁₀alkyl; C₂₋₁₀alkenyl; C₂₋₁₀alkynyl; C₆₋₁₂aryl; C₃₋₈cycloalkyl; or C₆₋₁₂arylC₁₋₆alkyl, can be unsubstituted or substituted with one or more Z¹; preferably wherein said C₁₋₁₀alkyl; C₂₋₁₀alkenyl; C₆₋₁₂aryl; C₃₋₈cycloalkyl or C₆₋₁₂arylC₁₋₆alkyl, can be unsubstituted or substituted with one or more Z¹; preferably wherein said C₁₋₁₀alkyl; C₆₋₁₂aryl; C₃₋₈cycloalkyl or C₆₋₁₂arylC₁₋₆alkyl, can be unsubstituted or substituted with one or more Z¹; preferably wherein said C₁₋₁₀alkyl or C₃₋₈cycloalkyl, can be unsubstituted or substituted with one or more Z¹; preferably wherein said C₁₋₆alkyl; C₆₋₁₂aryl; C₃₋₈cycloalkyl; or C₆₋₁₂arylC₁₋₆alkyl, can be unsubstituted or substituted with one or more Z¹; preferably wherein said C₁₋₆alkyl or C₃₋₈cycloalkyl, can be unsubstituted or substituted with one or more Z¹; preferably wherein said C₁₋₁₀alkyl, for example C₁₋₆alkyl, for example C₁₋₆alkyl, can be unsubstituted or substituted with one or more Z¹;

and wherein for R¹ said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group; preferably wherein said polymeric group is selected from the group consisting of polyurethane; isocyanate containing prepolymer, hydroxyl containing prepolymer, polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; ethylene propylene diene monomer (M-class) rubber (EPDM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group; preferably wherein said polymeric group is selected from the group consisting of polyurethane; isocyanate containing prepolymer, hydroxyl containing prepolymer, polyacrylate; polymethacrylate; polystyrene; copolymer of acrylate; poly(methyl methacrylate); methacrylate and/or styrene; poly(isobornyl acrylate); polyacrylonitrile; polyamide; polyester amide; ethylene propylene diene monomer (M-class) rubber (EPDM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;

or R¹ is a group of formula (i);

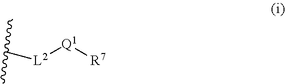

wherein the wavy line ( [symbol] ) indicates the point of attachment to the indole ring of formula (I);

L² is selected from the group consisting of C₁₋₁₀alkylene; C₃₋₆cycloalkylene; C₂₋₁₀alkenylene; C₂₋₁₀alkynylene; C₆₋₁₂arylene; and C₆₋₁₂arylC₁₋₆alkylene; preferably L² is selected from C₁₋₁₀alkylene; C₃₋₆cycloalkylene; C₆₋₁₂arylene; and C₆₋₁₂arylC₁₋₆alkylene; preferably L² is selected from C₁₋₁₀alkylene; C₃₋₆cycloalkylene; and C₆₋₁₂arylene; preferably L² is selected from C₁₋₁₀alkylene; C₃₋₆cycloalkylene; and C₆₋₁₂arylene; preferably L² is selected from C₁₋₆alkylene; C₃₋₆cycloalkylene; and C₆₋₁₂arylene; preferably L² is selected from C₁₋₆alkylene; C₃₋₆cycloalkylene; and C₆₋₁₂arylene; and wherein said C₁₋₁₀alkylene; C₃₋₆cycloalkylene; C₂₋₁₀alkenylene; C₂₋₁₀alkynylene; C₆₋₁₂arylene or C₆₋₁₂arylC₁₋₆alkylene, can be unsubstituted or substituted with one or more Z²; preferably said C₁₋₁₀alkylene; C₃₋₆cycloalkylene; C₆₋₁₂arylene or C₆₋₁₂arylC₁₋₆alkylene, can be unsubstituted or substituted with one or more Z²; preferably said C₁₋₁₀alkylene; C₃₋₆cycloalkylene; C₆₋₁₂arylene, can be unsubstituted or substituted with one or more Z²; preferably said C₁₋₆alkylene; C₃₋₆cycloalkylene; C₆₋₁₂arylene, can be unsubstituted or substituted with one or more Z²; preferably said C₁₋₆alkylene; C₃₋₆cycloalkylene; C₆₋₁₂arylene, can be unsubstituted or substituted with one or more Z²;

Q¹ is a single bond or selected from the group consisting of C₁₋₁₀alkylene; C₃₋₆cycloalkylene; —O—; —S—; —O—C₁₋₁₀alkylene; and —S—C₁₋₁₀alkylene; preferably Q¹ is a single bond or selected from the group consisting of C₁₋₁₀alkylene; C₃₋₆cycloalkylene; —O—; —S—; —O—C₁₋₁₀alkylene; preferably Q¹ is a single bond or selected from the group consisting of C₁₋₁₀alkylene; C₃₋₆cycloalkylene; —O—; —S—; preferably Q¹ is a single bond or selected from the group consisting of C₁₋₁₀alkylene; C₃₋₆cycloalkylene; wherein each left side of said groups is attached to L² and the right side thereof is attached to R⁷;

and wherein said C₁₋₁₀alkylene and said C₃₋₆cycloalkylene; can be unsubstituted or substituted with one or more Z³;

R⁷ is selected from the group consisting of hydrogen; OH, SH, COOH, NH₂; NHR²⁰, halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group; preferably R⁷ is selected from the group consisting of hydrogen; OH, SH, COOH, NH₂; NHR²⁰, —C(O)NR²⁰R²¹; C₁₋₂₀alkyl, C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;

wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl; can be unsubstituted or substituted with one or more Z⁴; preferably said C₁₋₂₀alkyl, C₆₋₁₂aryl, C₃₋₈cycloalkyl; C₆₋₁₂arylC₁₋₆alkyl; heterocyclyl; heteroaryl; heterocyclylC₁₋₆alkyl; or heteroarylC₁₋₆alkyl; can be unsubstituted or substituted with one or more Z⁴; preferably said C₁₋₂₀alkyl, C₆₋₁₂aryl, C₃₋₈cycloalkyl; C₆₋₁₂arylC₁₋₆alkyl; can be unsubstituted or substituted with one or more Z⁴;

and wherein for R⁷ said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group; preferably wherein said polymeric group is selected from the group consisting of polyurethane; isocyanate containing prepolymer, hydroxyl containing prepolymer, polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; ethylene propylene diene monomer (M-class) rubber (EPDM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group; preferably wherein said polymeric group is selected from the group consisting of polyurethane; isocyanate containing prepolymer, hydroxyl containing prepolymer, polyacrylate; polymethacrylate; polystyrene; copolymer of acrylate; poly(methyl methacrylate); methacrylate and/or styrene; poly(isobornyl acrylate); polyacrylonitrile; polyamide; polyester amide; ethylene propylene diene monomer (M-class) rubber (EPDM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;

or R⁷ is a group of formula (ii);

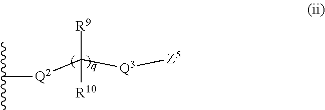

wherein the wavy line ( [symbol] ) indicates the point of attachment to Q¹;
q is an integer selected from 0; 1, 2, 3 or 4; preferably q is selected from 0; 1, 2, or 3; preferably q is selected from 0; 1, or 2;
Q² is a single bond or selected from the group consisting of CR¹¹R¹²; O, S, and NR¹³; preferably Q² is selected from the group consisting of CR¹¹R¹²; O, S, and NR¹³; preferably Q² is selected from the group consisting of CR¹¹R¹²; O, S;

Q³ is a single bond or selected from the group consisting of hydroxyC₁₋₁₀alkylene; aminoC₁₋₁₀alkylene; mercaptoC₁₋₁₀alkylene; C₁₋₂₀alkylene, C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₆alkylene; and heteroarylC₁₋₆alkylene; preferably Q³ is a single bond or selected from the group consisting of hydroxyC₁₋₁₀alkylene; aminoC₁₋₁₀alkylene; mercaptoC₁₋₁₀alkylene; C₁₋₂₀alkylene, C₆₋₁₂arylene, C₃₋₈cycloalkylene; C₆₋₁₂arylC₁₋₆alkylene; heterocyclylene; heteroarylene; heterocyclylC₁₋₆alkylene; and heteroarylC₁₋₆alkylene; preferably Q³ is a single bond or selected from the group consisting of hydroxyC₁₋₁₀alkylene; aminoC₁₋₁₀alkylene; mercaptoC₁₋₁₀alkylene; C₁₋₂₀alkylene, C₃₋₈cycloalkylene; C₆₋₁₂arylC₁₋₆alkylene; heterocyclylC₁₋₆alkylene; and heteroarylC₁₋₆alkylene; preferably Q³ is a single bond or selected from the group consisting of hydroxyC₁₋₆alkylene; aminoC₁₋₆alkylene; mercaptoC₁₋₈alkylene; C₁₋₆alkylene, C₃₋₈cycloalkylene; C₆₋₁₂arylC₁₋₆alkylene; heterocyclylC₁₋₆alkylene; and heteroarylC₁₋₆alkylene; preferably Q³ is a single bond or selected from the group consisting of hydroxyC₁₋₆alkylene; aminoC₁₋₆alkylene; mercaptoC₁₋₆alkylene; C₁₋₆alkylene, C₃₋₈cycloalkylene; C₆₋₁₂arylC₁₋₆alkylene; heterocyclylC₁₋₆alkylene; and heteroarylC₁₋₆alkylene;

wherein said C₁₋₂₀alkylene, C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₆alkylene; or heteroarylC₁₋₆alkylene, can be unsubstituted or substituted with one or more Z^{5a}; preferably said C₁₋₂₀alkylene, C₆₋₁₂arylene, C₃₋₈cycloalkylene; C₆₋₁₂arylC₁₋₆alkylene; heterocyclylene; heteroarylene; heterocyclylC₁₋₆alkylene; or heteroarylC₁₋₆alkylene, can be unsubstituted or substituted with one or more Z^{5a}; preferably said C₁₋₂₀alkylene, C₆₋₁₂arylene, C₃₋₈cycloalkylene; heterocyclylene; or heteroarylene; can be unsubstituted or substituted with one or more Z^{5a};
wherein if q is 0, then one of Q² and Q³ is not a single bond;
each R⁹ and R¹⁰ is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group; preferably each R⁹ and R¹⁰ is independently selected from the group consisting of hydrogen; C₁₋₂₀alkyl, hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group; preferably each R⁹ and R¹⁰ is independently selected from the group consisting of hydrogen; C₁₋₂₀alkyl, hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclylC₁₋₆alkyl; heteroarylC₁₋₆alkyl; and a polymeric group; preferably each R⁹ and R¹⁰ is independently selected from the group consisting of hydrogen; C₁₋₁₈alkyl, hydroxyC₁₋₈alkoxy; aminoC₁₋₈alkoxy; mercaptoC₁₋₈alkoxy; C₆₋₁₂aryl, C₃₋₈cycloalkyl; C₆₋₁₂arylC₁₋₆alkyl; heterocyclylC₁₋₆alkyl; heteroarylC₁₋₆alkyl; and a polymeric group; preferably each R⁹ and R¹³ is independently selected from the group consisting of hydrogen; C₁₋₁₀alkyl, hydroxyC₁₋₆alkoxy; aminoC₁₋₆alkoxy; mercaptoC₁₋₆alkoxy; C₆₋₁₂aryl, C₃₋₈cycloalkyl; C₆₋₁₂arylC₁₋₆alkyl; heterocyclylC₁₋₆alkyl; heteroarylC₁₋₆alkyl; and a polymeric group;
wherein for each R⁹ and R¹⁰ said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group; preferably wherein said polymeric group is selected from the group consisting of polyurethane; isocyanate containing prepolymer, hydroxyl containing prepolymer, polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; ethylene propylene diene monomer (M-class) rubber (EPDM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group; preferably wherein said polymeric group is selected from the group consisting of polyurethane; isocyanate containing prepolymer, hydroxyl containing prepolymer, polyacrylate; polymethacrylate; polystyrene; copolymer of acrylate; poly(methyl methacrylate); methacrylate and/or styrene; poly(isobornyl acrylate); polyacrylonitrile; polyamide; polyester amide; ethylene propylene diene monomer (M-class) rubber (EPDM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
each R¹¹ and R¹² is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group; preferably each R¹¹ and R¹² is independently selected from the group consisting of hydrogen; C₁₋₂₀alkyl, hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group; preferably each R¹¹ and R¹² is independently selected from the group consisting of hydrogen; C₁₋₂₀alkyl, hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclylC₁₋₆alkyl; heteroarylC₁₋₆alkyl; and a polymeric group; preferably each R¹¹ and R¹² is independently selected from the group consisting of hydrogen; C₁₋₁₈alkyl, hydroxyC₁₋₈alkoxy; aminoC₁₋₈alkoxy; mercaptoC₁₋₈alkoxy; C₆₋₁₂aryl, C₃₋₈cycloalkyl; C₆₋₁₂arylC₁₋₆alkyl; heterocyclylC₁₋₆alkyl; heteroarylC₁₋₆alkyl; and a polymeric group; preferably each R¹¹ and R¹² is independently selected from the group consisting of hydrogen; C₁₋₁₀alkyl, hydroxyC₁₋₆alkoxy; aminoC₁₋₆alkoxy; mercaptoC₁₋₆alkoxy; C₆₋₁₂aryl, C₃₋₈cycloalkyl; C₆₋₁₂arylC₁₋₆alkyl; heterocyclylC₁₋₆alkyl; heteroarylC₁₋₆alkyl; and a polymeric group;
wherein for each R¹¹ and R¹² said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group; preferably wherein said polymeric group is selected from the group consisting of polyurethane; isocyanate containing prepolymer, hydroxyl containing prepolymer, polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; ethylene propylene diene monomer (M-class) rubber (EPDM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group; preferably wherein said polymeric group is selected from the group consisting of polyurethane; isocyanate containing prepolymer, hydroxyl containing prepolymer, polyacrylate; polymethacrylate; polystyrene; copolymer of acrylate; poly(methyl methacrylate); methacrylate and/or styrene; poly(isobornyl acrylate); polyacrylonitrile; polyamide; polyester amide; ethylene propylene diene monomer (M-class) rubber (EPDM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
R¹³ is selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; and heteroarylC₁₋₆alkyl; preferably R¹³ is selected from the group consisting of hydrogen; C₁₋₂₀alkyl, hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; and heteroarylC₁₋₆alkyl; preferably R¹³ is selected from the group consisting of hydrogen; C₁₋₂₀alkyl, hydroxyC₁₋₁₀alkoxy; aminoC₁₋₁₀alkoxy; mercaptoC₁₋₁₀alkoxy; C₃₋₈cycloalkyl; C₆₋₁₂arylC₁₋₆alkyl; heterocyclylC₁₋₆alkyl; and heteroarylC₁₋₆alkyl; preferably R¹³ is selected from the group consisting of hydrogen; C₁₋₁₈alkyl, hydroxyC₁₋₈alkoxy; aminoC₁₋₈alkoxy; mercaptoC₁₋₈alkoxy; C₃₋₈cycloalkyl; C₆₋₁₂arylC₁₋₆alkyl; heterocyclylC₁₋₆alkyl; and heteroarylC₁₋₆alkyl; preferably R¹³ is selected from the group consisting of hydrogen; C₁₋₁₀alkyl, hydroxyC₁₋₆alkoxy; aminoC₁₋₆alkoxy; mercaptoC₁₋₆alkoxy; C₃₋₈cycloalkyl; C₆₋₁₂arylC₁₋₆alkyl; heterocyclylC₁₋₆alkyl; and heteroarylC₁₋₆alkyl;

R² is selected from the group consisting of halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; heteroarylC₁₋₆alkyl; and a polymeric group; preferably R² is selected from the group consisting halo; nitro; cyano; —C(O)NR²⁰R²¹; C₁₋₂₀alkyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; heteroarylC₁₋₆alkyl; and a polymeric group; preferably R² is selected from the group consisting halo; nitro; cyano; —C(O)NR²⁰R²¹; C₁₋₂₀alkyl; C₆₋₁₂aryl; C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group; preferably R² is selected from the group consisting halo; nitro; cyano; —C(O)NR²⁰R²¹; C₁₋₂₀alkyl; C₆₋₁₂aryl; C₃₋₈cycloalkyl; C₆₋₁₂arylC₁₋₆alkyl; heterocyclyl; heteroaryl; and a polymeric group; preferably R² is selected from the group consisting halo; C₁₋₂₀alkyl; C₆₋₁₂aryl; C₃₋₈cycloalkyl; C₆₋₁₂arylC₁₋₆alkyl; heterocyclyl; heteroaryl; and a polymeric group;
wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl, heteroarylC₁₋₆alkyl, can be unsubstituted or substituted with one or more Z⁶; wherein said C₁₋₂₀alkyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; and heteroarylC₁₋₆alkyl can be unsubstituted or substituted with one or more Z⁶; wherein said C₁₋₂₀alkyl; C₆₋₁₂aryl; C₃₋₈cycloalkyl; C₆₋₁₂arylC₁₋₆alkyl; heterocyclyl; heteroaryl; heterocyclylC₁₋₆alkyl; and heteroarylC₁₋₆alkyl, can be unsubstituted or substituted with one or more Z⁶; wherein said C₁₋₂₀alkyl; C₆₋₁₂aryl; C₃₋₈cycloalkyl; C₆₋₁₂arylC₁₋₆alkyl; heterocyclyl; heteroaryl, can be unsubstituted or substituted with one or more Z⁶; wherein said C₁₋₂₀alkyl; C₆₋₁₂aryl; C₃₋₈cycloalkyl; C₆₋₁₂arylC₁₋₆alkyl; heterocyclyl; heteroaryl; can be unsubstituted or substituted with one or more Z⁶;

R³ is selected from the group consisting of halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; heteroarylC₁₋₆alkyl; and a polymeric group; preferably R³ is selected from the group consisting halo; nitro; cyano; —C(O)NR²⁰R²¹; C₁₋₂₀alkyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; heteroarylC₁₋₆alkyl; and a polymeric group; preferably R³ is selected from the group consisting halo; nitro; cyano; —C(O)NR²⁰R²¹; C₁₋₂₀alkyl; C₆₋₁₂aryl; C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group; preferably R³ is selected from the group consisting halo; nitro; cyano; —C(O)NR²⁰R²¹; C₁₋₂₀alkyl; C₆₋₁₂aryl; C₃₋₈cycloalkyl; C₆₋₁₂arylC₁₋₆alkyl; heterocyclyl; heteroaryl; and a polymeric group; preferably R³ is selected from the group consisting halo; C₁₋₂₀alkyl; C₆₋₁₂aryl; C₃₋₈cycloalkyl; C₆₋₁₂arylC₁₋₆alkyl; heterocyclyl; heteroaryl;
wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl, heteroarylC₁₋₆alkyl, can be unsubstituted or substituted with one or more Z⁶; wherein said C₁₋₂₀alkyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; and heteroarylC₁₋₆alkyl can be unsubstituted or substituted with one or more Z⁶; wherein said C₁₋₂₀alkyl; C₆₋₁₂aryl; C₃₋₈cycloalkyl; C₆₋₁₂arylC₁₋₆alkyl; heterocyclyl; heteroaryl; heterocyclylC₁₋₆alkyl; and heteroarylC₁₋₆alkyl, can be unsubstituted or substituted with one or more Z⁶; wherein said C₁₋₂₀alkyl; C₆₋₁₂aryl; C₃₋₈cycloalkyl; C₆₋₁₂arylC₁₋₆alkyl; heterocyclyl; heteroaryl, can be unsubstituted or substituted with one or more Z⁶; wherein said C₁₋₂₀alkyl; C₆₋₁₂aryl; C₃₋₈cycloalkyl; C₆₋₁₂arylC₁₋₆alkyl; heterocyclyl; heteroaryl; can be unsubstituted or substituted with one or more Z⁶;

R⁴ is selected from the group consisting of halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; heteroarylC₁₋₆alkyl; and a polymeric group; preferably R⁴ is selected from the group consisting of halo; nitro; cyano; —C(O)NR²⁰R²¹; C₁₋₂₀alkyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; heteroarylC₁₋₆alkyl; and a polymeric group; preferably R⁴ is selected from the group consisting of halo; nitro; cyano; —C(O)NR²⁰R²¹; C₁₋₂₀alkyl; C₆₋₁₂aryl; C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group; preferably R⁴ is selected from the group consisting of halo; nitro; cyano; —C(O)NR²⁰R²¹; C₁₋₂₀alkyl; C₆₋₁₂aryl; C₃₋₈cycloalkyl; C₆₋₁₂arylC₁₋₆alkyl; heterocyclyl; heteroaryl; and a polymeric group; preferably R⁴ is selected from the group consisting of halo; C₁₋₂₀alkyl; C₆₋₁₂aryl; C₃₋₈cycloalkyl; C₆₋₁₂arylC₁₋₆alkyl; heterocyclyl; heteroaryl; and a polymeric group;
wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl, heteroarylC₁₋₆alkyl, can be unsubstituted or substituted with one or more Z⁶; wherein said C₁₋₂₀alkyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; and heteroarylC₁₋₆alkyl can be unsubstituted or substituted with one or more Z⁶; wherein said C₁₋₂₀alkyl; C₆₋₁₂aryl; C₃₋₈cycloalkyl; C₆₋₁₂arylC₁₋₆alkyl; heterocyclyl; heteroaryl; heterocyclylC₁₋₆alkyl; and heteroarylC₁₋₆alkyl, can be unsubstituted or substituted with one or more Z⁶; wherein said C₁₋₂₀alkyl; C₆₋₁₂aryl; C₃₋₈cycloalkyl; C₆₋₁₂arylC₁₋₆alkyl; heterocyclyl; heteroaryl, can be unsubstituted or substituted with one or more Z⁶; wherein said C₁₋₂₀alkyl; C₆₋₁₂aryl; C₃₋₈cycloalkyl; C₆₋₁₂arylC₁₋₆alkyl; heterocyclyl; heteroaryl; can be unsubstituted or substituted with one or more Z⁶;
and wherein for each of R² and R³ and R⁴ said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group; preferably wherein said polymeric group is selected from the group consisting of polyurethane; isocyanate containing prepolymer, hydroxyl containing prepolymer, polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; ethylene propylene diene monomer (M-class) rubber (EPDM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group; preferably wherein said polymeric group is selected from the group consisting of polyurethane; isocyanate containing prepolymer, hydroxyl containing prepolymer, polyacrylate; polymethacrylate; polystyrene; copolymer of acrylate; poly(methyl methacrylate); methacrylate and/or styrene; poly(isobornyl acrylate); polyacrylonitrile; polyamide; polyester amide; ethylene propylene diene monomer (M-class) rubber (EPDM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;

or R² and R³ and R⁴ together with the carbon atom to which they are attached can form a saturated or unsaturated 4-, 5-, 6-, 7- or 8-membered ring; preferably R² and R³ and R⁴ together with the carbon atom to which they are attached can form a saturated or unsaturated 4-, 5-, 6-, 7-membered ring; preferably R² and R³ and R⁴ together with the carbon atom to which they are attached can form a saturated or unsaturated 5-, 6-, or 7-membered ring; preferably R² and R³ and R⁴ together with the carbon atom to which they are attached can form an unsaturated 5-, 6-, or 7-membered ring; preferably R² and R³ and R⁴ together with the carbon atom to which they are attached can form a saturated or unsaturated 5-, or 6-membered ring; preferably R² and R³ and R⁴ together with the carbon atom to which they are attached can form an unsaturated 5-, or 6-membered ring; preferably R² and R³ and R⁴ together with the carbon atom to which they are attached can form a saturated or unsaturated 6-membered ring; preferably R² and R³ and R⁴ together with the carbon atom to which they are attached can form an unsaturated 6-membered ring; preferably R² and R³ and R⁴ together with the carbon atom to which they are attached can form an aryl ring; for example a phenyl ring;
wherein said saturated or unsaturated 4-, 5-, 6-, 7- or 8-membered ring optionally comprises one or more heteroatoms, said heteroatoms being each independently selected from O, S and N; preferably said saturated or unsaturated 4-, 5-, 6-, 7-membered ring optionally comprises one or more heteroatoms, said heteroatoms being each independently selected from O, S and N; preferably said saturated or unsaturated 5-, 6-, 7-membered ring optionally comprises one or more heteroatoms, said heteroatoms being each independently selected from O, S and N; preferably said unsaturated 5-, 6-, or 7-membered ring optionally comprises one or more heteroatoms, said heteroatoms being each independently selected from O, S and N; preferably said saturated or unsaturated 5-, or 6-membered ring optionally comprises one or more heteroatoms, said heteroatoms being each independently selected from O, S and N; preferably said unsaturated 5-, or 6-membered ring optionally comprises one or more heteroatoms, said heteroatoms being each independently selected from O, S and N; preferably said saturated or unsaturated 6-membered ring optionally comprises one or more heteroatoms, said heteroatoms being each independently selected from O, S and N; preferably said unsaturated 6-membered ring optionally comprises one or more heteroatoms, said heteroatoms being each independently selected from O, S and N; preferably said phenyl ring optionally comprises one or more heteroatoms, said heteroatoms being each independently selected from O, S and N;
wherein said saturated or unsaturated 4-, 5-, 6-, 7- or 8-membered ring can be unsubstituted or substituted with one or more R⁸; wherein said saturated or unsaturated 4-, 5-, 6-, 7- or 8-membered ring can be unsubstituted or substituted with one, two, or three R⁸; preferably said saturated or unsaturated 4-, 5-, 6-, 7-membered ring can be unsubstituted or substituted with one or more R⁸; preferably said saturated or unsaturated 5-, 6-, 7-membered ring can be unsubstituted or substituted with one or more R⁸; preferably said unsaturated 5-, 6-, or 7-membered ring can be unsubstituted or substituted with one or more R⁸; preferably said saturated or unsaturated 5-, or 6-membered can be unsubstituted or substituted with one or more R⁸; preferably said saturated or unsaturated 5-, or 6-membered can be unsubstituted or substituted with one, two or three R⁸; preferably said unsaturated 5-, or 6-membered ring can be unsubstituted or substituted with one or more R⁸; preferably said saturated or unsaturated 6-membered ring can be unsubstituted or substituted with one or more R⁸; preferably said unsaturated 6-membered ring can be unsubstituted or substituted with one or more R⁸; preferably said phenyl ring can be unsubstituted or substituted with one or more R⁸; preferably said phenyl ring can be unsubstituted or substituted with one, two or three R⁸;
each R⁸ is independently selected from the group consisting of hydroxyC₁₋₁₀alkoxy; aminoC₁₋₁₀alkoxy; mercaptoC₁₋₁₀alkoxy; C₁₋₁₀alkyl; C₂₋₁₀alkenyl; C₂₋₁₀alkynyl; C₆₋₁₂arylC₁₋₆alkyl; C₃₋₈cycloalkyl; C₆₋₁₂arylC₁₋₆alkyl; heterocyclyl; heteroaryl; heterocyclylC₁₋₆alkyl; heteroarylC₁₋₆alkyl; OH; SH; COOH; NH₂; NHR²⁰; halogen; haloC₁₋₆alkyl; haloC₁₋₆alkyloxy; —OR¹⁹; —SR¹⁹; nitro; NR²⁰R²¹; —C(O)NR²⁰R²¹; cyano; and a polymeric group; preferably each R⁸ is independently selected from the group consisting of hydroxyC₁₋₁₀alkoxy; aminoC₁₋₁₀alkoxy; mercaptoC₁₋₁₀alkoxy; C₁₋₁₀alkyl; C₆₋₁₂arylC₁₋₆alkyl; C₃₋₈cycloalkyl; C₆₋₁₂arylC₁₋₆alkyl; heterocyclyl; heteroaryl; heterocyclylC₁₋₆alkyl; heteroarylC₁₋₆alkyl; OH; SH; COOH; NH₂, NHR²⁰; halogen; haloC₁₋₆alkyl; haloC₁₋₆alkyloxy; —OR¹⁹; —SR¹⁹; nitro; NR²⁰R²¹; —C(O)NR²⁰R²¹; cyano; and a polymeric group; preferably each R⁸ is independently selected from the group consisting of hydroxyC₁₋₁₀alkoxy; aminoC₁₋₁₀alkoxy; mercaptoC₁₋₁₀alkoxy; C₁₋₁₀alkyl; C₆₋₁₂arylC₁₋₆alkyl; C₃₋₈cycloalkyl; C₆₋₁₂arylC₁₋₆alkyl; heterocyclylC₁₋₆alkyl; heteroarylC₁₋₆alkyl; OH; SH; COOH; NH₂; NHR²⁰; —OR¹⁹; —SR¹⁹; and a polymeric group; preferably each R⁸ is independently selected from the group consisting of hydroxyC₁₋₈alkoxy; aminoC₁₋₈alkoxy; mercaptoC₁₋₈alkoxy; C₁₋₆alkyl; C₆₋₁₂arylC₁₋₆alkyl; C₃₋₈cycloalkyl; C₆₋₁₂arylC₁₋₆alkyl; heterocyclylC₁₋₆alkyl; heteroarylC₁₋₆alkyl; OH; SH; COOH; NH₂; NHR²⁰; —OR¹⁹; —SR¹⁹; and a polymeric group; preferably each R⁸ is independently selected from the group consisting of hydroxyC₁₋₆alkoxy; aminoC₁₋₆alkoxy; mercaptoC₁₋₆alkoxy; C₁₋₆alkyl; C₆₋₁₂arylC₁₋₆alkyl; C₃₋₈cycloalkyl; C₆₋₁₂arylC₁₋₆alkyl; heterocyclylC₁₋₆alkyl; heteroarylC₁₋₆alkyl; OH; SH; COOH; NH₂; NHR²⁰; and a polymeric group;
   wherein said hydroxyC₁₋₁₀alkoxy; aminoC₁₋₁₀alkoxy; mercaptoC₁₋₁₀alkoxy; C₁₋₁₀alkyl; C₂₋₁₀alkenyl; C₂₋₁₀alkynyl; C₆₋₁₂arylC₁₋₆alkyl; C₃₋₈cycloalkyl; C₆₋₁₂arylC₁₋₆alkyl; heterocyclyl; heteroaryl; heterocyclylC₁₋₆alkyl; or heteroarylC₁₋₆alkyl, can be unsubstituted or substituted with one or more Z⁷; preferably said hydroxyC₁₋₁₀alkoxy; aminoC₁₋₁₀alkoxy; mercaptoC₁₋₁₀alkoxy; C₁₋₁₀alkyl; C₆₋₁₂arylC₁₋₆alkyl; C₃₋₈cycloalkyl; C₆₋₁₂arylC₁₋₆alkyl; heterocyclyl; heteroaryl; heterocyclylC₁₋₆alkyl; or heteroarylC₁₋₆alkyl, can be unsubstituted or substituted with one or more Z⁷; preferably said hydroxyC₁₋₁₀alkoxy; aminoC₁₋₁₀alkoxy; mercaptoC₁₋₁₀alkoxy; C₁₋₁₀alkyl; C₆₋₁₂arylC₁₋₆alkyl; C₃₋₈cycloalkyl; C₆₋₁₂arylC₁₋₆alkyl; heterocyclylC₁₋₆alkyl; or heteroarylC₁₋₆alkyl, can be unsubstituted or substituted with one or more Z⁷; preferably said hydroxyC₁₋₆alkoxy; aminoC₁₋₆alkoxy; mercaptoC₁₋₆alkoxy; C₁₋₆alkyl; C₆₋₁₂arylC₁₋₆alkyl; C₃₋₈cycloalkyl; C₆₋₁₂arylC₁₋₆alkyl; heterocyclylC₁₋₆alkyl; or heteroarylC₁₋₆alkyl, can be unsubstituted or substituted with one or more Z⁷;
   and wherein for R⁸ said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group; preferably wherein said polymeric group is selected from the group consisting of polyurethane; isocyanate containing prepolymer, hydroxyl containing prepolymer, polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; ethylene propylene diene monomer (M-class) rubber (EPDM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group; preferably wherein said polymeric group is selected from the group consisting of polyurethane; isocyanate containing prepolymer, hydroxyl containing prepolymer, polyacrylate; polymethacrylate; polystyrene; copolymer of acrylate; poly(methyl methacrylate); methacrylate and/or styrene; poly(isobornyl acrylate); polyacrylonitrile; polyamide; polyester amide; ethylene propylene diene monomer (M-class) rubber (EPDM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;

or R⁸ is a group of formula (iii);

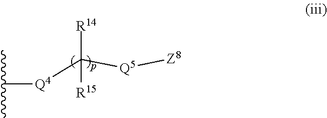

wherein the wavy line ( [symbol] ) indicates the point of attachment to the ring formed by R² and R³ and R⁴ together;
p is an integer selected from 0; 1, 2, 3 or 4; preferably p is selected from 0; 1, 2, or 3; preferably p is selected from 0; 1, or 2; preferably p is 1 or 2;
Q⁴ is a single bond or selected from the group consisting of CR¹⁶R¹⁷; O, S, and NR¹⁸; preferably Q⁴ is selected from the group consisting of CR¹⁶R¹⁷; O, S;
Q⁵ is a single bond or selected from the group consisting of hydroxyC₁₋₁₀alkylene; aminoC₁₋₁₀alkylene; mercaptoC₁₋₁₀alkylene; C₁₋₂₀alkylene, C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₆alkylene; and heteroarylC₁₋₆alkylene; preferably Q⁵ is a single bond or selected from the group consisting of hydroxyC₁₋₁₀alkylene; aminoC₁₋₁₀alkylene; mercaptoC₁₋₁₀alkylene; C₁₋₂₀alkylene, C₆₋₁₂arylene, C₃₋₈cycloalkylene; C₆₋₁₂arylC₁₋₆alkylene; heterocyclylene; heteroarylene; heterocyclylC₁₋₆alkylene; and heteroarylC₁₋₆alkylene; preferably Q⁵ is a single bond or selected from the group consisting of hydroxyC₁₋₁₀alkylene; aminoC₁₋₁₀alkylene; mercaptoC₁₋₁₀alkylene; C₁₋₂₀alkylene, C₃₋₈cycloalkylene; C₆₋₁₂arylC₁₋₆alkylene; heterocyclylC₁₋₆alkylene; and heteroarylC₁₋₆alkylene; preferably Q⁵ is a single bond or selected from the group consisting of hydroxyC₁₋₆alkylene; aminoC₁₋₆alkylene; mercaptoC₁₋₈alkylene; C₁₋₆alkylene, C₃₋₈cycloalkylene; C₆₋₁₂arylC₁₋₆alkylene; heterocyclylC₁₋₆alkylene; and heteroarylC₁₋₆alkylene; preferably Q⁵ is a single bond or selected from the group consisting of hydroxyC₁₋₆alkylene; aminoC₁₋₆alkylene; mercaptoC₁₋₆alkylene; C₁₋₆alkylene, C₃₋₈cycloalkylene; C₆₋₁₂arylC₁₋₆alkylene; heterocyclylC₁₋₆alkylene; and heteroarylC₁₋₆alkylene;
wherein said C₁₋₂₀alkylene, C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₆alkylene; or heteroarylC₁₋₆alkylene, can be unsubstituted or substituted with one or more Z^{5a}; preferably said C₁₋₂₀alkylene, C₆₋₁₂arylene, C₃₋₈cycloalkylene; C₆₋₁₂arylC₁₋₆alkylene; heterocyclylene; heteroarylene; heterocyclylC₁₋₆alkylene; or heteroarylC₁₋₆alkylene, can be unsubstituted or substituted with one or more Z^{5a}; preferably said C₁₋₂₀alkylene, C₆₋₁₂arylene, C₃₋₈cycloalkylene; heterocyclylene; or heteroarylene; can be unsubstituted or substituted with one or more Z^{5a};
wherein if p is 0, then one of Q⁴ and Q⁵ is not a single bond;
each R¹⁴ and R¹⁵ is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group; preferably each R¹⁴ and R¹⁵ is independently selected from the group consisting of hydrogen; C₁₋₂₀alkyl, hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group; preferably each R¹⁴ and R¹⁵ is independently selected from the group consisting of hydrogen; C₁₋₂₀alkyl, hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclylC₁₋₆alkyl; heteroarylC₁₋₆alkyl; and a polymeric group; preferably each R¹⁴ and R¹⁵ is independently selected from the group consisting of hydrogen; C₁₋₁₈alkyl, hydroxyC₁₋₈alkoxy; aminoC₁₋₈alkoxy; mercaptoC₁₋₈alkoxy; C₆₋₁₂aryl, C₃₋₈cycloalkyl; C₆₋₁₂arylC₁₋₆alkyl; heterocyclylC₁₋₆alkyl; heteroarylC₁₋₆alkyl; and a polymeric group; preferably each R¹⁴ and R¹⁵ is independently selected from the group consisting of hydrogen; C₁₋₁₀alkyl, hydroxyC₁₋₆alkoxy; aminoC₁₋₆alkoxy; mercaptoC₁₋₆alkoxy; C₆₋₁₂aryl, C₃₋₈cycloalkyl; C₆₋₁₂arylC₁₋₆alkyl; heterocyclylC₁₋₆alkyl; heteroarylC₁₋₆alkyl; and a polymeric group;
wherein for each R¹⁴ and R¹⁵ said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group; preferably wherein said polymeric group is selected from the group consisting of polyurethane; isocyanate containing prepolymer, hydroxyl containing prepolymer, polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; ethylene propylene diene monomer (M-class) rubber (EPDM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group; preferably wherein said polymeric group is selected from the group consisting of polyurethane; isocyanate containing prepolymer, hydroxyl containing prepolymer, polyacrylate; polymethacrylate; polystyrene; copolymer of acrylate; poly(methyl methacrylate); methacrylate and/or styrene; poly(isobornyl acrylate); polyacrylonitrile; polyamide; polyester amide; ethylene propylene diene monomer (M-class) rubber (EPDM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
each R¹⁶ and R¹⁷ is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group; preferably each R¹⁶ and R¹⁷ is independently selected from the group consisting of hydrogen; C₁₋₂₀alkyl, hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group; preferably each R¹⁶ and R¹⁷ is independently selected from the group consisting of hydrogen; C₁₋₂₀alkyl, hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclylC₁₋₆alkyl; heteroarylC₁₋₆alkyl; and a polymeric group; preferably each R¹⁶ and R¹⁷ is independently selected from the group consisting of hydrogen; C₁₋₁₈alkyl, hydroxyC₁₋₈alkoxy; aminoC₁₋₈alkoxy; mercaptoC₁₋₈alkoxy; C₆₋₁₂aryl, C₃₋₈cycloalkyl; C₆₋₁₂arylC₁₋₆alkyl; heterocyclylC₁₋₆alkyl; heteroarylC₁₋₆alkyl; and a polymeric group; preferably each R¹⁶ and R¹⁷ is independently selected from the group consisting of hydrogen; C₁₋₁₀alkyl, hydroxyC₁₋₆alkoxy; aminoC₁₋₆alkoxy; mercaptoC₁₋₆alkoxy; C₆₋₁₂aryl, C₃₋₈cycloalkyl; C₆₋₁₂arylC₁₋₆alkyl; heterocyclylC₁₋₆alkyl; heteroarylC₁₋₆alkyl; and a polymeric group;
wherein for each R¹⁶ and R¹⁷ said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group; preferably wherein said polymeric group is selected from the group consisting of polyurethane; isocyanate containing prepolymer, hydroxyl containing prepolymer, polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; ethylene propylene diene monomer (M-class) rubber (EPDM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group; preferably wherein said polymeric group is selected from the group consisting of polyurethane; isocyanate containing prepolymer, hydroxyl containing prepolymer, polyacrylate; polymethacrylate; polystyrene; copolymer of acrylate; poly(methyl methacrylate); methacrylate and/or styrene; poly(isobornyl acrylate); polyacrylonitrile; polyamide; polyester amide; ethylene propylene diene monomer (M-class) rubber (EPDM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
R¹⁸ is selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; and heteroarylC₁₋₆alkyl; preferably R¹⁸ is selected from the group consisting of hydrogen; C₁₋₂₀alkyl, hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; and heteroarylC₁₋₆alkyl; preferably R¹⁸ is selected from the group consisting of hydrogen; C₁₋₂₀alkyl, hydroxyC₁₋₁₀alkoxy; aminoC₁₋₁₀alkoxy; mercaptoC₁₋₁₀alkoxy; C₃₋₈cycloalkyl; C₆₋₁₂arylC₁₋₆alkyl; heterocyclylC₁₋₆alkyl; and heteroarylC₁₋₆alkyl; preferably R¹⁸ is selected from the group consisting of hydrogen; C₁₋₁₀alkyl, hydroxyC₁₋₈alkoxy; aminoC₁₋₈alkoxy; mercaptoC₁₋₈alkoxy; C₃₋₈cycloalkyl; C₆₋₁₂arylC₁₋₆alkyl; heterocyclylC₁₋₆alkyl; and heteroarylC₁₋₆alkyl; preferably R¹⁸ is selected from the group consisting of hydrogen; C₁₋₁₀alkyl, hydroxyC₁₋₆alkoxy; aminoC₁₋₆alkoxy; mercaptoC₁₋₆alkoxy; C₃₋₈cycloalkyl; C₆₋₁₂arylC₁₋₆alkyl; heterocyclylC₁₋₆alkyl; and heteroarylC₁₋₆alkyl;

L¹ is selected from the group consisting of C₁₋₁₀alkyleneC₃₋₈cycloalkylene; C₃₋₈cycloalkyleneC₁₋₁₀alkylene; C₁₋₁₀alkylene; C₃₋₈cycloalkylene; C₃₋₈cycloalkyleneC₁₋₁₀alkylC₃₋₈cycloalkylene; C₂₋₁₀alkenylene; C₂₋₁₀alkynylene; C₆₋₁₂arylene; C₆₋₁₂aryleneC₁₋₆alkylene; C₆₋₁₂aryleneC₁₋₆alkylC₆₋₁₂arylene; and C₁₋₆alkyleneC₆₋₁₂arylC₁₋₆alkylene; preferably L¹ is selected from the group consisting of C₁₋₁₀alkyleneC₃₋₈cycloalkylene; C₃₋₈cycloalkyleneC₁₋₁₀alkylene; C₁₋₁₀alkylene; C₃₋₈cycloalkylene; C₃₋₈cycloalkyleneC₁₋₁₀alkylC₃₋₈cycloalkylene; C₆₋₁₂arylene; C₆₋₁₂aryleneC₁₋₆alkylene; C₆₋₁₂aryleneC₁₋₆alkylC₆₋₁₂arylene; and C₁₋₆alkyleneC₆₋₁₂arylC₁₋₆alkylene; preferably L¹ is selected from the group consisting of C₁₋₁₀alkyleneC₃₋₈cycloalkylene; C₃₋₈cycloalkyleneC₁₋₁₀alkylene; C₁₋₁₀alkylene; C₃₋₈cycloalkylene; C₃₋₈cycloalkyleneC₁₋₁₀alkylC₃₋₈cycloalkylene; C₆₋₁₂arylene; C₆₋₁₂aryleneC₁₋₆alkylene; preferably L¹ is selected from the group consisting of C₁₋₁₀alkyleneC₃₋₈cycloalkylene; C₃₋₈cycloalkyleneC₁₋₁₀alkylene; C₁₋₁₀alkylene; C₃₋₈cycloalkylene; preferably L¹ is selected from the group consisting of C₁₋₈alkyleneC₃₋₈cycloalkylene; C₃₋₈cycloalkyleneC₁₋₆alkylene; C₁₋₆alkylene; C₃₋₈cycloalkylene; preferably L¹ is selected from the group consisting of C₁₋₆alkyleneC₃₋₈cycloalkylene; C₃₋₈cycloalkyleneC₁₋₆alkylene; C₁₋₆alkylene; C₃₋₈cycloalkylene;
wherein said C₁₋₁₀alkyleneC₃₋₈cycloalkylene; C₃₋₈cycloalkyleneC₁₋₁₀alkylene; C₁₋₁₀alkylene; C₃₋₈cycloalkylene; C₃₋₈cycloalkyleneC₁₋₁₀alkylC₃₋₈cycloalkylene; C₂₋₁₀alkenylene; C₂₋₁₀alkynylene; C₆₋₁₂arylene; C₆₋₁₂aryleneC₁₋₆alkylene; C₆₋₁₂aryleneC₁₋₆alkylC₆₋₁₂arylene; or C₁₋₆alkyleneC₆₋₁₂arylC₁₋₆alkylene, can be unsubstituted or substituted with one or more Z^{1a};
preferably said C₁₋₁₀alkyleneC₃₋₈cycloalkylene; C₃₋₈cycloalkyleneC₁₋₁₀alkylene; C₁₋₁₀alkylene; C₃₋₈cycloalkylene; C₃₋₈cycloalkyleneC₁₋₁₀alkylC₃₋₈cycloalkylene; C₆₋₁₂arylene; C₆₋₁₂aryleneC₁₋₈alkylene; C₆₋₁₂aryleneC₁₋₆alkylC₆₋₁₂arylene; and C₁₋₆alkyleneC₆₋₁₂arylC₁₋₆alkylene, can be unsubstituted or substituted with one or more Z^{1a}; preferably said C₁₋₁₀alkyleneC₃₋₈cycloalkylene; C₃₋₈cycloalkyleneC₁₋₁₀alkylene; C₁₋₁₀alkylene; C₃₋₈cycloalkylene; C₃₋₈cycloalkyleneC₁₋₁₀alkylC₃₋₈cycloalkylene; C₆₋₁₂arylene; C₆₋₁₂aryleneC₁₋₆alkylene, can be unsubstituted or substituted with one or more Z^{1a}; preferably said C₁₋₁₀alkyleneC₃₋₈cycloalkylene; C₃₋₈cycloalkyleneC₁₋₁₀alkylene; C₁₋₁₀alkylene; C₃₋₈cycloalkylene, can be unsubstituted or substituted with one or more Z^{1a}; preferably said C₁₋₆alkyleneC₃₋₈cycloalkylene; C₃₋₈cycloalkyleneC₁₋₆alkylene; C₁₋₆alkylene; C₃₋₈cycloalkylene, can be unsubstituted or substituted with one or more Z^{1a}; preferably said C₁₋₆alkyleneC₃₋₈cycloalkylene; C₃₋₈cycloalkyleneC₁₋₆alkylene; C₁₋₆alkylene; C₃₋₈cycloalkylene, can be unsubstituted or substituted with one or more Z^{1a};

or L¹ is selected from a group of formula (iv);

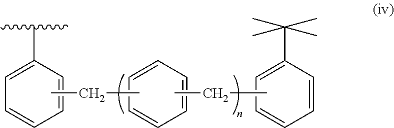

wherein the wavy line ( [symbol] ) indicates the point of attachment to the nitrogen of the formula (I) and the crossed line ( [symbol] ) indicates the point of attachment to R⁵;

wherein n is an integer selected from 0, 1, 2, 3, 4, 5, 6, 7, 8, 9, or 10; preferably n is an integer selected from 1, 2, 3, 4, 5, 6, 7, 8, 9, or 10; preferably n is an integer selected from 1, 2, 3, 4, 5, 6, 7, 8, or 9, preferably n is an integer selected from 1, 2, 3, 4, 5, 6, 7, or 8; preferably n is an integer selected from 1, 2, 3, 4, 5, 6, or 7; preferably n is an integer selected from 1, 2, 3, 4, 5, or 6; preferably n is an integer selected from 1, 2, 3, 4, or 5;

R⁵ is selected from the group consisting of OH, SH, COOH, NH₂, and NHR²⁰;

or R⁵ is a group of formula (v);

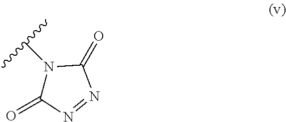 [SMILES: *N1C(=O)N=NC1=O]

wherein the wavy line ( [symbol] ) indicates the point of attachment to L¹;

or R⁵ is a group of formula (vi);

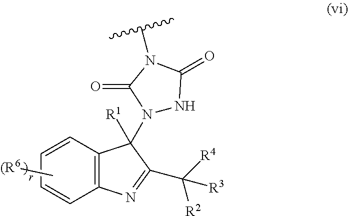

wherein the wavy line ( [symbol] ) indicates the point of attachment to L¹, and r, R¹, R², R³, R⁴ and R⁶ are as defined for structure (I);

each R⁶ is independently selected from the group consisting of hydrogen, halogen; C₁₋₁₀alkyl; C₂₋₁₀alkenyl; C₂₋₁₀alkynyl; C₆₋₁₂arylC₁₋₆alkyl; haloC₁₋₆alkyl; haloC₁₋₆alkyloxy; —OR¹⁹; —SR¹⁹; nitro; NR²⁰R²¹; —C(O)NR²⁰R²¹; cyano; and a polymeric group; preferably each R⁶ is independently selected from the group consisting of hydrogen, halogen; C₁₋₁₀alkyl; C₆₋₁₂arylC₁₋₆alkyl; haloC₁₋₆alkyl; haloC₁₋₆alkyloxy; —OR¹⁹; —SR¹⁹; nitro; NR²⁰R²¹; —C(O)NR²⁰R²¹; cyano; and a polymeric group; preferably each R⁶ is independently selected from the group consisting of hydrogen, halogen; C₁₋₁₀alkyl; C₆₋₁₂arylC₁₋₆alkyl; haloC₁₋₆alkyl; —OR¹⁹; NR²⁰R²¹; —C(O)NR²⁰R²¹; cyano; and a polymeric group; preferably each R⁶ is independently selected from the group consisting of hydrogen, halogen; C₁₋₁₀alkyl; C₆₋₁₂arylC₁₋₆alkyl; haloC₁₋₆alkyl; —OR¹⁹; —C(O)NR²⁰R²¹; and a polymeric group;
wherein said C₁₋₁₀alkyl; C₂₋₁₀alkenyl; C₂₋₁₀alkynyl; or C₆₋₁₂arylC₁₋₆alkyl, can be unsubstituted or substituted with one or more Z⁹; wherein said C₁₋₁₀alkyl; or C₆₋₁₂arylC₁₋₆alkyl, can be unsubstituted or substituted with one or more Z⁹; wherein said C₁₋₁₀alkyl; or C₆₋₁₂arylC₁₋₆alkyl, can be unsubstituted or substituted with one, two or three Z⁹;
and wherein for R⁶ said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group; preferably wherein said polymeric group is selected from the group consisting of polyurethane; isocyanate containing prepolymer, hydroxyl containing prepolymer, polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; ethylene propylene diene monomer (M-class) rubber (EPDM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group; preferably wherein said polymeric group is selected from the group consisting of polyurethane; isocyanate containing prepolymer, hydroxyl containing prepolymer, polyacrylate; polymethacrylate; polystyrene; copolymer of acrylate; poly(methyl methacrylate); methacrylate and/or styrene; poly(isobornyl acrylate); polyacrylonitrile; polyamide; polyester amide; ethylene propylene diene monomer (M-class) rubber (EPDM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;

each R¹⁹ is independently selected from the group consisting of C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl; wherein each group optionally comprise one or more heteroatoms in the alkyl, alkenyl or alkynyl moiety, said heteroatoms being each independently selected from O, S and N; preferably wherein each R¹⁹ is independently selected from the group consisting of C₁₋₁₀alkyl, C₂₋₁₀alkenyl, C₆₋₁₂aryl, C₆₋₁₂arylC₁₋₆alkyl; wherein each group optionally comprise one or more heteroatoms in the alkyl, alkenyl or alkynyl moiety, said heteroatoms being each independently selected from O, S and N preferably wherein each R¹⁹ is independently selected from the group consisting of C₁₋₆alkyl, C₂₋₆alkenyl, C₆₋₁₂aryl; wherein each group optionally comprise one or more heteroatoms in the alkyl, alkenyl or alkynyl moiety, said heteroatoms being each independently selected from O, and N;

wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl, can be unsubstituted or substituted with one or more OH, SH, COOH, or NH₂; preferably said C₁₋₂₀alkyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl, can be unsubstituted or substituted with one or more OH, SH, COOH, or NH₂; preferably said C₁₋₂₀alkyl, C₆₋₁₂aryl, C₃₋₈cycloalkyl, C₆₋₁₂arylC₁₋₆alkyl, heterocyclyl; heteroaryl, can be unsubstituted or substituted with one or more OH, SH, COOH, or NH₂; preferably said C₁₋₂₀alkyl, C₆₋₁₂aryl, C₃₋₈cycloalkyl, C₆₋₁₂arylC₁₋₆alkyl, heterocyclyl; heteroaryl, can be unsubstituted or substituted with one or more OH, SH, or NH₂; preferably said C₁₋₂₀alkyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl, can, can be unsubstituted or substituted with one or more OH, SH, or NH₂; preferably said C₁₋₂₀alkyl, C₆₋₁₂aryl, C₃₋₈cycloalkyl, C₆₋₁₂arylC₁₋₆alkyl, can be unsubstituted or substituted with one or more OH, SH, or NH₂;

each R²⁰ is independently selected from the group consisting of C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl; wherein each group optionally comprise one or more heteroatoms in the alkyl, alkenyl or alkynyl moiety, said heteroatoms being each independently selected from O, S and N; preferably wherein each R²⁰ is independently selected from the group consisting of C₁₋₁₀alkyl, C₂₋₁₀alkenyl, C₆₋₁₂aryl, C₆₋₁₂arylC₁₋₆alkyl; wherein each group optionally comprise one or more heteroatoms in the alkyl, alkenyl or alkynyl moiety, said heteroatoms being each independently selected from O, S and N; preferably wherein each R²⁰ is independently selected from the group consisting of C₁₋₆alkyl, C₂₋₆alkenyl, C₆₋₁₂aryl; wherein each group optionally comprise one or more heteroatoms in the alkyl, alkenyl or alkynyl moiety, said heteroatoms being each independently selected from O, and N;

wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl, can be unsubstituted or substituted with one or more OH, SH, COOH, or NH₂; preferably said C₁₋₂₀alkyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl, can be unsubstituted or substituted with one or more OH, SH, COOH, or NH₂; preferably said C₁₋₂₀alkyl, C₆₋₁₂aryl, C₃₋₈cycloalkyl, C₆₋₁₂arylC₁₋₆alkyl, heterocyclyl; heteroaryl, can be unsubstituted or substituted with one or more OH, SH, COOH, or NH₂; preferably said C₁₋₂₀alkyl, C₆₋₁₂aryl, C₃₋₈cycloalkyl, C₆₋₁₂arylC₁₋₆alkyl, heterocyclyl; heteroaryl, can be unsubstituted or substituted with one or more OH, SH, or NH₂; preferably said C₁₋₂₀alkyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl, can, can be unsubstituted or substituted with one or more OH, SH, or NH₂; preferably said C₁₋₂₀alkyl, C₆₋₁₂aryl, C₃₋₈cycloalkyl, C₆₋₁₂arylC₁₋₆alkyl, can be unsubstituted or substituted with one or more OH, SH, or NH₂;

each R²¹ is independently selected from the group consisting of hydrogen; C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl; wherein each group optionally comprise one or more heteroatoms in the alkyl, alkenyl or alkynyl moiety, said heteroatoms being each independently selected from O, S and N; preferably wherein each R²¹ is independently selected from the group consisting of hydrogen; C₁₋₁₀alkyl, C₂₋₁₀alkenyl, C₆₋₁₂aryl, C₆₋₁₂arylC₁₋₆alkyl; wherein each group optionally comprise one or more heteroatoms in the alkyl, alkenyl or alkynyl moiety, said heteroatoms being each independently selected from O, S and N; preferably wherein each R²¹ is independently selected from the group consisting of hydrogen; C₁₋₆alkyl, C₂₋₆alkenyl, C₆₋₁₂aryl; wherein each group optionally comprise one or more heteroatoms in the alkyl, alkenyl or alkynyl moiety, said heteroatoms being each independently selected from 0, and N;

wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl, can be unsubstituted or substituted with one or more OH, SH, COOH, or NH₂; preferably said C₁₋₂₀alkyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl, can be unsubstituted or substituted with one or more OH, SH, COOH, or NH₂; preferably said C₁₋₂₀alkyl, C₆₋₁₂aryl, C₃₋₈cycloalkyl, C₆₋₁₂arylC₁₋₆alkyl, heterocyclyl; heteroaryl, can be unsubstituted or substituted with one or more OH, SH, COOH, or NH₂; preferably said C₁₋₂₀alkyl, C₆₋₁₂aryl, C₃₋₈cycloalkyl, C₆₋₁₂arylC₁₋₆alkyl, heterocyclyl; heteroaryl, can be unsubstituted or substituted with one or more OH, SH, or NH₂; preferably said C₁₋₂₀alkyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl, can, can be unsubstituted or substituted with one or more OH, SH, or NH₂; preferably said C₁₋₂₀alkyl, C₆₋₁₂aryl, C₃₋₈cycloalkyl, C₆₋₁₂arylC₁₋₆alkyl, can be unsubstituted or substituted with one or more OH, SH, or NH₂;

or wherein R²⁰ and R²¹ together with the atom to which they are attached form a 5-, 6-, or 7-membered heterocycle;

each Z^{1a} is selected from the group consisting of C₁₋₆alkyl; C₂₋₆alkenyl; C₂₋₆alkynyl; haloC₁₋₆alkyl; haloC₁₋₆alkyloxy; halogen; isocyanate; preferably each Z^{1a} is selected from the group consisting of C₁₋₆alkyl; C₂₋₆alkenyl; haloC₁₋₆alkyl; haloC₁₋₆alkyloxy; halogen; isocyanate; preferably each Z^{1a} is selected from the group consisting of C₁₋₆alkyl; C₂₋₆alkynyl; haloC₁₋₆alkyl; isocyanate; preferably each Z^{1a} is selected from the group consisting of C₁₋₆alkyl; and isocyanate;

or Z^{1a} is a group of formula (vii);

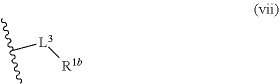

wherein the wavy line ( [symbol] ) indicates the point of attachment to L¹;
   L³ is a single bond or is selected from the group consisting of C₁₋₁₀alkylene; C₂₋₁₀alkenylene; C₂₋₁₀alkynylene; and C₆₋₁₂aryl; preferably L³ is a single bond or is C₁₋₁₀alkylene; preferably L³ is a single bond or is C₁₋₆alkylene;
   R^{1b} is a group of formula (viii);

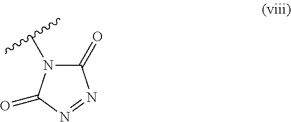 [SMILES: *N1C(=O)N=NC1=O]

wherein the wavy line ( [symbol] ) indicates the point of attachment to L³;
or R^{1b} is a group of formula (ix);

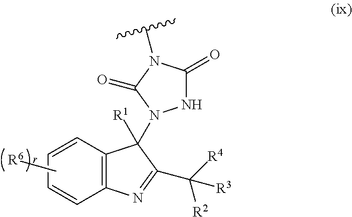

wherein the wavy line ( [symbol] ) indicates the point of attachment to L³ and r, R¹, R², R³, R⁴ and R⁶ are as defined for structure (I);

Z¹, Z², Z³, Z⁴, Z⁶ are each independently selected from the group consisting of OH; SH; COOH; NH₂; NHR²⁰; halogen; C₁₋₆alkyl; haloC₁₋₆alkyl; haloC₁₋₆alkyloxy; —OR¹⁹; —SR¹⁹; nitro; NR²⁰R²¹; —C(O)NR²⁰R²¹; cyano; preferably Z¹, Z², Z³, Z⁴, Z⁶ are each independently selected from the group consisting of OH; SH; COOH; NH₂; NHR²⁰; halogen; C₁₋₆alkyl; haloC₁₋₆alkyl; haloC₁₋₆alkyloxy; —OR¹⁹; —SR¹⁹; —C(O)NR²⁰R²¹; cyano; preferably Z¹, Z², Z³, Z⁴, Z⁶ are each independently selected from the group consisting of OH; SH; COOH; NH₂; NHR²⁰; halogen; C₁₋₆alkyl; haloC₁₋₆alkyl; —OR¹⁹; —SR¹⁹; —C(O)NR²⁰R²¹; cyano; preferably Z¹, Z², Z³, Z⁴, Z⁶ are each independently selected from the group consisting of OH; SH; COOH; NH₂; NHR²⁰; halogen; C₁₋₆alkyl; haloC₁₋₆alkyl; haloC₁₋₆alkyloxy; —OR¹⁹; —SR¹⁹; preferably Z¹, Z², Z³, Z⁴, Z⁶ are each independently selected from the group consisting of OH; SH; COOH; NH₂; NHR²⁰; halogen; C₁₋₆alkyl; haloC₁₋₆alkyl; haloC₁₋₆alkyloxy; preferably Z¹, Z², Z³, Z⁴, Z⁶ are each independently selected from the group consisting of OH; SH; COOH; NH₂; NHR²⁰; halogen; C₁₋₆alkyl; haloC₁₋₆alkyl;

Z⁵, Z^{5a}, Z⁷, Z⁸, Z^{8a}, Z⁹ are each independently selected from the group consisting of OH; hydrogen; SH; COOH; NH₂; NHR²⁰; halogen; C₁₋₆alkyl; haloC₁₋₆alkyl; haloC₁₋₆alkyloxy; —OR¹⁹; —SR¹⁹; nitro; NR²⁰R²¹; cyano; and a polymeric group; preferably Z⁵, Z^{5a}, Z⁷, Z⁸, Z^{8a}, Z⁹ are each independently selected from the group consisting of OH; hydrogen; SH; COOH; NH₂; NHR²⁰; halogen; C₁₋₆alkyl; haloC₁₋₆alkyl; haloC₁₋₆alkyloxy; —OR¹⁹; —SR¹⁹; cyano; and a polymeric group; preferably Z⁵, Z^{5a}, Z⁷, Z⁸, Z^{8a}, Z⁹ are each independently selected from the group consisting of OH; hydrogen; SH; COOH; NH₂; NHR²⁰; halogen; C₁₋₆alkyl; haloC₁₋₆alkyl; —OR¹⁹; —SR¹⁹; cyano; and a polymeric group; preferably Z⁵, Z^{5a}, Z⁷, Z⁸, Z^{8a}, Z⁹ are each independently selected from the group consisting of OH; hydrogen; SH; COOH; NH₂; NHR²⁰; halogen; C₁₋₆alkyl; haloC₁₋₆alkyl; haloC₁₋₆alkyloxy; —OR¹⁹; —SR¹⁹; and a polymeric group; preferably Z⁵, Z^{5a}, Z⁷, Z⁸, Z^{8a}, Z⁹ are each independently selected from the group consisting of OH; hydrogen; SH; COOH; NH₂; NHR²⁰; halogen; C₁₋₆alkyl; haloC₁₋₆alkyl; haloC₁₋₆alkyloxy; and a polymeric group; preferably Z⁵, Z^{5a}, Z⁷, Z⁸, Z^{8a}, Z⁹ are each independently selected from the group consisting of OH; hydrogen;

SH; COOH; NH₂; NHR²⁰; halogen; C₁₋₆alkyl; haloC₁₋₆alkyl; and a polymeric group;

and wherein for each Z⁵, Z^{5′}, Z⁷, Z⁸, Z^{8a}, Z⁹ said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group; preferably wherein said polymeric group is selected from the group consisting of polyurethane; isocyanate containing prepolymer, hydroxyl containing prepolymer, polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; ethylene propylene diene monomer (M-class) rubber (EPDM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group; preferably wherein said polymeric group is selected from the group consisting of polyurethane; isocyanate containing prepolymer, hydroxyl containing prepolymer, polyacrylate; polymethacrylate; polystyrene; copolymer of acrylate; poly(methyl methacrylate); methacrylate and/or styrene; poly(isobornyl acrylate); polyacrylonitrile; polyamide; polyester amide; ethylene propylene diene monomer (M-class) rubber (EPDM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;

and when none of R¹, R², R³, R⁴, R⁶, R⁷, R⁵, R⁹, R¹⁰, R¹¹, R¹², R¹⁴, R¹⁵, R¹⁶, R¹⁷, Z⁵, Z^{5a}, Z⁷, Z⁵, Z^{5a} or Z⁹ is a polymeric group, then at least one of R⁷, R⁸, Z¹, Z², Z³, Z⁴, Z⁵, Z^{5a}, Z⁶, Z⁷, Z⁵, Z^{5a} or Z⁹ is selected from the group consisting of OH, SH, COOH, NH₂, and NHR²⁰; preferably when none R¹, R², R³, R⁴, R⁶, R⁷, R⁸, R⁹, R¹⁰, R¹¹, R¹², R¹⁴, R¹⁵, R¹⁶, R¹⁷, Z⁵, Z^{5a}, Z⁷, Z⁵, Z^{5a} or Z⁹ is a polymeric group, then at least two of R⁷, R⁸, Z¹, Z², Z³, Z⁴, Z⁵, Z^{5a}, Z⁶, Z⁷, Z⁵, Z^{5a} or Z⁹ are selected from the group consisting of OH, SH, COOH, NH₂, and NHR²⁰; preferably two or more of R⁷, R⁸, Z¹, Z², Z³, Z⁴, Z⁵, Z^{5a}, Z⁶, Z⁷, Z⁸, Z^{5a} or Z⁹ are selected from the group consisting of OH, SH, COOH, NH₂, and NHR²⁰; preferably three R⁷, R⁸, Z¹, Z², Z³, Z⁴, Z⁵, Z^{5a}, Z⁶, Z⁷, Z⁵, Z^{5a} or Z⁹ are selected from the group consisting of OH, SH, COOH, NH₂, and NHR²⁰; preferably four of R⁷, R⁸, Z¹, Z², Z³, Z⁴, Z⁵, Z^{5a}, Z⁶, Z⁷, Z⁸, Z^{5a} or Z⁹ are selected from the group consisting of OH, SH, COOH, NH₂, and NHR²⁰.

The present invention also encompasses compositions comprising 4-[[5-(3,5-dioxo-1,2,4-triazol-4-yl)-1,3,3-trimethyl-cyclohexyl]methyl]-1,2,4-triazole-3,5-dione and 3-(4-(3-methyl-1H-indol-2-yl)phenoxy)propane-1,2-diol, as well as the reaction product obtained by contacting said 4-[[5-(3,5-dioxo-1,2,4-triazol-4-yl)-1,3,3-trimethyl-cyclohexyl]methyl]-1,2,4-triazole-3,5-dione with 3-(4-(3-methyl-1H-indol-2-yl)phenoxy)propane-1,2-diol. The present invention also encompasses a polymer prepared using said composition, for example a polyurethane or polyurethane prepolymer obtainable by contacting at least one isocyanate with said composition.

According to another embodiment, the present invention provides compounds of formula (A), (A2); and any subgroup thereof wherein,

r is an integer selected from 0, 1, 2, 3 or 4; preferably r is selected from 0, 1, 2, or 3; preferably r is selected from 0, 1 or 2; preferably r is selected from 0, or 1; more preferably r is 0;

R¹ is selected from the group consisting of C₁₋₂₀alkyl, C₂₋₂₀alkenyl; C₂₋₂₀alkynyl; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; —OR¹⁹; —SR¹⁹; NR²⁰R²¹; —C(O)NR²⁰R²¹; OH, SH, COOH, NH₂; NHR²⁰, halo; nitro; cyano; and a polymeric group; preferably R¹ is selected from the group consisting of C₁₋₂₀alkyl, C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; —OR¹⁹; —SR¹⁹; NR²⁰R²¹; —C(O)NR²⁰R²¹; OH, SH, COOH, NH₂; NHR²⁰, and a polymeric group; preferably R¹ is selected from the group consisting of C₁₋₁₀alkyl; C₂₋₁₀alkenyl; C₂₋₁₀alkynyl; C₆₋₁₂aryl; C₃₋₈cycloalkyl; C₆₋₁₂arylC₁₋₆alkyl; —OR¹⁹; —SR¹⁹; NR²⁰R²¹; —C(O)NR²⁰R²¹; OH, SH, COOH, NH₂; NHR²⁰, halo; nitro; cyano; and a polymeric group; preferably R¹ is selected from the group consisting of C₁₋₁₀alkyl; C₂₋₁₀alkenyl; C₆₋₁₂aryl; C₃₋₈cycloalkyl; C₆₋₁₂arylC₁₋₆alkyl; —OR¹⁹; —SR¹⁹; NR²⁰R²¹; and a polymeric group; preferably R¹ is selected from the group consisting of C₁₋₁₀alkyl; C₆₋₁₂aryl; C₃₋₈cycloalkyl; C₆₋₁₂arylC₁₋₆alkyl; —OR¹⁹; —SR¹⁹; NR²⁰R²¹; and a polymeric group; preferably R¹ is selected from the group consisting of C₁₋₁₀alkyl, C₃₋₈cycloalkyl; —OR¹⁹; —SR¹⁹; and NR²⁰R²¹; preferably R¹ is selected from the group consisting of C₁₋₆alkyl; C₆₋₁₂aryl; C₃₋₈cycloalkyl; and C₆₋₁₂arylC₁₋₆alkyl; preferably R¹ is selected from the group consisting of C₁₋₆alkyl and C₃₋₈cycloalkyl; preferably R¹ is C₁₋₁₀alkyl; for example R¹ is C₁₋₆alkyl; for example R¹ is C₁₋₆alkyl; and wherein said C₁₋₁₀alkyl; C₂₋₁₀alkenyl; C₂₋₁₀alkynyl, C₆₋₁₂aryl, C₃₋₈cycloalkyl; C₆₋₁₂arylC₁₋₆alkyl; heterocyclyl; heteroaryl; heterocyclylC₁₋₆alkyl; or heteroarylC₁₋₆alkyl, can be unsubstituted or substituted with one or more Z¹; preferably wherein said C₁₋₂₀alkyl, C₆₋₁₂aryl, C₃₋₈cycloalkyl; C₆₋₁₂arylC₁₋₆alkyl; heterocyclyl; heteroaryl; heterocyclylC₁₋₆alkyl; or heteroarylC₁₋₆alkyl, can be unsubstituted or substituted with one or more Z¹; preferably wherein said C₁₋₁₀alkyl; C₂₋₁₀alkenyl; C₂₋₁₀alkynyl; C₆₋₁₂aryl; C₃₋₈cycloalkyl; or C₆₋₁₂arylC₁₋₆alkyl, can be unsubstituted or substituted with one or more Z¹; preferably wherein said C₁₋₁₀alkyl; C₂₋₁₀alkenyl; C₆₋₁₂aryl; C₃₋₈cycloalkyl or C₆₋₁₂arylC₁₋₆alkyl, can be unsubstituted or substituted with one or more Z¹; preferably wherein said C₁₋₁₀alkyl; C₆₋₁₂aryl; C₃₋₈cycloalkyl or C₆₋₁₂arylC₁₋₆alkyl, can be unsubstituted or substituted with one or more Z¹; preferably wherein said C₁₋₁₀alkyl or C₃₋₈cycloalkyl, can be unsubstituted or substituted with one or more Z¹; preferably wherein said C₁₋₆alkyl; C₆₋₁₂aryl; C₃₋₈cycloalkyl; or C₆₋₁₂arylC₁₋₆alkyl, can be unsubstituted or substituted with one or more Z¹; preferably wherein said C₁₋₆alkyl or C₃₋₈cycloalkyl, can be unsubstituted or substituted with one or more Z¹; preferably wherein said C₁₋₁₀alkyl, for example C₁₋₆alkyl, for example C₁₋₆alkyl, can be unsubstituted or substituted with one or more Z¹;

and wherein for R¹ said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group; preferably wherein said polymeric group is selected from the group consisting of polyurethane; isocyanate containing prepolymer, hydroxyl containing prepolymer, polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; ethylene propylene diene monomer (M-class) rubber (EPDM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group; preferably wherein said polymeric group is selected from the group consisting of polyurethane; isocyanate containing prepolymer, hydroxyl containing prepolymer, polyacrylate; polymethacrylate; polystyrene; copolymer of acrylate; poly(methyl methacrylate); methacrylate and/or styrene; poly(isobornyl acrylate); polyacrylonitrile; polyamide; polyester amide; ethylene propylene diene monomer (M-class) rubber (EPDM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;

or R¹ is a group of formula (i);

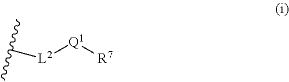

wherein the wavy line ( [symbol] ) indicates the point of attachment to the indole ring of formula (I);

L² is selected from the group consisting of C₁₋₁₀alkylene; C₃₋₆cycloalkylene; C₂₋₁₀alkenylene; C₂₋₁₀alkynylene; C₆₋₁₂arylene; and C₆₋₁₂arylC₁₋₆alkylene; preferably L² is selected from C₁₋₁₀alkylene; C₃₋₆cycloalkylene; C₆₋₁₂arylene; and C₆₋₁₂arylC₁₋₆alkylene; preferably L² is selected from C₁₋₁₀alkylene; C₃₋₆cycloalkylene; and C₆₋₁₂arylene; preferably L² is selected from C₁₋₁₀alkylene; C₃₋₆cycloalkylene; and C₆₋₁₂arylene; preferably L² is selected from C₁₋₆alkylene; C₃₋₆cycloalkylene; and C₆₋₁₂arylene; preferably L² is selected from C₁₋₆alkylene; C₃₋₆cycloalkylene; and C₆₋₁₂arylene; and wherein said C₁₋₁₀alkylene; C₃₋₆cycloalkylene; C₂₋₁₀alkenylene; C₂₋₁₀alkynylene; C₆₋₁₂arylene or C₆₋₁₂arylC₁₋₆alkylene, can be unsubstituted or substituted with one or more Z²; preferably said C₁₋₁₀alkylene; C₃₋₆cycloalkylene; C₆₋₁₂arylene or C₆₋₁₂arylC₁₋₆alkylene, can be unsubstituted or substituted with one or more Z²; preferably said C₁₋₁₀alkylene; C₃₋₆cycloalkylene; C₆₋₁₂arylene, can be unsubstituted or substituted with one or more Z²; preferably said C₁₋₆alkylene; C₃₋₆cycloalkylene; C₆₋₁₂arylene, can be unsubstituted or substituted with one or more Z²; preferably said C₁₋₆alkylene; C₃₋₆cycloalkylene; C₆₋₁₂arylene, can be unsubstituted or substituted with one or more Z²;

Q¹ is a single bond or selected from the group consisting of C₁₋₁₀alkylene; C₃₋₆cycloalkylene; —O—; —S—; —O—C₁₋₁₀alkylene; and —S—C₁₋₁₀alkylene; preferably Q¹ is a single bond or selected from the group consisting of C₁₋₁₀alkylene; C₃₋₆cycloalkylene; —O—; —S—; —O—C₁₋₁₀alkylene; preferably Q¹ is a single bond or selected from the group consisting of C₁₋₁₀alkylene; C₃₋₆cycloalkylene; —O—; —S—; preferably Q¹ is a single bond or selected from the group consisting of C₁₋₁₀alkylene; C₃₋₆cycloalkylene; wherein each left side of said groups is attached to L² and the right side thereof is attached to R⁷;

and wherein said C₁₋₁₀alkylene and said C₃₋₆cycloalkylene; can be unsubstituted or substituted with one or more Z³;

R⁷ is selected from the group consisting of hydrogen; OH, SH, COOH, NH₂; NHR²⁰, halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group; preferably R⁷ is selected from the group consisting of hydrogen; OH, SH, COOH, NH₂; NHR²⁰, —C(O)NR²⁰R²¹; C₁₋₂₀alkyl, C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;

wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl; can be unsubstituted or substituted with one or more Z⁴; preferably said C₁₋₂₀alkyl, C₆₋₁₂aryl, C₃₋₈cycloalkyl; C₆₋₁₂arylC₁₋₆alkyl; heterocyclyl; heteroaryl; heterocyclylC₁₋₆alkyl; or heteroarylC₁₋₆alkyl; can be unsubstituted or substituted with one or more Z⁴; preferably said C₁₋₂₀alkyl, C₆₋₁₂aryl, C₃₋₈cycloalkyl; C₆₋₁₂arylC₁₋₆alkyl; can be unsubstituted or substituted with one or more Z⁴;

and wherein for R⁷ said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group; preferably wherein said polymeric group is selected from the group consisting of polyurethane; isocyanate containing prepolymer, hydroxyl containing prepolymer, polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; ethylene propylene diene monomer (M-class) rubber (EPDM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group; preferably wherein said polymeric group is selected from the group consisting of polyurethane; isocyanate containing prepolymer, hydroxyl containing prepolymer, polyacrylate; polymethacrylate; polystyrene; copolymer of acrylate; poly(methyl methacrylate); methacrylate and/or styrene; poly(isobornyl acrylate); polyacrylonitrile; polyamide; polyester amide; ethylene propylene diene monomer (M-class) rubber (EPDM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;

or R⁷ is a group of formula (ii);

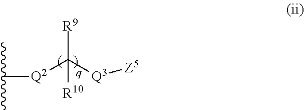

wherein the wavy line ( [symbol] ) indicates the point of attachment to Q¹;
q is an integer selected from 0; 1, 2, 3 or 4; preferably q is selected from 0; 1, 2, or 3; preferably q is selected from 0; 1, or 2;
Q² is a single bond or selected from the group consisting of CR¹¹R¹²; O, S, and NR¹³; preferably Q² is selected from the group consisting of CR¹¹R¹²; O, S, and NR¹³; preferably Q² is selected from the group consisting of CR¹¹R¹²; O, S;
Q³ is a single bond or selected from the group consisting of hydroxyC₁₋₁₀alkylene; aminoC₁₋₁₀alkylene; mercaptoC₁₋₁₀alkylene; C₁₋₂₀alkylene, C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₆alkylene; and heteroarylC₁₋₆alkylene; preferably Q³ is a single bond or selected from the group consisting of hydroxyC₁₋₁₀alkylene; aminoC₁₋₁₀alkylene; mercaptoC₁₋₁₀alkylene; C₁₋₂₀alkylene, C₆₋₁₂arylene, C₃₋₈cycloalkylene; C₆₋₁₂arylC₁₋₆alkylene; heterocyclylene; heteroarylene; heterocyclylC₁₋₆alkylene; and heteroarylC₁₋₆alkylene; preferably Q³ is a single bond or selected from the group consisting of hydroxyC₁₋₁₀alkylene; aminoC₁₋₁₀alkylene; mercaptoC₁₋₁₀alkylene; C₁₋₂₀alkylene, C₃₋₈cycloalkylene; C₆₋₁₂arylC₁₋₆alkylene; heterocyclylC₁₋₆alkylene; and heteroarylC₁₋₆alkylene; preferably Q³ is a single bond or selected from the group consisting of hydroxyC₁₋₆alkylene; aminoC₁₋₆alkylene; mercaptoC₁₋₈alkylene; C₁₋₆alkylene, C₃₋₈cycloalkylene; C₆₋₁₂arylC₁₋₆alkylene; heterocyclylC₁₋₆alkylene; and heteroarylC₁₋₆alkylene; preferably Q³ is a single bond or selected from the group consisting of hydroxyC₁₋₆alkylene; aminoC₁₋₆alkylene; mercaptoC₁₋₆alkylene; C₁₋₆alkylene, C₃₋₈cycloalkylene; C₆₋₁₂arylC₁₋₆alkylene; heterocyclylC₁₋₆alkylene; and heteroarylC₁₋₆alkylene;
wherein said C₁₋₂₀alkylene, C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₆alkylene; or heteroarylC₁₋₆alkylene, can be unsubstituted or substituted with one or more Z^{5a}; preferably said C₁₋₂₀alkylene, C₆₋₁₂arylene, C₃₋₈cycloalkylene; C₆₋₁₂arylC₁₋₆alkylene; heterocyclylene; heteroarylene; heterocyclylC₁₋₆alkylene; or heteroarylC₁₋₆alkylene, can be unsubstituted or substituted with one or more Z^{5a}; preferably said C₁₋₂₀alkylene, C₆₋₁₂arylene, C₃₋₈cycloalkylene; heterocyclylene; or heteroarylene; can be unsubstituted or substituted with one or more Z^{5a};
wherein if q is 0, then one of Q² and Q³ is not a single bond;
each R⁹ and R¹⁰ is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group; preferably each R⁹ and R¹⁰ is independently selected from the group consisting of hydrogen; C₁₋₂₀alkyl, hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group; preferably each R⁹ and R¹⁰ is independently selected from the group consisting of hydrogen; C₁₋₂₀alkyl, hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclylC₁₋₆alkyl; heteroarylC₁₋₆alkyl; and a polymeric group; preferably each R⁹ and R¹⁰ is independently selected from the group consisting of hydrogen; C₁₋₁₈alkyl, hydroxyC₁₋₈alkoxy; aminoC₁₋₈alkoxy; mercaptoC₁₋₈alkoxy; C₆₋₁₂aryl, C₃₋₈cycloalkyl; C₆₋₁₂arylC₁₋₆alkyl; heterocyclylC₁₋₆alkyl; heteroarylC₁₋₆alkyl; and a polymeric group; preferably each R⁹ and R¹³ is independently selected from the group consisting of hydrogen; C₁₋₁₀alkyl, hydroxyC₁₋₆alkoxy; aminoC₁₋₆alkoxy; mercaptoC₁₋₆alkoxy; C₆₋₁₂aryl, C₃₋₈cycloalkyl; C₆₋₁₂arylC₁₋₆alkyl; heterocyclylC₁₋₆alkyl; heteroarylC₁₋₆alkyl; and a polymeric group;
wherein for each R⁹ and R¹⁰ said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group; preferably wherein said polymeric group is selected from the group consisting of polyurethane; isocyanate containing prepolymer, hydroxyl containing prepolymer, polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; ethylene propylene diene monomer (M-class) rubber (EPDM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group; preferably wherein said polymeric group is selected from the group consisting of polyurethane; isocyanate containing prepolymer, hydroxyl containing prepolymer, polyacrylate; polymethacrylate; polystyrene; copolymer of acrylate; poly(methyl methacrylate); methacrylate and/or styrene; poly(isobornyl acrylate); polyacrylonitrile; polyamide; polyester amide; ethylene propylene diene monomer (M-class) rubber (EPDM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
each R¹¹ and R¹² is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group; preferably each R¹¹ and R¹² is independently selected from the group consisting of hydrogen; C₁₋₂₀alkyl, hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group; preferably each R¹¹ and R¹² is independently selected from the group consisting of hydrogen; C₁₋₂₀alkyl, hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclylC₁₋₆alkyl; heteroarylC₁₋₆alkyl; and a polymeric group; preferably each R¹¹ and R¹² is independently selected from the group consisting of hydrogen; C₁₋₁₈alkyl, hydroxyC₁₋₈alkoxy; aminoC₁₋₈alkoxy; mercaptoC₁₋₈alkoxy; C₆₋₁₂aryl, C₃₋₈cycloalkyl; C₆₋₁₂arylC₁₋₆alkyl; heterocyclylC₁₋₆alkyl; heteroarylC₁₋₆alkyl; and a polymeric group; preferably each R¹¹ and R¹² is independently selected from the group consisting of hydrogen; C₁₋₁₀alkyl, hydroxyC₁₋₆alkoxy; aminoC₁₋₆alkoxy; mercaptoC₁₋₆alkoxy; C₆₋₁₂aryl, C₃₋₈cycloalkyl; C₆₋₁₂arylC₁₋₆alkyl; heterocyclylC₁₋₆alkyl; heteroarylC₁₋₆alkyl; and a polymeric group;
wherein for each R¹¹ and R¹² said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group; preferably wherein said polymeric group is selected from the group consisting of polyurethane; isocyanate containing prepolymer, hydroxyl containing prepolymer, polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; ethylene propylene diene monomer (M-class) rubber (EPDM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group; preferably wherein said polymeric group is selected from the group consisting of polyurethane; isocyanate containing prepolymer, hydroxyl containing prepolymer, polyacrylate; polymethacrylate; polystyrene; copolymer of acrylate; poly(methyl methacrylate); methacrylate and/or styrene; poly(isobornyl acrylate); polyacrylonitrile; polyamide; polyester amide; ethylene propylene diene monomer (M-class) rubber (EPDM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
R¹³ is selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; and heteroarylC₁₋₆alkyl; preferably R¹³ is selected from the group consisting of hydrogen; C₁₋₂₀alkyl, hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; and heteroarylC₁₋₆alkyl; preferably R¹³ is selected from the group consisting of hydrogen; C₁₋₂₀alkyl, hydroxyC₁₋₁₀alkoxy; aminoC₁₋₁₀alkoxy; mercaptoC₁₋₁₀alkoxy; C₃₋₈cycloalkyl; C₆₋₁₂arylC₁₋₆alkyl; heterocyclylC₁₋₆alkyl; and heteroarylC₁₋₆alkyl; preferably R¹³ is selected from the group consisting of hydrogen; C₁₋₁₈alkyl, hydroxyC₁₋₈alkoxy; aminoC₁₋₈alkoxy; mercaptoC₁₋₈alkoxy; C₃₋₈cycloalkyl; C₆₋₁₂arylC₁₋₆alkyl; heterocyclylC₁₋₆alkyl; and heteroarylC₁₋₆alkyl; preferably R¹³ is selected from the group consisting of hydrogen; C₁₋₁₀alkyl, hydroxyC₁₋₆alkoxy; aminoC₁₋₆alkoxy; mercaptoC₁₋₆alkoxy; C₃₋₈cycloalkyl; C₆₋₁₂arylC₁₋₆alkyl; heterocyclylC₁₋₆alkyl; and heteroarylC₁₋₆alkyl;

R² is selected from the group consisting of halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; heteroarylC₁₋₆alkyl; and a polymeric group; preferably R² is selected from the group consisting halo; nitro; cyano; —C(O)NR²⁰R²¹; C₁₋₂₀alkyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; heteroarylC₁₋₆alkyl; and a polymeric group; preferably R² is selected from the group consisting halo; nitro; cyano; —C(O)NR²⁰R²¹; C₁₋₂₀alkyl; C₆₋₁₂aryl; C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group; preferably R² is selected from the group consisting halo; nitro; cyano; —C(O)NR²⁰R²¹; C₁₋₂₀alkyl; C₆₋₁₂aryl; C₃₋₈cycloalkyl; C₆₋₁₂arylC₁₋₆alkyl; heterocyclyl; heteroaryl; and a polymeric group; preferably R² is selected from the group consisting halo; C₁₋₂₀alkyl; C₆₋₁₂aryl; C₃₋₈cycloalkyl; C₆₋₁₂arylC₁₋₆alkyl; heterocyclyl; heteroaryl; and a polymeric group;
wherein said O₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl, heteroarylC₁₋₆alkyl, can be unsubstituted or substituted with one or more Z⁶; wherein said C₁₋₂₀alkyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; and heteroarylC₁₋₆alkyl can be unsubstituted or substituted with one or more Z⁶; wherein said C₁₋₂₀alkyl; C₆₋₁₂aryl; C₃₋₈cycloalkyl; C₆₋₁₂arylC₁₋₆alkyl; heterocyclyl; heteroaryl; heterocyclylC₁₋₆alkyl; and heteroarylC₁₋₆alkyl, can be unsubstituted or substituted with one or more Z⁶; wherein said C₁₋₂₀alkyl; C₆₋₁₂aryl; C₃₋₈cycloalkyl; C₆₋₁₂arylC₁₋₆alkyl; heterocyclyl; heteroaryl, can be unsubstituted or substituted with one or more Z⁶; wherein said C₁₋₂₀alkyl; C₆₋₁₂aryl; C₃₋₈cycloalkyl; C₆₋₁₂arylC₁₋₆alkyl; heterocyclyl; heteroaryl; can be unsubstituted or substituted with one or more Z⁶;

R³ is selected from the group consisting of halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; heteroarylC₁₋₆alkyl; and a polymeric group; preferably R³ is selected from the group consisting halo; nitro; cyano; —C(O)NR²⁰R²¹; C₁₋₂₀alkyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; heteroarylC₁₋₆alkyl; and a polymeric group; preferably R³ is selected from the group consisting halo; nitro; cyano; —C(O)NR²⁰R²¹; C₁₋₂₀alkyl; C₆₋₁₂aryl; C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group; preferably R³ is selected from the group consisting halo; nitro; cyano; —C(O)NR²⁰R²¹; C₁₋₂₀alkyl; C₆₋₁₂aryl; C₃₋₈cycloalkyl; C₆₋₁₂arylC₁₋₆alkyl; heterocyclyl; heteroaryl; and a polymeric group; preferably R³ is selected from the group consisting halo; C₁₋₂₀alkyl; C₆₋₁₂aryl; C₃₋₈cycloalkyl; C₆₋₁₂arylC₁₋₆alkyl; heterocyclyl; heteroaryl;
wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl, heteroarylC₁₋₆alkyl, can be unsubstituted or substituted with one or more Z⁶; wherein said C₁₋₂₀alkyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; and heteroarylC₁₋₆alkyl can be unsubstituted or substituted with one or more Z⁶; wherein said C₁₋₂₀alkyl; C₆₋₁₂aryl; C₃₋₈cycloalkyl; C₆₋₁₂arylC₁₋₆alkyl; heterocyclyl; heteroaryl; heterocyclylC₁₋₆alkyl; and heteroarylC₁₋₆alkyl, can be unsubstituted or substituted with one or more Z⁶; wherein said C₁₋₂₀alkyl; C₆₋₁₂aryl; C₃₋₈cycloalkyl; C₆₋₁₂arylC₁₋₆alkyl; heterocyclyl; heteroaryl, can be unsubstituted or substituted with one or more Z⁶; wherein said C₁₋₂₀alkyl; C₆₋₁₂aryl; C₃₋₈cycloalkyl; C₆₋₁₂arylC₁₋₆alkyl; heterocyclyl; heteroaryl; can be unsubstituted or substituted with one or more Z⁶;

R⁴ is selected from the group consisting of halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; heteroarylC₁₋₆alkyl; and a polymeric group; preferably R⁴ is selected from the group consisting of halo; nitro; cyano; —C(O)NR²⁰R²¹; C₁₋₂₀alkyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; heteroarylC₁₋₆alkyl; and a polymeric group; preferably R⁴ is selected from the group consisting of halo; nitro; cyano; —C(O)NR²⁰R²¹; C₁₋₂₀alkyl; C₆₋₁₂aryl; C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group; preferably R⁴ is selected from the group consisting of halo; nitro; cyano; —C(O)NR²⁰R²¹; C₁₋₂₀alkyl; C₆₋₁₂aryl; C₃₋₈cycloalkyl; C₆₋₁₂arylC₁₋₆alkyl; heterocyclyl; heteroaryl; and a polymeric group; preferably R⁴ is selected from the group consisting of halo; C₁₋₂₀alkyl; C₆₋₁₂aryl; C₃₋₈cycloalkyl; C₆₋₁₂arylC₁₋₆alkyl; heterocyclyl; heteroaryl; and a polymeric group;
wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl, heteroarylC₁₋₆alkyl, can be unsubstituted or substituted with one or more Z⁶; wherein said C₁₋₂₀alkyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; and heteroarylC₁₋₆alkyl can be unsubstituted or substituted with one or more Z⁶; wherein said C₁₋₂₀alkyl; C₆₋₁₂aryl; C₃₋₈cycloalkyl; C₆₋₁₂arylC₁₋₆alkyl; heterocyclyl; heteroaryl; heterocyclylC₁₋₆alkyl; and heteroarylC₁₋₆alkyl, can be unsubstituted or substituted with one or more Z⁶; wherein said C₁₋₂₀alkyl; C₆₋₁₂aryl; C₃₋₈cycloalkyl; C₆₋₁₂arylC₁₋₆alkyl; heterocyclyl; heteroaryl, can be unsubstituted or substituted with one or more Z⁶; wherein said C₁₋₂₀alkyl; C₆₋₁₂aryl; C₃₋₈cycloalkyl; C₆₋₁₂arylC₁₋₆alkyl; heterocyclyl; heteroaryl; can be unsubstituted or substituted with one or more Z⁶;
and wherein for each of R² and R³ and R⁴ said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group; preferably wherein said polymeric group is selected from the group consisting of polyurethane; isocyanate containing prepolymer, hydroxyl containing prepolymer, polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; ethylene propylene diene monomer (M-class) rubber (EPDM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group; preferably wherein said polymeric group is selected from the group consisting of polyurethane; isocyanate containing prepolymer, hydroxyl containing prepolymer, polyacrylate; polymethacrylate; polystyrene; copolymer of acrylate; poly(methyl methacrylate); methacrylate and/or styrene; poly(isobornyl acrylate); polyacrylonitrile; polyamide; polyester amide; ethylene propylene diene monomer (M-class) rubber (EPDM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;

or R² and R³ and R⁴ together with the carbon atom to which they are attached can form a saturated or unsaturated 4-, 5-, 6-, 7- or 8-membered ring; preferably R² and R³ and R⁴ together with the carbon atom to which they are attached can form a saturated or unsaturated 4-, 5-, 6-, 7-membered ring; preferably R² and R³ and R⁴ together with the carbon atom to which they are attached can form a saturated or unsaturated 5-, 6-, or 7-membered ring; preferably R² and R³ and R⁴ together with the carbon atom to which they are attached can form an unsaturated 5-, 6-, or 7-membered ring; preferably R² and R³ and R⁴ together with the carbon atom to which they are attached can form a saturated or unsaturated 5-, or 6-membered ring; preferably R² and R³ and R⁴ together with the carbon atom to which they are attached can form an unsaturated 5-, or 6-membered ring; preferably R² and R³ and R⁴ together with the carbon atom to which they are attached can form a saturated or unsaturated 6-membered ring; preferably R² and R³ and R⁴ together with the carbon atom to which they are attached can form an unsaturated 6-membered ring; preferably R² and R³ and R⁴ together with the carbon atom to which they are attached can form an aryl ring; for example a phenyl ring;
wherein said saturated or unsaturated 4-, 5-, 6-, 7- or 8-membered ring optionally comprises one or more heteroatoms, said heteroatoms being each independently selected from O, S and N; preferably said saturated or unsaturated 4-, 5-, 6-, 7-membered ring optionally comprises one or more heteroatoms, said heteroatoms being each independently selected from O, S and N; preferably said saturated or unsaturated 5-, 6-, 7-membered ring optionally comprises one or more heteroatoms, said heteroatoms being each independently selected from O, S and N; preferably said unsaturated 5-, 6-, or 7-membered ring optionally comprises one or more heteroatoms, said heteroatoms being each independently selected from O, S and N; preferably said saturated or unsaturated 5-, or 6-membered ring optionally comprises one or more heteroatoms, said heteroatoms being each independently selected from O, S and N; preferably said unsaturated 5-, or 6-membered ring optionally comprises one or more heteroatoms, said heteroatoms being each independently selected from O, S and N; preferably said saturated or unsaturated 6-membered ring optionally comprises one or more heteroatoms, said heteroatoms being each independently selected from O, S and N; preferably said unsaturated 6-membered ring optionally comprises one or more heteroatoms, said heteroatoms being each independently selected from O, S and N; preferably said phenyl ring optionally comprises one or more heteroatoms, said heteroatoms being each independently selected from O, S and N;
wherein said saturated or unsaturated 4-, 5-, 6-, 7- or 8-membered ring can be unsubstituted or substituted with one or more R⁸; wherein said saturated or unsaturated 4-, 5-, 6-, 7- or 8-membered ring can be unsubstituted or substituted with one, two, or three R⁸; preferably said saturated or unsaturated 4-, 5-, 6-, 7-membered ring can be unsubstituted or substituted with one or more R⁸; preferably said saturated or unsaturated 5-, 6-, 7-membered ring can be unsubstituted or substituted with one or more R⁸; preferably said unsaturated 5-, 6-, or 7-membered ring can be unsubstituted or substituted with one or more R⁸; preferably said saturated or unsaturated 5-, or 6-membered can be unsubstituted or substituted with one or more R⁸; preferably said saturated or unsaturated 5-, or 6-membered can be unsubstituted or substituted with one, two or three R⁸; preferably said unsaturated 5-, or 6-membered ring can be unsubstituted or substituted with one or more R⁸; preferably said saturated or unsaturated 6-membered ring can be unsubstituted or substituted with one or more R⁸; preferably said unsaturated 6-membered ring can be unsubstituted or substituted with one or more R⁸; preferably said phenyl ring can be unsubstituted or substituted with one or more R⁸; preferably said phenyl ring can be unsubstituted or substituted with one, two or three R⁸;
each R⁸ is independently selected from the group consisting of hydroxyC₁₋₁₀alkoxy; aminoC₁₋₁₀alkoxy; mercaptoC₁₋₁₀alkoxy; C₁₋₁₀alkyl; C₂₋₁₀alkenyl; C₂₋₁₀alkynyl; C₆₋₁₂arylC₁₋₆alkyl; C₃₋₈cycloalkyl; C₆₋₁₂arylC₁₋₆alkyl; heterocyclyl; heteroaryl; heterocyclylC₁₋₆alkyl; heteroarylC₁₋₈alkyl; OH; SH; COOH; NH₂; NHR²⁰; halogen; haloC₁₋₆alkyl; haloC₁₋₆alkyloxy; —OR¹⁹; —SR⁹; nitro; NR²⁰R²¹; —C(O)NR²⁰R²¹; cyano; and a polymeric group; preferably each R⁸ is independently selected from the group consisting of hydroxyC₁₋₁₀alkoxy; aminoC₁₋₁₀alkoxy; mercaptoC₁₋₁₀alkoxy; C₁₋₁₀alkyl; C₆₋₁₂arylC₁₋₆alkyl; C₃₋₈cycloalkyl; C₆₋₁₂arylC₁₋₆alkyl; heterocyclyl; heteroaryl; heterocyclylC₁₋₆alkyl; heteroarylC₁₋₆alkyl; OH; SH; COOH; NH₂, NHR²⁰; halogen; haloC₁₋₆alkyl; haloC₁₋₆alkyloxy; —OR¹⁹; —SR⁹; nitro; NR²⁰R²¹; —C(O)NR²⁰R²¹; cyano; and a polymeric group; preferably each R⁸ is independently selected from the group consisting of hydroxyC₁₋₁₀alkoxy; aminoC₁₋₁₀alkoxy; mercaptoC₁₋₁₀alkoxy; C₁₋₁₀alkyl; C₆₋₁₂arylC₁₋₆alkyl; C₃₋₈cycloalkyl; C₆₋₁₂arylC₁₋₆alkyl; heterocyclylC₁₋₆alkyl; heteroarylC₁₋₆alkyl; OH; SH; COOH; NH₂; NHR²⁰; —OR¹⁹; —SR¹⁹; and a polymeric group; preferably each R⁸ is independently selected from the group consisting of hydroxyC₁₋₈alkoxy; aminoC₁₋₈alkoxy; mercaptoC₁₋₈alkoxy; C₁₋₆alkyl; C₆₋₁₂arylC₁₋₆alkyl; C₃₋₈cycloalkyl; C₆₋₁₂arylC₁₋₆alkyl; heterocyclylC₁₋₆alkyl; heteroarylC₁₋₆alkyl; OH; SH; COOH; NH₂; NHR²⁰; —OR¹⁹; —SR⁹; and a polymeric group; preferably each R⁸ is independently selected from the group consisting of hydroxyC₁₋₈alkoxy; aminoC₁₋₈alkoxy; mercaptoC₁₋₈alkoxy; C₁₋₆alkyl; C₈₋₁₂arylC₁₋₆alkyl; C₃₋₈cycloalkyl; C₆₋₁₂arylC₁₋₆alkyl; heterocyclylC₁₋₆alkyl; heteroarylC₁₋₆alkyl; OH; SH; COOH; NH₂; NHR²⁰; and a polymeric group;
   wherein said hydroxyC₁₋₁₀alkoxy; aminoC₁₋₁₀alkoxy; mercaptoC₁₋₁₀alkoxy; C₁₋₁₀alkyl; C₂₋₁₀alkenyl; C₂₋₁₀alkynyl; C₆₋₁₂arylC₁₋₆alkyl; C₃₋₈cycloalkyl; C₆₋₁₂arylC₁₋₆alkyl; heterocyclyl; heteroaryl; heterocyclylC₁₋₆alkyl; or heteroarylC₁₋₆alkyl, can be unsubstituted or substituted with one or more Z⁷; preferably said hydroxyC₁₋₁₀alkoxy; aminoC₁₋₁₀alkoxy; mercaptoC₁₋₁₀alkoxy; C₁₋₁₀alkyl; C₆₋₁₂arylC₁₋₆alkyl; C₃₋₈cycloalkyl; C₆₋₁₂arylC₁₋₆alkyl; heterocyclyl; heteroaryl; heterocyclylC₁₋₆alkyl; or heteroarylC₁₋₆alkyl, can be unsubstituted or substituted with one or more Z⁷; preferably said hydroxyC₁₋₁₀alkoxy; aminoC₁₋₁₀alkoxy; mercaptoC₁₋₁₀alkoxy; C₁₋₁₀alkyl; C₆₋₁₂arylC₁₋₆alkyl; C₃₋₈cycloalkyl; C₆₋₁₂arylC₁₋₆alkyl; heterocyclylC₁₋₆alkyl; or heteroarylC₁₋₆alkyl, can be unsubstituted or substituted with one or more Z⁷; preferably said hydroxyC₁₋₆alkoxy; aminoC₁₋₆alkoxy; mercaptoC₁₋₆alkoxy; C₁₋₆alkyl; C₆₋₁₂arylC₁₋₆alkyl; C₃₋₈cycloalkyl; C₆₋₁₂arylC₁₋₆alkyl; heterocyclylC₁₋₆alkyl; or heteroarylC₁₋₆alkyl, can be unsubstituted or substituted with one or more Z⁷;
   and wherein for R⁸ said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group; preferably wherein said polymeric group is selected from the group consisting of polyurethane; isocyanate containing prepolymer, hydroxyl containing prepolymer, polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; ethylene propylene diene monomer (M-class) rubber (EPDM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group; preferably wherein said polymeric group is selected from the group consisting of polyurethane; isocyanate containing prepolymer, hydroxyl containing prepolymer, polyacrylate; polymethacrylate; polystyrene; copolymer of acrylate; poly(methyl methacrylate); methacrylate and/or styrene; poly(isobornyl acrylate); polyacrylonitrile; polyamide; polyester amide; ethylene propylene diene monomer (M-class) rubber (EPDM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
   or R⁸ is a group of formula (iii);

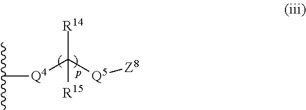

wherein the wavy line ( [symbol] ) indicates the point of attachment to the ring formed by R² and R³ and R⁴ together;
p is an integer selected from 0; 1, 2, 3 or 4; preferably p is selected from 0; 1, 2, or 3; preferably p is selected from 0; 1, or 2; preferably p is 1 or 2;
Q⁴ is a single bond or selected from the group consisting of CR¹⁶R¹⁷; O, S, and NR¹⁸; preferably Q⁴ is selected from the group consisting of CR¹⁶R¹⁷; O, S;
Q⁵ is a single bond or selected from the group consisting of hydroxyC₁₋₁₀alkylene; aminoC₁₋₁₀alkylene; mercaptoC₁₋₁₀alkylene; C₁₋₂₀alkylene, C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₆alkylene; and heteroarylC₁₋₆alkylene; preferably Q⁵ is a single bond or selected from the group consisting of hydroxyC₁₋₁₀alkylene; aminoC₁₋₁₀alkylene; mercaptoC₁₋₁₀alkylene; C₁₋₂₀alkylene, C₆₋₁₂arylene, C₃₋₈cycloalkylene; C₆₋₁₂arylC₁₋₆alkylene; heterocyclylene; heteroarylene; heterocyclylC₁₋₆alkylene; and heteroarylC₁₋₆alkylene; preferably Q⁵ is a single bond or selected from the group consisting of hydroxyC₁₋₁₀alkylene; aminoC₁₋₁₀alkylene; mercaptoC₁₋₁₀alkylene; C₁₋₂₀alkylene, C₃₋₈cycloalkylene; C₆₋₁₂arylC₁₋₆alkylene; heterocyclylC₁₋₆alkylene; and heteroarylC₁₋₆alkylene; preferably Q⁵ is a single bond or selected from the group consisting of hydroxyC₁₋₆alkylene; aminoC₁₋₆alkylene; mercaptoC₁₋₈alkylene; C₁₋₆alkylene, C₃₋₈cycloalkylene; C₆₋₁₂arylC₁₋₆alkylene; heterocyclylC₁₋₆alkylene; and heteroarylC₁₋₆alkylene; preferably Q⁵ is a single bond or selected from the group consisting of hydroxyC₁₋₆alkylene; aminoC₁₋₆alkylene; mercaptoC₁₋₆alkylene; C₁₋₆alkylene, C₃₋₈cycloalkylene; C₆₋₁₂arylC₁₋₆alkylene; heterocyclylC₁₋₆alkylene; and heteroarylC₁₋₆alkylene; wherein said C₁₋₂₀alkylene, C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₆alkylene; or heteroarylC₁₋₆alkylene, can be unsubstituted or substituted with one or more Z^{8a}; preferably said C₁₋₂₀alkylene, C₆₋₁₂arylene, C₃₋₈cycloalkylene; C₆₋₁₂arylC₁₋₆alkylene; heterocyclylene; heteroarylene; heterocyclylC₁₋₆alkylene; or heteroarylC₁₋₆alkylene, can be unsubstituted or substituted with one or more Z^{8a}; preferably said C₁₋₂₀alkylene, C₆₋₁₂arylene, C₃₋₈cycloalkylene; heterocyclylene; or heteroarylene; can be unsubstituted or substituted with one or more Z^{5a};
wherein if p is 0, then one of Q⁴ and Q⁵ is not a single bond;
each R¹⁴ and R¹⁵ is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group; preferably each R¹⁴ and R¹⁵ is independently selected from the group consisting of hydrogen; C₁₋₂₀alkyl, hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group; preferably each R¹⁴ and R¹⁵ is independently selected from the group consisting of hydrogen; C₁₋₂₀alkyl, hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclylC₁₋₆alkyl; heteroarylC₁₋₆alkyl; and a polymeric group; preferably each R¹⁴ and R¹⁵ is independently selected from the group consisting of hydrogen; C₁₋₁₈alkyl, hydroxyC₁₋₈alkoxy; aminoC₁₋₈alkoxy; mercaptoC₁₋₈alkoxy; C₆₋₁₂aryl, C₃₋₈cycloalkyl; C₆₋₁₂arylC₁₋₈alkyl; heterocyclylC₁₋₆alkyl; heteroarylC₁₋₆alkyl; and a polymeric group; preferably each R¹⁴ and R¹⁵ is independently selected from the group consisting of hydrogen; C₁₋₁₀alkyl, hydroxyC₁₋₈alkoxy; aminoC₁₋₈alkoxy; mercaptoC₁₋₈alkoxy; C₆₋₁₂aryl, C₃₋₈cycloalkyl; C₆₋₁₂arylC₁₋₈alkyl; heterocyclylC₁₋₆alkyl; heteroarylC₁₋₆alkyl; and a polymeric group;
wherein for each R¹⁴ and R¹⁵ said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group; preferably wherein said polymeric group is selected from the group consisting of polyurethane; isocyanate containing prepolymer, hydroxyl containing prepolymer, polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; ethylene propylene diene monomer (M-class) rubber (EPDM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group; preferably wherein said polymeric group is selected from the group consisting of polyurethane; isocyanate containing prepolymer, hydroxyl containing prepolymer, polyacrylate; polymethacrylate; polystyrene; copolymer of acrylate; poly(methyl methacrylate); methacrylate and/or styrene; poly(isobornyl acrylate); polyacrylonitrile; polyamide; polyester amide; ethylene propylene diene monomer (M-class) rubber (EPDM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
each R¹⁶ and R¹⁷ is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group; preferably each R¹⁶ and R¹⁷ is independently selected from the group consisting of hydrogen; C₁₋₂₀alkyl, hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group; preferably each R¹⁶ and R¹⁷ is independently selected from the group consisting of hydrogen; C₁₋₂₀alkyl, hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclylC₁₋₆alkyl; heteroarylC₁₋₆alkyl; and a polymeric group; preferably each R¹⁶ and R¹⁷ is independently selected from the group consisting of hydrogen; C₁₋₁₈alkyl, hydroxyC₁₋₈alkoxy; aminoC₁₋₈alkoxy; mercaptoC₁₋₈alkoxy; C₆₋₁₂aryl, C₃₋₈cycloalkyl; C₆₋₁₂arylC₁₋₆alkyl; heterocyclylC₁₋₆alkyl; heteroarylC₁₋₆alkyl; and a polymeric group; preferably each R¹⁶ and R¹⁷ is independently selected from the group consisting of hydrogen; C₁₋₁₀alkyl, hydroxyC₁₋₆alkoxy; aminoC₁₋₆alkoxy; mercaptoC₁₋₆alkoxy; C₆₋₁₂aryl, C₃₋₈cycloalkyl; C₆₋₁₂arylC₁₋₆alkyl; heterocyclylC₁₋₆alkyl; heteroarylC₁₋₆alkyl; and a polymeric group;
wherein for each R¹⁶ and R¹⁷ said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group; preferably wherein said polymeric group is selected from the group consisting of polyurethane; isocyanate containing prepolymer, hydroxyl containing prepolymer, polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; ethylene propylene diene monomer (M-class) rubber (EPDM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group; preferably wherein said polymeric group is selected from the group consisting of polyurethane; isocyanate containing prepolymer, hydroxyl containing prepolymer, polyacrylate; polymethacrylate; polystyrene; copolymer of acrylate; poly(methyl methacrylate); methacrylate and/or styrene; poly(isobornyl acrylate); polyacrylonitrile; polyamide; polyester amide; ethylene propylene diene monomer (M-class) rubber (EPDM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
R¹⁸ is selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; and heteroarylC₁₋₆alkyl; preferably R¹⁸ is selected from the group consisting of hydrogen; C₁₋₂₀alkyl, hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; and heteroarylC₁₋₆alkyl; preferably R¹⁸ is selected from the group consisting of hydrogen; C₁₋₂₀alkyl, hydroxyC₁₋₁₀alkoxy; aminoC₁₋₁₀alkoxy; mercaptoC₁₋₁₀alkoxy; C₃₋₈cycloalkyl; C₆₋₁₂arylC₁₋₆alkyl; heterocyclylC₁₋₆alkyl; and heteroarylC₁₋₆alkyl; preferably R¹⁸ is selected from the group consisting of hydrogen; C₁₋₁₈alkyl, hydroxyC₁₋₈alkoxy; aminoC₁₋₈alkoxy; mercaptoC₁₋₈alkoxy; C₃₋₈cycloalkyl; C₆₋₁₂arylC₁₋₆alkyl; heterocyclylC₁₋₆alkyl; and heteroarylC₁₋₆alkyl; preferably R¹⁸ is selected from the group consisting of hydrogen; C₁₋₁₀alkyl, hydroxyC₁₋₆alkoxy; aminoC₁₋₆alkoxy; mercaptoC₁₋₆alkoxy; C₃₋₈cycloalkyl; C₆₋₁₂arylC₁₋₆alkyl; heterocyclylC₁₋₆alkyl; and heteroarylC₁₋₆alkyl;

each R⁶ is independently selected from the group consisting of hydrogen, halogen; C₁₋₁₀alkyl; C₂₋₁₀alkenyl; C₂₋₁₀alkynyl; C₆₋₁₂arylC₁₋₆alkyl; haloC₁₋₆alkyl; haloC₁₋₆alkyloxy; —OR¹⁹; —SR¹⁹; nitro; NR²⁰R²¹; —C(O)NR²⁰R²¹; cyano; and a polymeric group; preferably each R⁶ is independently selected from the group consisting of hydrogen, halogen; C₁₋₁₀alkyl; C₆₋₁₂arylC₁₋₆alkyl; haloC₁₋₆alkyl; haloC₁₋₆alkyloxy; —OR¹⁹; —SR¹⁹; nitro; NR²⁰R²¹; —C(O)NR²⁰R²¹; cyano; and a polymeric group; preferably each R⁶ is independently selected from the group consisting of hydrogen, halogen; C₁₋₁₀alkyl; C₆₋₁₂arylC₁₋₆alkyl; haloC₁₋₆alkyl; —OR¹⁹; NR²⁰R²¹; —C(O)NR²⁰R²¹; cyano; and a polymeric group; preferably each R⁶ is independently selected from the group consisting of hydrogen, halogen; C₁₋₁₀alkyl; C₆₋₁₂arylC₁₋₆alkyl; haloC₁₋₆alkyl; —OR¹⁹; —C(O)NR²⁰R²¹; and a polymeric group;
wherein said C₁₋₁₀alkyl; C₂₋₁₀alkenyl; C₂₋₁₀alkynyl; or C₆₋₁₂arylC₁₋₆alkyl, can be unsubstituted or substituted with one or more Z⁹; wherein said C₁₋₁₀alkyl; or C₆₋₁₂arylC₁₋₆alkyl, can be unsubstituted or substituted with one or more Z⁹; wherein said C₁₋₁₀alkyl; or C₆₋₁₂arylC₁₋₆alkyl, can be unsubstituted or substituted with one, two or three Z⁹;
and wherein for R⁶ said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group; preferably wherein said polymeric group is selected from the group consisting of polyurethane; isocyanate containing prepolymer, hydroxyl containing prepolymer, polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; ethylene propylene diene monomer (M-class) rubber (EPDM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group; preferably wherein said polymeric group is selected from the group consisting of polyurethane; isocyanate containing prepolymer, hydroxyl containing prepolymer, polyacrylate; polymethacrylate; polystyrene; copolymer of acrylate; poly(methyl methacrylate); methacrylate and/or styrene; poly(isobornyl acrylate); polyacrylonitrile; polyamide; polyester amide; ethylene propylene diene monomer (M-class) rubber (EPDM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;

each R¹⁹ is independently selected from the group consisting of C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl; wherein each group optionally comprise one or more heteroatoms in the alkyl, alkenyl or alkynyl moiety, said heteroatoms being each independently selected from O, S and N; preferably wherein each R¹⁹ is independently selected from the group consisting of C₁₋₁₀alkyl, C₂₋₁₀alkenyl, C₆₋₁₂aryl, C₆₋₁₂arylC₁₋₆alkyl; wherein each group optionally comprise one or more heteroatoms in the alkyl, alkenyl or alkynyl moiety, said heteroatoms being each independently selected from O, S and N preferably wherein each R¹⁹ is independently selected from the group consisting of C₁₋₆alkyl, C₂₋₆alkenyl, C₆₋₁₂aryl; wherein each group optionally comprise one or more heteroatoms in the alkyl, alkenyl or alkynyl moiety, said heteroatoms being each independently selected from O, and N;

wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl, can be unsubstituted or substituted with one or more OH, SH, COOH, or NH₂; preferably said C₁₋₂₀alkyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₈alkyl, can be unsubstituted or substituted with one or more OH, SH, COOH, or NH₂; preferably said C₁₋₂₀alkyl, C₆₋₁₂aryl, C₃₋₈cycloalkyl, C₆₋₁₂arylC₁₋₆alkyl, heterocyclyl; heteroaryl, can be unsubstituted or substituted with one or more OH, SH, COOH, or NH₂; preferably said C₁₋₂₀alkyl, C₆₋₁₂aryl, C₃₋₈cycloalkyl, C₆₋₁₂arylC₁₋₆alkyl, heterocyclyl; heteroaryl, can be unsubstituted or substituted with one or more OH, SH, or NH₂; preferably said C₁₋₂₀alkyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl, can, can be unsubstituted or substituted with one or more OH, SH, or NH₂; preferably said C₁₋₂₀alkyl, C₆₋₁₂aryl, C₃₋₈cycloalkyl, C₆₋₁₂arylC₁₋₆alkyl, can be unsubstituted or substituted with one or more OH, SH, or NH₂;

each R²⁰ is independently selected from the group consisting of C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₈alkyl; and heteroarylC₁₋₆alkyl; wherein each group optionally comprise one or more heteroatoms in the alkyl, alkenyl or alkynyl moiety, said heteroatoms being each independently selected from O, S and N; preferably wherein each R²⁰ is independently selected from the group consisting of C₁₋₁₀alkyl, C₂₋₁₀alkenyl, C₆₋₁₂aryl, C₆₋₁₂arylC₁₋₆alkyl; wherein each group optionally comprise one or more heteroatoms in the alkyl, alkenyl or alkynyl moiety, said heteroatoms being each independently selected from O, S and N; preferably wherein each R²⁰ is independently selected from the group consisting of C₁₋₆alkyl, C₂₋₈alkenyl, C₆₋₁₂aryl; wherein each group optionally comprise one or more heteroatoms in the alkyl, alkenyl or alkynyl moiety, said heteroatoms being each independently selected from O, and N;

wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl, can be unsubstituted or substituted with one or more OH, SH, COOH, or NH₂; preferably said C₁₋₂₀alkyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₈alkyl, can be unsubstituted or substituted with one or more OH, SH, COOH, or NH₂; preferably said C₁₋₂₀alkyl, C₆₋₁₂aryl, C₃₋₈cycloalkyl, C₆₋₁₂arylC₁₋₆alkyl, heterocyclyl; heteroaryl, can be unsubstituted or substituted with one or more OH, SH, COOH, or NH₂; preferably said C₁₋₂₀alkyl, C₆₋₁₂aryl, C₃₋₈cycloalkyl, C₆₋₁₂arylC₁₋₆alkyl, heterocyclyl; heteroaryl, can be unsubstituted or substituted with one or more OH, SH, or NH₂; preferably said C₁₋₂₀alkyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl, can, can be unsubstituted or substituted with one or more OH, SH, or NH₂; preferably said C₁₋₂₀alkyl, C₆₋₁₂aryl, C₃₋₈cycloalkyl, C₆₋₁₂arylC₁₋₆alkyl, can be unsubstituted or substituted with one or more OH, SH, or NH₂;

each R²¹ is independently selected from the group consisting of hydrogen; C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl; wherein each group optionally comprise one or more heteroatoms in the alkyl, alkenyl or alkynyl moiety, said heteroatoms being each independently selected from O, S and N; preferably wherein each R²¹ is independently selected from the group consisting of hydrogen; C₁₋₁₀alkyl, C₂₋₁₀alkenyl, C₆₋₁₂aryl, C₆₋₁₂arylC₁₋₆alkyl; wherein each group optionally comprise one or more heteroatoms in the alkyl, alkenyl or alkynyl moiety, said heteroatoms being each independently selected from O, S and N; preferably wherein each R²¹ is independently selected from the group consisting of hydrogen; C₁₋₆alkyl, C₂-6alkenyl, C₆₋₁₂aryl; wherein each group optionally comprise one or more heteroatoms in the alkyl, alkenyl or alkynyl moiety, said heteroatoms being each independently selected from 0, and N;

wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl, can be unsubstituted or substituted with one or more OH, SH, COOH, or NH₂; preferably said C₁₋₂₀alkyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₈alkyl, can be unsubstituted or substituted with one or more OH, SH, COOH, or NH₂; preferably said C₁₋₂₀alkyl, C₆₋₁₂aryl, C₃₋₈cycloalkyl, C₆₋₁₂arylC₁₋₆alkyl, heterocyclyl; heteroaryl, can be unsubstituted or substituted with one or more OH, SH, COOH, or NH₂; preferably said C₁₋₂₀alkyl, C₆₋₁₂aryl, C₃₋₈cycloalkyl, C₆₋₁₂arylC₁₋₆alkyl, heterocyclyl; heteroaryl, can be unsubstituted or substituted with one or more OH, SH, or NH₂; preferably said C₁₋₂₀alkyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl, can, can be unsubstituted or substituted with one or more OH, SH, or NH₂; preferably said C₁₋₂₀alkyl, C₆₋₁₂aryl, C₃₋₈cycloalkyl, C₆₋₁₂arylC₁₋₆alkyl, can be unsubstituted or substituted with one or more OH, SH, or NH₂;

or wherein R²⁰ and R²¹ together with the atom to which they are attached form a 5-, 6-, or 7-membered heterocycle;

Z¹, Z², Z³, Z⁴, Z⁶ are each independently selected from the group consisting of OH; SH; COOH; NH₂; NHR²⁰; halogen; C₁₋₆alkyl; haloC₁₋₆alkyl; haloC₁₋₆alkyloxy; —OR¹⁹; —SR¹⁹; nitro; NR²⁰R²¹; —C(O)NR²⁰R²¹; cyano; preferably Z¹, Z², Z³, Z⁴, Z⁶ are each independently selected from the group consisting of OH; SH; COOH; NH₂; NHR²⁰; halogen; C₁₋₆alkyl; haloC₁₋₆alkyl; haloC₁₋₈alkyloxy; —OR¹⁹; —SR¹⁹; —C(O)NR²⁰R²¹; cyano; preferably Z¹, Z², Z³, Z⁴, Z⁶ are each independently selected from the group consisting of OH; SH; COOH; NH₂; NHR²⁰; halogen; C₁₋₈alkyl; haloC₁₋₆alkyl; —OR¹⁹; —SR¹⁹; —C(O)NR²⁰R²¹; cyano; preferably Z¹, Z², Z³, Z⁴, Z⁶ are each independently selected from the group consisting of OH; SH; COOH; NH₂; NHR²⁰; halogen; C₁₋₈alkyl; haloC₁₋₆alkyl; haloC₁₋₆alkyloxy; —OR¹⁹; —SR¹⁹; preferably Z¹, Z², Z³, Z⁴, Z⁶ are each independently selected from the group consisting of OH; SH; COOH; NH₂; NHR²⁰; halogen; C₁₋₆alkyl; haloC₁₋₆alkyl; haloC₁₋₆alkyloxy; preferably Z¹, Z², Z³, Z⁴, Z⁶ are each independently selected from the group consisting of OH; SH; COOH; NH₂; NHR²⁰; halogen; C₁₋₆alkyl; haloC₁₋₆alkyl;

Z⁵, Z^{5a}, Z⁷, Z⁸, Z^{8a}, Z⁹ are each independently selected from the group consisting of OH; hydrogen; SH; COOH; NH₂; NHR²⁰; halogen; C₁₋₆alkyl; haloC₁₋₆alkyl; haloC₁₋₆alkyloxy; —OR¹⁹; —SR¹⁹; nitro; NR²⁰R²¹; cyano; and a polymeric group; preferably Z⁵, Z^{5a}, Z⁷, Z⁸, Z^{8a}, Z⁹ are each independently selected from the group consisting of OH; hydrogen; SH; COOH; NH₂; NHR²⁰; halogen; C₁₋₆alkyl; haloC₁₋₆alkyl; haloC₁₋₆alkyloxy; —OR¹⁹; —SR¹⁹; cyano; and a polymeric group; preferably Z⁵, Z^{5a}, Z⁷, Z⁸, Z^{8a}, Z⁹ are each independently selected from the group consisting of OH; hydrogen; SH; COOH; NH₂; NHR²⁰; halogen; C₁₋₆alkyl; haloC₁₋₆alkyl; —OR¹⁹; —SR¹⁹; cyano; and a polymeric group; preferably Z⁵, Z^{5a}, Z⁷, Z⁸, Z^{8a}, Z⁹ are each independently selected from the group consisting of OH; hydrogen; SH; COOH; NH₂; NHR²⁰; halogen; C₁₋₆alkyl; haloC₁₋₆alkyl; haloC₁₋₆alkyloxy; —OR¹⁹; —SR¹⁹; and a polymeric group; preferably Z⁵, Z^{5a}, Z⁷, Z⁸, Z^{8a}, Z⁹ are each independently selected from the group consisting of OH; hydrogen; SH; COOH; NH₂; NHR²⁰; halogen; C₁₋₆alkyl; haloC₁₋₆alkyl; haloC₁₋₆alkyloxy; and a polymeric group; preferably Z⁵, Z^{5a}, Z⁷, Z⁸, Z^{8a}, Z⁹ are each independently selected from the group consisting of OH; hydrogen;

SH; COOH; NH₂; NHR²⁰; halogen; C₁₋₆alkyl; haloC₁₋₆alkyl; and a polymeric group;

and when none of R¹, R², R³, R⁴, R⁶, R⁷, R⁸, R⁹, R¹⁰, R¹¹, R¹², R¹⁴, R¹⁵, R¹⁶, R¹⁷, Z⁵, Z^{5a}, Z⁷, Z⁸, Z^{8a}, or Z⁹ is a polymeric group, then at least two of R⁷, R⁸, Z¹, Z², Z³, Z⁴, Z⁵, Z^{5a}, Z⁶, Z⁷, Z⁸, Z^{8a} or Z⁹ are selected from the group consisting of OH, SH, COOH, NH₂, and NHR²⁰; preferably two or more of R⁷, R⁸, Z¹, Z², Z³, Z⁴, Z⁵, Z^{5a}, Z⁶, Z⁷, Z⁸, Z^{8a} or Z⁹ are selected from the group consisting of OH, SH, COOH, NH₂, and NHR²⁰; preferably three R⁷, R⁸, Z¹, Z², Z³, Z⁴, Z⁵, Z^{5a}, Z⁶, Z⁷, Z⁸, Z^{8a} or Z⁹ are selected from the group consisting of OH, SH, COOH, NH₂, and NHR²⁰; preferably four of R⁷, R⁸, Z¹, Z², Z³, Z⁴, Z⁵, Z^{5a}, Z⁶, Z⁷, Z⁸, Z^{8a} or Z⁹ are selected from the group consisting of OH, SH, COOH, NH₂, and NHR²⁰.

According to another embodiment, the present invention provides compounds of formula (A) and any subgroup thereof wherein,

r is selected from 0, 1, 2, or 3; preferably r is selected from 0, 1 or 2; preferably r is selected from 0, or 1; more preferably r is 0;

R¹ is C₁₋₁₀alkyl; for example R¹ is C₁₋₆alkyl; for example R¹ is C₁₋₆alkyl; for example C₁₋₄alkyl, for example C₁₋₂alkyl,

R² and R³ and R⁴ together with the carbon atom to which they are attached can form an unsaturated 5-, or 6-membered ring; preferably R² and R³ and R⁴ together with the carbon atom to which they are attached can form a saturated or unsaturated 6-membered ring; preferably R² and R³ and R⁴ together with the carbon atom to which they are attached can form an unsaturated 6-membered ring; preferably R² and R³ and R⁴ together with the carbon atom to which they are attached can form an aryl ring; for example a phenyl ring;

wherein said unsaturated 5-, or 6-membered ring can be unsubstituted or substituted with one or more R⁸; preferably said saturated or unsaturated 6-membered ring can be unsubstituted or substituted with one or more R⁸; preferably said unsaturated 6-membered ring can be unsubstituted or substituted with one or more R⁸; preferably said phenyl ring can be unsubstituted or substituted with one or more R⁸; preferably said phenyl ring can be unsubstituted or substituted with one, two or three R⁸;

each R⁸ is independently selected from the group consisting of hydroxyC₁₋₆alkoxy; aminoC₁₋₆alkoxy; mercaptoC₁₋₆alkoxy; C₁₋₆alkyl; C₆₋₁₂arylC₁₋₆alkyl; C₃₋₈cycloalkyl; C₆₋₁₂arylC₁₋₆alkyl; heterocyclylC₁₋₆alkyl; heteroarylC₁₋₆alkyl; OH; SH; COOH; NH₂; and a polymeric group, more preferably hydroxyC₁₋₆alkoxy;

wherein said hydroxyC₁₋₆alkoxy; aminoC₁₋₆alkoxy; mercaptoC₁₋₆alkoxy; C₁₋₆alkyl; C₆₋₁₂arylC₁₋₆alkyl; C₃₋₈cycloalkyl; C₆₋₁₂arylC₁₋₆alkyl; heterocyclylC₁₋₆alkyl; or heteroarylC₁₋₆alkyl, can be unsubstituted or substituted with one or more Z⁷;

each Z⁷ is independently selected from the group consisting of OH; hydrogen; SH; COOH; NH₂; halogen; C₁₋₆alkyl; haloC₁₋₆alkyl; and a polymeric group.

According to another embodiment, the present invention provides compounds of formula (A2) and any subgroup thereof wherein,

r is selected from 0, 1, 2, or 3; preferably r is selected from 0, 1 or 2; preferably r is selected from 0, or 1; more preferably r is 0;

R¹ is C₁₋₁₀alkyl; for example R¹ is C₁₋₆alkyl; for example R¹ is C₁₋₆alkyl; for example C₁₋₄alkyl, for example C₁₋₂alkyl,

each R⁸ is independently selected from the group consisting of hydroxyC₁₋₆alkoxy; aminoC₁₋₆alkoxy; mercaptoC₁₋₆alkoxy; C₁₋₆alkyl; C₆₋₁₂arylC₁₋₆alkyl; C₃₋₈cycloalkyl; C₆₋₁₂arylC₁₋₆alkyl; heterocyclylC₁₋₆alkyl; heteroarylC₁₋₆alkyl; OH; SH; COOH; NH₂; and a polymeric group, preferably hydroxyC₁₋₆alkoxy;

wherein said hydroxyC₁₋₆alkoxy; aminoC₁₋₆alkoxy; mercaptoC₁₋₆alkoxy; C₁₋₆alkyl; C₆₋₁₂arylC₁₋₆alkyl; C₃₋₈cycloalkyl; C₆₋₁₂arylC₁₋₆alkyl; heterocyclylC₁₋₆alkyl; or heteroarylC₁₋₆alkyl, can be unsubstituted or substituted with one or more Z⁷;

each Z⁷ is independently selected from the group consisting of OH; hydrogen; SH; COOH; NH₂; halogen; C₁₋₆alkyl; haloC₁₋₆alkyl; and a polymeric group.

A non-limiting example of a compound of formula (A) and (A2) is 3-(4-(3-methyl-1H-indol-2-yl)phenoxy)propane-1,2-diol. The present invention therefore also encompasses said compound as well compositions comprising said 3-(4-(3-methyl-1H-indol-2-yl)phenoxy)propane-1,2-diol, as well as the use thereof in the preparation of polymers, such as polyurethane, and the polymers obtained therefrom.

The present invention also encompasses processes for the preparation of compounds Formula (I) and any subgroup thereof. In the reactions described, it can be necessary to protect reactive functional groups, for example hydroxy, amino, or carboxy groups, where these are desired in the final product, to avoid their unwanted participation in the reactions. Conventional protecting groups can be used in accordance with standard practice, for example, see T. W. Greene and P. G. M. Wuts in “Protective Groups in Organic Chemistry”, John Wiley and Sons, 1999.

The compounds of the invention can be prepared by different ways with reactions known by the person skilled in the art. Reaction schemes as described in the examples section illustrate by way of example different possible approaches.

Compounds of formula (I) can be prepared by contacting a compound of formula (II) with a compound of formula (III), thereby obtaining a compound of formula (I), wherein r, R¹, R², R³, R⁴, R⁵, R⁶, L¹, have the same meaning as defined herein,

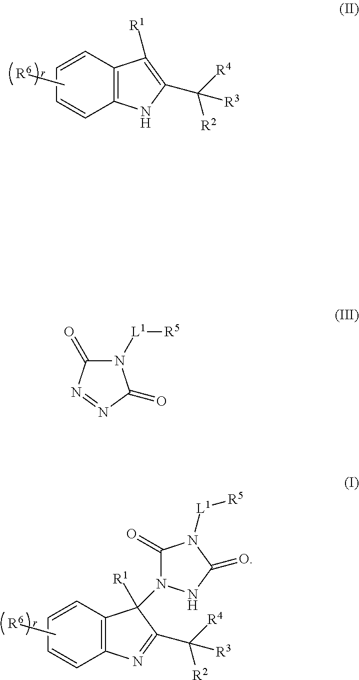

In some embodiments, the compounds of formula (I) are prepared by contacting a compound of formula (II) with a compound of formula (IIIA), thereby obtaining a compound of formula (I), wherein r, R¹, R², R³, R⁴, R⁵, R⁶, L¹, have the same meaning as defined herein,

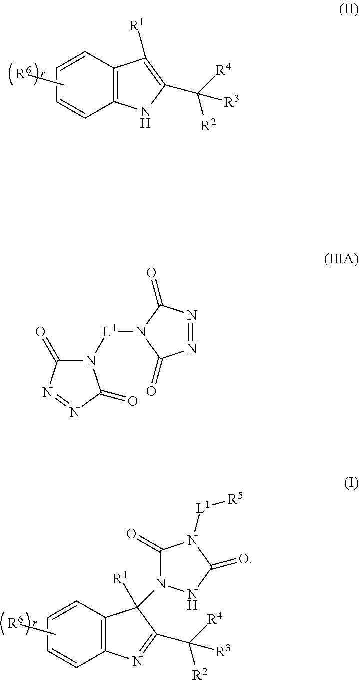

The process for the preparation of a compound of formula (I) can take place at low temperature (<20° C.), without the need for a catalyst. Preferably, the process for the preparation of a compound of formula (I) takes place at room temperature. In another embodiment, the process comprising the step of contacting a compound of formula (II) with a compound of formula (III) or (IIIA) occurs at a temperature of preferably lower then 50° C., and more preferably lower then 30° C., thereby forming a compound of formula (I).

In some embodiments, the compound of formula (II) is a compound of formula (A).

In an embodiment, the step of contacting a compound of formula (II) with a compound of formula (III) or (IIIA) is performed at equimolar conditions. In another preferred embodiment, the molar ratio of compound (II) to compound (III) or (IIIA) is at least 2.0, preferably at least 2.5; preferably at least 3.0; preferably at least 3.5; more preferably at least 4.0.

The compound of formula (III) or (IIIA) may be synthesized by the reaction of a polyisocyanate with ethylcarbazate, followed by cyclization to produce urazole rings, and finally oxidation of the urazole rings to produce the 1,2,4-triazoline-3,5-dione (TAD) of compound (III).

Suitable polyisocyanates for use in the preparation of compound (III) or (IIIA) may be aromatic, cycloaliphatic, heterocyclic, araliphatic or aliphatic organic polyisocyanates, as well as modified isocyanates.

Suitable polyisocyanates comprise polyisocyanates of the type R^{a}—(NCO)ₓ with x being at least 1 and R^{a} being an aromatic or aliphatic group, such as diphenylmethane, toluene, dicyclohexylmethane, hexamethylene, or a similar polyisocyanate. Preferably, said polyisocyanate comprises at least two isocyanate groups.

Suitable isocyanates are the aliphatic, cycloaliphatic, araliphatic and/or aromatic isocyanates known per se, preferably diisocyanates, which may have been, if desired, biuretized and/or isocyanuratized by generally known methods. Specific examples are: alkylene diisocyanate having from 4 to 12 carbon atoms in the alkylene radical, for example dodecane 1,12-diisocyanate, 2-ethyltetramethylene 1,4-diisocyanate, 2-methylpentamethylene 1,5-diisocyanate, tetramethylene 1,4-diisocyanate, lysine ester diisocyanates (LDI), hexamethylene 1,6-diisocyanate (HDI), cyclohexane 1,3- and/or 1,4-diisocyanate, hexahydrotolylene 2,4- and 2,6-diisocyanate and also the corresponding isomer mixtures, dicyclohexylmethane 4,4′-, 2,2′- and 2,4′-diisocyanate and also the corresponding isomer mixtures and 1-isocyanato-3,3,5-trimethyl-5-isocyanatomethylcyclohexane (IPDI), and also tolylene 2,4- and/or 2,6-diisocyanate, diphenylmethane 4,4′-, 2,4′- and/or 2,2′-diisocyanate, polyphenylpoly-methylene polyisocyanates and/or mixtures comprising at least two of the abovementioned isocyanates. Furthermore, diisocyanates and/or polyisocyanates containing ester, urea, allophanate, carbodiimide, uretdione and/or urethane groups can be used in the process of the present invention.

Non-limiting examples of suitable organic polyisocyanates which may be used in the present invention include aliphatic isocyanates such as isophorone diisocyanate (IPDI) or hexamethylene diisocyanate; and aromatic isocyanates such as diphenylmethane diisocyanate (MDI), in the form of mixtures of its 2,4′-, 2,2′- and 4,4′-isomers and mixtures of diphenylmethane diisocyanates (MDI) and oligomers thereof, as well as polymeric methylene diphenyl diisocyanate, m- and p-phenylene diisocyanate, tolylene-2,4- and tolylene-2,6-diisocyanate (also known as toluene diisocyanate, and referred to as TDI, such as 2,4 TDI and 2,6 TDI) in any suitable isomer mixture, chlorophenylene-2,4-diisocyanate, naphthylene-1,5-diisocyanate, 4,4′-diisocyanate-3,3′-dimethyl-diphenyl, 3-methyl-diphenylmethane-4,4′-diisocyanate and cycloaliphatic diisocyanates such as cyclohexane-1,3- and -1,4-diisocyanate, 1-methylcyclohexyl-2,4- and -2,6-diisocyanate and mixtures thereof and bis-(isocyanatocyclohexyl)methane (e.g. 4,4′-diisocyanatodicyclohexylmethane (H12MDI)), triisocyanates such as 2,4,6-triisocyanatotoluene and 2,4,4-triisocyanatodiphenylether, butylene diisocyanate, trimethylhexamethylene diisocyanate, isocyanatomethyl-1,8-octane diisocyanate, tetramethylxylene diisocyanate (TMXDI), tolidine diisocyanate (TODI); m-xylylene diisocyanate; p-xylylene diisocyanate; any suitable mixture of these polyisocyanates.

Suitable polyisocyanates that can be used in the present invention may be diisocyanates and/or polyisocyanates selected from the group comprising toluene diisocyanates (TDI), diphenylmethane diisocyanate (MDI)—type isocyanates, trimers or prepolymers of these isocyanates, as well as modified isocyanates such as diisocyanates and/or polyisocyanates containing ester, urea, allophanate, carbodiimide, uretdione and/or urethane groups.

The polymeric methylene diphenyl diisocyanate can be any mixture of MDI (as defined herein above) and higher homologues of formula (Ax):

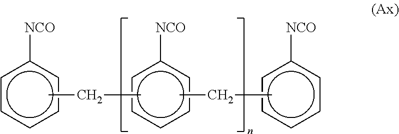

wherein n is an integer which can be from 0 to 10, preferably from 1 to 5.

The invention also encompasses a composition comprising a compound of formula (A) as described herein.

The invention also encompasses a composition comprising a compound of formula (I) as described herein. In some embodiments, the composition further comprises a compound of formula (A),

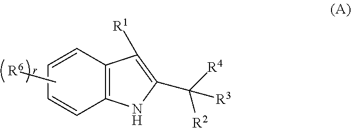

wherein r, R¹, R², R³, R⁴ and R⁶ are the same as defined hereinabove for structure (A).

In some embodiments, the compound of formula (A) is a compound of formula (II).

In some embodiments, the composition comprises at least 2.0 moles of compound (A) for every mole of compound (I); preferably, the composition comprises a molar ratio of compound (A) to compound (I) of 2.0; preferably the molar ratio of compound (A) to compound (I) is 1.0 or more; preferably more than 1.0; preferably more than 1.2; preferably more than 1.4; preferably more than 1.6; preferably more than 2.0; preferably 2.0 or more.

In some embodiments, the composition further comprises a polyol; preferably said polyol is a bifunctional polyol; preferably a polyol with a molecular weight (Mn) of less than 2000 g/mol; preferably a molecular weight of less than 1500 g/mol; preferably a molecular weight of less than 1000 g/mol; preferably a molecular weight of less than 600 g/mol; more preferably the polyol is a bifunctional polyol with a molecular weight of less than 2000 g/mol; preferably a molecular weight of less than 1500 g/mol; preferably a molecular weight of less than 1000 g/mol; preferably a molecular weight of less than 600 g/mol.

Suitable polyols for the composition include diethylene glycol, polypropylene glycol and polyethylene glycol.

The composition according to the present invention may also comprise at least one of the following components: dispersing agents, surfactants, catalysts (amines or metallic catalysts), surfactants, fire retardants, blowing agents, anti-oxidants, fillers, solvents, chain extenders, or combinations thereof.

The present invention also encompasses polyurethane obtained by contacting at least one isocyanate with at least one isocyanate-reactive compound and with at least one compound of formula (I), or with a composition comprising a compound of formula (I) as defined hereinabove.

Suitable isocyanates for use in the preparation of polyurethane may be aromatic, cycloaliphatic, heterocyclic, araliphatic or aliphatic organic polyisocyanates. Suitable isocyanates are preferably polyisocyanates.

Suitable polyisocyanates comprise polyisocyanates of the type R^{a}—(NCO)ₓ with x being at least 1 and R^{a} being an aromatic or aliphatic group, such as diphenylmethane, toluene, dicyclohexylmethane, hexamethylene, or a similar polyisocyanate. Preferably, said polyisocyanate comprises at least two isocyanate groups.

Non-limiting examples of suitable organic polyisocyanates which may be used in the present invention include aliphatic isocyanates such as hexamethylene diisocyanate; and aromatic isocyanates such as diphenylmethane diisocyanate (MDI), in the form of mixtures of its 2,4′-, 2,2′- and 4,4′-isomers and mixtures of diphenylmethane diisocyanates (MDI) and oligomers thereof, as well as polymeric methylene diphenyl diisocyanate, m- and p-phenylene diisocyanate, tolylene-2,4- and tolylene-2,6-diisocyanate (also known as toluene diisocyanate, and referred to as TDI, such as 2,4 TDI and 2,6 TDI) in any suitable isomer mixture, chlorophenylene-2,4-diisocyanate, naphthylene-1,5-diisocyanate, diphenylene-4,4′-diisocyanate, 4,4′-diisocyanate-3,3′-dimethyl-diphenyl, 3-methyl-diphenylmethane-4,4′-diisocyanate and diphenyl ether diisocyanate; and cycloaliphatic diisocyanates such as cyclohexane-2,4- and -2,3-diisocyanate, 1-methylcyclohexyl-2,4- and -2,6-diisocyanate and mixtures thereof and bis-(isocyanatocyclohexyl)methane (e.g. 4,4′-diisocyanatodicyclohexylmethane (H12MDI)), triisocyanates such as 2,4,6-triisocyanatotoluene and 2,4,4-triisocyanatodiphenylether, isophorone diisocyanate (IPDI), butylene diisocyanate, trimethylhexamethylene diisocyanate, isocyanatomethyl-1,8-octane diisocyanate, tetramethylxylene diisocyanate (TMXDI), 1,4-cyclohexanediisocyanate (CDI), and tolidine diisocyanate (TODI); pentamethylene 1,5-diisocyanate (so-called biobased isocyanate), any suitable mixture of these polyisocyanates. Furthermore, diisocyanates and/or polyisocyanates containing ester, urea, allophanate, carbodiimide, uretdione and/or urethane groups can be used in the process of the present invention. Preferably, the polyisocyanates that can be used in the present invention may be diisocyanates and/or a polyisocyanates selected from the group comprising toluene diisocyanates (TDI), diphenylmethane diisocyanate (MDI)-type isocyanates, and trimers or prepolymers of these isocyanates.

The polymeric methylene diphenyl diisocyanate can be any mixture of MDI (as defined herein above) and higher homologues of formula (Ax):

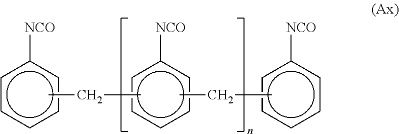

wherein n is an integer which can be from 0 to 10, preferably from 1 to 5.

In some embodiments, the at least one isocyanate-reactive compound may be a component containing isocyanate-reactive hydrogen atoms. As used herein, the term “isocyanate-reactive hydrogen atom” refers to compounds that contain acidic hydrogen atoms, which make the compounds that bear them, susceptible to electrophilic attack by an isocyanate group.

Examples of suitable isocyanate-reactive compounds containing isocyanate-reactive hydrogen atoms include polyols such as glycols or even relatively high molecular weight polyether polyols and polyester polyols, thiols (mercaptans), carboxylic acids such as polybasic acids, amines, polyamines, components comprising at least one alcohol group and at least one amine group, such as polyaminepolyols, urea and amides.

In some embodiments, said at least one isocyanate-reactive compound may have a number average molecular weight of at least 200 g/mol; preferably of at least 400 g/mol; more preferably of at least 700 g/mol. In some embodiments, said at least one isocyanate-reactive compound may have a number average molecular weight of at most 8000 g/mol; preferably of at most 7000 g/mol; more preferably of at most 6000 g/mol, most preferably of at most 4000 g/mol. In some embodiments, said at least one isocyanate-reactive compound may have a number average molecular weight of at least 200 to at most 8000 g/mol; preferably of at least 300 to at most 7000 g/mol; more preferably of at least 400 to at most 6000 g/mol.

Particularly preferred are polyols and polyol mixtures having average molecular weights of from 200 to 8000 g/mol, and hydroxyl functionalities of from 2 to 8. Suitable polyols include reaction products of alkylene oxides, for example ethylene oxide and/or propylene oxide, with initiators containing from 2 to 8 active hydrogen atoms per molecule. Particularly preferred polyols are at least partially based on ethylene oxide, preferably in an amount of less than 75 wt %. Suitable initiators include: polyols, for example glycerol, trimethylolpropane, triethanolamine, pentaerythritol, sorbitol and sucrose; polyamines, for example ethylene diamine, tolylene diamine (TDA), diaminodiphenylmethane (DADPM) and polymethylene polyphenylene polyamines; and aminoalcohols, for example ethanolamine and diethanolamine; and mixtures of such initiators. Other suitable polymeric polyols include polyesters obtained by the condensation of appropriate proportions of glycols and higher functionality polyols with dicarboxylic or polycarboxylic acids, DMT-scrap or digestion of PET by glycols. Still further suitable polymeric polyols include hydroxyl-terminated polythioethers, polycarbonate polyether polyols, polycaprolactone based polyethers, polyols derived from CO₂, polyamides, polyesteramides, polycarbonates, polyacetals and polysiloxanes.

Other suitable polymeric polyols include polyols derived from vegetable oils and other bio-based polyols. Suitable vegetable oils include castor oil, soy bean oil, peanut oil, canola oil. Other suitable polymeric polyols include polyols derived from chemolytic recycling of polyurethane.

As compounds which are reactive toward isocyanates, use can advantageously be made of those having a functionality of from 2 to 8, preferably from 2 to 6, and a molecular weight (Mn) of from 60 to 10,000 g/mol and having hydroxyl, thiol and/or primary and/or secondary amino groups as groups which are reactive toward isocyanates. Compounds which have been found to be useful are, for example, polyols selected from the group consisting of polyether polyols, for example polytetrahydrofuran, polyester polyols, polythioether polyols, hydroxyl-containing polyacetals and hydroxyl-containing aliphatic polycarbonates or mixtures of at least two of the polyols mentioned. Preference is given to using polyester polyols and/or polyether polyols. In some embodiments, the hydroxyl number of the polyhydroxyl compounds can be generally from 28 to 850 mg KOH/g and preferably from 35 to 600 mg KOH/g.

Furthermore, it has been found to be advantageous in the preparation of polyisocyanate polyaddition products to use prepolymers containing isocyanate groups.

The present invention also encompasses polyurethane or polyurethane prepolymer obtained by contacting at least one isocyanate, with at least one compound of formula (I) as described herein, or with at least one compound of formula (A) as described herein, or with a composition comprising a compound of formula (I) as defined hereinabove.

The present invention also encompasses polyurethane or polyurethane prepolymer obtained by contacting at least one isocyanate, with at least one isocyanate reactive compound, with at least one compound of formula (I) as described herein, or with at least one compound of formula (A) as described herein, or with a composition comprising a compound of formula (I) as defined hereinabove.

The invention encompasses the use of a compound of formula (I) as described herein, or of a composition comprising a compound of formula (I), or of a compound of formula (A) as described herein, or of a polyurethane or polyurethane prepolymer as described hereinabove, for the preparation of polyurethane foams, including but not limited to flexible foams, semi-rigid foams or rigid foams, adhesives, coatings, sealants, aerogels or elastomers (including non cellular and microcellular elastomers).

The present invention also encompasses an article comprising a compound of formula (I) as described herein, or a compound of formula (A) as described herein, or a composition comprising a compound of formula (I), or a polyurethane or polyurethane prepolymer as defined hereinabove, or an isocyanate-containing prepolymer as defined hereinabove.

In some other embodiments, the article may be a foam such as a rigid foam as used for structural or thermal and/or acoustical insulation or packaging, or such as a flexible foam as used for comfort applications such as for bedding and seating furniture, or such as a flexible foam as used for a technical application (acoustical damping, sealing, filtering). In some embodiments, the article may be an adhesive. In some embodiments, the article may be an elastomer, such as a non cellular or micro-cellular elastomer as used in applications such as gaskets, seals, automotive interior panels. In yet other embodiments, the article may be a coating. In yet other embodiments, the article may be an aerogel. In yet other embodiments, the article may be a sealant.

The following examples are provided for the purpose of illustrating the present invention and by no means should be interpreted to limit the scope of the present invention.

### EXAMPLES

Instrumentation

The Nuclear Magnetic Resonance spectra were recorded on a Bruker AVANCE 300 (300 MHz) and Bruker DRX500 (500 MHz) spectrometers. Chemical shifts are presented in parts per million (6) relative to DMSO-d6 (2.50 ppm) as internal standards. The resonance multiplicities are described as s (singlet), d (doublet), t (triplet), or m (multiplet).

Methods

The following methods were used in the examples:

Density: measured according to ISO845 (the weight and dimensions of a parallelepiped sample were determined).

Density of the reprocessed sample in water: measured according to NBN EN ISO 1183-1, part A.

Determination of the hydroxyl number IOH of the TAD-indole/PEG400 blend: hydroxyl value is a measure of the content of free hydroxyl groups in a chemical substance, usually expressed in units of the mass of potassium hydroxide (KOH) in milligrams equivalent to the hydroxyl content of one gram of the chemical substance. The analytical method used to determine hydroxyl value involved esterification of the free hydroxyl groups of the substance with phthalic anhydride in pyridine solvent. After completion of the reaction, water was added, and the remaining unreacted phthalic anhydride was converted to phthalic acid and measured by titration with potassium or sodium hydroxide.

Air permeability: measured according to EN ISO 9237 M4.

Reprocessing behavior: a foam sample (2.5 cm*2.5 cm*5 cm) was put in a heated press (top and bottom heating) under 6 bars of pressure, using a 1 mm spacer between the top and bottom plates of the press, at a predefined temperature for a controlled amount of time. The reprocessing was tested applying 4 temperatures (100-120-140-160° C.) and 2 heating times (5-10 min). The density of the reprocessed samples was then measured and compared with the density of the corresponding comparative sample in function of the reprocessing temperature.

Besides the density, also the height of the foam samples after reprocessing was systematically measured to have a control, the target being density >1000 kg/m³ and height around 1 mm.

Materials

4′-hydroxypropiophenone (CAS No. 70-70-2, TCI, 98%), isophorone diisocyanate (CAS No. 4098-71-9, IPDI), phenyl hydrazine (CAS No. 100-63-0, TCI, 99%), zinc(II) chloride (CAS No. 7646-85-7, Acros, 99%).

PPG 2000 (Arcol PPG 2000): polypropylene glycol with number average molecular weight (Mn)=2000 g/mol, CAS No. 25322-69-4, IOH=56.1 mg KOH/g, commercially available from Covestro.

PEG 400: polyethylene glycol with number average molecular weight (Mn)=400 g/mol, CAS No. 25322-68-3, IOH=280.5 mg KOH/g, commercially available from Ineos.

POLYOL 4290: tetrafunctional alkoxylated polyols with primary hydroxyl groups, number average molecular weight (Mn)=800 g/mol, IOH=290 mg KOH/g, commercially available from Perstorp.

PPG4000: polyoxypropyleneglycol, number average molecular weight (Mn)=4000 g/mol, IOH=28 mg KOH/g, commercially available from Shell Chemicals as Caradol© ED28-200.

Niax L-620: a high-performance alkyl-pendant type organosilicone surfactant, commercially available from Momentive™ Performance Materials.

Additive 9447 is an organosilicon surfactant. Dabco DC198, commercially available from Evonik can also be used.

Dabco© 33LV: Triethylenediamine/dipropylene glycol mixture (33%/67%), commercially available from Air products.

Niax© A133: bis(2-dimethylamino-ethyl)ether (CAS No. 3033-62-3)/dipropylene glycol (CAS No. 25265-71-8) mixture (23%/77%), commercially available from Momentive™ Performance Materials.

SO: stannous octoate (CAS No. 301-10-0), commercially available from Momentive™ Performance Materials.

Suprasec 2020: uretonimine-modified pure MDI, average functionality=2.1, NCO %=29.5, commercially available from Huntsman.

Suprasec 2609: isocyanate terminated prepolymer based on crude MDI and a polyether with number average molecular weight (Mn)=6000 g/mol, average functionality=2.97, NCO %=29, commercially available from Huntsman.

Unless otherwise indicated, all parts and all percentages in the following examples, as well as throughout the specification, are parts by weight or percentages by weight respectively.

### Example 1. Synthesis of 4-(3-methyl-1H-indol-2-yl)phenol

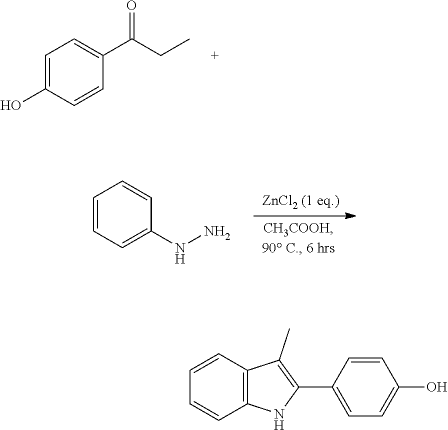 [SMILES: CCC(=O)c1ccc(O)cc1 ; NNc1ccccc1 ; Cc1c(-c2ccc(O)cc2)[nH]c2ccccc12]

A three-neck 1 L flask was charged with 4′-hydroxypropiophenone (100 g, 0.67 mol) in acetic acid (500 mL). The content of this flask was purged with argon for 10 min. To this solution was added phenyl hydrazine (86.4 g, 0.8 mol) at room temperature while stirring. A slight exotherm (to 38° C.) was noticed. Then, zinc(II) chloride (91 g, 0.67 mol) was added as a single portion. Again, slight exotherm to around 40° C. was observed. The temperature control was increased to 70° C. while stirring was kept constant at 400-500 rpm. Special care must be taken because the reaction is exothermic (cooling of the flask in a water bath can be needed). When the exotherm was over, the temperature was then stabilized and kept at 90° C.

After six hours at 90° C., the set-up was cooled down to room temperature with an ice bath. The resulting mixture was filtrated (on a glass filter) to remove as much salt as possible and then, some water ( 1/10 of the total volume: 50 mL here) was added and the bulk acetic acid was removed in vacuo. The resulting residue was redissolved in diethylether (750 mL)+75 mL of water to help the dissolution, and transferred to a big beaker (3 L). Then, solid K₂CO₃ was slowly added until no CO₂ formation was detected (efficient stirring). The content of the beaker was then decanted to remove the formed potassium acetate (in the water phase) and the organic phase was collected in a separation funnel. It was washed with sodium bicarbonate (2 times around 50 mL of saturated solution), water (2 times around 50 mL) and then brine (saturated NaCl solution, 50 mL). The washed organic phase was then dried over magnesium sulphate, filtered over a glass filter and the solvent was removed under reduced pressure affording a dark yellow oil (approximately 130 g, around 85-90% yield).

¹H-NMR (DMSO-d₆, 300 MHz) δ=10.99 (s (br.), 1H, —OH), 9.63 (s (br.), 1H, —NH), 7.51 (d, 1H, J=8.7 Hz, —CH_{Ar}), 7.53-7.49 (band, 2H, —CH_{Ar}), 7.34 (dt, 1H, J=7.5 and 0.9 Hz, —CH_{Ar}), 7.05 (m, 2H, J=7.5 Hz, —CH_{Ar}), 6.94 (d, 1H, J=8.7 Hz, —CH_{Ar}), 6.94 (m, 1H, —CH_{Ar}) and 2.39 (s, 3H, —CH₃) ppm.

¹H-NMR (CDCl₃, 300 MHz) δ=7.93 (s (br.), 1H, —NH), 7.57 (d(br.), 1H, J=7.6 Hz, —H_{Ar}), 7.45 (m, 2H, J=8.7 Hz, 2×—H_{Ar}), 7.34 (d(br.), 1H, J=7.9 Hz, —H_{Ar}), 7.15 (app. dtd, 2H, J=14.5, 7.0 and 1.3 Hz, —H_{Ar}), 6.93 (m, 2H, —H_{Ar}), 4.98 (s(br.), 1H, —OH), 2.42 (s, 3H, —CH₃) ppm.

### Example 2. Synthesis of 3-(4-(3-methyl-1H-indol-2-yl)phenoxy)propane-1,2-diol (Hereinafter Referred to as “Indole Diol”)

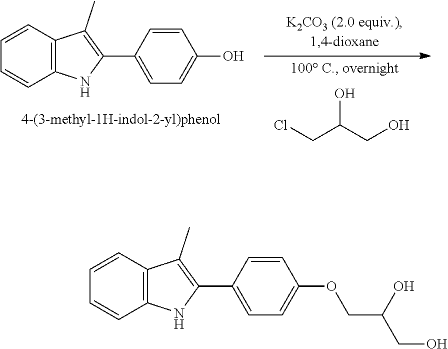 [SMILES: Cc1c(-c2ccc(O)cc2)[nH]c2ccccc12 ; OCC(O)CCl ; Cc1c(-c2ccc(OCC(O)CO)cc2)[nH]c2ccccc12]

To the same 1 L-flask containing the indole phenol obtained in Example 1 (+/−150 g, 0.67 mol) dioxane (600 mL), magnesium sulphate (75 g, 0.62 mol) and potassium carbonate (180 g, 1.3 mol, 2 equivalents) were added. Then, nitrogen was bubbled through the solution for 10 min before 3-chloro-1,2-propanediol (1.5 equivalents: 111.5 g, 84.5 mL) was added drop wise (the nitrogen flux was kept constant during the entire procedure). The suspension was then heated to 100° C. Stirring was kept constant for 24 h at 400-500 rpm. Then, the flask was allowed to cool down, the solids (Mg₂SO₄+K₂CO₃) were filtered off (glass filter) and the dioxane was removed in vacuo affording a dark yellow oil.

The resulting residue was redissolved in diethylether (750 mL)+a little water to help the dissolution (about 50 mL). It was washed with distilled water (3 times around 75 mL) and then brine (saturated NaCl solution, 2 times 50 mL). The organic phase was dried over magnesium sulphate, filtered over a glass filter and the solvent was removed under reduced pressure affording a dark yellow oil. 194.9 g of the title compound were obtained, 86% yield.

¹H-NMR (DMSO-d₆, 300 MHz) δ=11.02 (s(br.), 1H, —NH), 7.58 (d, 2H, J=9.0 Hz, 2×—H_{Ar}), 7.48 (d, 1H, J=7.5 Hz, —H_{Ar}), 7.32 (d, 1H, J=7.5 Hz, —H_{Ar}), 7.11-6.96 (band, 4H, 4×—H_{Ar}), 4.97 (d, 1H, J=5.1 Hz, —CH—OH), 4.69 (t, 1H, J=6.0 Hz, —CH₂OH), 4.07 (dd, 1H, J=4.2 and 9.9 Hz, —CHHOH), 3.93 (dd, 1H, J=6.0 and 9.9 Hz, —CHHOH), 3.83 (app. quint., 1H, J=5.1 Hz, —CH—OH), 3.50 (t, 2H, J=5.4, —CH₂OH), 2.38 (s, 3H, —CH₃) ppm.

### Example 3. Synthesis of IPDI-Bis-(Carbethoxysemicarbazide)

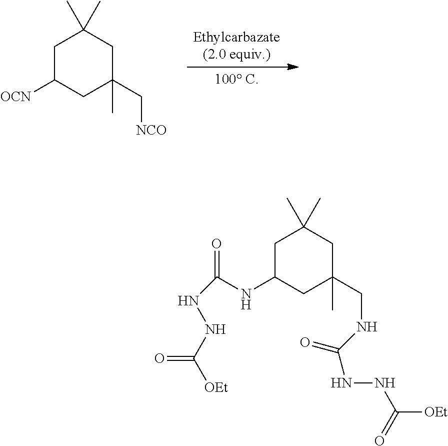

A mixture of ethyl carbazate (250 g, 2.4 mol, 2 equivalents) and toluene (1550 mL) was placed in a three-neck flask (2 L). The flask was equipped with an addition funnel, containing isophorone diisocyanate (267 g, 252 mL, 1.2 mol, 1 equivalent), a mechanical stirrer and a condenser. The mixture was put under inert atmosphere and the isocyanate was added slowly under vigorous stirring (250 rpm, added over the course of an hour). After addition, the mixture was stirred at room temperature for two hours, followed by two hours at 100° C. After that, the heater was turned off, the stirrer was removed and the reaction mixture was cooled down to room temperature. The top layer (mainly toluene) was decanted and removed. The moist organic compound (gel like material) was first dried on the rotary evaporator followed by overnight drying in the vacuum oven (40° C.). In the morning, the top part of the flask could be scratched out; the other half needed an extra night before reaching full dryness.

### Example 4. Synthesis of IPDI-bis-urazole

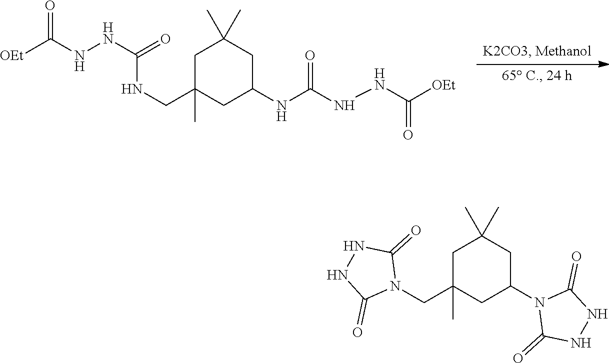 [SMILES: CCOC(=O)NNC(=O)NCC1(C)CC(NC(=O)NNC(=O)OCC)CC(C)(C)C1 ; CC1(C)CC(n2c(=O)[nH][nH]c2=O)CC(C)(Cn2c(=O)[nH][nH]c2=O)C1]

In a 6 L-flask, compound obtained in Example 3 (200 g, 0.46 mol) and potassium carbonate (300 g) were dissolved in methanol (3 L) under inert atmosphere. The flask was equipped with a T-piece, a mechanical stirrer with a big shaft, 1 or 2 condensers and a T-probe connected to the thermocouple. This mixture was refluxed for 8 hours (65° C.) under inert atmosphere, cooled down to room temperature and acidified until pH 1 by adding a 5-6 M hydrogen chloride solution in isopropanol (around 860 mL). Then, a white powder was filtered off (formed salts). By removing the solvent of the resulting clear solution in vacuo, the desired product (IPDI-bis-urazole) was obtained as a fine powder.

### Example 5. Synthesis of 4-[[5-(3,5-dioxo-1,2,4-triazol-4-yl)-1,3,3-trimethyl-cyclohexyl]methyl]-1,2,4-triazole-3,5-dione (IPDI-bis-TAD)

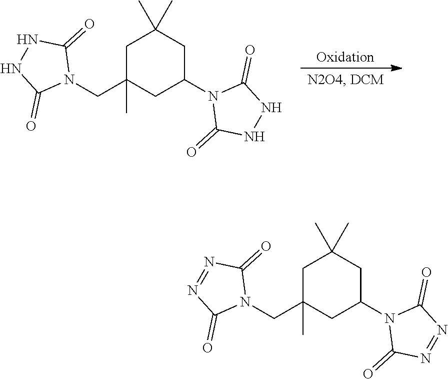 [SMILES: CC1(C)CC(n2c(=O)[nH][nH]c2=O)CC(C)(Cn2c(=O)[nH][nH]c2=O)C1 ; CC1(C)CC(N2C(=O)N=NC2=O)CC(C)(CN2C(=O)N=NC2=O)C1]

In a last step, a 2 L flask was filled with compound obtained in Example 4 (25 g, 74 mmol, 1 equivalent), magnesium sulfate (150 g, 1.04 mol, 7 equivalents) and dichloromethane (1.2 L).

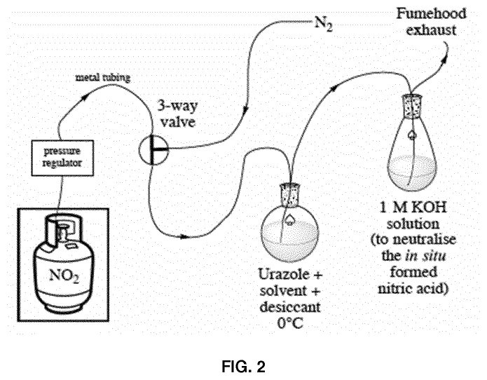

The flask was cooled to 0° C. using an ice bath, put under magnetic stirring and connected to an oxidation set up (with fitting inlets) as illustrated on FIG. 2. Then the T-valve of the oxidation set up was set to nitrogen and the contents were flushed with N₂ for 10-15 min with a gentle flux visible in the washing bottle/flask. Then, the T-valve was set to NO₂+N₂, and only after that the N₂O₄ bottle was opened (enough to see the brown gas flowing in the tubes; in the winter, a heater was sometimes used to heat up the bottle). This flux was maintained as long as needed (the conversion was followed by NMR by taking heterogeneous samples, drying them and dissolving them again in DMSO-D₆. The NO₂ flux was maintained as long as the urazole signal was visible around 10 ppm). After closing the N₂O₄ bottle, the T-valve was set again to nitrogen only for at least 2 h to flush the set up thoroughly (switch the ice bath to a water bath or no bath during the purging step). The reaction mixture was filtered, the residue washed with acetone and the filtrate was concentrated in vacuo to obtain IPDI-bis-TAD as dark red crystals. Yield was around 100%.

¹H NMR (400 MHz, DMSO-d₆): δ ppm 0.85-1.12 (br.s, 3×3 H, 9H, C CH₃) 1.17-1.36 (m, 2H, C CH₂ C) 1.44-1.63 (m, 2H, N CH CH2) 1.70-1.87 (m, 2H, N CH CH₂) 3.23 (br.d, 2H, N CH₂) 3.99-4.14 (m, 1H, N CH).

### Example 6. Preparation of the TAD-Indole Composition

4 equivalents of the indole diol prepared in Example 2 were dissolved in acetone (typically 250-300 mL for 50 g of indole diol). The flask was put in an ultrasound bath for a few minutes to help the dissolution. 1 equivalent of IPDI-bis-TAD obtained in Example 5 was dissolved in acetone (around 300 mL for 15 g of IPDI-bis-TAD). The IPDI-bis-TAD solution was added dropwise to the indole diol solution put under good magnetic stirring (a dropping funnel was used to put the IPDI-bis-TAD solution and the system was flushed with argon beforehand). The pink colour instantly disappeared leaving a light brown solution. After complete addition, the flask was kept stirring for a few minutes and the solvent was then completely removed in vacuo to reach a yellow/light brown powder that was further dried in the vacuum oven. THE can also be used instead of acetone. The composition comprised TAD-indole tetraol as well as an excess of the indole diol obtained in Example 2. This composition was obtained in the form of a powder.

### Example 7. Dissolution of the TAD-Indole Composition in PEG400

The TAD-indole composition obtained in Example 6 (41.7 wt % of the final mixture) was added to PEG400 (58.3 wt % of the final mixture). The mixture was initially a “slurry”. This mixture was then put under magnetic stirring on a heating plate at 60 to 70° C. After 2 h stirring, everything was dissolved, affording a viscous and stable blend at room temperature.

### Example 8. Preparation of Polyurethane Foams

The hydroxyl number (IOH) of the blend obtained in Example 7 was measured as described hereinabove. This value was used to calculate the required amount of isocyanate needed in the formulation for a specific NCO index.

All raw materials (including the TAD-indole/PEG400 blend described in Example 7) were used at room temperature (around 20° C.). Polyols, additives, catalysts, TAD-indole/PEG400 blend (according to Example 7) and water were combined and mixed for 30 sec at 3000 rpm.

Isocyanate(s) were added and the complete formulation was mixed for 10 more seconds at 3000 rpm.

The complete reaction mixture was then poured into a mould where it was allowed to cure in about 2-3 min (reaction to full rise).

The comparative examples of the different foam samples were made according to the same formulation with exception of the TAD-Indole composition (Example 6) which was replaced by the corresponding molar amounts of a Polyol 4290 and PEG400 composition. Polyol 4290 is a conventional crosslinker, i.e. giving permanent crosslinks to the polyurethane network. The amount of Polyol 4290 was selected so that the same degree of crosslinking was obtained in the PU foam compared to the amount of the TAD-indol tetraol in the TAD-indole composition. The amount of PEG400 was selected so as to obtain the same degree of chain extension in the PU foam compared to the amount of the free indole in the TAD-indol composition.

Table 1 shows the formulations of the produced foams.

TABLE 1

| | | Comparative | | Comparative | | Comparative | | Comparative |
|---|---|---|---|---|---|---|---|---|
| FORMULATIONS | Foam 1 | Foam 1 | Foam 2 | Foam 2 | Foam 3 | Foam 3 | Foam 4 | Foam 4 |
| TAD-indole composition - | 11 | — | 3.5 | — | 5.6 | — | 11.1 | — |
| (Example 6) (wt %) | | | | | | | | |
| TAD-indole tetraol (g) | 12.7 | | 3.5 | | 5.8 | | 12.7 | |
| Indole diol (g) | 8.2 | | 2.2 | | 3.7 | | 8.1 | |
| PPG 2000 (g) | 50.0 | 50.0 | | | | | | |
| PEG 400 (g) | 29.2 | 29.2 | 29.1 | 29.1 | 29.2 | 29.1 | 29.2 | 29.1 |
| Conventional crosslinker | | 11.4 | | 3.6 | | 5.8 | | 11.4 |
| composition (wt. %) | | | | | | | | |
| POLYOL 4290 (g) | | 11.0 | | 3.0 | | 5.0 | | 11.0 |
| PEG400 (g) | | 11.0 | | 3.0 | | 5.0 | | 11.0 |
| PPG4000 (g) | | | 50.0 | 50.0 | 50.0 | 50.0 | 50.0 | 50.0 |
| Niax L620 (g) | 1.5 | 1.5 | | | | | | |
| Additive 9447 (g) | | | 3.0 | 3.0 | 3.0 | 3.0 | 3.0 | 3.0 |
| Dabco 33LV (g) | 0.15 | 0.15 | 0.15 | 0.15 | 0.15 | 0.15 | 0.15 | 0.15 |
| Niax A133 (g) | 0.25 | 0.15 | 0.1 | 0.1 | 0.1 | 0.1 | 0.1 | 0.1 |
| SO (g) | 0.6 | 0.6 | 0.2 | 0.2 | 0.2 | 0.2 | 0.2 | 0.2 |
| Water (g) | 2.8 | 2.8 | 2.8 | 2.8 | 2.8 | 2.8 | 2.8 | 2.8 |
| TOTAL NON ISO | 105.3 | 106.3 | 91.0 | 91.4 | 94.9 | 95.4 | 106.3 | 107.3 |
| COMPONENTS (g) | | | | | | | | |
| ISOCYANATE INDEX | 96.9 | 96.9 | 100 | 100 | 100 | 100 | 100 | 100 |
| avg % NCO | 29.5 | 29.5 | 29.3 | 29.3 | 29.3 | 29.3 | 29.3 | 29.3 |
| Suprasec 2020 (g) | 85.1 | 85.3 | 36.3 | 36.8 | 37.5 | 38.2 | 41.0 | 42.6 |
| Suprasec 2609 (g) | | | 36.3 | 36.8 | 37.5 | 38.2 | 41.0 | 42.6 |
| TOTAL ISOCYANATE (g) | 85.1 | 85.3 | 72.6 | 73.6 | 75 | 76.5 | 81.9 | 85.2 |
| TOTAL FORMULATION (g) | 190.3 | 191.6 | 163.6 | 164.9 | 169.9 | 171.8 | 188.2 | 192.4 |
| WEIGHT RATIO ISO/Non-ISO | 0.8 | 0.8 | 0.8 | 0.8 | 0.8 | 0.8 | 0.8 | 0.8 |
| TAD/indole (g) | 20.9 | | 5.7 | | 9.4 | | 20.9 | |

The density, air permeability and reprocessing behavior of the produced foams was determined as described hereinabove. The results are depicted in Table 2.

TABLE 2

| | | | Comparative | | Comparative | | Comparative | | Comparative |
|---|---|---|---|---|---|---|---|---|---|
| | | Foam 1 | Foam 1 | Foam 2 | Foam 2 | Foam 3 | Foam 3 | Foam 4 | Foam 4 |
| Measured density (kg/m³) | | | 44.6 | 36.8 | 41.3 | 43.1 | 41.7 | 47.6 | 42.5 |
| Air permeability | 10 mm avg | | 86.4 | 34.6-61.3 | 68.8-80.9 | 5.5-9.1 | 66.3-74.4 | 37.4-74.2 | >85 |
| (EN ISO 9237 M4) | l/dm² · min | | | | | | | | |
| Density after | Heating for | | | | | | | | |
| reprocessing | 5 minutes | | | | | | | | |
| (kg/m³) | 100° C. | | | 572 | 504 | 922 | 487 | 629 | 231 |
| | 120° C. | 992 | | 647 | 617 | 935 | 711 | 703 | 582 |
| | 140° C. | 1040 | | 686 | 672 | 871 | 721 | 800 | 644 |
| | 150° C. | | 661 | | | | | | |
| | 160° C. | | | 721 | 732 | 905 | 785 | 818 | 735 |
| | Heating for | | | | | | | | |
| | 10 minutes | | | | | | | | |
| | 100° C. | | | 614 | 549 | 917 | 676 | 630 | 419 |
| | 120° C. | 1017 | | 647 | 656 | 894 | 709 | 745 | 635 |
| | 140° C. | 1037 | | 723 | 716 | 900 | 736 | 815 | 673 |
| | 150° C. | | 677 | | | | | | |
| | 160° C. | | | 711 | 800 | 948 | 861 | 874 | 777 |

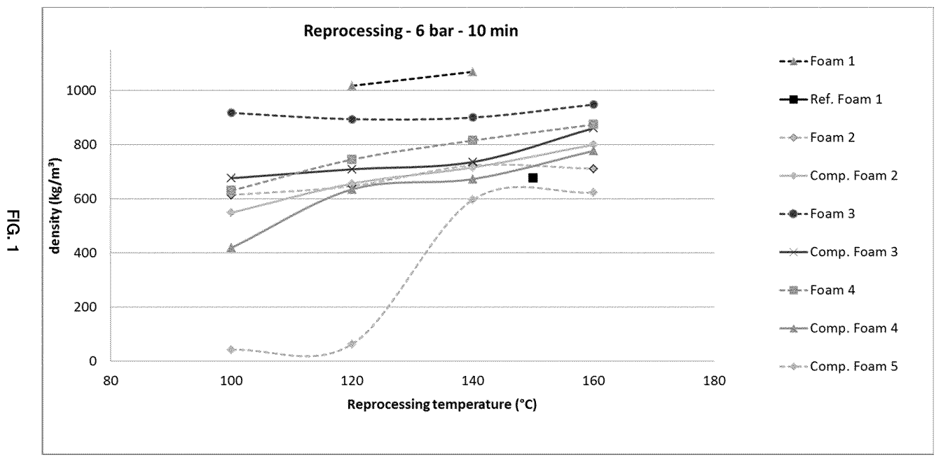

FIG. 1 shows the reprocessing behavior of the prepared foams and their corresponding comparative examples, when subjected to a pressure of 6 bar, and heating at different temperatures for 10 minutes. FIG. 1 shows an additional comparative foam, Comparative Foam 5, which corresponds to a commercially available flexible polyurethane comfort foam (Bultex, available from Recticel).

FIG. 1 shows that Foam 1 exhibited a much improved reprocessing compared to Comparative Foam 1 and Comparative Foam 5. The density difference between reprocessed Foam 1 and the reprocessed Comparative Foam 5 is 950 kg/m³.

1. A compound of formula (I) or a stereoisomer, enantiomer, racemic, or tautomer thereof, 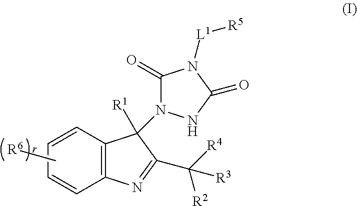
wherein,
r is an integer selected from 0, 1, 2, 3 or 4;
R¹ is selected from the group consisting of C₁₋₁₀alkyl; C₂₋₁₀alkenyl; C₂₋₁₀alkynyl; C₆₋₁₂aryl; C₃₋₈cycloalkyl; C₆₋₁₂arylC₁₋₆alkyl; heterocyclyl; heteroaryl; heterocyclylC₁₋₆alkyl; heteroarylC₁₋₆alkyl; —OR¹⁹; —SR¹⁹; NR²⁰R²¹; —C(O)NR²⁰R²¹; OH, SH, COOH, NH₂; NHR²⁰; and a polymeric group;
and wherein said C₁₋₁₀alkyl; C₂₋₁₀alkenyl; C₂₋₁₀alkynyl, C₆₋₁₂aryl, C₃₋₈cycloalkyl; C₆₋₁₂arylC₁₋₆alkyl, heterocyclyl; heteroaryl; heterocyclylC₁₋₆alkyl; or heteroarylC₁₋₆alkyl; can be unsubstituted or substituted with one or more Z¹;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
or R¹ is a group of formula (i); 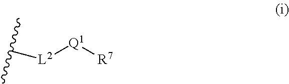
wherein the wavy line ( [symbol] ) indicates the point of attachment to the indole ring of formula (I);
L² is selected from the group consisting of C₁₋₁₀alkylene; C₃₋₆cycloalkylene; C₂₋₁₀alkenylene; C₂₋₁₀alkynylene; C₆₋₁₂arylene; and C₆₋₁₂arylC₁₋₆alkylene;
and wherein said C₁₋₁₀alkylene; C₃₋₆cycloalkylene; C₂₋₁₀alkenylene; C₂₋₁₀alkynylene; C₆₋₁₂arylene or C₆₋₁₂arylC₁₋₆alkylene, can be unsubstituted or substituted with one or more Z²;
Q¹ is a single bond or selected from the group consisting of C₁₋₁₀alkylene; C₃₋₆cycloalkylene; —O—; —S—; —O—C₁₋₁₀alkylene; and —S—C₁₋₁₀alkylene; wherein each left side of said groups is attached to L² and the right side thereof is attached to R⁷;
and wherein said C₁₋₁₀alkylene and said C₃₋₆cycloalkylene; can be unsubstituted or substituted with one or more Z³;
R⁷ is selected from the group consisting of hydrogen; OH, SH, COOH, NH₂; NHR²⁰, halo; nitro; cyano; —C(O)NR²⁰R²¹; C₁₋₂₀alkyl, C₂₋₂₀alkenyl; C₂₋₂₀alkynyl; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl; can be unsubstituted or substituted with one or more Z⁴;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
or R⁷ is a group of formula (ii); 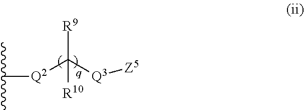
wherein the wavy line ( [symbol] ) indicates the point of attachment to Q¹;
q is an integer selected from 0; 1, 2, 3 or 4;
Q² is a single bond or selected from the group consisting of CR¹¹R¹²; O, S, and NR¹³;
Q³ is a single bond or selected from the group consisting of hydroxyC₁₋₁₀alkylene; aminoC₁₋₁₀alkylene; mercaptoC₁₋₁₀alkylene; C₁₋₂₀alkylene; C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₆alkylene; and heteroarylC₁₋₆alkylene;
wherein said C₁₋₂₀alkylene, C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₆alkylene; or heteroarylC₁₋₆alkylene, can be unsubstituted or substituted with one or more Z^{5a};
wherein if q is 0, then one of Q² and Q³ is not a single bond;
each R⁹ and R¹⁰ is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
each R¹¹ and R¹² is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
R¹³ is selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; and heteroarylC₁₋₆alkyl;
R² is selected from the group consisting of halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; heteroarylC₁₋₆alkyl; and a polymeric group;
R³ is selected from the group consisting of halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; heteroarylC₁₋₆alkyl; and a polymeric group;
R⁴ is selected from the group consisting of halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; heteroarylC₁₋₆alkyl; and a polymeric group;
wherein for R², R³ and R⁴, each independently; said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl, heteroarylC₁₋₆alkyl, can be unsubstituted or substituted with one or more Z⁶;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
or R² and R³ and R⁴ together with the carbon atom to which they are attached can form a saturated or unsaturated 4-, 5-, 6-, 7- or 8-membered ring;
wherein said saturated or unsaturated 4-, 5-, 6-, 7- or 8-membered ring optionally comprises one or more heteroatoms, said heteroatoms being each independently selected from O, S and N;
wherein said saturated or unsaturated 4-, 5-, 6-, 7- or 8-membered ring can be unsubstituted or substituted with one or more R⁸;
each R⁸ is independently selected from the group consisting of hydroxyC₁₋₁₀alkoxy; aminoC₁₋₁₀alkoxy; mercaptoC₁₋₁₀alkoxy; C₁₋₁₀alkyl; C₂₋₁₀alkenyl; C₂₋₁₀alkynyl; C₆₋₁₂arylC₁₋₆alkyl; C₃₋₈cycloalkyl; C₆₋₁₂arylC₁₋₆alkyl; heterocyclyl; heteroaryl; heterocyclylC₁₋₆alkyl; heteroarylC₁₋₆alkyl; OH; SH; COOH; NH₂; NHR²⁰; halogen; haloC₁₋₆alkyl; haloC₁₋₆alkyloxy; —OR¹⁹; —SR¹⁹; nitro; NR²⁰R²¹; —C(O)NR²⁰R²¹; cyano; and a polymeric group;
wherein said hydroxyC₁₋₁₀alkoxy; aminoC₁₋₁₀alkoxy; mercaptoC₁₋₁₀alkoxy; C₁₋₁₀alkyl; C₂₋₁₀alkenyl; C₂₋₁₀alkynyl; C₆₋₁₂arylC₁₋₆alkyl; C₃₋₈cycloalkyl; C₆₋₁₂arylC₁₋₆alkyl; heterocyclyl; heteroaryl; heterocyclylC₁₋₆alkyl; or heteroarylC₁₋₆alkyl, can be unsubstituted or substituted with one or more Z⁷;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
or R⁸ is a group of formula (iii); 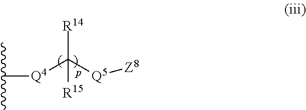
wherein the wavy line ( [symbol] ) indicates the point of attachment to the ring formed by R² and R³ and R⁴ together;
p is an integer selected from 0; 1, 2, 3 or 4;
Q⁴ is a single bond or selected from the group consisting of CR¹⁶R¹⁷; O, S, and NR¹⁸;
Q⁵ is a single bond or selected from the group consisting of hydroxyC₁₋₁₀alkylene; aminoC₁₋₁₀alkylene; mercaptoC₁₋₁₀alkylene; C₁₋₂₀alkylene, C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₆alkylene; and heteroarylC₁₋₆alkylene;
wherein said C₁₋₂₀alkylene, C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₆alkylene; or heteroarylC₁₋₆alkylene, can be unsubstituted or substituted with one or more Z^{8a};
wherein if p is 0, then one of Q⁴ and Q⁵ is not a single bond;
each R¹⁴ and R¹⁵ is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
each R¹⁶ and R¹⁷ is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
R¹⁸ is selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; and heteroarylC₁₋₆alkyl;
each R⁶ is independently selected from the group consisting of hydrogen, halogen; C₁₋₁₀alkyl; C₂₋₁₀alkenyl; C₂₋₁₀alkynyl; C₆₋₁₂arylC₁₋₆alkyl; haloC₁₋₆alkyl; haloC₁₋₆alkyloxy; —OR¹⁹; —SR¹⁹; nitro; NR²⁰R²¹; —C(O)NR²⁰R²¹; cyano; and a polymeric group;
wherein said C₁₋₁₀alkyl; C₂₋₁₀alkenyl; C₂₋₁₀alkynyl; or C₆₋₁₂arylC₁₋₆alkyl, can be unsubstituted or substituted with one or more Z⁹;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
L¹ is selected from the group consisting of C₁₋₁₀alkyleneC₃₋₈cycloalkylene; C₃₋₈cycloalkyleneC₁₋₁₀alkylene; C₁₋₁₀alkylene; C₃₋₈cycloalkylene; C₃₋₈cycloalkyleneC₁₋₁₀alkylC₃₋₈cycloalkylene; C₂₋₁₀alkenylene; C₂₋₁₀alkynylene; C₆₋₁₂arylene; C₆₋₁₂aryleneC₁₋₆alkylene; C₆₋₁₂aryleneC₁₋₆alkylC₆₋₁₂arylene; and C₁₋₆alkyleneC₆₋₁₂arylC₁₋₆alkylene;
wherein said C₁₋₁₀alkyleneC₃₋₈cycloalkylene; C₃₋₈cycloalkyleneC₁₋₁₀alkylene; C₁₋₁₀alkylene; C₃₋₈cycloalkylene; C₃₋₈cycloalkyleneC₁₋₁₀alkylC₃₋₈cycloalkylene; C₂₋₁₀alkenylene; C₂₋₁₀alkynylene; C₆₋₁₂arylene; C₆₋₁₂aryleneC₁₋₆alkylene; C₆₋₁₂aryleneC₁₋₆alkylC₆₋₁₂arylene; or C₁₋₆alkyleneC₆₋₁₂arylC₁₋₆alkylene, can be unsubstituted or substituted with one or more Z^{1a};
or L¹ is selected from a group of formula (iv); 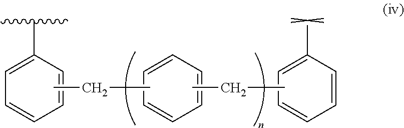
wherein the wavy line ( [symbol] ) indicates the point of attachment to the nitrogen of the formula (I) and the crossed line ( [symbol] ) indicates the point of attachment to R⁵;
wherein n is an integer selected from 0, 1, 2, 3, 4, 5, 6, 7, 8, 9, or 10;
R⁵ is selected from the group consisting of OH, SH, COOH, NH₂, and NHR²⁰;
or R⁵ is a group of formula (v); 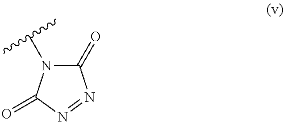 [SMILES: *N1C(=O)N=NC1=O]
wherein the wavy line ( [symbol] ) indicates the point of attachment to L¹;
or R⁵ is a group of formula (vi); 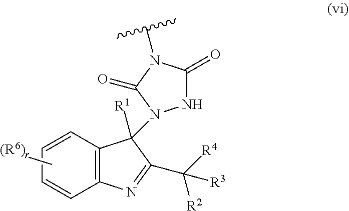
wherein the wavy line ( [symbol] ) indicates the point of attachment to L¹;
each R¹⁹ is independently selected from the group consisting of C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl;
wherein said C₁₋₁₀alkyl, C₂₋₂₀alkenyl, C₂₋₂₀alkynyl, C₆₋₁₂arylC₁₋₆alkyl, heterocyclylC₁₋₆alkyl; heteroarylC₁₋₆alkyl, optionally comprise one or more heteroatoms in the alkyl, alkenyl or alkynyl moiety, said heteroatoms being each independently selected from O, S and N;
wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl, can be unsubstituted or substituted with one or more OH, SH, COOH, or NH₂;
each R²⁰ is independently selected from the group consisting of C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl;
wherein said C₁₋₁₀alkyl, C₂₋₂₀alkenyl, C₂₋₂₀alkynyl, C₆₋₁₂arylC₁₋₆alkyl, heterocyclylC₁₋₆alkyl; heteroarylC₁₋₆alkyl, optionally comprise one or more heteroatoms in the alkyl, alkenyl or alkynyl moiety, said heteroatoms being each independently selected from O, S and N;
wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl, can be unsubstituted or substituted with one or more OH, SH, COOH, or NH₂;
each R²¹ is independently selected from the group consisting of hydrogen, C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl;
wherein said C₁₋₁₀alkyl, C₂₋₂₀alkenyl, C₂₋₂₀alkynyl, C₆₋₁₂arylC₁₋₆alkyl, heterocyclylC₁₋₆alkyl; heteroarylC₁₋₆alkyl, optionally comprise one or more heteroatoms in the alkyl, alkenyl or alkynyl moiety, said heteroatoms being each independently selected from O, S and N;
wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl, can be unsubstituted or substituted with one or more OH, SH, COOH, or NH₂;
or wherein R²⁰ and R²¹ together with the atom to which they are attached form a 5-, 6-, or 7-membered heterocycle.
each Z^{1a} is selected from the group consisting of C₁₋₆alkyl; C₂₋₆alkenyl; C₂₋₆alkynyl; haloC₁₋₆alkyl; haloC₁₋₆alkyloxy; halogen; isocyanate;
or Z^{1a} is a group of formula (vii); 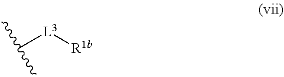
wherein the wavy line ( [symbol] ) indicates the point of attachment to L¹;
L³ is a single bond or is selected from the group consisting of C₁₋₁₀alkylene; C₂₋₁₀alkenylene; C₂₋₁₀alkynylene; and C₆₋₁₂aryl;
R^{1b} is a group of formula (viii); 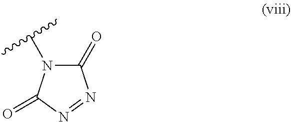 [SMILES: *N1C(=O)N=NC1=O]
wherein the wavy line ( [symbol] ) indicates the point of attachment to L³;
or R^{1b} is a group of formula (ix); 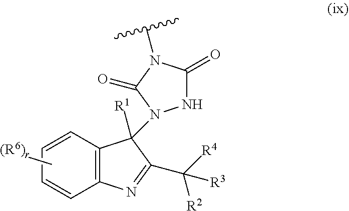
wherein the wavy line ( [symbol] ) indicates the point of attachment to L³;
Z¹, Z², Z³, Z⁴, Z⁶ are each independently selected from the group consisting of OH; SH; COOH; NH₂; NHR²⁰; halogen; C₁₋₆alkyl; haloC₁₋₆alkyl; haloC₁₋₆alkyloxy; —OR¹⁹; —SR¹⁹; nitro; NR²⁰R²¹; —C(O)NR²⁰R²¹; cyano;
Z⁵, Z^{5a}, Z⁷, Z⁸, Z^{8a}, Z⁹ are each independently selected from the group consisting of OH; hydrogen; SH; COOH; NH₂; NHR²⁰; halogen; C₁₋₆alkyl; haloC₁₋₆alkyl; haloC₁₋₆alkyloxy; —OR¹⁹; —SR¹⁹; nitro; NR²⁰R²¹; —C(O)NR²⁰R²¹; cyano; and a polymeric group;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
and when none of R¹, R², R³, R⁴, R⁶, R⁷, R⁸, R⁹, R¹⁹, R¹¹, R¹², R¹⁴, R¹⁵, R¹⁶, R¹⁷, Z⁵, Z^{5a}, Z⁷, Z⁸, Z^{8a} or Z⁹ is a polymeric group, then at least one of R⁷, R⁸, Z¹, Z², Z³, Z⁴, Z⁵, Z^{5a}, Z⁶, Z⁷, Z⁸, Z^{8a} or Z⁹ is selected from the group consisting of OH, SH, COOH, NH₂, and NHR²⁰.

2. The compound according to claim 1, wherein,
R¹ is selected from the group consisting of C₁₋₁₀alkyl; C₆₋₁₂aryl; C₃₋₈cycloalkyl; C₆₋₁₂arylC₁₋₆alkyl; —OR¹⁹; —SR¹⁹; NR²⁰R²¹; —C(O)NR²⁰R²¹; OH, SH, COOH, NH₂; NHR²⁰; and a polymeric group, wherein said C₁₋₁₀alkyl; C₆₋₁₂aryl; C₃₋₈cycloalkyl; or C₆₋₁₂arylC₁₋₆alkyl, can be unsubstituted or substituted with one or more Z¹;
R² is selected from the group consisting of halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; heteroarylC₁₋₆alkyl; and a polymeric group,
R³ is selected from the group consisting of halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; heteroarylC₁₋₆alkyl; and a polymeric group,
R⁴ is selected from the group consisting of halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; heteroarylC₁₋₆alkyl; and a polymeric group,
wherein for R², R³ and R⁴, each independently; said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, can be unsubstituted or substituted with one or more Z⁶;
or R² and R³ and R⁴ together with the carbon atom to which they are attached form a saturated or unsaturated 4-, 5-, or 6-membered ring;
wherein said saturated or unsaturated 4-, 5-, or 6-membered ring can be unsubstituted or substituted with one or more R⁸;
each R⁸ is independently selected from the group consisting of hydroxyC₁₋₁₀alkoxy; aminoC₁₋₁₀alkoxy; mercaptoC₁₋₁₀alkoxy; C₁₋₁₀alkyl; C₂₋₁₀alkenyl; C₂₋₁₀alkynyl; C₆₋₁₂arylC₁₋₆alkyl; C₃₋₈cycloalkyl; C₆₋₁₂arylC₁₋₆alkyl; heterocyclyl; heteroaryl; heterocyclylC₁₋₆alkyl; heteroarylC₁₋₆alkyl; —OR¹⁹; —SR¹⁹; and a polymeric group;
wherein said hydroxyC₁₋₁₀alkoxy; aminoC₁₋₁₀alkoxy; mercaptoC₁₋₁₀alkoxy; C₁₋₁₀alkyl; C₂₋₁₀alkenyl; C₂₋₁₀alkynyl; C₆₋₁₂arylC₁₋₆alkyl; C₃₋₈cycloalkyl; C₆₋₁₂arylC₁₋₆alkyl; heterocyclyl; heteroaryl; heterocyclylC₁₋₆alkyl; or heteroarylC₁₋₆alkyl, can be unsubstituted or substituted with one or more Z⁷;
or R⁸ is a group of formula (iii); 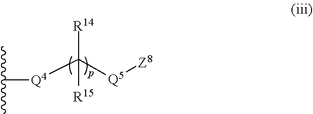
wherein the wavy line ( [symbol] ) indicates the point of attachment to the ring formed by R² and R³ and R⁴ together;
p is an integer selected from 0; 1, 2, or 3;
Q⁴ is a single bond or selected from the group consisting of CR¹⁶R¹⁷; O, S, and NR¹⁸;
Q⁵ is a single bond or selected from the group consisting of hydroxyC₁₋₁₀alkylene; aminoC₁₋₁₀alkylene; mercaptoC₁₋₁₀alkylene; C₁₋₂₀alkylene, C₆₋₁₂arylene, C₃₋₈cycloalkylene; C₆₋₁₂arylC₁₋₆alkylene; heterocyclylene; heteroarylene; heterocyclylC₁₋₆alkylene; and heteroarylC₁₋₆alkylene;
wherein said C₁₋₂₀alkylene, C₆₋₁₂arylene, C₃₋₈cycloalkylene; C₆₋₁₂arylC₁₋₆alkylene; heterocyclylene; heteroarylene; heterocyclylC₁₋₆alkylene; or hetero arylC₁₋₆alkylene; can be unsubstituted or substituted with one or more Z^{8a};
wherein if p is 0, then one of Q⁴ and Q⁵ is not a single bond;
each R¹⁴ and R¹⁵ is independently selected from the group consisting of hydrogen; C₁₋₂₀alkyl, hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
each R¹⁶ and R¹⁷ is independently selected from the group consisting of hydrogen; C₁₋₂₀alkyl, hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
R¹⁸ is selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; and heteroarylC₁₋₆alkyl;
each R⁶ is independently selected from the group consisting of hydrogen, halogen; C₁₋₁₀alkyl; C₆₋₁₂arylC₁₋₆alkyl; haloC₁₋₆alkyl; haloC₁₋₆alkyloxy; —OR¹⁹; —SR¹⁹; nitro; NR²⁰R²¹; —C(O)NR²⁰R²¹; cyano; and a polymeric group;
wherein said C₁₋₁₀alkyl; or C₆₋₁₂arylC₁₋₆alkyl, can be unsubstituted or substituted with one or more Z⁹;
L¹ is selected from the group consisting of C₁₋₁₀alkyleneC₃₋₈cycloalkylene; C₃₋₈cycloalkyleneC₁₋₁₀alkylene; C₁₋₁₀alkylene; C₃₋₈cycloalkylene; C₃₋₈cycloalkyleneC₁₋₁₀alkylC₃₋₈cycloalkylene; C₆₋₁₂arylene; C₆₋₁₂aryleneC₁₋₆alkylene; C₆₋₁₂aryleneC₁₋₆alkylC₆₋₁₂arylene; and C₁₋₆alkyleneC₆₋₁₂arylC₁₋₆alkylene;
wherein said C₁₋₁₀alkyleneC₃₋₈cycloalkylene; C₃₋₈cycloalkyleneC₁₋₁₀alkylene; C₁₋₁₀alkylene; C₃₋₈cycloalkylene; C₃₋₈cycloalkyleneC₁₋₁₀alkylC₃₋₈cycloalkylene; C₆₋₁₂aryleneC₁₋₆alkylene; C₆₋₁₂aryleneC₁₋₆alkyleneC₆₋₁₂arylene; or C₁₋₆alkyleneC₆₋₁₂arylC₁₋₆alkylene, can be unsubstituted or substituted with one or more Z^{1a};
R⁵ is selected from the group consisting of OH, SH, COOH, NH₂, and NHR²⁰;
or R⁵ is a group of formula (v); 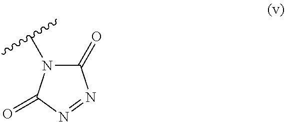 [SMILES: *N1C(=O)N=NC1=O]
wherein the wavy line ( [symbol] ) indicates the point of attachment to L¹;
or R⁵ is a group of formula (vi); 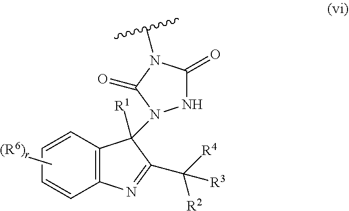
wherein the wavy line ( [symbol] ) indicates the point of attachment to L¹;
each R¹⁹ is independently selected from the group consisting of C₁₋₂₀alkyl, C₆₋₁₂aryl, C₃₋₈cycloalkyl, and C₆₋₁₂arylC₁₋₆alkyl;
wherein said C₁₋₂₀alkyl, C₆₋₁₂aryl, C₃₋₈cycloalkyl, C₆₋₁₂arylC₁₋₆alkyl, can be unsubstituted or substituted with one or more OH, SH, COOH, or NH₂;
each R²⁰ is independently selected from the group consisting of C₁₋₂₀alkyl, C₆₋₁₂aryl, C₃₋₈cycloalkyl, and C₆₋₁₂arylC₁₋₆alkyl;
wherein said C₁₋₂₀alkyl, C₆₋₁₂aryl, C₃₋₈cycloalkyl, C₆₋₁₂arylC₁₋₆alkyl, can be unsubstituted or substituted with one or more OH, SH, COOH, or NH₂;
each R²¹ is independently selected from the group consisting of hydrogen; C₁₋₂₀alkyl, C₆₋₁₂aryl, C₃₋₈cycloalkyl, and C₆₋₁₂arylC₁₋₆alkyl;
wherein said C₁₋₂₀alkyl, C₆₋₁₂aryl, C₃₋₈cycloalkyl, C₆₋₁₂arylC₁₋₆alkyl, can be unsubstituted or substituted with one or more OH, SH, COOH, or NH₂;
each Z^{1a} is selected from the group consisting of C₁₋₆alkyl; haloC₁₋₆alkyl; haloC₁₋₆alkyloxy; halogen; isocyanate;
or Z^{1a} is a group of formula (vii); 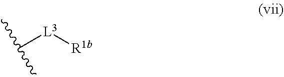
wherein the wavy line ( [symbol] ) indicates the point of attachment to L¹;
L³ is a single bond or is selected from the group consisting of C₁₋₁₀alkylene; C₂₋₁₀alkenylene; C₂₋₁₀alkynylene; and C₆₋₁₂aryl;
R^{1b} is a group of formula (viii); 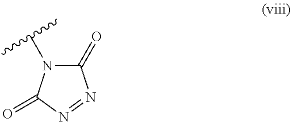 [SMILES: *N1C(=O)N=NC1=O]
wherein the wavy line ( [symbol] ) indicates the point of attachment to L³;
or R^{1b} is a group of formula (ix); 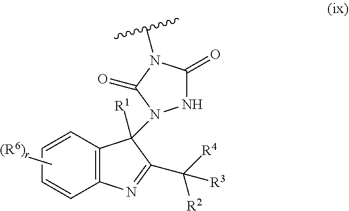
wherein the wavy line ( [symbol] ) indicates the point of attachment to L³;
Z¹, Z⁶ are each independently selected from the group consisting of OH; SH; COOH; NH₂; NHR²⁰; halogen; C₁₋₆alkyl; haloC₁₋₆alkyl; haloC₁₋₆alkyloxy; —OR¹⁹; —SR¹⁹; nitro; NR²⁰R²¹; —C(O)NR²⁰R²¹; cyano;
Z⁷, Z⁸, Z^{8a}, Z⁹ are each selected from the group consisting of OH; hydrogen; SH; COOH; NH₂; NHR²⁰; halogen; C₁₋₆alkyl; haloC₁₋₆alkyl; haloC₁₋₆alkyloxy; —OR¹⁹; —SR¹⁹; nitro; NR²⁰R²¹; —C(O)NR²⁰R²¹; cyano; and a polymeric group.

3. The compound according to claim 1, wherein,
R¹ is selected from the group consisting of C₁₋₁₀alkyl; C₆₋₁₂aryl; C₃₋₈cycloalkyl; C₆₋₁₂arylC₁₋₆alkyl; and a polymeric group, wherein said C₁₋₁₀alkyl; C₆₋₁₂aryl; C₃₋₈cycloalkyl; or C₆₋₁₂arylC₁₋₆alkyl, can be unsubstituted or substituted with one or more Z¹;
R² is selected from the group consisting of halo; nitro; cyano; —C(O)NR²⁰R²¹; C₆₋₁₂aryl; C₃₋₈cycloalkyl; C₆₋₁₂arylC₁₋₆alkyl; and a polymeric group,
R³ is selected from the group consisting of halo; nitro; cyano; —C(O)NR²⁰R²¹; C₆₋₁₂aryl; C₃₋₈cycloalkyl; C₆₋₁₂arylC₁₋₆alkyl; and a polymeric group,
R⁴ is selected from the group consisting of halo; nitro; cyano; —C(O)NR²⁰R²¹; C₆₋₁₂aryl; C₃₋₈cycloalkyl; C₆₋₁₂arylC₁₋₆alkyl; and a polymeric group,
wherein for R², R³ and R⁴, each independently; said C₁₋₂₀alkyl, C₆₋₁₂aryl, C₃₋₈cycloalkyl, C₆₋₁₂arylC₁₋₆alkyl, can be unsubstituted or substituted with one or more Z⁶;
or R² and R³ and R⁴ together with the carbon atom to which they are attached form a saturated or unsaturated 5-, or 6-membered ring;
wherein said saturated or unsaturated 5-, or 6-membered ring can be unsubstituted or substituted with one or more R⁸;
each R⁸ is independently selected from the group consisting of hydroxyC₁₋₁₀alkoxy; aminoC₁₋₁₀alkoxy; mercaptoC₁₋₁₀alkoxy; C₁₋₁₀alkyl; C₆₋₁₂arylC₁₋₆alkyl; C₃₋₈cycloalkyl; C₆₋₁₂arylC₁₋₆alkyl; heterocyclyl; heteroaryl; heterocyclylC₁₋₆alkyl; heteroarylC₁₋₆alkyl; —OR¹⁹; —SR¹⁹; and a polymeric group;
wherein said hydroxyC₁₋₁₀alkoxy; aminoC₁₋₁₀alkoxy; mercaptoC₁₋₁₀alkoxy; C₁₋₁₀alkyl; C₆₋₁₂arylC₁₋₆alkyl; C₃₋₈cycloalkyl; C₆₋₁₂arylC₁₋₆alkyl; heterocyclyl; heteroaryl; heterocyclylC₁₋₆alkyl; or heteroarylC₁₋₆alkyl, can be unsubstituted or substituted with one or more Z⁷;
or R⁸ is a group of formula (iii); 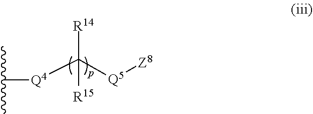
wherein the wavy line ( [symbol] ) indicates the point of attachment to the ring formed by R² and R³ and R⁴ together;
p is an integer selected from 1, 2, or 3;
Q⁴ is a single bond or selected from the group consisting of O, S, and NR¹⁸;
Q⁵ is a single bond or selected from the group consisting of hydroxyC₁₋₁₀alkylene; aminoC₁₋₁₀alkylene; mercaptoC₁₋₁₀alkylene; C₁₋₂₀alkylene, C₆₋₁₂arylene, C₃₋₈cycloalkylene;
wherein said C₁₋₂₀alkylene, C₆₋₁₂arylene, or C₃₋₈cycloalkylene; can be unsubstituted or substituted with one or more Z^{8a};
each R¹⁴ and R¹⁵ is independently selected from the group consisting of hydrogen; C₁₋₂₀alkyl, hydroxyC₁₋₁₀alkoxy; aminoC₁₋₁₀alkoxy; mercaptoC₁₋₁₀alkoxy; and a polymeric group;
R¹⁸ is selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; and a polymeric group;
each R⁶ is independently selected from the group consisting of hydrogen, halogen; C₁₋₁₀alkyl; C₆₋₁₂arylC₁₋₆alkyl; haloC₁₋₆alkyl; haloC₁₋₆alkyloxy; —OR¹⁹; —SR¹⁹; nitro; NR²⁰R²¹; —C(O)NR²⁰R²¹; cyano; and a polymeric group;
wherein said C₁₋₁₀alkyl; or C₆₋₁₂arylC₁₋₆alkyl, can be unsubstituted or substituted with one or more Z⁹;
L¹ is selected from the group consisting of C₁₋₁₀alkyleneC₃₋₈cycloalkylene; C₃₋₈cycloalkyleneC₁₋₁₀alkylene; C₁₋₁₀alkylene; C₃₋₈cycloalkylene; C₃₋₈cycloalkyleneC₁₋₁₀alkylC₃₋₈cycloalkylene; C₆₋₁₂arylene; C₆₋₁₂aryleneC₁₋₆alkylene; and C₆₋₁₂aryleneC₁₋₆alkylC₆₋₁₂arylene;
wherein said C₁₋₁₀alkyleneC₃₋₈cycloalkylene; C₃₋₈cycloalkyleneC₁₋₁₀alkylene; C₁₋₁₀alkylene; C₃₋₈cycloalkylene; C₃₋₈cycloalkyleneC₁₋₁₀alkylC₃₋₈cycloalkylene; C₆₋₁₂arylene; C₆₋₁₂aryleneC₁₋₆alkylene; or C₆₋₁₂aryleneC₁₋₆alkylC₆₋₁₂arylene, can be unsubstituted or substituted with one or more Z^{1a};
R⁵ is selected from the group consisting of OH, SH, COOH, NH₂, and NHR²⁰;
or R⁵ is a group of formula (v); 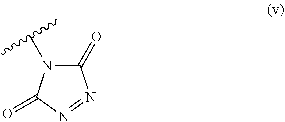 [SMILES: *N1C(=O)N=NC1=O]
wherein the wavy line ( [symbol] ) indicates the point of attachment to L¹;
or R⁵ is a group of formula (vi); 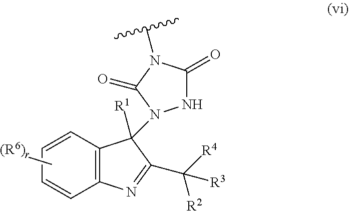
wherein the wavy line ( [symbol] ) indicates the point of attachment to L¹;
each R¹⁹ is independently selected from the group consisting of C₁₋₂₀alkyl, C₆₋₁₂aryl, C₃₋₈cycloalkyl, and C₆₋₁₂arylC₁₋₆alkyl;
wherein said C₁₋₂₀alkyl, C₆₋₁₂aryl, C₃₋₈cycloalkyl, C₆₋₁₂arylC₁₋₆alkyl, can be unsubstituted or substituted with one or more OH, SH, COOH, or NH₂;
each R²⁰ is independently selected from the group consisting of C₁₋₂₀alkyl, C₆₋₁₂aryl, C₃₋₈cycloalkyl, and C₆₋₁₂arylC₁₋₆alkyl;
wherein said C₁₋₂₀alkyl, C₆₋₁₂aryl, C₃₋₈cycloalkyl, C₆₋₁₂arylC₁₋₆alkyl, can be unsubstituted or substituted with one or more OH, SH, COOH, or NH₂;
each R²¹ is independently selected from the group consisting of hydrogen; C₁₋₂₀alkyl, C₆₋₁₂aryl, C₃₋₈cycloalkyl, and C₆₋₁₂arylC₁₋₆alkyl;
wherein said C₁₋₂₀alkyl, C₆₋₁₂aryl, C₃₋₈cycloalkyl, C₆₋₁₂arylC₁₋₆alkyl, can be unsubstituted or substituted with one or more OH, SH, COOH, or NH₂;
each Z^{1a} is selected from the group consisting of C₁₋₆alkyl; haloC₁₋₆alkyl; haloC₁₋₆alkyloxy; halogen;
or Z^{1a} is a group of formula (vii); 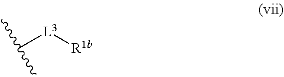
wherein the wavy line ( [symbol] ) indicates the point of attachment to L¹;
L³ is a single bond or is selected from the group consisting of C₁₋₁₀alkylene; C₂₋₁₀alkenylene; C₂₋₁₀alkynylene; and C₆₋₁₂aryl;
R^{1b} is a group of formula (viii); 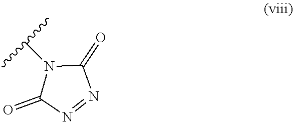 [SMILES: *N1C(=O)N=NC1=O]
wherein the wavy line ( [symbol] ) indicates the point of attachment to L³;
or R^{1b} is a group of formula (ix); 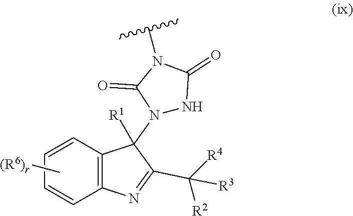
wherein the wavy line ( [symbol] ) indicates the point of attachment to L³;
Z¹ are each independently selected from the group consisting of OH; SH; COOH; NH₂; NHR²⁰; halogen; C₁₋₆alkyl; —OR¹⁹; —SR¹⁹; nitro; NR²⁰R²¹; —C(O)NR²⁰R²¹; cyano;
Z⁷, Z⁸, Z^{8a}, Z⁹ are each selected from the group consisting of OH; hydrogen; SH; COOH; NH₂; NHR²⁰; halogen; C₁₋₆alkyl; —OR¹⁹; —SR¹⁹; nitro; NR²⁰R²¹; —C(O)NR²⁰R²¹; cyano; and a polymeric group.

4. The compound according to claim 1, wherein
R¹ is selected from the group consisting of C₁₋₁₀alkyl; C₃₋₈cycloalkyl; and a polymeric group, wherein said C₁₋₁₀alkyl; or C₃₋₈cycloalkyl; can be unsubstituted or substituted with one or more Z¹;
R² is selected from the group consisting of halo; nitro; cyano; —C(O)NR²⁰R²¹; C₆₋₁₂aryl; C₃₋₈cycloalkyl; and a polymeric group,
R³ is selected from the group consisting of halo; nitro; cyano; —C(O)NR²⁰R²¹; C₆₋₁₂aryl; C₃₋₈cycloalkyl; and a polymeric group,
R⁴ is selected from the group consisting of halo; nitro; cyano; —C(O)NR²⁰R²¹; C₆₋₁₂aryl; C₃₋₈cycloalkyl; and a polymeric group,
wherein for R², R³ and R⁴, each independently; said C₁₋₂₀alkyl, C₆₋₁₂aryl, C₃₋₈cycloalkyl, C₆₋₁₂arylC₁₋₆alkyl, can be unsubstituted or substituted with one or more Z⁶;
or R² and R³ and R⁴ together with the carbon atom to which they are attached form a saturated or unsaturated 5-, or 6-membered ring;
wherein said saturated or unsaturated 5-, or 6-membered ring can be unsubstituted or substituted with one or more R⁸;
each R⁸ is independently selected from the group consisting of hydroxyC₁₋₁₀alkoxy; aminoC₁₋₁₀alkoxy; mercaptoC₁₋₁₀alkoxy; C₁₋₁₀alkyl; C₆₋₁₂arylC₁₋₆alkyl; C₃₋₈cycloalkyl; C₆₋₁₂arylC₁₋₆alkyl; heterocyclylC₁₋₆alkyl; heteroarylC₁₋₆alkyl; —OR¹⁹; —SR¹⁹; and a polymeric group;
wherein said hydroxyC₁₋₁₀alkoxy; aminoC₁₋₁₀alkoxy; mercaptoC₁₋₁₀alkoxy; C₁₋₁₀alkyl; C₆₋₁₂arylC₁₋₆alkyl; C₃₋₈cycloalkyl; C₆₋₁₂arylC₁₋₆alkyl; heterocyclylC₁₋₆alkyl; or heteroarylC₁₋₆alkyl, can be unsubstituted or substituted with one or more Z⁷;
or R⁸ is a group of formula (iii); 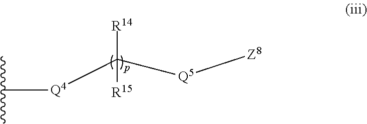
wherein the wavy line ( [symbol] ) indicates the point of attachment to the ring formed by R² and R³ and R⁴ together;
p is an integer selected from 1, 2, or 3;
Q⁴ is a single bond or selected from the group consisting of O, S, and NR¹⁸;
Q⁵ is a single bond or selected from the group consisting of hydroxyC₁₋₁₀alkylene; aminoC₁₋₁₀alkylene; mercaptoC₁₋₁₀alkylene; C₁₋₂₀alkylene, C₃₋₈cycloalkylene;
wherein said C₁₋₂₀alkylene, or C₃₋₈cycloalkylene; can be unsubstituted or substituted with one or more Z^{8a};
each R¹⁴ and R¹⁵ is independently selected from the group consisting of hydrogen; C₁₋₂₀alkyl, hydroxyC₁₋₁₀alkoxy; and a polymeric group;
R¹⁸ is selected from the group consisting of hydrogen; C₁₋₂₀alkyl, C₂₋₂₀alkenyl; C₂₋₂₀alkynyl; hydroxyC₁₋₁₀alkoxy; and a polymeric group;
each R⁶ is independently selected from the group consisting of hydrogen, halogen; C₁₋₁₀alkyl; C₆₋₁₂arylC₁₋₆alkyl; haloC₁₋₆alkyl; haloC₁₋₆alkyloxy; —C(O)NR²⁰R²¹; and a polymeric group;
wherein said C₁₋₁₀alkyl; or C₆₋₁₂arylC₁₋₆alkyl, can be unsubstituted or substituted with one or more Z⁹;
L¹ is selected from the group consisting of C₁₋₁₀alkyleneC₃₋₈cycloalkylene; C₃₋₈cycloalkyleneC₁₋₁₀alkylene; C₁₋₁₀alkylene; C₃₋₈cycloalkylene; C₃₋₈cycloalkyleneC₁₋₁₀alkylC₃₋₈cycloalkylene; and C₆₋₁₂arylene;
wherein said C₁₋₁₀alkyleneC₃₋₈cycloalkylene; C₃₋₈cycloalkyleneC₁₋₁₀alkylene; C₁₋₁₀alkylene; C₃₋₈cycloalkylene; C₃₋₈cycloalkyleneC₁₋₁₀alkylC₃₋₈cycloalkylene; or C₆-12arylene, can be unsubstituted or substituted with one or more Z^{1a};
R⁵ is selected from the group consisting of OH, SH, COOH, NH₂, and NHR²⁰;
or R⁵ is a group of formula (v); 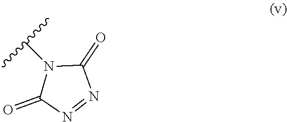 [SMILES: *N1C(=O)N=NC1=O]
wherein the wavy line ( [symbol] ) indicates the point of attachment to L¹;
or R⁵ is a group of formula (vi); 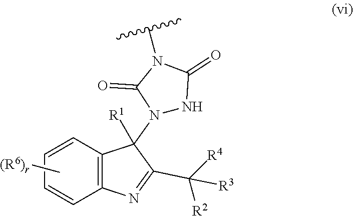
wherein the wavy line ( [symbol] ) indicates the point of attachment to L¹;
each R¹⁹ is independently selected from the group consisting of C₁₋₂₀alkyl, C₃₋₈cycloalkyl, and C₆₋₁₂arylC₁₋₆alkyl;
wherein said C₁₋₂₀alkyl, C₃₋₈cycloalkyl, C₆₋₁₂arylC₁₋₆alkyl, can be unsubstituted or substituted with one or more OH, SH, COOH, or NH₂;
each R²⁰ is independently selected from the group consisting of C₁₋₂₀alkyl, C₃₋₈cycloalkyl, and C₆₋₁₂arylC₁₋₆alkyl;
wherein said C₁₋₂₀alkyl, C₃₋₈cycloalkyl, C₆₋₁₂arylC₁₋₆alkyl, can be unsubstituted or substituted with one or more OH, SH, COOH, or NH₂;
each R²¹ is independently selected from the group consisting of hydrogen; C₁₋₂₀alkyl, C₃₋₈cycloalkyl, and C₆₋₁₂arylC₁₋₆alkyl;
wherein said C₁₋₂₀alkyl, C₃₋₈cycloalkyl, C₆₋₁₂arylC₁₋₆alkyl, can be unsubstituted or substituted with one or more OH, SH, COOH, or NH₂;
each Z^{1a} is selected from the group consisting of C₁₋₆alkyl; haloC₁₋₆alkyl; haloC₁₋₆alkyloxy; halogen;
or Z^{1a} is a group of formula (vii); 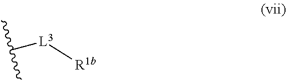
wherein the wavy line ( [symbol] ) indicates the point of attachment to L¹;
L³ is a single bond or is selected from the group consisting of C₁₋₁₀alkylene; C₂₋₁₀alkenylene; C₂₋₁₀alkynylene; and C₆₋₁₂aryl;
R^{1b} is a group of formula (viii); 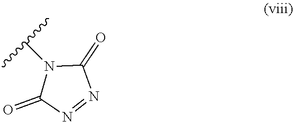 [SMILES: *N1C(=O)N=NC1=O]
wherein the wavy line ( [symbol] ) indicates the point of attachment to L³;
or R^{1b} is a group of formula (ix); 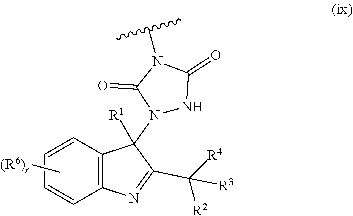
wherein the wavy line ( [symbol] ) indicates the point of attachment to L³;
Z¹ are each independently selected from the group consisting of OH; SH; COOH; NH₂; NHR²⁰; halogen; C₁₋₆alkyl; —OR¹⁹; —SR¹⁹; nitro; NR²⁰R²¹; —C(O)NR²⁰R²¹; cyano;
Z⁷, Z⁸, Z^{8a}, Z⁹ are each selected from the group consisting of OH; hydrogen; SH; COOH; NH₂; NHR²⁰; halogen; C₁₋₆alkyl; —OR¹⁹; —SR¹⁹; nitro; NR²⁰R²¹; —C(O)NR²⁰R²¹; cyano; and a polymeric group.

5. The compound according to claim 1, having structural formula (IB) 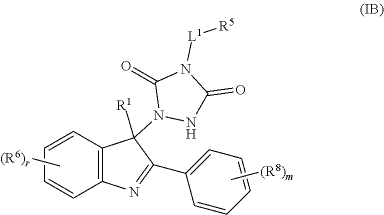
wherein m is an integer selected from 0, 1, 2, 3 or 4.

6. The compound according to claim 1, wherein Z⁸ and Z^{8a} are independently selected from a polymeric group; wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group.

7. A process for the preparation of a compound according to claim 1, comprising the step of contacting at least one compound of formula (II) with at least one compound of formula (III), thereby obtaining a compound of formula (I), 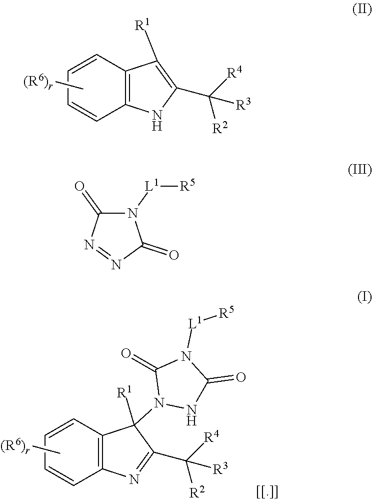
wherein r is an integer selected from 0, 1, 2, 3 or 4;
R¹ is selected from the group consisting of C₁₋₁₀alkyl; C₂₋₁₀alkenyl; C₂₋₁₀alkynyl; C₆₋₁₂aryl; C₃₋₈cycloalkyl; C₆₋₁₂arylC₁₋₆alkyl; heterocyclyl; heteroaryl; heterocyclylC₁₋₆alkyl; heteroarylC₁₋₆alkyl; —OR¹⁹; —SR¹⁹; NR²⁰R²¹; —C(O)NR²⁰R²¹; OH, SH, COOH, NH₂; NHR²⁰; and a polymeric group;
and wherein said C₁₋₁₀alkyl; C₂₋₁₀alkenyl; C₂₋₁₀alkynyl, C₆₋₁₂aryl, C₃₋₈cycloalkyl; C₆₋₁₂arylC₁₋₆alkyl, heterocyclyl; hetero aryl; heterocyclylC₁₋₆alkyl; or heteroarylC₁₋₆alkyl; can be unsubstituted or substituted with one or more Z¹;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
or R¹ is a group of formula (i); 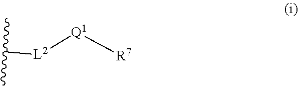
wherein the wavy line ( [symbol] ) indicates the point of attachment to the indole ring of formula (I);
L² is selected from the group consisting of C₁₋₁₀alkylene; C₃₋₆cycloalkylene; C₂₋₁₀alkenylene; C₂₋₁₀alkynylene; C₆₋₁₂arylene; and C₆₋₁₂arylC₁₋₆alkylene;
and wherein said C₁₋₁₀alkylene; C₃₋₆cycloalkylene; C₂₋₁₀alkenylene; C₂₋₁₀alkynylene; C₆₋₁₂arylene or C₆₋₁₂arylC₁₋₆alkylene, can be unsubstituted or substituted with one or more Z²;
Q¹ is a single bond or selected from the group consisting of C₁₋₁₀alkylene; C₃₋₆cycloalkylene; —O—; —S—; —O—C₁₋₁₀alkylene; and —S—C₁₋₁₀alkylene; wherein each left side of said groups is attached to L² and the right side thereof is attached to R⁷;
and wherein said C₁₋₁₀alkylene and said C₃₋₆cycloalkylene; can be unsubstituted or substituted with one or more Z³;
R⁷ is selected from the group consisting of hydrogen; OH, SH, COOH, NH₂; NHR²⁰, halo; nitro; cyano; —C(O)NR²⁰N²¹; C₁₋₂₀alkyl, C₂₋₂₀alkenyl, C₂₋₂₀alkynyl, C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl; can be unsubstituted or substituted with one or more Z⁴;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
or R⁷ is a group of formula (ii); 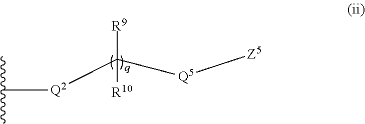
wherein the wavy line ( [symbol] ) indicates the point of attachment to Q¹;
q is an integer selected from 0; 1, 2, 3 or 4;
Q² is a single bond or selected from the group consisting of CR¹¹R¹²; O, S, and NR¹³;
Q³ is a single bond or selected from the group consisting of hydroxyC₁₋₁₀alkylene; aminoC₁₋₁₀alkylene; mercaptoC₁₋₁₀alkylene; C₁₋₂₀alkylene; C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₆alkylene; and heteroarylC₁₋₆alkylene;
wherein said C₁₋₂₀alkylene, C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₆alkylene; or heteroarylC₁₋₆alkylene, can be unsubstituted or substituted with one or more Z^{5a};
wherein if q is 0, then one of Q² and Q³ is not a single bond;
each R⁹ and R¹⁰ is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
each R¹¹ and R¹² is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
R¹³ is selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; and heteroarylC₁₋₆alkyl;
R² is selected from the group consisting of halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; heteroarylC₁₋₆alkyl; and a polymeric group;
R³ is selected from the group consisting of halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; heteroarylC₁₋₆alkyl; and a polymeric group;
R⁴ is selected from the group consisting of halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; heteroarylC₁₋₆alkyl; and a polymeric group;
wherein for R², R³ and R⁴, each independently; said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl, heteroarylC₁₋₆alkyl, can be unsubstituted or substituted with one or more Z⁶;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
or R² and R³ and R⁴ together with the carbon atom to which they are attached can form a saturated or unsaturated 4-, 5-, 6-, 7- or 8-membered ring;
wherein said saturated or unsaturated 4-, 5-, 6-, 7- or 8-membered ring optionally comprises one or more heteroatoms, said heteroatoms being each independently selected from O, S and N;
wherein said saturated or unsaturated 4-, 5-, 6-, 7- or 8-membered ring can be unsubstituted or substituted with one or more R⁸;
each R⁸ is independently selected from the group consisting of hydroxyC₁₋₁₀alkoxy; aminoC₁₋₁₀alkoxy; mercaptoC₁₋₁₀alkoxy; C₁₋₁₀alkyl; C₂₋₁₀alkenyl; C₂₋₁₀alkynyl; C₆₋₁₂arylC₁₋₆alkyl; C₃₋₈cycloalkyl; C₆₋₁₂arylC₁₋₆alkyl; heterocyclyl; heteroaryl; heterocyclylC₁₋₆alkyl; heteroarylC₁₋₆alkyl; OH; SH; COOH; NH₂; NHR²⁰; halogen; haloC₁₋₆alkyl; haloC₁₋₆alkyloxy; —OR¹⁹; —SR¹⁹; nitro; NR²⁰R²¹; —C(O)NR²⁰R²¹; cyano; and a polymeric group;
wherein said hydroxyC₁₋₁₀alkoxy; aminoC₁₋₁₀alkoxy; mercaptoC₁₋₁₀alkoxy; C₁₋₁₀alkyl; C₂₋₁₀alkenyl; C₂₋₁₀alkynyl; C₆₋₁₂arylC₁₋₆alkyl; C₃₋₈cycloalkyl; C₆₋₁₂arylC₁₋₆alkyl; heterocyclyl; heteroaryl; heterocyclylC₁₋₆alkyl; or heteroarylC₁₋₆alkyl, can be unsubstituted or substituted with one or more Z⁷;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
or R⁸ is a group of formula (iii); 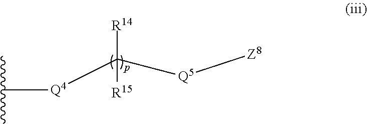
wherein the wavy line ( [symbol] ) indicates the point of attachment to the ring formed by R² and R³ and R⁴ together;
p is an integer selected from 0; 1, 2, 3 or 4;
Q⁴ is a single bond or selected from the group consisting of CR¹⁶R¹⁷; O, S, and NR¹⁸;
Q⁵ is a single bond or selected from the group consisting of hydroxyC₁₋₁₀alkylene; aminoC₁₋₁₀alkylene; mercaptoC₁₋₁₀alkylene; C₁₋₂₀alkylene, C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₆alkylene; and heteroarylC₁₋₆alkylene;
wherein said C₁₋₂₀alkylene, C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₆alkylene; or heteroarylC₁₋₆alkylene, can be unsubstituted or substituted with one or more Z^{8a};
wherein if p is 0, then one of Q⁴ and Q⁵ is not a single bond;
each R¹⁴ and R¹⁵ is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
each R¹⁶ and R¹⁷ is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
R¹⁸ is selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; and heteroarylC₁₋₆alkyl;
each R⁶ is independently selected from the group consisting of hydrogen, halogen; C₁₋₁₀alkyl; C₂₋₁₀alkenyl; C₂₋₁₀alkynyl; C₆₋₁₂arylC₁₋₆alkyl; haloC₁₋₆alkyl; haloC₁₋₆alkyloxy; —OR¹⁹; —SR¹⁹; nitro; NR²⁰R²¹; C(O)NR²⁰R²¹; cyano; and a polymeric group;
wherein said C₁₋₁₀alkyl; C₂₋₁₀alkenyl; C₂₋₁₀alkynyl; or C₆₋₁₂arylC₁₋₆alkyl, can be unsubstituted or substituted with one or more Z⁹;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
L¹ is selected from the group consisting of C₁₋₁₀alkyleneC₃₋₈cycloalkylene; C₃₋₈cycloalkyleneC₁₋₁₀alkylene; C₁₋₁₀alkylene; C₃₋₈cycloalkylene; C₃₋₈cycloalkyleneC₁₋₁₀alkylC₃₋₈cycloalkylene; C₂₋₁₀alkenylene; C₂₋₁₀alkynylene; C₆₋₁₂arylene; C₆₋₁₂aryleneC₁₋₆alkylene; C₆₋₁₂aryleneC₁₋₆alkylC₆₋₁₂arylene; and C₁₋₆alkyleneC₆₋₁₂arylC₁₋₆alkylene;
wherein said C₁₋₁₀alkyleneC₃₋₈cycloalkylene; C₃₋₈cycloalkyleneC₁₋₁₀alkylene; C₁₋₁₀alkylene; C₃₋₈cycloalkylene; C₃₋₈cycloalkyleneC₁₋₁₀alkylC₃₋₈cycloalkylene; C₂₋₁₀alkenylene; C₂₋₁₀alkynylene; C₆₋₁₂ arylene; C₆₋₁₂aryleneC₁₋₆alkylene; C₆₋₁₂aryleneC₁₋₆alkylC₆₋₁₂arylene; or C₁₋₆alkyleneC₆₋₁₂arylC₁₋₆alkylene, can be unsubstituted or substituted with one or more Z^{1a};
or L¹ is selected from a group of formula (iv); 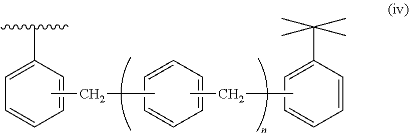
wherein the wavy line ( [symbol] ) indicates the point of attachment to the nitrogen of the formula (I) and the crossed line ( [symbol] ) indicates the point of attachment to R⁵;
wherein n is an integer selected from 0, 1, 2, 3, 4, 5, 6, 7, 8, 9, or 10;
R⁵ is selected from the group consisting of OH, SH, COOH, NH₂, and NHR²⁰;
or R⁵ is a group of formula (v); 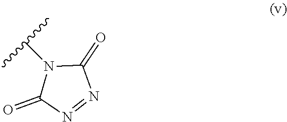 [SMILES: *N1C(=O)N=NC1=O]
wherein the wavy line ( [symbol] ) indicates the point of attachment to L¹;
or R⁵ is a group of formula (vi); 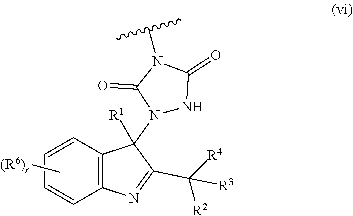
wherein the wavy line ( [symbol] ) indicates the point of attachment to L¹;
each R¹⁹ is independently selected from the group consisting of C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl;
wherein said C₁₋₁₀alkyl, C₂₋₂₀alkenyl, C₂₋₂₀alkynyl, C₆₋₁₂arylC₁₋₆alkyl, heterocyclylC₁₋₆alkyl; heteroarylC₁₋₆alkyl, optionally comprise one or more heteroatoms in the alkyl, alkenyl or alkynyl moiety, said heteroatoms being each independently selected from O, S and N;
wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl, can be unsubstituted or substituted with one or more OH, SH, COOH, or NH₂;
each R²⁰ is independently selected from the group consisting of C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl;
wherein said C₁₋₁₀alkyl, C₂₋₂₀alkenyl, C₂₋₂₀alkynyl, C₆₋₁₂arylC₁₋₆alkyl, heterocyclylC₁₋₆alkyl; heteroarylC₁₋₆alkyl, optionally comprise one or more heteroatoms in the alkyl, alkenyl or alkynyl moiety, said heteroatoms being each independently selected from O, S and N;
wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl, can be unsubstituted or substituted with one or more OH, SH, COOH, or NH₂;
each R²¹ is independently selected from the group consisting of hydrogen, C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl;
wherein said C₁₋₁₀alkyl, C₂₋₂₀alkenyl, C₂₋₂₀alkynyl, C₆₋₁₂arylC₁₋₆alkyl, heterocyclylC₁₋₆alkyl; heteroarylC₁₋₆alkyl, optionally comprise one or more heteroatoms in the alkyl, alkenyl or alkynyl moiety, said heteroatoms being each independently selected from O, S and N;
wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl, can be unsubstituted or substituted with one or more OH, SH, COOH, or NH₂;
or wherein R²⁰ and R²¹ together with the atom to which they are attached form a 5-, 6-, or 7-membered heterocycle.
each Z^{1a} is selected from the group consisting of C₁₋₆alkyl; C₂₋₆alkenyl; C₂₋₆alkynyl; haloC₁₋₆alkyl; haloC₁₋₆alkyloxy; halogen; isocyanate;
or Z^{1a} is a group of formula (vii); 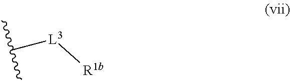
wherein the wavy line ( [symbol] ) indicates the point of attachment to L¹;
L³ is a single bond or is selected from the group consisting of C₁₋₁₀alkylene; C₂₋₁₀alkenylene; C₂₋₁₀alkynylene; and C₆₋₁₂aryl;
R^{1b} is a group of formula (viii); 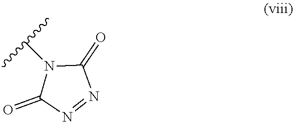 [SMILES: *N1C(=O)N=NC1=O]
wherein the wavy line ( [symbol] ) indicates the point of attachment to L³;
or R^{1b} is a group of formula (ix); 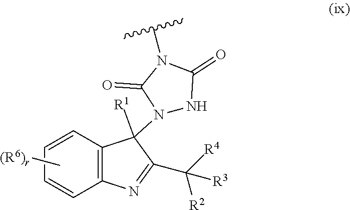
wherein the wavy line ( [symbol] ) indicates the point of attachment to L³;
Z¹, Z², Z³, Z⁴, Z⁶ are each independently selected from the group consisting of OH; SH; COOH; NH₂; NHR²⁰; halogen; C₁₋₆alkyl; haloC₁₋₆alkyl; haloC₁₋₆alkyloxy; —OR¹⁹; —SR¹⁹; nitro; NR²⁰R²¹; —C(O)NR²⁰R²¹; cyano;
Z⁵, Z^{5a}; Z⁷, Z⁸; Z^{8a}; Z⁹ are each independently selected from the group consisting of OH; hydrogen; SH; COOH; NH₂; NHR²⁰; halogen; C₁₋₆alkyl; haloC₁₋₆alkyl; haloC₁₋₆alkyloxy; —OR¹⁹; —SR¹⁹; nitro; NR²⁰R²¹; C(O)NR²⁰R²¹; cyano; and a polymeric group;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
and when none of R¹, R², R³, R⁴, R⁶, R⁷, R⁸, R⁹, R¹⁹, R¹¹, R¹², R¹⁴, R¹⁵, R¹⁶, R¹⁷, Z⁵, Z^{5a}, Z⁷, Z⁸, Z^{8a} or Z⁹ is a polymeric group, then at least one of R⁷, R⁸, Z¹, Z², Z³, Z⁴, Z⁵, Z^{5a}, Z⁶, Z⁷, Z⁸, Z^{8a} or Z⁹ is selected from the group consisting of OH, SH, COOH, NH₂, and NHR²⁰.

8. A process according to claim 7, wherein the compound of formula (III) has structural formula (IIIA)

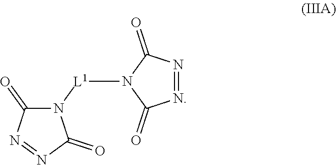

9. A compound of formula (A) or a stereoisomer, enantiomer, racemic, or tautomer thereof, 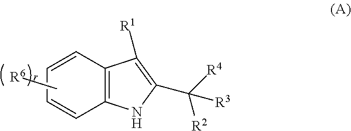
wherein,
r is an integer selected from 0, 1, 2, 3 or 4;
R¹ is selected from the group consisting of C₁₋₁₀alkyl; C₂₋₁₀alkenyl; C₂₋₁₀alkynyl; C₆₋₁₂aryl; C₃₋₈cycloalkyl; C₆₋₁₂arylC₁₋₆alkyl; heterocyclyl; heteroaryl; heterocyclylC₁₋₆alkyl; heteroarylC₁₋₆alkyl; —OR¹⁹; —SR¹⁹; NR²⁰R²¹; —C(O)NR²⁰R²¹; OH, SH, COOH, NH₂; NHR²⁰; and a polymeric group;
and wherein said C₁₋₁₀alkyl; C₂₋₁₀alkenyl; C₂₋₁₀alkynyl, C₆₋₁₂aryl, C₃₋₈cycloalkyl; C₆₋₁₂arylC₁₋₆alkyl, heterocyclyl; heteroaryl; heterocyclylC₁₋₆alkyl; or heteroarylC₁₋₆alkyl; can be unsubstituted or substituted with one or more Z¹;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
or R¹ is a group of formula (i); 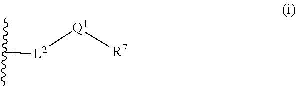
wherein the wavy line ( [symbol] ) indicates the point of attachment to the indole ring of formula (I);
L² is selected from the group consisting of C₁₋₁₀alkylene; C₃₋₆cycloalkylene; C₂₋₁₀alkenylene; C₂₋₁₀alkynylene; C₆₋₁₂arylene; and C₆₋₁₂arylC₁₋₆alkylene;
and wherein said C₁₋₁₀alkylene; C₃₋₆cycloalkylene; C₂₋₁₀alkenylene; C₂₋₁₀alkynylene; C₆₋₁₂arylene or C₆₋₁₂arylC₁₋₆alkylene, can be unsubstituted or substituted with one or more Z²;
Q¹ is a single bond or selected from the group consisting of C₁₋₁₀alkylene; C₃₋₆cycloalkylene; —O—; —S—; —O—C₁₋₁₀alkylene; and —S—C₁₋₁₀alkylene; wherein each left side of said groups is attached to L² and the right side thereof is attached to R⁷;
and wherein said C₁₋₁₀alkylene and said C₃₋₆cycloalkylene; can be unsubstituted or substituted with one or more Z³;
R⁷ is selected from the group consisting of hydrogen; OH, SH, COOH, NH₂; NHR²⁰, halo; nitro; cyano; —C(O)NR²⁰R²¹; C₁₋₂₀alkyl, C₂₋₂₀alkenyl; C₂₋₂₀alkynyl; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl; can be unsubstituted or substituted with one or more Z⁴;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
or R⁷ is a group of formula (ii); 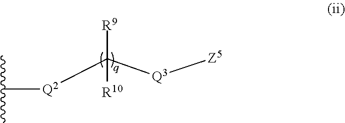
wherein the wavy line ( [symbol] ) indicates the point of attachment to Q¹;
q is an integer selected from 0; 1, 2, 3 or 4;
Q² is a single bond or selected from the group consisting of CR¹¹R¹²; O, S, and NR¹³;
Q³ is a single bond or selected from the group consisting of hydroxyC₁₋₁₀alkylene; aminoC₁₋₁₀alkylene; mercaptoC₁₋₁₀alkylene; C₁₋₂₀alkylene; C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₆alkylene; and heteroarylC₁₋₆alkylene;
wherein said C₁₋₂₀alkylene, C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₆alkylene; or heteroarylC₁₋₆alkylene, can be unsubstituted or substituted with one or more Z^{5a};
wherein if q is 0, then one of Q² and Q³ is not a single bond;
each R⁹ and R¹⁰ is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
each R¹¹ and R¹² is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
R¹³ is selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; and heteroarylC₁₋₆alkyl;
R² is selected from the group consisting of halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; heteroarylC₁₋₆alkyl; and a polymeric group;
R³ is selected from the group consisting of halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; heteroarylC₁₋₆alkyl; and a polymeric group;
R⁴ is selected from the group consisting of halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; heteroarylC₁₋₆alkyl; and a polymeric group;
wherein for R², R³ and R⁴, each independently; said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl, heteroarylC₁₋₆alkyl, can be unsubstituted or substituted with one or more Z⁶;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
or R² and R³ and R⁴ together with the carbon atom to which they are attached can form a saturated or unsaturated 4-, 5-, 6-, 7- or 8-membered ring;
wherein said saturated or unsaturated 4-, 5-, 6-, 7- or 8-membered ring optionally comprises one or more heteroatoms, said heteroatoms being each independently selected from O, S and N;
wherein said saturated or unsaturated 4-, 5-, 6-, 7- or 8-membered ring can be unsubstituted or substituted with one or more R⁸;
each R⁸ is independently selected from the group consisting of hydroxyC₁₋₁₀alkoxy; aminoC₁₋₁₀alkoxy; mercaptoC₁₋₁₀alkoxy; C₁₋₁₀alkyl; C₂₋₁₀alkenyl; C₂₋₁₀alkynyl; C₆₋₁₂arylC₁₋₆alkyl; C₃₋₈cycloalkyl; C₆₋₁₂arylC₁₋₆alkyl; heterocyclyl; heteroaryl; heterocyclylC₁₋₆alkyl; heteroarylC₁₋₆alkyl; OH; SH; COOH; NH₂; NHR²⁰; halogen; haloC₁₋₆alkyl; haloC₁₋₆alkyloxy; —OR¹⁹; —SR¹⁹; nitro; NR²⁰R²¹; —C(O)NR²⁰R²¹; cyano; and a polymeric group;
wherein said C₁₋₁₀alkyl; C₂₋₁₀alkenyl; C₂₋₁₀alkynyl; C₆₋₁₂arylC₁₋₆alkyl; C₃₋₈cycloalkyl; C₆-12arylC₁₋₆alkyl; heterocyclyl; heteroaryl; heterocyclylC₁₋₆alkyl; or heteroarylC₁₋₆alkyl, can be unsubstituted or substituted with one or more Z⁷;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
or R⁸ is a group of formula (iii); 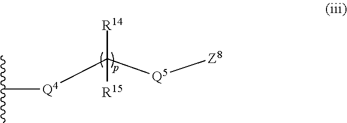
wherein the wavy line ( [symbol] ) indicates the point of attachment to the ring formed by R² and R³ and R⁴ together;
p is an integer selected from 0; 1, 2, 3 or 4;
Q⁴ is a single bond or selected from the group consisting of CR¹⁶R¹⁷; O, S, and NR¹⁸;
Q⁵ is a single bond or selected from the group consisting of hydroxyC₁₋₁₀alkylene; aminoC₁₋₁₀alkylene; mercaptoC₁₋₁₀alkylene; C₁₋₂₀alkylene, C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₆alkylene; and heteroarylC₁₋₆alkylene;
wherein said C₁₋₂₀alkylene, C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₆alkylene; or heteroarylC₁₋₆alkylene, can be unsubstituted or substituted with one or more Z^{8a};
wherein if p is 0, then one of Q⁴ and Q⁵ is not a single bond;
each R¹⁴ and R¹⁵ is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
each R¹⁶ and R¹⁷ is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
R¹⁸ is selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; and heteroarylC₁₋₆alkyl;
each R⁶ is independently selected from the group consisting of hydrogen, halogen; C₁₋₁₀alkyl; C₂₋₁₀alkenyl; C₂₋₁₀alkynyl; C₆₋₁₂arylC₁₋₆alkyl; haloC₁₋₆alkyl; haloC₁₋₆alkyloxy; —OR¹⁹; —SR¹⁹; nitro; NR²⁰R²¹; —C(O)NR²⁰R²¹; cyano; and a polymeric group;
wherein said C₁₋₁₀alkyl; C₂₋₁₀alkenyl; C₂₋₁₀alkynyl; or C₆₋₁₂arylC₁₋₆alkyl, can be unsubstituted or substituted with one or more Z⁹;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
each R¹⁹ is independently selected from the group consisting of C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl;
wherein said C₁₋₁₀alkyl, C₂₋₂₀alkenyl, C₂₋₂₀alkynyl, C₆₋₁₂arylC₁₋₆alkyl, heterocyclylC₁₋₆alkyl; heteroarylC₁₋₆alkyl, optionally comprise one or more heteroatoms in the alkyl, alkenyl or alkynyl moiety, said heteroatoms being each independently selected from O, S and N;
wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl, can be unsubstituted or substituted with one or more OH, SH, COOH, or NH₂;
each R²⁰ is independently selected from the group consisting of C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl;
wherein said C₁₋₁₀alkyl, C₂₋₂₀alkenyl, C₂₋₂₀alkynyl, C₆₋₁₂arylC₁₋₆alkyl, heterocyclylC₁₋₆alkyl; heteroarylC₁₋₆alkyl, optionally comprise one or more heteroatoms in the alkyl, alkenyl or alkynyl moiety, said heteroatoms being each independently selected from O, S and N;
wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl, can be unsubstituted or substituted with one or more OH, SH, COOH, or NH₂;
each R²¹ is independently selected from the group consisting of hydrogen; C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl;
wherein said C₁₋₁₀alkyl, C₂₋₂₀alkenyl, C₂₋₂₀alkynyl, C₆₋₁₂arylC₁₋₆alkyl, heterocyclylC₁₋₆alkyl; heteroarylC₁₋₆alkyl, optionally comprise one or more heteroatoms in the alkyl, alkenyl or alkynyl moiety, said heteroatoms being each independently selected from O, S and Nor wherein R²⁰ and R²¹ together with the atom to which they are attached form a 5-, 6-, or 7-membered heterocycle;
wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl, can be unsubstituted or substituted with one or more OH, SH, COOH, or NH₂;
Z¹, Z², Z³, Z⁴, Z⁶ are each independently selected from the group consisting of OH; SH; COOH; NH₂; NHR²⁰; halogen; C₁₋₆alkyl; haloC₁₋₆alkyl; haloC₁₋₆alkyloxy; —OR¹⁹; —SR¹⁹; nitro; NR²⁰R²¹; —C(O)NR²⁰R²¹; cyano;
Z⁵, Z^{5a}; Z⁷, Z⁸; Z^{8a}; Z⁹ are independently selected from the group consisting of OH; hydrogen; SH; COOH; NH₂; NHR²⁰; halogen; C₁₋₆alkyl; haloC₁₋₆alkyl; haloC₁₋₆alkyloxy; —OR¹⁹; —SR¹⁹; nitro; NR²⁰R²¹; —C(O)NR²⁰R²¹; cyano; and a polymeric group;
and wherein said polymeric group selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; ethylene propylene rubber (EPM); polybutadiene; polysiloxanes; and/or hydrogenated derivatives thereof; and/or block, comb and/or star copolymers of the polymeric group;
and when none of R¹, R², R³, R⁴, R⁶, R⁷, R⁸, R⁹, R¹⁹, R¹¹, R¹², R¹⁴, R¹⁵, R¹⁶, R¹⁷, Z⁵, Z^{5a}, Z⁷, Z⁸, Z^{8a} or Z⁹ is a polymeric group, then at least one of R⁷, R⁸, Z¹, Z², Z³, Z⁴, Z⁵, Z^{5a}, Z⁶, Z⁷, Z⁸, Z^{8a} or Z⁹ is selected from the group consisting of OH, SH, COOH, NH₂, and NHR²⁰.

10. The compound according to claim 9, having structural formula (A2) 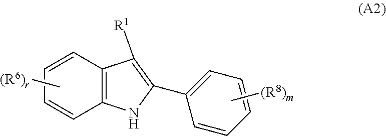
wherein m is an integer selected from 0, 1, 2, 3 or 4;
R¹ is selected from the group consisting of C₁₋₁₀alkyl; C₂₋₁₀alkenyl; C₂₋₁₀alkynyl; C₆₋₁₂aryl; C₃₋₈cycloalkyl; C₆₋₁₂arylC₁₋₆alkyl; heterocyclyl; heteroaryl; heterocyclylC₁₋₆alkyl; heteroarylC₁₋₆alkyl; —OR¹⁹; —SR¹⁹; NR²⁰R²¹; —C(O)NR²⁰R²¹; OH, SH, COOH, NH₂; NHR²⁰; and a polymeric group;
and wherein said C₁₋₁₀alkyl; C₂₋₁₀alkenyl; C₂₋₁₀alkynyl, C₆₋₁₂aryl, C₃₋₈cycloalkyl; C₆₋₁₂arylC₁₋₆alkyl, heterocyclyl; heteroaryl; heterocyclylC₁₋₆alkyl; or heteroarylC₁₋₆alkyl; can be unsubstituted or substituted with one or more Z¹;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
or R¹ is a group of formula (i); 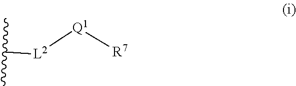
wherein the wavy line ( [symbol] ) indicates the point of attachment to the indole ring of formula (I);
L² is selected from the group consisting of C₁₋₁₀alkylene; C₃₋₆cycloalkylene; C₂₋₁₀alkenylene; C₂₋₁₀alkynylene; C₆₋₁₂arylene; and C₆₋₁₂arylC₁₋₆alkylene;
and wherein said C₁₋₁₀alkylene; C₃₋₆cycloalkylene; C₂₋₁₀alkenylene; C₂₋₁₀alkynylene; C₆₋₁₂arylene or C₆₋₁₂arylC₁₋₆alkylene, can be unsubstituted or substituted with one or more Z²;
Q¹ is a single bond or selected from the group consisting of C₁₋₁₀alkylene; C₃₋₆cycloalkylene; —O—; —S—; —O—C₁₋₁₀alkylene; and —S—C₁₋₁₀alkylene; wherein each left side of said groups is attached to L² and the right side thereof is attached to R⁷;
and wherein said C₁₋₁₀alkylene and said C₃₋₆cycloalkylene; can be unsubstituted or substituted with one or more Z³;
R⁷ is selected from the group consisting of hydrogen; OH, SH, COOH, NH₂; NHR²⁰, halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl; can be unsubstituted or substituted with one or more Z⁴;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
or R⁷ is a group of formula (ii); 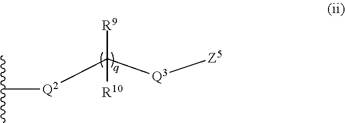
wherein the wavy line ( [symbol] ) indicates the point of attachment to Q¹;
q is an integer selected from 0; 1, 2, 3 or 4;
Q² is a single bond or selected from the group consisting of CR¹¹R¹²; O, S, and NR¹³;
Q³ is a single bond or selected from the group consisting of hydroxyC₁₋₁₀alkylene; aminoC₁₋₁₀alkylene; mercaptoC₁₋₁₀alkylene; C₁₋₂₀alkylene; C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₆alkylene; and heteroarylC₁₋₆alkylene;
wherein said C₁₋₂₀alkylene, C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₆alkylene; or heteroarylC₁₋₆alkylene, can be unsubstituted or substituted with one or more Z^{5a};
wherein if q is 0, then one of Q² and Q³ is not a single bond;
each R⁹ and R¹⁰ is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
each R¹¹ and R¹² is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
R¹³ is selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; and heteroarylC₁₋₆alkyl;
each R⁸ is independently selected from the group consisting of hydroxyC₁₋₁₀alkoxy; aminoC₁₋₁₀alkoxy; mercaptoC₁₋₁₀alkoxy; C₁₋₁₀alkyl; C₂₋₁₀alkenyl; C₂₋₁₀alkynyl; C₆₋₁₂arylC₁₋₆alkyl; C₃₋₈cycloalkyl; C₆₋₁₂arylC₁₋₆alkyl; heterocyclyl; heteroaryl; heterocyclylC₁₋₆alkyl; heteroarylC₁₋₆alkyl; OH; SH; COOH; NH₂; NHR²⁰; halogen; haloC₁₋₆alkyl; haloC₁₋₆alkyloxy; —OR¹⁹; —SR¹⁹; nitro; NR²⁰R²¹; —C(O)NR²⁰R²¹; cyano; and a polymeric group;
wherein said C₁₋₁₀alkyl; C₂₋₁₀alkenyl; C₂₋₁₀alkynyl; C₆₋₁₂arylC₁₋₆alkyl; C₃₋₈cycloalkyl; C₆₋₁₂arylC₁₋₆alkyl; heterocyclyl; heteroaryl; heterocyclylC₁₋₆alkyl; or heteroarylC₁₋₆alkyl, can be unsubstituted or substituted with one or more Z⁷;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
or R⁸ is a group of formula (iii); 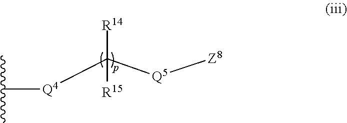
wherein the wavy line ( [symbol] ) indicates the point of attachment to the ring formed by R² and R³ and R⁴ together;
p is an integer selected from 0; 1, 2, 3 or 4;
Q⁴ is a single bond or selected from the group consisting of CR¹⁶R¹⁷; O, S, and NR¹⁸;
Q⁵ is a single bond or selected from the group consisting of hydroxyC₁₋₁₀alkylene; aminoC₁₋₁₀alkylene; mercaptoC₁₋₁₀alkylene; C₁₋₂₀alkylene, C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₆alkylene; and heteroarylC₁₋₆alkylene;
wherein said C₁₋₂₀alkylene, C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₆alkylene; or heteroarylC₁₋₆alkylene, can be unsubstituted or substituted with one or more Z^{8a};
wherein if p is 0, then one of Q⁴ and Q⁵ is not a single bond;
each R¹⁴ and R¹⁵ is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
each R¹⁶ and R¹⁷ is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
R¹⁸ is selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; and heteroarylC₁₋₆alkyl;
each R⁶ is independently selected from the group consisting of hydrogen, halogen; C₁₋₁₀alkyl; C₂₋₁₀alkenyl; C₂₋₁₀alkynyl; C₆₋₁₂arylC₁₋₆alkyl; haloC₁₋₆alkyl; haloC₁₋₆alkyloxy; —OR¹⁹; —SR¹⁹; nitro; NR²⁰R²¹; —C(O)NR²⁰R²¹; cyano; and a polymeric group;
wherein said C₁₋₁₀alkyl; C₂₋₁₀alkenyl; C₂₋₁₀alkynyl; or C₆₋₁₂arylC₁₋₆alkyl, can be unsubstituted or substituted with one or more Z⁹;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
each R¹⁹ is independently selected from the group consisting of C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl;
wherein said C₁₋₁₀alkyl, C₂₋₂₀alkenyl, C₂₋₂₀alkynyl, C₆₋₁₂arylC₁₋₆alkyl, heterocyclylC₁₋₆alkyl; heteroarylC₁₋₆alkyl, optionally comprise one or more heteroatoms in the alkyl, alkenyl or alkynyl moiety, said heteroatoms being each independently selected from O, S and N;
wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl, can be unsubstituted or substituted with one or more OH, SH, COOH, or NH₂;
each R²⁰ is independently selected from the group consisting of C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl;
wherein said C₁₋₁₀alkyl, C₂₋₂₀alkenyl, C₂₋₂₀alkynyl, C₆₋₁₂arylC₁₋₆alkyl, heterocyclylC₁₋₆alkyl; heteroarylC₁₋₆alkyl, optionally comprise one or more heteroatoms in the alkyl, alkenyl or alkynyl moiety, said heteroatoms being each independently selected from O, S and N;
wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl, can be unsubstituted or substituted with one or more OH, SH, COOH, or NH₂;
each R²¹ is independently selected from the group consisting of hydrogen; C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl;
wherein said C₁₋₁₀alkyl, C₂₋₂₀alkenyl, C₂₋₂₀alkynyl, C₆₋₁₂arylC₁₋₆alkyl, heterocyclylC₁₋₆alkyl; heteroarylC₁₋₆alkyl, optionally comprise one or more heteroatoms in the alkyl, alkenyl or alkynyl moiety, said heteroatoms being each independently selected from O, S and Nor wherein R²⁰ and R²¹ together with the atom to which they are attached form a 5-, 6-, or 7-membered heterocycle;
wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl, can be unsubstituted or substituted with one or more OH, SH, COOH, or NH₂;
Z¹, Z², Z³, Z⁴ are each independently selected from the group consisting of OH; SH; COOH;
NH₂; NHR²⁰; halogen; C₁₋₆alkyl; haloC₁₋₆alkyl; haloC₁₋₆alkyloxy; —OR¹⁹; —SR¹⁹; nitro; NR²⁰R²¹; —C(O)NR²⁰R²¹; cyano;
Z⁵, Z^{5a}; Z⁷, Z⁸; Z^{8a}; Z⁹ are independently selected from the group consisting of OH; hydrogen; SH; COOH; NH₂; NHR²⁰; halogen; C₁₋₆alkyl; haloC₁₋₆alkyl; haloC₁₋₆alkyloxy; —OR¹⁹; —SR¹⁹; nitro; NR²⁰R²¹; —C(O)NR²⁰R²¹; cyano; and a polymeric group;
and wherein said polymeric group selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; ethylene propylene rubber (EPM); polybutadiene; polysiloxanes; and/or hydrogenated derivatives thereof; and/or block, comb and/or star copolymers of the polymeric group;
and when none of R¹, R², R³, R⁴, R⁶, R⁷, R⁸, R⁹, R¹⁹, R¹¹, R¹², R¹⁴, R¹⁵, R¹⁶, R¹⁷, Z⁵, Z^{5a}, Z⁷, Z⁸, Z^{8a} or Z⁹ is a polymeric group, then at least one of R⁷, R⁸, Z¹, Z², Z³, Z⁴, Z⁵, Z^{5a}, Z⁶, Z⁷, Z⁸, Z^{8a} or Z⁹ is selected from the group consisting of OH, SH, COOH, NH₂, and NHR²⁰.

11. A composition comprising at least one compound of formula (I) as defined in claim 1.

12. The composition according to claim 11, wherein the composition further comprises at least one compound according to formula (A), wherein formula (A) has the structure: 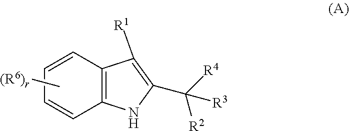
wherein
r is an integer selected from 0, 1, 2, 3 or 4;
R¹ is selected from the group consisting of C₁₋₁₀alkyl; C₂₋₁₀alkenyl; C₂₋₁₀alkynyl; C₆₋₁₂aryl; C₃₋₈cycloalkyl; C₆₋₁₂arylC₁₋₆alkyl; heterocyclyl; heteroaryl; heterocyclylC₁₋₆alkyl; heteroarylC₁₋₆alkyl; —OR¹⁹; —SR¹⁹; NR²⁰R²¹; —C(O)NR²⁰R²¹; OH, SH, COOH, NH₂; NHR²⁰; and a polymeric group;
and wherein said C₁₋₁₀alkyl; C₂₋₁₀alkenyl; C₂₋₁₀alkynyl, C₆₋₁₂aryl, C₃₋₈cycloalkyl; C₆₋₁₂arylC₁₋₆alkyl, heterocyclyl; heteroaryl; heterocyclylC₁₋₆alkyl; or heteroarylC₁₋₆alkyl; can be unsubstituted or substituted with one or more Z¹;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
or R¹ is a group of formula (i); 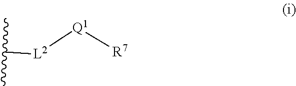
wherein the wavy line ( [symbol] ) indicates the point of attachment to the indole ring of formula (I);
L² is selected from the group consisting of C₁₋₁₀alkylene; C₃₋₆cycloalkylene; C₂₋₁₀alkenylene; C₂₋₁₀alkynylene; C₆₋₁₂arylene; and C₆₋₁₂arylC₁₋₆alkylene;
and wherein said C₁₋₁₀alkylene; C₃₋₆cycloalkylene; C₂₋₁₀alkenylene; C₂₋₁₀alkynylene; C₆₋₁₂arylene or C₆₋₁₂arylC₁₋₆alkylene, can be unsubstituted or substituted with one or more Z²;
Q¹ is a single bond or selected from the group consisting of C₁₋₁₀alkylene; C₃₋₆cycloalkylene; —O—; —S—; —O—C₁₋₁₀alkylene; and —S—C₁₋₁₀alkylene; wherein each left side of said groups is attached to L² and the right side thereof is attached to R⁷;
and wherein said C₁₋₁₀alkylene and said C₃₋₆cycloalkylene; can be unsubstituted or substituted with one or more Z³;
R⁷ is selected from the group consisting of hydrogen; OH, SH, COOH, NH₂; NHR²⁰, halo; nitro; cyano; —C(O)NR²⁰R²¹; C₁₋₂₀alkyl, C₂₋₂₀alkenyl; C₂₋₂₀alkynyl; C₆₋₁₂aryl; C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl; can be unsubstituted or substituted with one or more Z⁴;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
or R⁷ is a group of formula (ii); 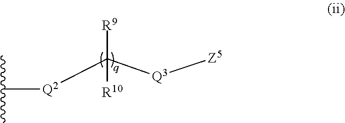
wherein the wavy line ( [symbol] ) indicates the point of attachment to Q¹;
q is an integer selected from 0; 1, 2, 3 or 4;
Q² is a single bond or selected from the group consisting of CR¹¹R¹²; O, S, and NR¹³;
Q³ is a single bond or selected from the group consisting of hydroxyC₁₋₁₀alkylene; aminoC₁₋₁₀alkylene; mercaptoC₁₋₁₀alkylene; C₁₋₂₀alkylene; C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₆alkylene; and heteroarylC₁₋₆alkylene;
wherein said C₁₋₂₀alkylene, C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₆alkylene; or heteroarylC₁₋₆alkylene, can be unsubstituted or substituted with one or more Z^{5a};
wherein if q is 0, then one of Q² and Q³ is not a single bond;
each R⁹ and R¹⁰ is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
each R¹¹ and R¹² is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
R¹³ is selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; and heteroarylC₁₋₆alkyl;
R² is selected from the group consisting of halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; heteroarylC₁₋₆alkyl; and a polymeric group;
R³ is selected from the group consisting of halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; heteroarylC₁₋₆alkyl; and a polymeric group;
R⁴ is selected from the group consisting of halo; nitro; cyano; —C(O)NR²⁰R²¹; C₁₋₂₀alkyl; C₂₋₂₀alkenyl; C₂₋₂₀alkynyl; C₆₋₁₂aryl; C₃₋₈cycloalkyl; C₆₋₁₂arylC₁₋₆alkyl; C₆₋₁₂arylC₁₋₆alkylC₆₋₁₂aryl; heterocyclyl; heteroaryl; heterocyclylC₁₋₆alkyl; heteroarylC₁₋₆alkyl; and a polymeric group;
wherein for R², R³ and R⁴, each independently; said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl, heteroarylC₁₋₆alkyl, can be unsubstituted or substituted with one or more Z⁶;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
or R² and R³ and R⁴ together with the carbon atom to which they are attached can form a saturated or unsaturated 4-, 5-, 6-, 7- or 8-membered ring;
wherein said saturated or unsaturated 4-, 5-, 6-, 7- or 8-membered ring optionally comprises one or more heteroatoms, said heteroatoms being each independently selected from O, S and N;
wherein said saturated or unsaturated 4-, 5-, 6-, 7- or 8-membered ring can be unsubstituted or substituted with one or more R⁸;
each R⁸ is independently selected from the group consisting of hydroxyC₁₋₁₀alkoxy; aminoC₁₋₁₀alkoxy; mercaptoC₁₋₁₀alkoxy; C₁₋₁₀alkyl; C₂₋₁₀alkenyl; C₂₋₁₀alkynyl; C₆₋₁₂arylC₁₋₆alkyl; C₃₋₈cycloalkyl; C₆₋₁₂arylC₁₋₆alkyl; heterocyclyl; heteroaryl; heterocyclylC₁₋₆alkyl; heteroarylC₁₋₆alkyl; OH; SH; COOH; NH₂; NHR²⁰; halogen; haloC₁₋₆alkyl; haloC₁₋₆alkyloxy; —OR¹⁹; —SR¹⁹; nitro; NR²⁰R²¹; —C(O)NR²⁰R²¹; cyano; and a polymeric group;
wherein said C₁₋₁₀alkyl; C₂₋₁₀alkenyl; C₂₋₁₀alkynyl; C₆₋₁₂arylC₁₋₆alkyl; C₃₋₈cycloalkyl; C₆₋₁₂arylC₁₋₆alkyl; heterocyclyl; heteroaryl; heterocyclylC₁₋₆alkyl; or heteroarylC₁₋₆alkyl, can be unsubstituted or substituted with one or more Z⁷;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
or R⁸ is a group of formula (iii); 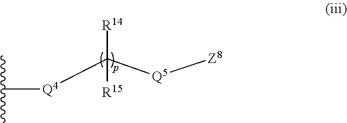
wherein the wavy line ( [symbol] ) indicates the point of attachment to the ring formed by R² and R³ and R⁴ together;
p is an integer selected from 0; 1, 2, 3 or 4;
Q⁴ is a single bond or selected from the group consisting of CR¹⁶R¹⁷; O, S, and NR¹⁸;
Q⁵ is a single bond or selected from the group consisting of hydroxyC₁₋₁₀alkylene; aminoC₁₋₁₀alkylene; mercaptoC₁₋₁₀alkylene; C₁₋₂₀alkylene, C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₆alkylene; and heteroarylC₁₋₆alkylene;
wherein said C₁₋₂₀alkylene, C₂₋₂₀alkenylene; C₂₋₂₀alkynylene; C₆₋₁₂arylene, C₃₋₈cycloalkylene; C₆₋₁₂arylC₁₋₆alkylene; heterocyclylene; heteroarylene; heterocyclylC₁₋₆alkylene; or heteroarylC₁₋₆alkylene, can be unsubstituted or substituted with one or more Z^{8a};
wherein if p is 0, then one of Q⁴ and Q⁵ is not a single bond;
each R¹⁴ and R¹⁵ is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
each R¹⁶ and R¹⁷ is independently selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; heteroarylC₁₋₆alkyl; and a polymeric group;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
R¹⁸ is selected from the group consisting of hydrogen; C₁₋₂₀alkyl, C₂₋₂₀alkenyl; C₂₋₂₀alkynyl; hydroxyC₁₋₁₀alkoxy; aminoC₁₋₁₀alkoxy; mercaptoC₁₋₁₀alkoxy; C₆₋₁₂aryl, C₃₋₈cycloalkyl; C₆₋₁₂arylC₁₋₆alkyl; heterocyclyl; heteroaryl; heterocyclylC₁₋₆alkyl; and heteroarylC₁₋₆alkyl;
each R⁶ is independently selected from the group consisting of hydrogen, halogen; C₁₋₁₀alkyl; C₂₋₁₀alkenyl; C₂₋₁₀alkynyl; C₆₋₁₂arylC₁₋₆alkyl; haloC₁₋₆alkyl; haloC₁₋₆alkyloxy; —OR¹⁹; —SR¹⁹; nitro; NR²⁰R²¹; —C(O)NR²⁰R²¹; cyano; and a polymeric group;
wherein said C₁₋₁₀alkyl; C₂₋₁₀alkenyl; C₂₋₁₀alkynyl; or C₆₋₁₂arylC₁₋₆alkyl, can be unsubstituted or substituted with one or more Z⁹;
and wherein said polymeric group is selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; poly-alpha-olefin; ethylene propylene diene monomer (M-class) rubber (EPDM) ethylene propylene rubber (EPM); polybutadienes; acrylonitrile butadiene styrene (ABS); styrene-butadiene rubber (SBR); polysiloxanes; and/or hydrogenated derivatives thereof; and/or block, comb and/or star copolymers of the polymeric group;
each R¹⁹ is independently selected from the group consisting of C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl;
wherein said C₁₋₁₀alkyl, C₂₋₂₀alkenyl, C₂₋₂₀alkynyl, C₆₋₁₂arylC₁₋₆alkyl, heterocyclylC₁₋₆alkyl; heteroarylC₁₋₆alkyl, optionally comprise one or more heteroatoms in the alkyl, alkenyl or alkynyl moiety, said heteroatoms being each independently selected from O, S and N;
wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl, can be unsubstituted or substituted with one or more OH, SH, COOH, or NH₂;
each R²⁰ is independently selected from the group consisting of C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl;
wherein said C₁₋₁₀alkyl, C₂₋₂₀alkenyl, C₂₋₂₀alkynyl, C₆₋₁₂arylC₁₋₆alkyl, heterocyclylC₁₋₆alkyl; heteroarylC₁₋₆alkyl, optionally comprise one or more heteroatoms in the alkyl, alkenyl or alkynyl moiety, said heteroatoms being each independently selected from O, S and N;
wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl, can be unsubstituted or substituted with one or more OH, SH, COOH, or NH₂;
each R²¹ is independently selected from the group consisting of hydrogen; C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl;
wherein said C₁₋₁₀alkyl, C₂₋₂₀alkenyl, C₂₋₂₀alkynyl, C₆₋₁₂arylC₁₋₆alkyl, heterocyclylC₁₋₆alkyl; heteroarylC₁₋₆alkyl, optionally comprise one or more heteroatoms in the alkyl, alkenyl or alkynyl moiety, said heteroatoms being each independently selected from O, S and Nor wherein R²⁰ and R²¹ together with the atom to which they are attached form a 5-, 6-, or 7-membered heterocycle;
wherein said C₁₋₂₀alkyl, C₂₋₂₀alkenyl, C₂₋₂₀alkynyl, C₆₋₁₂aryl, C₃₋₈cycloalkyl, C₆₋₁₂arylC₁₋₆alkyl, heterocyclyl; heteroaryl; heterocyclylC₁₋₆alkyl; and heteroarylC₁₋₆alkyl, can be unsubstituted or substituted with one or more OH, SH, COOH, or NH₂;
Z¹, Z², Z³, Z⁴, Z⁶ are each independently selected from the group consisting of OH; SH; COOH; NH₂; NHR²⁰; halogen; C₁₋₆alkyl; haloC₁₋₆alkyl; haloC₁₋₆alkyloxy; —OR¹⁹; —SR¹⁹; nitro; NR²⁰R²⁰; —C(O)NR²⁰R²¹; cyano;
Z⁵, Z^{5a}; Z⁷, Z⁸; Z^{8a}; Z⁹ are independently selected from the group consisting of OH; hydrogen; SH; COOH; NH₂; NHR²⁰; halogen; C₁₋₆alkyl; haloC₁₋₆alkyl; haloC₁₋₆alkyloxy; —OR¹⁹; —SR¹⁹; nitro; NR²⁰R²¹; —C(O)NR²⁰R²¹; cyano; and a polymeric group;
and wherein said polymeric group selected from the group consisting of polyurethane; isocyanate containing prepolymer, hydroxyl containing prepolymer, polyethylene oxide; polyacrylate; polymethacrylate; polystyrene; copolymer of acrylate; poly(methyl methacrylate); methacrylate and/or styrene; poly(isobornyl acrylate); polyacrylonitrile; polyether; polyester; polylactic acid; polyamide; polyester amide; polycarbonate; ethylene propylene rubber (EPM); polybutadiene; polysiloxanes; and/or hydrogenated derivatives thereof; and/or block, comb and/or star copolymers of the polymeric group;
and when none of R¹, R², R³, R⁴, R⁶, R⁷, R⁸, R⁹, R¹⁹, R¹¹, R¹², R¹⁴, R¹⁵, R¹⁶, R¹⁷, Z⁵, Z^{5a}, Z⁷, Z⁸, Z^{8a} or Z⁹ is a polymeric group, then at least one of R⁷, R⁸, Z¹, Z², Z³, Z⁴, Z⁵, Z^{5a}, Z⁶, Z⁷, Z⁸, Z^{8a} or Z⁹ is selected from the group consisting of OH, SH, COOH, NH₂, and NHR²⁰.

13. A method of obtaining a polyurethane or a polyurethane prepolymer comprising contacting at least one isocyanate with at least one compound of formula (I) as defined in claim 1, and thereby obtaining the polyurethane or the polyurethane prepolymer.

14. A method of preparing foams, adhesives, coatings, sealants, aerogels or elastomers comprising the use of a compound of formula (I) as defined in claim 1, and thereby obtaining the foams, the adhesives, the coatings, the sealants, the aerogels or the elastomers.

15. An article of manufacture comprising a compound of formula (I) according to claim 1.

16. A method of obtaining a polyurethane or a polyurethane prepolymer comprising contacting at least one isocyanate with at least one compound of formula (A) as defined in claim 9, and thereby obtaining the polyurethane or the polyurethane prepolymer.

17. A method of obtaining a polyurethane or a polyurethane prepolymer comprising contacting at least one isocyanate with a composition defined in claim 11, and thereby obtaining the polyurethane or the polyurethane prepolymer.

18. A method of preparing foams, adhesives, coatings, sealants, aerogels or elastomers comprising the use of a compound of formula (A) as defined in claim 9, and thereby obtaining the foams, the adhesives, the coatings, the sealants, the aerogels or the elastomers.

19. An article of manufacture comprising a compound of formula (A) according to claim 9.

20. An article or manufacture comprising a composition according to claim 11.
